(19) United States
(12) Patent Application Publication
(10) Pub. No.: US 2007/0254903 A1
(43) Pub. Date: Nov. 1, 2007

(54) Novel Spiroindoline or Spiroisoquinoline Compounds, Methods of Use and Compositions Thereof

(75) Inventors: P. Douglas Boatman, San Diego, CA (US); John W. Adams, San Diego, CA (US); Jeanne V. Moody, San Diego, CA (US); Eric D. Babych, Streamwood, IL (US); Thomas O. Schrader, San Diego, CA (US)

Correspondence Address: BOZICEVIC, FIELD & FRANCIS LLP;(ARENA PHARMACEUTICALS, INC.), 1900 UNIVERSITY AVENUE, SUITE 200, EAST PALO ALTO, CA, 94303, US

(73) Assignee: ARENA PHARMACEUTICALS, INC., SAN DIEGO, CA (US)

(21) Appl. No.: 10/583,839

(22) PCT Filed: Dec. 22, 2004
(86) PCT No.: PCT/US04/43609

§ 371 Date: Mar. 8, 2007

Related U.S. Application Data
(60) Provisional application No. 60/532,546, filed on Dec. 23, 2003.
(60) Provisional application No. 60/539,554, filed on Jan. 26, 2004.
(60) Provisional application No. 60/565,251, filed on Apr. 23, 2004.

(51) Int. Cl. A61K 31/438; A61P 25/00; A61P 9/00; C07D 401/04; C12N 5/08; G01N 33/566

(52) U.S. Cl. 514/278; 435/375; 436/501; 546/17; 546/18

(57) ABSTRACT

The invention provides compounds of Formula (I): 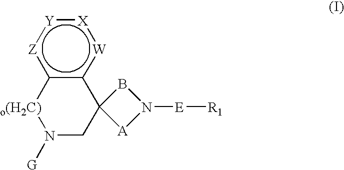
and pharmaceutically acceptable salts, solvates and stereoisomers thereof, wherein A, B, E, G, W, X, Y, Z, o, and R₁ are as disclosed herein (“Compound(s) of the Invention”), which are useful as cardio-protective and/or neuro-protective agents. The invention also provides pharmaceutical compositions comprising a Compound of the Invention and methods for treating, preventing and/or managing a vascular, cardiovascular or neurological disease or disorder, comprising administering to a patient in need thereof a Compound of the Invention.

### 1. FIELD OF THE INVENTION

The present invention relates to novel Spiroindoline and Spiroisoquinoline Compounds and pharmaceutically acceptable salts, free bases, solvates, hydrates, stereoisomers, clathrates or prodrugs thereof, which are useful, for example, as cardio-protective or neuro-protective agents in mammals. The invention encompasses compositions comprising a Spiroindoline or Spiroisoquinoline Compound and methods for treating or preventing a disease or disorder comprising the administration of a Spiroindoline or Spiroisoquinoline Compound to a patient in need thereof. Such a disease or disorder includes, for example, a vascular or cardiovascular disease or disorder such as atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic neuropathy, glomerulonephritis, scleroderma, glomerular sclerosis, renal failure, renal transplant therapy, diabetic retinopathy, migraine, and neurological diseases or disorders such as diabetic peripheral neuropathy, pain, stroke, cerebral ischemia and Parkinson's disease. The invention also relates to a modulator of the Mas G-protein coupled receptor including, for example, a Spiroindoline or Spiroisoquinoline Compound as disclosed herein.

### 2. BACKGROUND OF THE INVENTION

G protein-coupled receptors (GPCRs) share the common structural motif of having seven sequences of between 22 to 24 hydrophobic amino acids that form seven alpha helices, each of which spans the cell membrane. The transmembrane helices are joined by strands of amino acids having a larger loop between the fourth and fifth transmembrane helix on the extracellular side of the membrane. Another larger loop, composed primarily of hydrophilic amino acids, joins transmembrane helices five and six on the intracellular side of the membrane. The carboxy terminus of the receptor lies intracellularly with the amino terminus residing in the extracellular space. It is thought that the loop joining helices five and six, as well as the carboxy terminus, interact with the G protein. Currently, the G proteins that have been identified are Gq, Gs, Gi, and Go.

Under physiological conditions, GPCRs exist in the cell membrane in equilibrium between two different states or conformations: an “inactive” state and an “active” state. A receptor in an inactive state is unable to link to the intracellular transduction pathway to produce a biological response. Change of the receptor conformation to the active state allows linkage to the transduction pathway and produces a biological response. Physiologically, these conformational changes are induced in response to binding of a molecule to the receptor. Several types of biological molecules can bind to specific receptors, such as peptides, hormones or lipids, and can cause a cellular response. Modulation of particular cellular responses can be extremely useful for the treatment of disease states, and a number of chemical agents that act on GPCRs are useful for the treatment of disease.

The Mas protooncogene encodes a GPCR protein (Mas) and was first detected in vivo by its tumorogenic properties which originate from rearrangement of its 5′ flanking region (Young, D. et al., Cell 45:711-719 (1996)). Subsequent studies have indicated that the tumorogenic properties of Mas appear to be negligible. The lack of an identified activating ligand for the Mas receptor has made definition of its biological role difficult.

Originally, the angiotensin II (Ang II) peptide was thought to be a ligand for the Mas receptor (Jackson et al., Nature 335:437-440 (1988)). However, it was subsequently determined that intracellular calcium responses in Mas receptor-transfected cells only occurred in cells that already express an Ang II receptor (Ambroz et al. Biochem. Biophys. Acta 1133:107-111 (1991)). Other experiments demonstrated a possible role for Mas receptor in modulating intracellular signaling of an Ang II receptor after Ang II stimulation (von Bohlen und Halbech et al., J. Neurophysiol. 83:2012-2020 (2000)). In addition, Dong et al. reported that the Mas receptor did not bind to angiotensins I and II, but the Mas receptor did bind to a peptide called NPFF, although fairly weakly (EC₅₀ about 400 nM) (Dong et al., Cell 106:619-632 (2001)). A recent report that the biologically relevant angiotensin fragment Ang (1-7) (H-Asp-Arg-Val-Tyr-Ile-His-Pro-OH) is a high affinity ligand for the Mas receptor (K_{d}=0.33 nM) (Santos, R. A. S. et al., PNAS 100:8258-8263 (2003)) may point to a possible role for the Mas receptor in blood pressure regulation and thrombus production.

The renin/angiotensin system is one of the major pathways by which blood pressure is regulated. Renin is produced in the kidneys in response to a decrease in renal perfusion pressure when catecholamines or angiotensin II are present, or when sodium or chloride ion concentrations in the blood decline. Renin catalyzes the conversion of angiotensinogen to its inactive metabolite, angiotensin I. Angiotensin converting enzyme catalyzes the conversion of angiotensin I to angiotensin II, a powerful vasoconstrictor which acts on the angiotensin II receptor. The cardiovascular and baroreflex actions of Ang (1-7) are reported to counteract those of angiotensin II. Whereas, angiotensin II, acting at the AT₂ receptor causes vasoconstriction and concurrent increase in blood pressure, Ang (1-7) acting at the Mas receptor has been reported to cause vasodilation and blood pressure decrease (Santos, R. A. et al., Regul. Pept. 91:45-62(2000)).

The standard treatment for myocardial infarction is reperfusion of the ischemic area by thrombolysis or percutaneous coronary angioplasty. Release of the blockage and return of blood flow to the affected area is crucial for heart tissue survival; however, damage beyond that generated by ischemia is typically observed in the reperfused heart tissue. The manifestations of reperfusion injury include arrhythmia, reversible contractile dysfunction-myocardial stunning, endothelial dysfunction and cell death. Currently, there is no effective treatment for reperfusion injury available. Ang (1-7) has been shown to improve post-ischemic myocardial function in an ischemia/reperfusion model using isolated rat hearts. (Ferreira, A. J. et al., Braz. J. of Med. and Biol. Res. 35(9):1083-1090 (2002)).

In addition to the immediate adverse effects of myocardial infarction, subsequent loss of contractile function, scarring and tissue remodeling often lead to congestive heart failure (CHF). A follow-up to the Framingham Heart Study indicates that 22% of male and 46% of female myocardial infarction victims will be disabled with CHF within six years following their heart attack. Despite significant advances in the treatment and prevention of congestive heart disease, the prognosis for patients with CHF remains poor. A recent study reported that 12% of patients die within three months of diagnosis, 33% die within one year and approximately 60% die within five years.

Hypertension is the most common factor contributing to CHF. The American Heart Association estimates that 75% of CHF cases have antecedent hypertension. In most hypertensive individuals, cardiac output is normal but there is an increase in resistance in the arteriole circulation causing the heart to pump harder to overcome the peripheral resistance and perfuse the peripheral tissues. The left ventricle develops pressure hypertrophy, which leads to myocardial remodeling and reduced pumping capacity resulting in a cycle of reduced cardiac function. Control of blood pressure is an effective treatment for chronic CHF and considerable effort has been focused on the development of therapies for hypertension. Foremost among these, are the angiotensin converting enzyme inhibitors (ACEIs). ACEIs block the conversion of angiotensin I to angiotensin II, thus, decreasing the hypertensive effects resulting from angiotensin II. Additionally, beta blockers, which act on the beta adrenergic receptor and inhibit sympathetic innervation of the heart, are used to treat chronic hypertension. Although these therapies are effective, there can be severe side effects associated with their use. As such, they are not tolerated by all individuals and there is a need for new and effective alternatives to these therapies.

Ang (1-7) has been shown to have a vasodilatory effect in many vascular beds, including canine and porcine coronary arteries, rat aorta, and feline mesenteric arteries. Chronic infusion of Ang (1-7) in spontaneously hypertensive rats and Dahl salt-sensitive rats has been shown to reduce mean arterial blood pressure. Ang (1-7) has been shown to block the Ang II induced vasoconstriction in isolated human arteries and antagonized vasoconstriction in forearm circulation by Ang II in normotensive men. Direct vasodilation to the same extent in basal forearm circulation of both normotensive and hypertensive patients by Ang (1-7) has been observed. Additionally, although the mechanism is undefined, it is believed that the vasodilation effects of bradykinin are potentiated by Ang (1-7).

The discovery that Ang (1-7) is an endogenous ligand for the Mas receptor has provided validation of the importance of the development of therapeutic entities which modulate Mas receptor activity. However, the inherent instability of Ang (1-7) and the likelihood that it is not absorbed upon oral administration make it ineffective as a therapeutic agent. These considerations highlight the importance of the development of pharmacologically useful modulators of the Mas receptor for the safe and effective treatment and/or prevention of human disease.

Citation of any reference throughout this application is not to be construed as an admission that such reference is prior art to the present application.

### 3. SUMMARY OF THE INVENTION

Applicants have generated novel Spiroindoline and Spiroisoquinoline Compounds and pharmaceutically acceptable salts, free bases, solvates, hydrates, stereoisomer, clathrates and prodrugs thereof, which are useful, for example, as cardio-protective or neuro-protective agents in mammals.

While the literature cited above may indicate that an agonist of the Mas receptor would be cardio-protective and decrease blood pressure, Applicants have unexpectedly identified compounds that can act as inverse agonists of the Mas receptor which are cardio-protective and do not raise blood pressure. For example, Compound 75 disclosed herein can act as an inverse agonist of the Mas receptor (see Example 23, FIG. 1 and Table 2), is cardio-protective (see Example 24 and FIGS. 2-5), and does not raise blood pressure (see Example 25 and FIG. 6).

The Mas receptor is a GPCR that couples to the Gq G-protein. Although some lines of evidence point to Ang (1-7) as a ligand for the Mas receptor (see Santos et al., supra, 2003), Applicants have advantageously chosen herein an assay that does not rely on using a ligand for the Mas receptor. Thus, this assay is not biased by the use of a particular ligand for the Mas receptor. Applicants have over-expressed the Mas receptor in cells such that the receptor is constitutively active in the absence of a ligand. Applicants have used an IP₃ assay to screen for compounds that decrease the amount of Mas receptor functionality and disclose herein several compounds that can significantly decrease Mas receptor functionality. The compounds can act as inverse agonists at a Mas receptor. An “inverse agonist” means a compound that binds to a receptor so as to reduce the baseline intracellular response of the receptor observed in the absence of agonist.

While the Compounds of the Invention have activity at the Mas receptor, it is understood that a Compound of the Invention may also act at another receptor or receptors which can elicit some of the biological properties of the compound such as, for example, effects on blood pressure, cardio-protection, or neuro-protection. For example, several genes related to the Mas receptor gene, called Mas-related genes or mrgs, are known in the art (Dong et al. supra, 2001). Also, as mentioned above, a peptide called NPFF has been found to bind to the Mas receptor, although weakly (Dong et al. supra, 2001). The NPFF peptide has been implicated in pain response and is also reported to have effects on the cardiovascular system (Allard et al. J. Pharmacol Exp. Ther. 274:577-583 (1995); Laguzzi et al., Brain Res. 711:193-202 (1996)). The NPFF peptide binds with high affinity to two neuropeptide-Y like GPCRs called NPFF1 (Kd=1.3 nM) and NPFF2 (Kd=0.3 nM) (Bonini et al., J. Biol. Chem. 275:39324-39331 (2000); Elshourbagy et al., J. Biol. Chem., 275:25965-25971 (2000)).

The present invention encompasses Spiroindoline and Spiroisoquinoline Compounds of Formula (I): 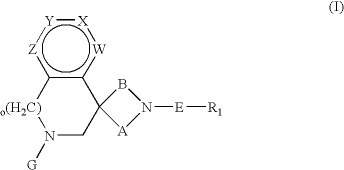
and pharmaceutically acceptable salts, free bases, solvates, hydrates, stereoisomers, clathrates or prodrugs thereof, wherein:

R₁ is H, halogen, hydroxy, nitro, cyano, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, substituted or unsubstituted -(3 to 7) membered heterocycle, substituted or unsubstituted -(7 to 10) membered bicycloheterocycle, substituted or unsubstituted -(5 to 10) membered heteroaryl, —NR₂R₂′, —C(═O)—R₇, —S(═O)₂—R₇, —C(═O)O—R₇, or —C(═O)N(R₇)(C₁₋₆ alkyl);

A is a substituted or unsubstituted C₁-C₃ alkylene;

B is a substituted or unsubstituted C₁-C₃ alkylene;

E is a bond, or a substituted or unsubstituted C₁-C₃ alkylene;

G is H, —Ar, —C(═O)—Ar, —C(═O)O—Ar, substituted or unsubstituted —C(═O)O—C₁₋₆alkyl, —C(═O)N(R₇)(Ar), substituted or unsubstituted —C(═O)N(R₇)(C₁₋₆ alkyl), —S(═O)₂—Ar, substituted or unsubstituted —S(═O)₂—C₁₋₆ alkyl, substituted or unsubstituted C₁₋₆ alkyl, substituted or unsubstituted C₁₋₆ alkyl-Ar, substituted or unsubstituted —C(═O)C₁₋₆ alkyl-Ar, or substituted or unsubstituted —C(═O)C₁₋₆ alkyl;

W is N or —CR₃—;

X is N or —CR₄—;

Y is N or —CR₅—;

Z is N or —CR₆—;

R₂, R₂′, R₃, R₄, R₅, R₆ and R₇ are at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, —C(═O)—O—C₁₋₆ alkyl, —O—C₁₋₆ alkyl, —C₁₋₆ alkyl-O—C₁₋₆ alkyl, —C₁₋₆ alkyl-NH₂, —C₀₋₆ alkyl-C(═O)—NH(C₁₋₆ alkyl), —C₀₋₆ alkyl-C(═O)—N(C₁₋₆ alkyl)(C₁₋₆ alkyl), —C₁₋₆ alkyl-NH-C(═O)—C₁₋₆ alkyl, —C₁₋₆ alkyl-S(═O)—C₁₋₆ alkyl, —C₀₋₆ alkyl-O—S(═O)₂—C₁₋₆ alkyl-S(═O)₂—C₁₋₆ alkyl, —C₁₋₆ alkyl-NR′—S(═O)₂—R′, —C₁₋₆ alkyl-SH, —C₁₋₆ alkyl-S—C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —(C(R′)₂)₀₋₆—O—(C(R′)₂)₁₋₅C(R′)₃, —(C(R′)₂)₁₋₅C(R′)₃, —(C(R′)₂)₀₋₆—S—(C(R′)₂)₁₋₅C(R′)₃, —(C(R′)₂)₀₋₆—S(═O)—C(R′)₂)₁₋₅C(R′)₃ or —(C(R′)₂)₀₋₆—S(═O)₂-(C(R′)₂)₁₋₅C(R′)₃;

o is 0 or 1;

R′ is at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted aryl, or substituted or unsubstituted C3-8 cycloalkyl; and

Ar is substituted or unsubstituted aryl, substituted or unsubstituted C₃₋₇ cycloalkyl, substituted or unsubstituted C₈₋₁₄ bicycloalkyl, substituted or unsubstituted C₈₋₁₄ tricycloalkyl, substituted or unsubstituted -(3 to 7) membered heterocycle, substituted or unsubstituted -(7 to 10) membered bicycloheterocycle, or substituted or unsubstituted -(5 to 10 membered)heteroaryl.

The compounds of Formula (I) are further described below.

The invention also relates to radio-labeled compounds of Formula (I) including, but not limited to, those containing one or more ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I or ¹³¹I atoms.

Spiroindoline and Spiroisoquinoline compounds of Formula (I) or pharmaceutically acceptable salts, free bases, solvates, hydrates, stereoisomers, clathrates or prodrugs thereof (“Compound(s) of the Invention”), are useful as a cardio-protective and/or neuroprotective agents. In one embodiment, a Compound of the Invention does not significantly increase blood pressure. The Compounds of the Invention are also useful for treating, preventing and/or managing vascular or cardiovascular diseases or disorders including, but not limited to, atherosclerosis, reperfusion injury, acute myocardial infarction, high blood pressure, hypertension, primary or secondary hypertension, renal vascular hypertension, acute or chronic congestive heart failure, left ventricular hypertrophy, vascular hypertrophy, glaucoma, primary or secondary hyperaldosteronism, diabetic neuropathy, glomerulonephritis, scleroderma, glomerular sclerosis, renal failure, renal transplant therapy, diabetic retinopathy, other vascular diseases or disorders and migraines. A Compound of the Invention is also useful for treating, preventing and/or managing neurological diseases or disorders including, but not limited to, diabetic peripheral neuropathy, pain, stroke, cerebral ischemia and Parkinson's disease in a patient in need thereof. The Compounds of the Invention can also be used in patients at risk of such diseases and disorders as cardio-protective or neuro-protective agents.

In one embodiment, a Compound of the Invention is used in combination with other compounds for the treatment of a vascular, cardiovascular or neurological disease or disorder. For example, in one embodiment, a Compound of the Invention is used in combination with, or in place of, angiotensin-converting enzyme (ACE) inhibitors to treat the diseases or disorders for which such ACE inhibitors are conventionally used.

The invention further relates to methods for assaying the ability of a Compound of the Invention or another compound to bind to a Mas receptor, comprising contacting a radio-labeled Compound of the Invention with a cell capable of expressing a Mas receptor. The invention also relates to methods for assaying the ability of a Compound of the Invention or another compound to modulate the functionality of a Mas receptor, comprising contacting a Compound of the Invention with a cell capable of expressing a Mas receptor.

The invention also relates to methods for treating or preventing a disorder treatable or preventable by inhibiting Mas receptor function, comprising administering to a patient in need thereof an effective amount of a Compound of the Invention. In one embodiment, the disorder is a vascular or cardiovascular disease or disorder and in another embodiment, the disorder is a neurological disease or disorder.

The invention further relates to methods for inhibiting Mas receptor function in a cell, comprising contacting a cell capable of expressing the Mas receptor with an effective amount of a Compound of the Invention.

The invention further relates to pharmaceutical compositions comprising a Compound of the Invention and a pharmaceutically acceptable vehicle or excipient. The compositions are useful as cardio-protective and/or neuro-protective agents and for treating or preventing a vascular or cardiovascular disorder and/or a neurological disorder in a patient.

The invention further relates to methods for treating a vascular or cardiovascular disorder and/or a neurological disorder, comprising administering to a patient in need thereof a Compound of the Invention.

The invention further relates to methods for preventing a vascular or cardiovascular disorder and/or a neurological disorder, comprising administering to a patient in need thereof a Compound of the Invention.

The invention further relates to methods for managing a vascular or cardiovascular disorder and/or a neurological disorder, comprising administering to a patient in need thereof a Compound of the Invention.

The invention further relates to a method for manufacturing a medicament, comprising the step of admixing a Compound of the Invention and a pharmaceutically acceptable vehicle or excipient. In a particular embodiment, a medicament comprising a Compound of the Invention is useful for treating, preventing and/or managing a vascular or cardiovascular disorder and/or a neurological disorder. In another embodiment, a medicament comprising a Compound of the Invention is useful as a cardio-protective or neuro-protective agent.

The invention further relates to a Compound of the Invention, as described herein, for use in a method of treatment of the human or animal body by therapy.

The invention also relates to a method for identifying a cardio-protective compound, comprising: a) contacting a candidate compound with a Mas receptor, and b) determining whether the receptor functionality is decreased, wherein a decrease in receptor functionality is indicative of the candidate compound being a cardio-protective compound. In one embodiment, the Mas receptor is human. In another embodiment, the cardio-protective compound is an inverse agonist or antagonist of the Mas receptor. In a further embodiment, the cardio-protective compound is an inverse agonist of the Mas receptor. In another embodiment, determining whether the receptor functionality is decreased comprises using an IP₃ assay. The invention further relates to a cardio-protective compound identified according to this method. In one embodiment, the cardio-protective compound is an inverse agonist. In another embodiment, the cardio-protective compound is an inverse agonist that does not significantly increase blood pressure.

The invention also relates to a method for identifying a cardio-protective compound, comprising: a) contacting a candidate compound with a Mas receptor, b) determining whether the receptor functionality is decreased, and c) determining the effect of the compound on blood pressure, wherein a decrease in receptor functionality and no significant increase in blood pressure is indicative of the candidate compound being a cardio-protective compound.

The invention further relates to a method for inhibiting Mas receptor function in a cell, comprising contacting a cell capable of expressing Mas with an effective amount of the cardio-protective compound identified by a method comprising: a) contacting a candidate compound with a Mas receptor, and b) determining whether the receptor functionality is decreased, wherein a decrease in receptor functionality is indicative of the candidate compound being a cardio-protective compound.

The invention also relates to a method for preparing a composition which comprises identifying a cardio-protective compound and then admixing said modulator and carrier, wherein the modulator is identified by a method comprising: a) contacting a candidate compound with a Mas receptor, and b) determining whether the receptor functionality is decreased, wherein a decrease in receptor functionality is indicative of the candidate compound being a cardio-protective compound.

The invention also relates to a pharmaceutical composition comprising, consisting essentially of, or consisting of an inverse agonist identified by a method comprising: a) contacting a candidate compound with a Mas receptor, and b) determining whether the receptor functionality is decreased, wherein a decrease in receptor functionality is indicative of the candidate compound being a cardio-protective compound. The invention further relates to a method for effecting cardio protection in an individual in need of said cardio protection, comprising administering to said individual an effective amount of this pharmaceutical composition. The invention also relates to a method for treating or preventing a vascular or cardiovascular disease or disorder in an individual in need of said treating or preventing, comprising administering an effective amount of this pharmaceutical composition to said individual. In one embodiment, said vascular or cardiovascular disease or disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine. In another embodiment, said vascular or cardiovascular disease or disorder is reperfusion injury, acute myocardial infarction, acute or chronic congestive heart failure, left ventricular hypertrophy or vascular hypertrophy.

The invention also relates to a method of effecting a needed change in cardiovascular function in an individual in need of said change, comprising administering an effective amount of a pharmaceutical composition comprising, consisting essentially of, or consisting of an inverse agonist identified by a method comprising: a) contacting a candidate compound with a Mas receptor, and b) determining whether the receptor functionality is decreased, wherein a decrease in receptor functionality is indicative of the candidate compound being a cardio-protective compound, and wherein said needed change in cardiovascular function is an increase in ventricular contractile function.

The invention also relates to a method for the manufacture of a medicament comprising this pharmaceutical composition, for use in the treatment of a vascular or cardiovascular disease. The invention farther relates to a method for the manufacture of a medicament comprising this pharmaceutical composition, for use as a cardio-protective agent.

In addition, the invention relates to a method for selectively inhibiting Mas receptor activity in a human host, comprising administering a compound that selectively inhibits activity of the Mas receptor gene product to a human host in need of such treatment. For example, the invention relates to a method for selectively inhibiting Mas receptor activity in a human host, comprising administering an inverse agonist of the Mas receptor that selectively inhibits activity of the Mas receptor gene product to a human host in need of such treatment. The invention also relates to a method for selectively inhibiting Mas receptor activity in a human host, comprising administering a compound of Formula (I) that selectively inhibits activity of the Mas receptor gene product to a human host in need of such treatment.

The invention still further relates to a kit comprising a container containing a Compound of the Invention. The kit may further comprise printed instructions for using the Compound of the Invention to treat, prevent and/or manage any of the aforementioned diseases or disorders.

The present invention may be understood more fully by reference to the following detailed description and illustrative examples, which are intended to exemplify non-limiting embodiments of the invention.

### 4. BRIEF DESCRIPTION OF THE DRAWINGS

FIG. 1 shows an IP₃ assay of Compound 75, disclosed herein, using HEK293 cells that over-express the human Mas receptor resulting in constitutive activity of the Mas receptor in these cells.

FIG. 2 shows the results of an ischemia-reperfusion assay in isolated rat hearts treated with Compound 75 or vehicle.

FIG. 3 shows the results of another ischemia-reperfusion assay in isolated rat hearts treated with Compound 75 or vehicle (control).

FIG. 4 shows end diastolic pressure (EDP) readings in the isolated rat hearts from the ischemia-reperfusion assay shown in FIG. 3.

FIG. 5 shows epicardial electrogram recordings in selected isolated rat hearts from the ischemia-reperfusion assay shown in FIG. 3.

FIG. 6 shows blood pressure measurements in rats treated with Compound 75, vehicle, or control compounds angiotensin II (AngII) and sodium nitroprusside (SNP).

### 5. DETAILED DESCRIPTION OF THE INVENTION

### 5.1 SPIROINDOLINE AND SPIROISOGUINOLINE COMPOUNDS OF FORMULA (I)

The present invention encompasses Spiroindoline and Spiroquinoline Compounds of Formula (I): 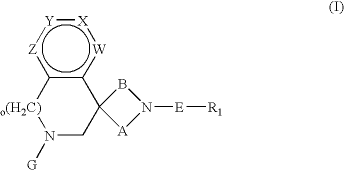
and pharmaceutically acceptable salts, free bases, solvates, hydrates, stereoisomers, clathrates or prodrugs thereof, wherein A, B, E, G, W, X, Y, Z, o, and R, are defined above (“Compound(s) of the Invention”).

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

In one embodiment, E is —(CH₂)ₚ— wherein p is 0, 1, or 2. In some embodiments, compounds of invention are represented by Formula (Ib) as shown below: 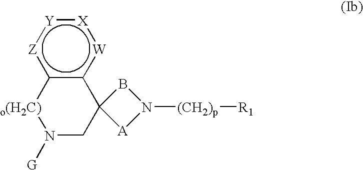
wherein each variable in Formula (Ib) has the same meaning as described herein, and p is 0, 1, or 2. In some embodiments, p is 0. In other embodiments, p is 1. In still other embodiments, p is 2.

In another embodiment, W, X, Y and Z are each —CH—.

In another embodiment, W, Y and Z are each —CH— and X is —C(halogen)-.

In another embodiment, W, Y and Z are each —CH— and X is —C(Cl)— or —C(F)—.

In another embodiment, W, Y and Z are each —CH— and X is —C(CH₃)—, —C(OCH₃)—, —C(OH)—, —C(OS(═O)₂CH₃) or —C(CF₃)—.

In another embodiment, W, Y and Z are each —CH— and X is —C(isopropyl)-.

In another embodiment, W, Y and Z are each —CH— and X is —C(tert-butyl)-.

In another embodiment, W, Y and Z are each —CH— and X is —C(CH₃)—.

In another embodiment, W, X and Z are each —CH— and Y is —C(F)— or —C(Cl)—.

W and Y may also each be —CH— while X and Z are substituted carbon atoms. Preferably, X and Z are substituted with lower alkyl, halogen, hydroxy or lower alkoxy. Most preferably, W and Y are each —CH— and X and Z are each —C(CH₃)— or —C(CF₃)—.

W and Y may also each be —CH— while X and Z are each independently —CH— or a substituted carbon atom. Preferably, X and Z are substituted with lower alkyl, halogen, hydroxy or lower alkoxy. Most preferably, W and Y are each —CH— and X and Z are each independently —CH—, —C(CH₃)—, —C(CF₃)—, —C(isopropyl)-, or —C(tert-butyl)-.

Another subclass is formed wherein A and B are each —(CH₂)₂— or one of A and B is —(CH₂)₂— and the other is —(CH₂)—.

In another embodiment, p is 1 or 2 and R₁ is —CH═CH₂.

In another embodiment, p is 1 or 2 and R₁ is cyclobutyl.

In another embodiment, p is 1 or 2 and R₁ is -cyclobutyl.

In another embodiment, p is 1 or 2 and R₁ is -cyclopropyl.

In another embodiment, p is 1 and R₁ is —CH₂CH₃.

In another embodiment, p is 1 and R₁ is —(CH₂)₂CH₃.

In another embodiment, p is 0 and R₁ is phenyl.

In another embodiment, p is 1 or 2 and R₁ is phenyl.

In another embodiment, p is 1 and R₁ is —CH(OH)CH₃.

In another embodiment, p is 1 and R₁ is —C(═CH₂)CH₃.

In another embodiment, p is 1 and R₁ is H.

In another embodiment, p is 0 and R₁ is H.

In another embodiment, p is 0 and R₁ is —C(═O)cyclobutyl.

In another embodiment, p is 0 and R₁ is —C(═O)CH(Ar)₂.

In another embodiment, p is 0 and R₁ is —C(═O)CH(CH₃)₂.

In another embodiment, p is 0 and R₁ is —S(═O)₂-4-chloro-phenyl.

In another embodiment, p is 0 and R₁ is —C(═O)naphtha-1-yl.

In another embodiment, p is 1 and R₁ is 3,4-dimethoxyphenyl.

In another embodiment, p is 1 and R₁ is 3,4-dichlorophenyl.

In another embodiment, p is 0 and R₁ is —C(═O)NH-phenyl.

In another embodiment, p is 0 and R₁ is 2-(4-methyl-3-nitro-benzoyloxy)-cyclohexyl.

In another embodiment, p is 0 and R₁ is 2-hydroxy-cyclohexyl.

In another embodiment, p is 0 and R₁ is —C(═O)-3-nitro4-methyl-phenyl.

In another embodiment, p is 0 and R₁ is —C(═O)-4-fluoro-phenyl.

In another embodiment, p is 0 and R₁ is —C(═O)-2-methoxy-phenyl.

In another embodiment, p is 0 and R₁ is —C(═O)benzo[1,3]dioxol-5-yl.

In another embodiment, p is 0 and R₁ is 2-cyclohexylcarbamoyloxy-cyclohexyl

In another embodiment, p is 0 and R₁ is 2-(3,4-difluoro-benzoyloxy)-propyl.

In another embodiment, p is 0 and R₁ is —C(═O)-3-nitro-phenyl.

In another embodiment, p is 0 and R₁ is —C(═O)-2-fluoro-phenyl.

In another embodiment, p is 0 and R₁ is —C(═O)-4-methoxy-phenyl.

In another embodiment, p is 1 and R₁ is 2,4-dimethylphenyl.

In another embodiment, p is 0 and R₁ is benzo[1,3]dioxol-5-ylmethyl.

In another embodiment, p is 0 and R₁ is —C(═O)O-tert-butyl.

In another embodiment, p is 0 and R₁ is —C(═O)-2-chloro-phenyl.

In another embodiment, p is 0 and R₁ is —S(═O)₂-4-nitro-phenyl.

In another embodiment, p is 0 and R₁ is 2-chlorophenyl.

In another embodiment, p is 0 and R₁ is 3-chlorophenyl.

In another embodiment, p is 0 and R₁ is 4-chlorophenyl.

In another embodiment, p is 0 and R₁ is 3,4-dichlorophenyl.

In another embodiment, p is 0 and R₁ is 4-methylphenyl.

In another embodiment, p is 0 and R₁ is 2-fluorophenyl.

In another embodiment, p is 0 and R₁ is 6-chloro-pyridin-3-yl.

In another embodiment, p is 0 and R₁ is 4-trifluoromethylphenyl.

In another embodiment, p is 0 and R₁ is 2-methoxycarbonyl-ethyl.

In another embodiment, p is 0 and R₁ is 2-carboxy-ethyl.

In another embodiment, p is 2 and R₁ is —CH₂CH═CH₂.

In another embodiment, p is 0 and R₁ is 2-phenylsulfanyl-ethyl, [i.e., —(CH₂)₂S-phenyl].

In another embodiment, p is 1 and R₁ is —C(═O)-tert-butyl

In another embodiment, p is 2 and R₁ is —S—CH₂CH₃.

In another embodiment, p is 0 and R₁ is 1-phenyl-ethyl, [i e., —CH(phenyl)CH₃].

In another embodiment, p is 0 and R₁ is 2-carboxy-allyl, [i.e., —CH₂C(CO₂H)═CH₂].

In another embodiment, p is 1 and R₁ is tetrahydro-pyran-2-yl.

In another embodiment, p is 0 and R₁ is 3-methyl-but-2-enyl, [i.e., CH₂CH═C(CH₃)₂].

In another embodiment, p is 1 and R₁ is —C(═O)CH₂CH₃.

In another embodiment, p is 1 and R₁ is —C(═O)phenyl.

In another embodiment, p is 0 and R₁ is 1-methyl-2-phenyl-ethyl, [i.e., CH(CH₃)CH₂-phenyl].

In another embodiment, p is 1 and R₁ is [1,3]dioxolan-2-yl.

In another embodiment, p is 1 and R₁ is —C(═O)-4-methoxy-phenyl.

In another embodiment, p is 1 and R₁ is —C(═O)O-ethyl.

In another embodiment, p is 1 and R₁ is —C(═O)-4-chloro-phenyl.

In another embodiment, p is 0 and R₁ is 3-(1,3-dioxo-1,3-dihydro-isoindol-2-yl)-propyl, can also be represented by the following formula:

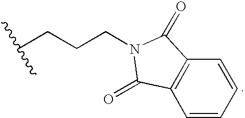

In another embodiment, p is 0 and R₁ is 4-(1,3-dioxo-1,3-dihydro-isoindol-2-yl)-butyl.

In another embodiment, p is 2 and R₁ is 1H-indol-3-yl

In another embodiment, p is 0 and R₁ is 2-methylsulfanyl-propyl, and can be represented by the formula: —CH₂CH(CH₃)SCH₃.

In another embodiment, p is 0 and R₁ is 3-methylsulfanyl-propyl.

In another embodiment, p is 1 and R₁ is 2-chloro4-fluorophenyl.

In another embodiment, p is 1 and R₁ is 2,4-dichlorophenyl.

In another embodiment, p is 1 and R₁ is 4-trifluorophenyl

In another embodiment, p is 1 and R₁ is 4-tert-butylphenyl.

In another embodiment, p is 1 and R₁ is 3-chlorophenyl.

In another embodiment, p is 0 and R₁ is but-3-ynyl, [i.e., —CH₂CH₂CH₂C≡CH

In another embodiment, p is 1 and R₁ is 1H-pyrrol-2-yl.

In another embodiment, p is 1 and R₁ is thiophen-3-yl.

In another embodiment, p is 1 and R₁ is thiophen-2-yl.

In another embodiment, p is 1 and R₁ is furan-3-yl.

In another embodiment, p is 2 and R₁ is —CH₂NH₂.

In another embodiment, p is 2 and R₁ is —CH₂CH₂NH₂.

In another embodiment, p is 0 and R₁ is -cyclobutyl.

In another embodiment, p is 1 and R₁ is cyclopentyl.

In another embodiment, p is 1 and R₁ is cyclohexyl.

In another embodiment, p is 1 and R₁ is cyclohex-3-enyl.

In another embodiment, p is 0 and R₁ is 3,4,4-trifluoro-but-3-enyl, and can be represented by the following formula:

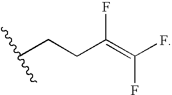

In another embodiment, p is 0 and R₁ is hex-5-enyl, [i.e., —(CH₂)₃CH₂CH═CH₂].

In another embodiment, G is —C(═O)—Ar.

In another embodiment, G is —C(═O)CH₂—Ar or G is —C(═O)CH(Ar)₂.

In another embodiment, G is —C(═O)NH—Ar or —C(═O)NH₂ or —C(═O)NH(alkyl).

In another embodiment, G is —S(═O)₂—Ar.

In another embodiment, Ar is substituted or unsubstituted phenyl; preferably mono or disubstituted phenyl; most preferably mono or disubstituted phenyl substituted with either halogen, lower alkyl or lower alkoxy.

In another embodiment, Ar is methoxy phenyl substituted in the para position.

In another embodiment, Ar is fluorophenyl substituted in the ortho position.

In another embodiment, Ar is fluorophenyl substituted in the para position.

In another embodiment, Ar is difluorophenyl substituted in the ortho and para positions.

In another embodiment, Ar is difluorophenyl substituted in the ortho and meta positions.

In another embodiment, Ar is difluorophenyl substituted in the ortho positions.

In another embodiment, Ar is difluorophenyl substituted in the meta positions.

In another embodiment, Ar is substituted or unsubstituted furan.

In another embodiment, Ar is substituted or unsubstituted pyridine.

In another embodiment, Ar is substituted or unsubstituted thiophene.

In another embodiment, Ar is substituted or unsubstituted adamantane.

In another embodiment, Ar is 2-chlorothiophene.

In another embodiment, Ar is benzo(1,3)dioxole.

In another embodiment, Ar is fluoren-9-one.

In another embodiment, Ar is morpholine.

In another embodiment, G is butyl.

In another embodiment, G is phenethyl, (i.e., —CH₂CH₂-phenyl).

In another embodiment, G is —C(═O)-cyclobutyl.

In another embodiment, G is —C(═O)O-tert-butyl.

In another embodiment, G is H.

In another embodiment, G is 2,4-dimethylbenzyl.

In another embodiment, G is benzo[1,3]dioxol-5-ylmethyl.

In another embodiment, G is 3,4-chlorobenzyl.

In another embodiment, G is cyclopropylmethyl.

In another embodiment, G is —C(═O)aryl, wherein the aryl is phenyl, naphthyl or fluorenyl and each aryl is optionally substituted with 1, 2, 3, 4, or 5 substituents independently selected from the group consisting of halo, C₁₋₆ alkoxy, nitro, C₁₋₆ alkyl, C₁₋₆ haloalkyl, and oxo (═O), or two adjacent substituents together with ring carbons to which they are bonded form a 5 or 6-member heterocyclic ring.

In another embodiment, G is —C(═O)aryl, wherein the aryl is phenyl, or naphthyl and each aryl is optionally substituted with 1, 2, 3, 4, or 5 substituents independently selected from the group consisting of chloro, fluoro, bromo, —OCH₃, —OCH₂CH₃, nitro, —CH₃, —CH₂CH₃, —CF₃, and —CHCl₂, or two adjacent substituents together with ring carbons to which they are bonded form a [1,3]dioxolane (for example, when aryl is phenyl, together aryl is a benzo[1,3]dioxolyl group).

In another embodiment, G is —C(═O)heteroaryl, wherein the aryl is pyridyl, thienyl, furanyl, imidazolyl, or pyrazolyl, and each heteroaryl is optionally substituted with 1, 2, 3, or 4 substituents independently selected from the group consisting of halo, C₁₋₆ alkyl, C₁₋₆ haloalkyl, C₁₋₆alkylthio, C₂₋₆ alkenylthio, nitro, and thiol.

In another embodiment, G is —C(═O)heteroaryl, wherein the aryl is pyridyl, thienyl, furanyl, imidazolyl, or pyrazolyl, and each heteroaryl is optionally substituted with 1, 2, 3, or 4 substituents independently selected from the group consisting of chloro, fluoro, bromo, —SCH₃, —SCH₂CH═CH₂, nitro, —CH₃, —CF₃, and —SH.

In another embodiment, G is —C(═O)cycloalkyl.

In another embodiment, G is —C(═O)NH-aryl optionally substituted with 1, 2, 3, 4, or 5 substituents independently selected from the group consisting of nitro, halo, and C₁₋₆ alkoxy.

In another embodiment, G is —C(═O)CH₂-phenyl.

In another embodiment, G is —C(═O)CH₂-thienyl.

In another embodiment, G is —C(═O)NH—CH₂-phenyl, wherein the phenyl is optionally with C₁₋₆ alkoxy.

In another embodiment, G is —C(═O)NH—C₁₋₆ alkyl. In some embodiments, the C₁₋₆ alkyl is ethyl. In some embodiments, the C₁₋₆ alkyl is iso-propyl.

In another embodiment, G is —C(═O)CH₂-phenyl.

In another embodiment, G is selected from the group consisting of:

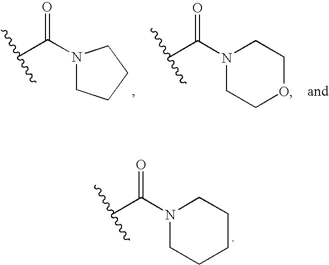

In another embodiment, G is —S(═O)₂phenyl wherein the phenyl is optionally substituted with 1, 2, 3, 4, or 5 substituents independently selected from the group consisting of halo, nitro, and C₁₋₆ haloalkyl.

In another embodiment, G is —S(═O)₂phenyl wherein the phenyl is optionally substituted with 1, 2, 3, 4, or 5 substituents independently selected from the group consisting of fluoro, chloro, nitro, and —CF₃.

In another embodiment, G is —S(═O)₂-thienyl optionally substituted with 1, 2, or 3 halogens.

In another embodiment, o is 0. In another specific embodiment, when o is 1, another subclass of compounds is formed.

In another embodiment, p is 0. In another specific embodiment, when p is 1, another subclass of compounds is formed.

In another embodiment, when X is —C(F)—, then G is preferably —C(═O)-substituted or unsubstituted phenyl.

In another embodiment, when X is —C(F)—, then G is preferably —C(═O)-substituted or unsubstituted -(3 to 7) membered heterocycle.

In another embodiment, when X is —C(F)—, then G is preferably —C(═O)N— substituted or unsubstituted phenyl.

In another embodiment, W is H.

In another embodiment, X is H.

In another embodiment, Y is H.

In another embodiment, Z is H.

In another embodiment, W is —C(CH₃)—.

In another embodiment, X is selected from the group consisting of —C(F)—, —C(OCH₃)—, —C(OH)—, —C(OS(═O)₂CH₃)—, —C(Cl)—, —C(CH₃)—, —C(CF₃)—, —C(CH(CH₃)₂)—, and —C(C(CH₃)₃)—. In some embodiments, X is —C(F)—, or —C(Cl)—. In some embodiments, X is —C(CH(CH₃)₂)—, or —C(C(CH₃)₃)—.

In another embodiment, Y is —C(CH₃)—.

In another embodiment, Z is —C(CH₃)—.

In another embodiment, the present invention encompasses compounds of Formula (I): 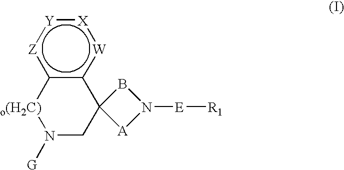
and pharmaceutically acceptable salts, free bases, solvates, hydrates, stereoisomers, clathrates or prodrugs thereof, wherein:

R₁ is H, halogen, hydroxy, nitro, cyano, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, substituted or unsubstituted -(3 to 7) membered heterocycle, substituted or unsubstituted -(7 to 10) membered bicycloheterocycle, substituted or unsubstituted -(5 to 10) membered heteroaryl, —NR₂R₂′, —C(═O)—R₇, —S(═O)₂—R₇, —C(═O)O—R₇, or —C(═O)N(R₇)(C₁₋₆ alkyl);

wherein the foregoing when substituted can be independently substituted with one or more substituents selected from halogen, hydroxy, nitro, cyano, C₁₋₆ alkyl, C₂₋₆ alkenyl, C₂₋₆ alkynyl, —C(═O)—C₁₋₆ alkyl, —C₀₋₆ alkyl-O—C₁₋₆ alkyl, —C₀₋₆ alkyl-C(═O)—NH(C₁₋₆ alkyl), —C₀₋₆ alkyl-C(═O)—N(C₁₋₆ alkyl)(C₁₋₆ alkyl), —C₀₋₆ alkyl-NH—C(═O)—C₁₋₆ alkyl, —C₀₋₆ alkyl-C(═S)—NH(C₁₋₆ alkyl), —C₀₋₆ alkyl-C(═S)—N(C₁₋₆ alkyl)(C₁₋₆ alkyl), —C₀₋₆ alkyl-NH—C(═S)—C₁₋₆ alkyl, —C₀₋₆ alkyl-S(═O)—C₁₋₆ alkyl, —C₀₋₆ alkyl-S(═O)₂—C₁₋₆ alkyl, —C₀₋₆ alkyl-SH, —C₀₋₆ alkyl-S—C₁₋₆ alkyl, —C₀₋₆ alkyl-NH—C(═S)—NH—C₁₋₆ alkyl, —C₀₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —C₀₋₆ alkyl-C(═O)OH, —(C(R′)₂)₀₋₆—O—(C(R′)₂)₁₋₅C(R′)₃, —(C(R′)₂)₀₋₅C(R′)₃, —(C(R′)₂)₀₋₆—S—(C(R′)₂)₁₋₅C(R′)₃, —(C(R′)₂)₀₋₆—S(═O)—C(R′)₂)₁₋₅C(R′)₃ or —(C(R′)₂)₀₋₆—S(═O)₂—(C(R′)₂)₀₋₅C(R′)₃;

A is substituted or unsubstituted C₁₋₃ alkylene;

B is substituted or unsubstituted C₁₋₃ alkylene;

E is a bond, or a substituted or unsubstituted C₁₋₃ alkylene;

G is H, —Ar, —C(═O)—Ar, —C(═O)O—Ar, substituted or unsubstituted —C(═O)O—C₁₋₆ alkyl, —C(═O)N(R₇)(Ar), substituted or unsubstituted —C(═O)N(R₇)(C₁₋₆ alkyl), —S(═O)₂—Ar, substituted or unsubstituted —S(═O)₂—C₁₋₆ alkyl, substituted or unsubstituted C₁₋₆ alkyl, substituted or unsubstituted C₁₋₆ alkyl-Ar, substituted or unsubstituted —C(═O)C₁₋₆ alkyl-Ar, or substituted or unsubstituted —C(═O)C₁₋₆ alkyl;

W is N or —CR₃—;

X is N or —CR₄—;

Y is N or —CR₅—;

Z is N or —CR₆—;

R₂, R₂′, R₃, R₄, R₅, R₆ and R₇ are at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted C₃₋₈ cycloalkyl, —C(═O)—O—C₁₋₆ alkyl, —O—C₁₋₆ alkyl, —C₁₋₆ alkyl-O—C₁₋₆ alkyl, —C₀₋₆ alkyl-C(═O)—NH(C₁₋₆ alkyl), —C₀₋₆ alkyl-C(═O)—N(C₁₋₆ alkyl)(C₁₋₆ alkyl), —C₁₋₆ alkyl-NH—C(═O)—C₁₋₆ alkyl, —C₁₋₆ alkyl-S(═O)-C₁₋₆ alkyl, —C₀₋₆ alkyl-O—S(═O)₂—C₁₋₆ alkyl, —C₁₋₆ alkyl-S(═O)₂—C₁₋₆ alkyl, —C₁₋₆ alkyl-SH, —C₁₋₆ alkyl-S—C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —(C(R′)₂)₀₋₆—O—(C(R′)₂)₁₋₅C(R′)₃, —(C(R′)₂)₁₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂—(C(R′)₂)₁₋₅C(R′)₃,

wherein when each C₁₋₈ alkyl, C₂₋₆ alkenyl, C₂₋₆ alkynyl or C₃₋₈ cycloalkyl is substituted, it can be individually substituted with one or more substituents selected from cyano, halogen, hydroxyl, nitro, —C(═O)—C₁₋₆ alkyl, —C₀₋₆ alkyl-O—C₁₋₆ alkyl, —C₀₋₆ alkyl-C(═O)—NH(C₁₋₆ alkyl), —C₀₋₆ alkyl-C(═O)—N(C₁₋₆ alkyl)(C₁₋₆ alkyl), —C₀₋₆ alkyl-NH—C(═O)—C₁₋₆ alkyl, —C₀₋₆ alkyl-C(═S)—NH(C₁₋₆ alkyl), —C₀₋₆ alkyl-C(═S)—N(C₁₋₆ alkyl)(C₁₋₆ alkyl), —C₀₋₆ alkyl-NH—C(═S)—C₁₋₆ alkyl, —C₀₋₆ alkyl-S(═O)—C₁₋₆ alkyl, —C₀₋₆ alkyl-S(═O)₂—C₁₋₆ alkyl, —C₀₋₆ alkyl-SH, —C₀₋₆ alkyl-S—C₁₋₆ alkyl, —C₀₋₆ alkyl-NH—C(═S)—NH—C₁₋₆ alkyl, —C₀₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —C₀₋₆ alkyl-C(═O)OH, —(C(R′)₂)₀₋₆—O—(C(R′)₂)₁₋₅C(R′)₃, —(C(R′)₂)₀₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂—(C(R′)₂)₀₋₅C(R′)₃;

o is 0 or 1;

R′ is at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted aryl, or substituted or unsubstituted C3-8 cycloalkyl; and

Ar is substituted or unsubstituted aryl, substituted or unsubstituted C₃₋₇ cycloalkyl, substituted or unsubstituted C₈₋₁₄ bicycloalkyl, substituted or unsubstituted C₈₋₁₄ tricycloalkyl, substituted or unsubstituted -(3 to 7) membered heterocycle, substituted or unsubstituted -(7 to 10) membered bicycloheterocycle or substituted or unsubstituted -(5 to 10 membered)heteroaryl, wherein when the foregoing is substituted, each is substituted with one or more substituents selected from cyano, halogen, hydroxyl, nitro, -(3- to 7-membered heterocycle), -(5-to 10 membered)heteroaryl, —O-phenyl, phenyl, —SO₃H, —C₁₋₈ alkyl, —C(═O)—C₁₋₆ alkyl, —C₀₋₆ alkyl-O—C₁₋₆ alkyl, —C₀₋₆ alkyl-C(═O)—NH(C₁₋₆ alkyl), —C₀₋₆ alkyl-C(═O)—N(C₁₋₆ alkyl)(C₁₋₆ alkyl), —C₀₋₆ alkyl-NH—C(═O)—C₁₋₆ alkyl, —C₀₋₆ alkyl-C(═S)—NH(C₁₋₆ alkyl), —C₀₋₆ alkyl-C(═S)—N(C₁₋₆ alkyl)(C₁₋₆ alkyl), —C₀₋₆ alkyl-NH—C(═S)—C₁₋₆ alkyl, —C₀₋₆ alkyl-S(═O)—C₁₋₆ alkyl, —C₀₋₆ alkyl-S(═O)₂—C₁₋₆ alkyl, —C₀₋₆ alkyl-SH, —C₀₋₆ alkyl-S—C₁₋₆ alkyl, —C₀₋₆ alkyl-NH—C(═S)—NH—C₁₋₆ alkyl, —C₀₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —C₀₋₆ alkyl-C(═O)OH, —(C(R′)₂)₀₋₆—O—(C(R′)₂)₁₋₅C(R′)₃, —(C(R′)₂)₀₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂—(C(R′)₂)₀₋₅C(R′)₃;

wherein each of the above substituents can be further substituted with one or more substituents independently selected from cyano, halogen, hydroxyl, nitro, -(3 to 7 membered heterocycle), -(5 to 10 membered)heteroaryl, —O-phenyl, phenyl, —SO₃H, —C(═O)—C₁₋₆ alkyl, —C₀₋₆ alkyl-O—C₁₋₆ alkyl, —C₀₋₆ alkyl-C(═O)—NH(C₁₋₆ alkyl), —C₀₋₆ alkyl-C(═O)—N(C₁₋₆ alkyl)(C₁₋₆ alkyl), —C₀₋₆ alkyl-NH—C(═O)—C₁₋₆ alkyl, —C₀₋₆ alkyl-C(═S)—NH(C₁₋₆ alkyl), —C₀₋₆ alkyl-C(═S)—N(C₁₋₆ alkyl)(C₁₋₆ alkyl), —C₀₋₆ alkyl-NH—C(═S)—C₁₋₆ alkyl, —C₀₋₆ alkyl-S(═O)—C₁₋₆ alkyl, —C₀₋₆ alkyl-S(═O)₂—C₁₋₆ alkyl, —C₀₋₆ alkyl-SH, —C₀₋₆ alkyl-S—C₁₋₆ alkyl, —C₀₋₆ alkyl-NH—C(═S)—NH—C₁₋₆ alkyl, —C₀₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —C₀₋₆ alkyl-C(═O)OH, —(C(R′)₂)₀₋₆—O—(C(R′)₂)₁₋₅C(R′)₃, —(C(R′)₂)₀₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂)—(C(R′)₂)₀₋₅C(R′)₃, or two adjacent substituents together with said aryl or -(5- to 10-membered)heteroaryl form a (C₃-₈) cycloalkyl, (C₅₋₁₀) cycloalkenyl or -(3- to 7-membered) heterocyclic group may optionally substituted with one or more halogens.

As used herein, the term “substituted” indicates that at least one hydrogen of the chemical group is replaced by a non-hydrogen substituent or group. When a chemical group herein is “substituted” it may have up to the full valance of substitution; for example a methyl group can be substituted by 1, 2, or 3 substituents, a methylene group group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like.

In some embodiments, when the group described is “substituted or unsubstituted,” when substituted, at least one hydrogen of the group is replaced by a non-hydrogen substituents selected from the group consisting of H, C₁₋₆ acyl, C₁₋₆ acyloxy, C₂₋₆ alkenyl, C₁₋₆ alkoxy, C₁₋₆ alkyl, C₁₋₆ alkylamino, C₁₋₆alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, arylsulfonyl, carbo-C₁₋₆-alkoxy, carboxamide, carboxy, cyano, C₃₋₈ cycloalkyl, C₁₋₆ dialkylamino, C₁₋₆ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heterocyclic, heterocyclicsulfonyl, hydroxyl, nitro, phenoxy, phenyl, sulfonamide, sulfonic acid, and thiol.

In another embodiment, the present invention encompasses compounds of Formula (I), wherein:

A, B, E, W, X, Y, Z, o, and R₁ are as defined above;

G is H, —Ar, —C(═O)—Ar, —C(═O)O—Ar, substituted or unsubstituted —C(═O)O—C₁₋₆ alkyl, —C(═O)N(R₇)(Ar), substituted or unsubstituted —C(═O)N(R₇)(C₁₋₆ alkyl), —S(═O)₂—Ar, substituted or unsubstituted —S(═O)₂—C₁₋₆ alkyl, substituted or unsubstituted C₁₋₆ alkyl, substituted or unsubstituted C₁₋₆ alkyl-Ar, substituted or unsubstituted —C(═O)C₁₋₆ alkyl-Ar, or substituted or unsubstituted —C(═O)C₁₋₆ alkyl; and

Ar is substituted or unsubstituted aryl, substituted or unsubstituted C₃₋₇ cycloalkyl, substituted or unsubstituted C₈₋₁₄ bicycloalkyl, substituted or unsubstituted C₈₋₁₄ tricycloalkyl, substituted or unsubstituted -(3 to 7) membered heterocycle, substituted or unsubstituted -(7 to 10) membered bicycloheterocycle or substituted or unsubstituted -(5 to 10 membered)heteroaryl, wherein when the foregoing is substituted, each is substituted with one or more substituents selected from cyano, halogen, hydroxyl, nitro, -(3- to 7-membered heterocycle), -(5-to 10 membered)heteroaryl, —O-phenyl, phenyl, —SO₃H, —C₁₋₈ alkyl, —C(═O)—C₁₋₆ alkyl, —C₀₋₆ alkyl-O—C₁₋₆ alkyl, —C₀₋₆ alkyl-C(═O)—NH(C₁₋₆ alkyl), —C₀₋₆ alkyl-C(═O)—N(C₁₋₆ alkyl)(C₁₋₆ alkyl), —C₀₋₆ alkyl-NH—C(═O)—C₁₋₆ alkyl, —C₀₋₆ alkyl-C(═S)—NH(C₁₋₆ alkyl), —C₀₋₆ alkyl-C(═S)—N(C₁₋₆ alkyl)(C₁₋₆ alkyl), —C₀₋₆ alkyl-NH—C(═S)—C₁₋₆ alkyl, —C₀₋₆ alkyl-S(═O)—C₁₋₆ alkyl, —C₀₋₆ alkyl-S(═O)₂—C₁₋₆ alkyl, —C₀₋₆ alkyl-SH, —C₀₋₆ alkyl-S—C₁₋₆ alkyl, —C₀₋₆ alkyl-NH—C(═S)—NH—C₁₋₆ alkyl, —C₀₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —C₀₋₆ alkyl-C(═O)OH, —(C(R′)₂)₀₋₆—O—(C(R′)₂)₁₋₅C(R′)₃, —(C(R′)₂)₀₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂)₀₋₅C(R′)₃;

wherein each of the above substituents can be further substituted with one or more substituents independently selected from cyano, halogen, hydroxyl, nitro, -(3 to 7 membered heterocycle), -(5 to 10 membered)heteroaryl, —O-phenyl, phenyl, —SO₃H, —C(═O)—C₁₋₆ alkyl, —C₀₋₆ alkyl-O—C₁₋₆ alkyl, —C₀₋₆ alkyl-C(═O)—NH(C₁₋₆ alkyl), —C₀₋₆ alkyl-C(═O)—N(C₁₋₆ alkyl)(C₁₋₆ alkyl), —C₀₋₆ alkyl-NH—C(═O)—C₁₋₆ alkyl, —C₀₋₆ alkyl-C(═S)—NH(C₁₋₆ alkyl), —C₀₋₆ alkyl-C(═S)—N(C₁₋₆ alkyl)(C₁₋₆ alkyl), —C₀₋₆ alkyl-NH—C(═S)—C₁₋₆ alkyl, —C₀₋₆ alkyl-S(═O)—C₁₋₆ alkyl, —C₀₋₆ alkyl-S(═O)₂—C₁₋₆ alkyl, —C₀₋₆ alkyl-SH, —C₀₋₆ alkyl-S—C₁₋₆ alkyl, —C₀₋₆ alkyl-NH—C(═S)—NH—C₁₋₆ alkyl, —C₀₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —C₀₋₆ alkyl-C(═O)OH, —(C(R′)₂)₀₋₆—O—(C(R′)₂)₁₋₅C(R′)₃, —(C(R′)₂)₀₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R ′)₃ or —(C(R′)₂)₀₋₆—S(═O)₂—(C(R′)₂)₀₋₅C(R′)₃, or two adjacent substituents together with said aryl or -(5- to 10-membered)heteroaryl form a (C₃₋₈) cycloalkyl, (C₅₋₁₀) cycloalkenyl or -(3- to 7-membered) heterocyclic group may optionally substituted with one or more halogens.

In another embodiment, the present invention encompasses compounds of Formula (I), wherein:

A, B, E, G, W, X, Y, Z, o, and R₁ are as defined above;

Ar is substituted or unsubstituted aryl, substituted or unsubstituted C₃₋₇ cycloalkyl, substituted or unsubstituted C₈₋₁₄ bicycloalkyl, substituted or unsubstituted C₈₋₁₄ tricycloalkyl, substituted or unsubstituted -(3 to 7) membered heterocycle, substituted or unsubstituted -(7 to 10) membered bicycloheterocycle or substituted or unsubstituted -(5 to 10 membered)heteroaryl, wherein when the foregoing is substituted, each is substituted with one or more substituents selected from cyano, halogen, hydroxyl, nitro, -(3- to 7-membered heterocycle), -(5-to 10 membered)heteroaryl, —O-phenyl, phenyl, —SO₃H, C₁₋₈ alkyl, —C(═O)—C₁₋₆ alkyl, —C₁₋₆ alkyl-O—C₁₋₆ alkyl, —C₁₋₆ alkyl-C(═O)—NH(C₁₋₆ alkyl), —C₁₋₆ alkyl-C(═O)—N(C₁₋₆ alkyl)(C₁₋₆ alkyl), —C₁₋₆ alkyl-NH—C(═O)—C₁₋₆ alkyl, —C₁₋₆ alkyl(═S)—NH(C₁₋₆ alkyl), —C₁₋₆ alkyl(═S)—N(C₁₋₆ alkyl)(C₁₋₆ alkyl), —C₁₋₆ alkyl-NH—C(═S)—C₁₋₆ alkyl, —C₁₋₆ alkyl-S(═O)—C₁₋₆ alkyl, —C₁₋₆ alkyl-S(═O)₂—C₁₋₆ alkyl, —C₁₋₆ alkyl-SH, —C₁₋₆ alkyl-S—C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₁₋₆ alkyl-C(═O)O—C₁₋₆ alkyl, —C₁₋₆ alkyl(═O)OH, —(C(R′)₂)₀₋₆—O—(C(R′)₂)₁₋₅C(R′)₃, —(C(R′)₂)₁₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂)₁₋₅C(R′)₃;

wherein each of the above substituents can be further substituted with one or more substituents independently selected from cyano, halogen, hydroxyl, nitro, -(3 to 7 membered heterocycle), -(5 to 10 membered)heteroaryl, —O-phenyl, phenyl, —SO₃H, —C(═O)—C₁₋₆ alkyl, —C₁₋₆ alkyl-O—C₁₋₆ allyl, —C₁₋₆ alkyl-C(═O)—NH(C₁₋₆ alkyl), —C₁₋₆ alkyl-C(═O)—N(C₁₋₆ alkyl)(C₁₋₆ alkyl —C₁₋₆ alkyl-NH—C(═O)—C₁₋₆ alkyl, —C₁₋₆ alkyl(═S)—NH(C₁₋₆ alkyl), —C₁₋₆ alkyl(═S)—N(C₁₋₆ alkyl)(C₁₋₆ alkyl), —C₁₋₆ alkyl-NH—C(═S)—C₁₋₆ alkyl, —C₁₋₆ alkyl-S(═O)—C₁₋₆ alkyl, —C₁₋₆ alkyl-S(═O)₂—C₁₋₆ alkyl, —C₁₋₆ alkyl-SH, —C₁₋₆ alkyl-S—C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₁₋₆ alkyl-C(═O)O—C₁₋₆ alkyl, —C₁₋₆ alkyl-C(═O)OH, —(C(R′)₂)₀₋₆—O—(C(R′)₂)₁₋₅C(R′)₃, —(C(R′)₂)₁₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂—(C(R′)₂)₁₋₅C(R′)₃, or two adjacent substituents together with said aryl or -(5- to 10-membered)heteroaryl form a (C₃₋₈) cycloalkyl, (C₅₋₁₀) cycloalkenyl or -(3- to 7-membered) heterocyclic group may optionally substituted with one or more halogens; and

when X is —CR₄—, R₄ is H, hydroxy, amino, cyano, nitro, Br, Cl, C₁-C₇ alkyl substituted with halogen, substituted C₁₋₈ alkyl, substituted or unsubstituted C₂₋₆ alkenyl, substituted or unsubstituted C₂₋₆ alkynyl, substituted or unsubstituted C₃₋₈ cycloalkyl, —C(═O)—O—C₁₋₆ alkyl, —C₁₋₆ alkyl-O—C₁₋₆ alkyl, —C₁₋₆ alkyl-C(═O)—NH(C₁₋₆ alkyl), —C₁₋₆ alkyl-C(═O)—N(C₁₋₆ alkyl)(C₁₋₆ alkyl), —C₁₋₆ alkyl-NH—C(═O)—C₁₋₆ alkyl, —C₁₋₆ alkyl-S(═O)—C₁₋₆ alkyl, —C₁₋₆ alkyl-S(═O)₂—C₁₋₆ alkyl, —C₁₋₆ alkyl-SH, —C₁₋₆ alkyl-S—C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₁₋₆ alkyl-C(═O)O—C₁₋₆ alkyl, —(C(R′)₂)₀₋₆—O—(C(R′)₂)₁₋₅C(R′)₃, —(C(R′)₂)₁₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂—(C(R′)₂)₁₋₅C(R′)₃.

### 5.2 COMPOUNDS OF THE INVENTION OF FORMULA (II)

In one embodiment, the Compounds of the Invention are those where W, X, Y and Z are —CR₃, —CR₄, —CR₅ and —CR₆, respectively; o is 0; and A and B are both unsubstituted —(CH₂)₂— as set forth in Formula (II): 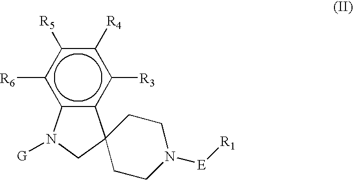
and pharmaceutically acceptable salts, free bases, solvates, hydrates, stereoisomers, clathrates or prodrugs thereof, where G, E, R₁, R₃, R₄, R₅, and R₆ are as defined above for the compounds of Formula (I).

In one embodiment, E is —(CH₂)ₚ— wherein p is 0, 1, or 2. In some embodiments, compounds of invention are represented by Formula (IIb) as shown below: 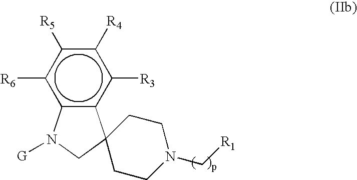
wherein each variable in Formula (IIb) has the same meaning as described herein, and p is 0, 1, or 2. In some embodiments, p is 0. In other embodiments, p is 1. In still other embodiments, p is 2.

In another embodiment, p is 1 or 2 and R₁ is —CH═CH₂.

In another embodiment, p is 1 or 2 and R₁ is -cyclopropyl.

In another embodiment, p is 1 or 2 and R₁ is —CH₂CH₃.

In another embodiment, p is 1 or 2 and R₁ is —(CH₂)₂CH₃.

In another embodiment, p is 0 or 1 and R₁ is substituted or unsubstituted phenyl.

In another embodiment, p is 1 and R₁ is —CH(OH)CH₃.

In another embodiment, p is 1 and R₁ is —C(═CH₂)CH₃.

In another embodiment, p is 1 and R₁ is H.

In another embodiment, G is —C(═O)—Ar, —C(═O)NH—Ar or —C(═O)NR₈R₈′ wherein R₈ and R₈′ taken together with the nitrogen to which they are attached form a 3 to 7 membered heterocyclic or heteroaromatic ring having one or more nitrogen, oxygen or sulfur atoms. Preferred groups are morphilino, pyrrolidano, piperidino or imidazolino rings which can be substituted or unsubstituted.

In another embodiment, G is —C(═O)CH₂—Ar.

In another embodiment, G is —C(═O)CH—(Ar)₂.

In another embodiment, G is —C(═O)NH—(Ar).

In another embodiment, G is —S(═O)₂—Ar.

In another embodiment, Ar is substituted or unsubstituted phenyl. Preferably Ar is mono or disubstituted phenyl wherein the substituents are selected from halogen, lower alkyl, lower alkenyl, lower alkoxy and C₃₋₇ cycloalkyl.

In another embodiment, Ar is methoxy phenyl substituted in the para position.

In another embodiment, Ar is fluorophenyl substituted in the ortho position.

In another embodiment, Ar is fluorophenyl substituted in the para position.

In another embodiment, Ar is difluorophenyl substituted in the ortho and para positions.

In another embodiment, Ar is difluorophenyl substituted in the ortho and meta positions.

In another embodiment, Ar is difluorophenyl substituted in the ortho positions.

In another embodiment, Ar is difluorophenyl substituted in the meta positions.

In another embodiment, Ar is substituted or unsubstituted furan.

In another embodiment, Ar is substituted or unsubstituted pyridine.

In another embodiment, Ar is substituted or unsubstituted thiophene.

In another embodiment, Ar is substituted or unsubstituted adamantane.

In another embodiment, Ar is 2-chlorothiophene.

In another embodiment, Ar is benzo(1,3)dioxole.

In another embodiment, Ar is fluoren-9-one.

In another embodiment, Ar is morpholine.

In another embodiment, p is 0; and in another embodiment, p is 1.

In another embodiment, one or more of R₃—R₆ is a substituent other than H.

In another embodiment, two or more of R₃—R₆ is a substituent other than H.

In another embodiment, three or more of R₃—R₆ is a substituent other than H.

In another embodiment, each of R₃—R₆ is a substituent other than H.

Preferred R₃—R₆ groups include halogen, preferably fluoro or chloro; —C₁₋₆ alkyl, preferably methyl; —O—C₁₋₆ alkyl, preferably methoxy; and hydroxy.

### 5.3 COMPOUNDS OF THE INVENTION OF FORMULA (III)

In one embodiment, the Compounds of the Invention are those where W, X, Y and Z are —CR₃, —CR₄, —CR₅ and —CR₆, respectively; o is 0; A and B are both unsubstituted —(CH₂)₂—; and G is —C(═O)—Ar as set forth in Formula (III): 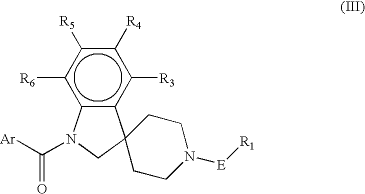
and pharmaceutically acceptable salts, free bases, solvates, hydrates, stereoisomers, clathrates or prodrugs thereof, where Ar, E, R₁, R₃, R₄, R₅, and R₆ are as defined above for the Compounds of the Invention of Formula (I).

In one embodiment, E is —(CH₂)ₚ— wherein p is 0, 1, or 2. In some embodiments, compounds of invention are represented by Formula (IIIb) as shown below: 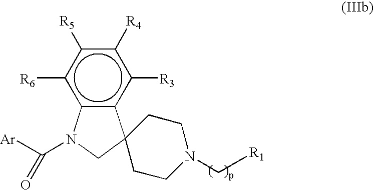
wherein each variable in Formula (IIIb) has the same meaning as described herein, and p is 0, 1, or 2. In some embodiments, p is 0. In other embodiments, p is 1. In still other embodiments, p is 2.

In another embodiment, p is 1 and R₁ is C₂-₆ alkenyl, preferably —CH═CH₂.

In another embodiment, p is 1 and R₁ is C₃-C₇ cycloalkyl, preferably—cyclopropyl or cyclobutyl.

In another embodiment, p is 1 and R₁ is C₁₋₆ alkyl, preferably —CH₂CH₃.

In another embodiment, p is 1 and R₁ is C₁₋₆ alkyl, preferably —(CH₂)₂CH₃.

In another embodiment, p is 0 and R₁ is substituted or unsubstituted phenyl.

In another embodiment, p is 1 and R₁ is —CH(OH)CH₃.

In another embodiment, p is 1 and R₁ is —C(═CH₂)CH₃.

In another embodiment, p is 0 and R₁ is H.

In another embodiment, p is 1 and R₁ is H.

In another embodiment, Ar is substituted or unsubstituted phenyl, substituted or unsubstituted naphthalene, substituted or unsubstituted thiophene, substituted or unsubstituted pyrindine, pyrazole, pyrrole, quinazoline, pyrazine or quinoline.

In another embodiment, Ar is methoxy phenyl substituted in the para position.

In another embodiment, Ar is fluorophenyl substituted in the ortho position.

In another embodiment, Ar is fluorophenyl substituted in the para position.

In another embodiment, Ar is difluorophenyl substituted in the ortho and para positions.

In another embodiment, Ar is difluorophenyl substituted in the ortho and meta positions.

In another embodiment, Ar is difluorophenyl substituted in the ortho positions.

In another embodiment, Ar is difluorophenyl substituted in the meta positions.

In another embodiment, Ar is substituted or unsubstituted furan.

In another embodiment, Ar is substituted or unsubstituted pyridine.

In another embodiment, Ar is substituted or unsubstituted thiophene.

In another embodiment, Ar is substituted or unsubstituted adamantane.

In another embodiment, Ar is 2-chlorothiophene.

In another embodiment, Ar is benzo(1,3)dioxole.

In another embodiment, Ar is fluoren-9-one.

In another embodiment, Ar is morpholine.

In another embodiment, p is 0; and in another embodiment, p is 1.

In another embodiment, one or more of R₃—R₆ is a substituent other than H.

In another embodiment, two or more of R₃—R₆ is a substituent other than H.

In another embodiment, three or more of R₃—R₆ is a substituent other than H.

In another embodiment, each of R₃—R₆ is a substituent other than H.

Preferred R₃—R₆ groups include halogen, preferably fluoro or chloro; —C₁₋₆ alkyl, preferably methyl; and —O—C₁₋₆ alkyl, preferably methoxy.

### 5.4 COMPOUNDS OF THE INVENTION OF FORMULA (IV)

In one embodiment, the Compounds of the Invention are those where W, X, Y and Z are —CR₃, —CR₄, —CR₅ and —CR₆, respectively; o is 0; A and B are both unsubstituted —(CH₂)₂—; and G is —S(═O)₂—Ar as set forth in Formula (IV): 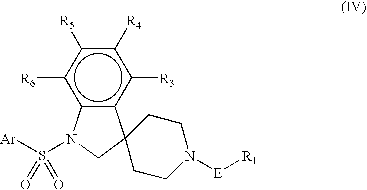
and pharmaceutically acceptable salts, free bases, solvates, hydrates, stereoisomers, clathrates or prodrugs thereof, where Ar, E, R₁, R₃, R₄, R₅, and R₆ are as defined above for the Compounds of the Invention of Formula (I).

In one embodiment, E is —(CH₂)ₚ— wherein p is 0, 1, or 2. In some embodiments, compounds of invention are represented by Formula (IVb) as shown below: 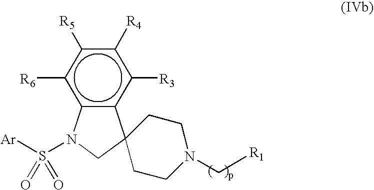
wherein each variable in Formula (IVb) has the same meaning as described herein, and p is 0, 1, or 2. In some embodiments, p is 0. In other embodiments, p is 1. In still other embodiments, p is2.

In another embodiment, p is 1 and R₁ is C₂₋₆ alkenyl, preferably —CH═CH₂.

In another embodiment, p is 1 and R₁ is C₃-C₇ cycloalkyl, preferably -cyclopropyl.

In another embodiment, p is 1 and R₁ is C₁₋₆ alkyl, preferably —CH₂CH₃.

In another embodiment, p is 1 and R₁ is C₁₋₆ alkyl, preferably —(CH₂)₂CH₃.

In another embodiment, p is 0 and R₁ is substituted or unsubstituted phenyl.

In another embodiment, p is 1 and R₁ is —CH(OH)CH₃.

In another embodiment, p is 1 and R₁ is —C(═CH₂)CH₃.

In another embodiment, p is 1 and R₁ is H.

In another embodiment, p is 0 and R₁ is H.

In another embodiment, Ar is substituted or unsubstituted phenyl.

In another embodiment, Ar is methoxy phenyl substituted in the para position.

In another embodiment, Ar is fluorophenyl substituted in the ortho position.

In another embodiment, Ar is fluorophenyl substituted in the para position.

In another embodiment, Ar is difluorophenyl substituted in the ortho and para positions.

In another embodiment, Ar is difluorophenyl substituted in the ortho and meta positions.

In another embodiment, Ar is difluorophenyl substituted in the ortho positions.

In another embodiment, Ar is difluorophenyl substituted in the meta positions.

In another embodiment, Ar is substituted or unsubstituted furan.

In another embodiment, Ar is substituted or unsubstituted pyridine.

In another embodiment, Ar is substituted or unsubstituted thiophene.

In another embodiment, Ar is substituted or unsubstituted adamantane.

In another embodiment, Ar is 2-chlorothiophene.

In another embodiment, Ar is benzo(1,3)dioxole.

In another embodiment, Ar is fluoren-9-one.

In another embodiment, Ar is morpholine.

In another embodiment, p is 0; and in another embodiment, p is 1.

In another embodiment, one or more of R₃—R₆ is a substituent other than H.

In another embodiment, two or more of R₃—R₆ is a substituent other than H.

In another embodiment, three or more of R₃—R₆ is a substituent other than H.

In another embodiment, each of R₃—R₆ is a substituent other than H.

Preferred R₃—R₆ groups include halogen, preferably fluoro or chloro; —C₁₋₆ alkyl, preferably methyl; and —O—C₁₋₆ alkyl, preferably methoxy.

### 5.5 COMPOUNDS OF THE INVENTION OF FORMULA (V)

In one embodiment, the Compounds of the Invention are those where W, X, Y and Z are —CR₃, —CR₄, —CR₅ and —CR₆, respectively; o is 0; A and B are both unsubstituted —(CH₂)₂—; and G is —C(═O)—Ar as set forth in Formula (V): 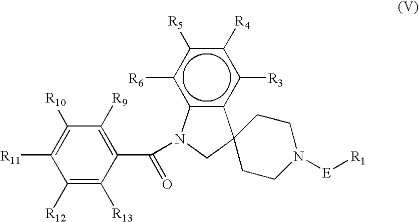
and pharmaceutically acceptable salts, free bases, solvates, hydrates, stereoisomers, clathrates or prodrugs thereof, where E, R₁, R₃, R₄, R₅, and R₆ are as defined above for the Compounds of the Invention of Formula (I), and R₉—R₁₃ are each independently H, halogen, nitro, substituted or unsubstituted C₁₋₆ alkyl, substituted or unsubstituted —O—C₁₋₆ alkyl or R₁₀ and R₁₁ taken together form —O—CH₂—O—.

In one embodiment, E is —(CH₂)ₚ— wherein p is 0, 1, or 2. In some embodiments, compounds of invention are represented by Formula (Vb) as shown below: 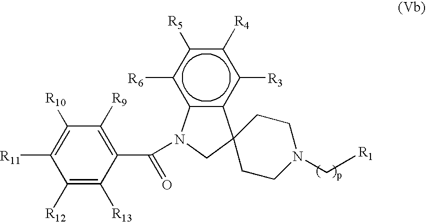
wherein each variable in Formula (Vb) has the same meaning as described herein, and p is 0, 1, or 2. hi some embodiments, p is 0. In other embodiments, p is 1. hi still other embodiments, p is2.

In another embodiment, R₉—R₁₃ are each H.

In another embodiment, R₁₀—R₁₃ are H and R₉ is halogen, preferably fluoro or chloro.

In another embodiment, R₉, R₁₀, R₁₂ and R₁₃ are H and R₁₁ is methoxy.

In another embodiment, R₉, R₁₀, R₁₂ and R₁₃ are H and R₁₁ is nitro.

In another embodiment, R₉, R₁₂ and R₁₃ are H, R₁₀ is nitro and R₁₁ is methyl.

In another embodiment, R₉, R₁₀, R₁₂ and R₁₃ are H and R₁₁ is halogen, preferably fluoro or chloro.

In one embodiment, R₁₀—R₁₃ are H and R₉ is methoxy.

In another embodiment, R₉, R₁₂ and R₁₃ are H and R₁₀ and R₁₁ taken together form —O—CH₂—O—.

In another embodiment, R₉, R₁₂ and R₁₃ are H and R₁₀ and R₁₁ are each halogen, preferably fluoro or chloro.

In another embodiment, R₉, R₁₀, R₁₂ and R₁₃ are H and R₁₁ is halogen, preferably fluoro or chloro.

In another embodiment, R₉, R₁₁ and R₁₃ are H, and R₁₀ and R₁₂ are each halogen, preferably fluoro or chloro.

In another embodiment, R₉, R₁₁ and R₁₃ are H, and R₁₀ and R₁₂ are each methoxy.

In another embodiment, R₉ and R₁₁—R₁₃ are H, and R₁₀ is halogen, preferably fluoro chloro.

In another embodiment, R₁₁—R₁₃ are H and R₉ and R₁₀ are each halogen, preferably fluoro or chloro.

In another embodiment, R₁₀, R₁₂ and R₁₃ are H and R₉ and R₁₁ are each halogen, preferably fluoro or chloro.

In another embodiment, R₉ and R₁₁—R₁₃ are H, and R₁₀ is trifluoromethyl.

In another embodiment, R₉, R₁₁ and R₁₃ are H, and R₁₀ and R₁₂ are each trifluoromethyl.

In another embodiment, R₉ and R₁₁—R₁₃ are H, and R₁₀ is nitro.

In another embodiment, R₉, R₁₂ and R₁₃ are H, R₁₀ is trifluoromethyl and R₁₁ is halogen, preferably fluoro or chloro.

In another embodiment, R₉ and R₁₁—R₁₃ are H, and R₁₀ is dichloromethyl.

In another embodiment, R₉ and R₁₃ are H, and R₁₀, R₁₁ and R₁₂ are each methoxy.

In another embodiment, R₁₀, R₁₁ and R₁₃ are H and R₉ and R₁₂ are each halogen, preferably fluoro or chloro.

In another embodiment, R₁₀—R₁₂ are H and R₉ and R₁₃ are each halogen, preferably fluoro or chloro.

In another embodiment, R₁₁—R₁₃ are H and R₉ and R₁₀ are each halogen, preferably fluoro or chloro.

In another embodiment, p is 1 and R₁ is C₂₋₆ alkenyl, preferably —CH═CH₂.

In another embodiment, p is 1 and R₁ is C₃₋₇ cycloalkyl, preferably -cyclopropyl or cyclobutyl.

In another embodiment, p is 1 and R₁ is C₁₋₆ alkyl, preferably —CH₂CH₃.

In another embodiment, p is 1 and R₁ is C₁₋₆ alkyl, preferably —(CH₂)₂CH₃.

In another embodiment, p is 0 and R₁ is substituted or unsubstituted phenyl.

In another embodiment, p is 1 and R₁ is —CH(OH)CH₃.

In another embodiment, p is 1 and R₁ is —C(═CH₂)CH₃.

In another embodiment, p is 0 and R₁ is H.

In another embodiment, p is 1 and R₁ is H.

In another embodiment, one or more of R₃—R₆is a substituent other than H.

In another embodiment, two or more of R₃—R₆ is a substituent other than H.

In another embodiment, three or more of R₃—R₆ is a substituent other than H.

In another embodiment, each of R₃—R₆is a substituent other than H.

Preferred R₃—R₆ groups include halogen, preferably fluoro or chloro; —C₁₋₆ alkyl, preferably methyl, isopropyl or t-butyl; and —O—C₁₋₆ alkyl, preferably methoxy.

In one embodiment, compounds of invention are represented by Formula (Vc) as shown below:

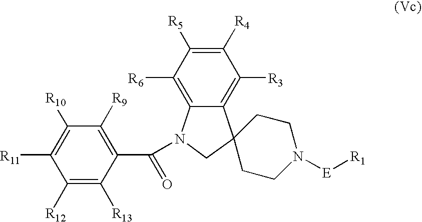

wherein:

R₁ is C₃₋₇ cycloalkyl;

E is a substituted or unsubstituted C₁₋₂ alkylene;

R₃, R₅ and R₆ are each independently selected from the group consisting of H, C₁₋₆ acyl, C₁₋₆ acyloxy, C₂₋₆ alkenyl, C₁₋₆ alkoxy, C₁₋₆ alkyl, C₁₋₆ alkylamino, C₁₋₆ alkylcarboxamide, alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, arylsulfonyl, carbo-C₁₋₆-alkoxy, carboxamide, carboxy, cyano, C₃₋₈ cycloalkyl, C₁₋₆ dialkylamino, C₁₋₆ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heterocyclic, heterocyclicsulfonyl, hydroxyl, nitro, phenoxy, phenyl, sulfonamide, sulfonic acid, and thiol;

R₄ is C₁₋₆ alkyl; and

R₉—R₁₃ are each independently selected from the group consisting of H, C₁₋₆ acyl, C₁₋₆ acyloxy, C₂₋₆ alkenyl, C₁₋₆ alkoxy, C₁₋₆ alkyl, C₁₋₆ alkylamino, C₁₋₆ alkylcarboxamide, C₂₋₆-alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, arylsulfonyl, carbo-C₁₋₆-alkoxy, carboxamide, carboxy, cyano, C₃₋₈ cycloalkyl, C₁₋₆ dialkylamino, C₁₋₆ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heterocyclic, heterocyclicsulfonyl, hydroxyl, nitro, phenoxy, phenyl, sulfonamide, sulfonic acid, and thiol.

In some embodiments, compounds are of Formula (Vc) wherein E is —CH₂— or —CH₂CH₂—.

In some embodiments, compounds are of Formula (Vc) wherein R₁ is cyclopropyl, cyclobutyl or cyclopentyl.

The invention also includes specific subclasses of the compounds of Formula (I) wherein G is —C(═O)—Ar, —(CH₂)ₒ— is absent and X is —C(F)—, —C(OCH₃)— or —C(CH₃)—, then W, Y and Z are not all —CH—. Similarly, the invention encompasses, in another embodiment, a specific subclass of the compounds of Formula (IIb) wherein when G is —C(═O)—Ar and R₄ is —OCH₃, —F or —CH₃, then R₃, R₅ and R₆ are not all hydrogen. Finally, the invention includes a specific subclass of the compounds of Formula (IIIb) wherein when R₄ is —F, —OCH₃ or —CH₃, then R₆, R₅ and R₃ are not all hydrogen, or p of —(CH₂)ₚ— is not 1, or when p is 0, R₁ is not cycloalkyl or —CH₃.

When the groups described herein are said to be “substituted or unsubstituted,” when substituted, they may be substituted with any desired substituent or substituents that do not adversely affect the desired activity of the compound. Examples of preferred substituents are those found in the exemplary compounds and embodiments disclosed herein, as well as halogen (chloro, iodo, bromo, or fluoro); C₁₋₆ alkyl; C₂₋₆ alkenyl; C₂₋₆ alkynyl; hydroxyl; C₁₋₆ alkoxyl; amino; nitro; thiol; thioether; imine; cyano; amido; phosphonato; phosphine; carboxyl; thiocarbonyl; sulfonyl; sulfonamide; ketone; aldehyde; ester; oxygen (═O); haloalkyl (e.g., trifluoromethyl); carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl); carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrinidinyl, benzimidazolyl, benzothiophenyl, or benzofuranyl); amino primary, secondary, or tertiary); O-lower alkyl; O-aryl, aryl; aryl-lower alkyl; CO₂CH₃; CONH₂; OCH₂CONH₂; NH₂; SO₂NH₂; OCHF₂; CF₃; OCF₃; may also be optionally substituted by a fused-ring structure or bridge, for example —OCH₂O—.

These substituents may optionally be further substituted with a substituent selected from such groups.

### 5.6 ILLUSTRATIVE COMPOUNDS OF THE INVENTION

Set forth below are illustrative Compounds of the Invention including their chemical names.

TABLE 1

| Cmpd | | |
|---|---|---|
| No. | COMPOUND STRUCTURE | CHEMICAL NAME |
| 1 | 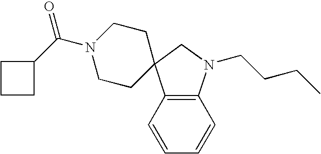 | 1′-(cyclobutylcarbonyl)- 1,2-dihydro-1-butyl- spiro[3H-indole-3,4′- piperidine] |
| 2 | 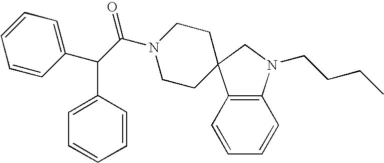 | 1′-(diphenylacetyl)-1,2- dihydro-1-butyl-spiro[3H- indole-3,4′-piperidine] |
| 3 | 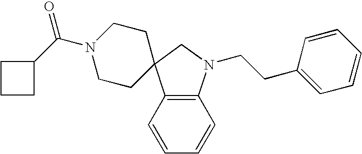 | 1′-(cyclobutylcarbonyl)- 1,2-dihydro-1-phenethyl- spiro[3H-indole-3,4′- piperidine] |
| 4 | 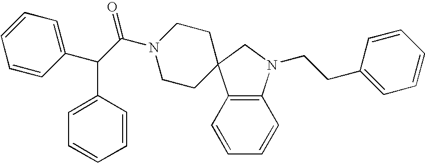 | 1′-(diphenylacetyl)-1,2- dihydro-1-phenethyl- spiro[3H-indole-3,4′- piperidine] |
| 5 | 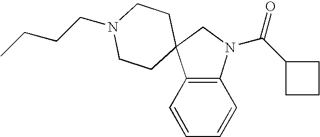 | 1′-(butyl)-1,2-dihydro-1- (cyclobutylcarbonyl)- spiro[3H-indole-3,4′- piperidine] |
| 6 | 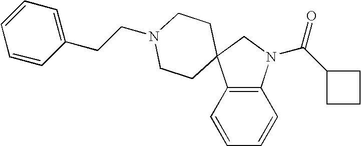 | 1′-(phenethyl)-1,2- dihydro-1- (cyclobutylcarbonyl)- spiro[3H-indole-3,4′- piperidine] |
| 7 | 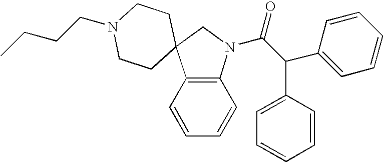 | 1′-(butyl)-1,2-dihydro-1- (diphenylacetyl)-spiro[3H- indole-3,4′-piperidine] |
| 8 | 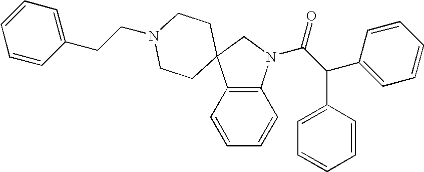 | 1′-(phenethyl)-1,2- dihydro-1- (diphenylacetyl)-spiro[3H- indole-3,4′-piperidine] |
| 9 | 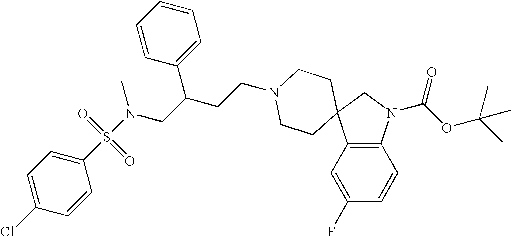 | 1′-{4-[(4-chloro- benzenesulfonyl)-methyl- amino]-3-phenyl-butyl}- 1,2-dihydro-1-(tert- butoxycarbonyl)-5-fluoro- spiro[3H-indole-3,4′- piperidine] |
| 10 | 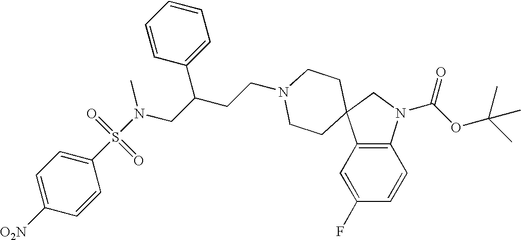 | 1′-{4-[(4-nitro- benzenesulfonyl)-methyl- amino]-3-phenyl-butyl}- 1,2-dihydro-1-(tert- butoxycarbonyl)-5-fluoro- spiro[3H-indole-3,4′- piperidine] |
| 11 | 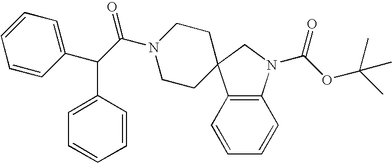 | 1′-(diphenylacetyl)-1,2- dihydro-1-(tert- butoxycarbonyl)-spiro[3H- indole-3,4′-piperidine] |
| 12 | 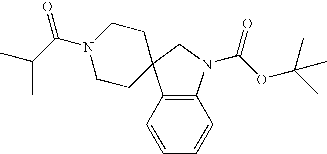 | 1′-(isobutyryl)-1,2- dihydro-1-(tert- butoxycarbonyl)-spiro[3H- indole-3,4′-piperidine] |
| 13 | 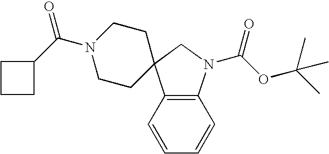 | 1′-(cyclobutylcarbonyl)- 1,2-dihydro-1-(tert- butoxycarbonyl)-spiro[3H- indole-3,4′-piperidine] |
| 14 | 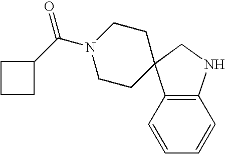 | 1′-(cyclobutylcarbonyl)- 1,2-dihydro-spiro[3H- indole-3,4′-piperidine] |
| 15 | 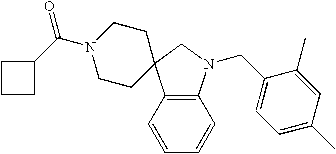 | 1′-(cyclobutylcarbonyl)- 1,2-dihydro-1-(2,4- dimethyl-benzyl)- spiro[3H-indole-3,4′- piperidine] |
| 16 | 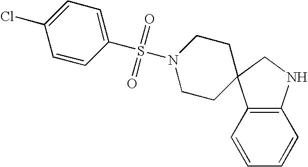 | 1′-(4-chloro- benzenesulfonyl)-1,2- dihydro-spiro[3H-indole- 3,4′-piperidine] |
| 17 | 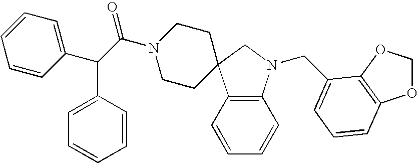 | 1′-(diphenylacetyl)-1,2- dihydro-1- (benzo[1,3]dioxol-4- ylmethyl)-spiro[3H- indole-3,4′-piperidine] |
| 18 | 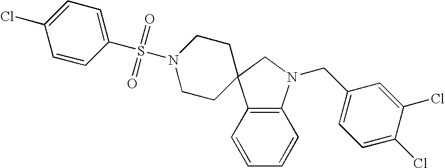 | 1′-(4-chloro- benzenesulfonyl)-1,2- dihydro-1-(3,4-dichloro- benzyl)-spiro[3H-indole- 3,4′-piperidine] |
| 19 | 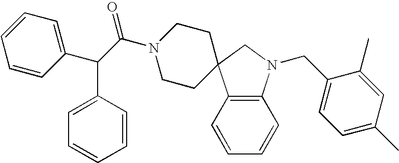 | 1′-(diphenylacetyl)-1,2- dihydro-1-(2,4-dimethyl- benzyl)-spiro[3H-indole- 3,4′-piperidine] |
| 20 | 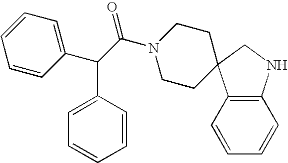 | 1′-(diphenylacetyl)-1,2- dihydro-spiro[3H-indole- 3,4′-piperidine] |
| 21 | 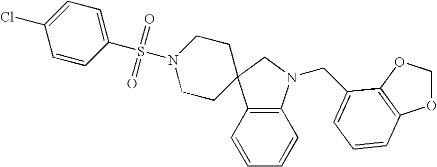 | 1′-(4-chloro- benzenesulfonyl)-1,2- dihydro-1- (benzo[1,3]dioxol-4- ylmethyl)-spiro[3H- indole-3,4′-piperidine] |
| 22 | 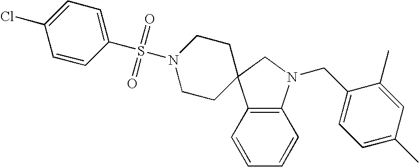 | 1′-(4-chloro- benzenesulfonyl)-1,2- dihydro-1-(2,4-dimethyl- benzyl)-spiro[3H-indole- 3,4′-piperidine] |
| 23 | 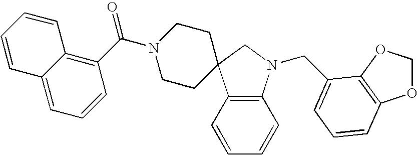 | 1′-(naphthalene-1- carbonyl)-1,2-dihydro-1- (benzo[1,3]dioxol-4- ylmethyl)-spiro[3H- indole-3,4′-piperidine] |
| 24 | 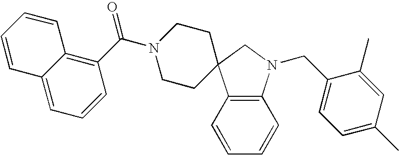 | 1′-(naphthalene-1- carbonyl)-1,2-dihydro-1- (2,4-dimethyl-benzyl)- spiro[3H-indole-3,4′- piperidine] |
| 25 | 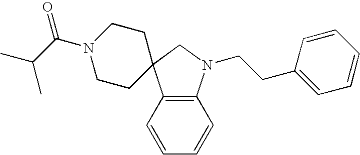 | 1′-(isobutyryl)-1,2- dihydro-1-(phenethyl)- spiro[3H-indole-3,4′- piperidine] |
| 26 | 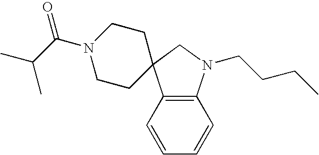 | 1′-(isobutyryl)-1,2- dihydro-1-(butyl)- spiro[3H-indole-3,4′- piperidine] |
| 27 | 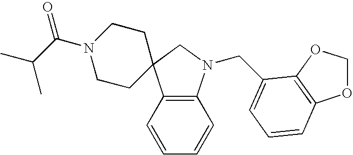 | 1′-(isobutyryl)-1,2- dihydro-1- (benzo[1,3]dioxol-4- ylmethyl)-spiro[3H- indole-3,4′-piperidine] |
| 28 | 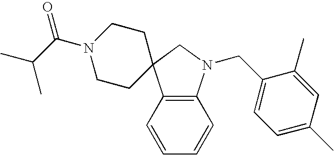 | 1′-(isobutyryl)-1,2- dihydro-1-(2,4-dimethyl- benzyl)-spiro[3H-indole- 3,4′-piperidine] |
| 29 | 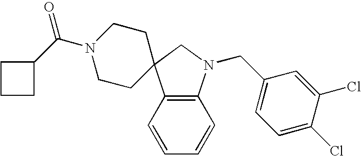 | 1′-(cyclobutylcarbonyl)- 1,2-dihydro-1-(3,4- dichloro-benzyl)- spiro[3H-indole-3,4′- piperidine] |
| 30 | 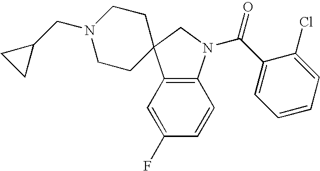 | 1′-(cyclopropylmethyl)- 1,2-dihydro-5-fluoro-1-(2- chloro-benzoyl)-spiro[3H- indole-3,4′-piperidine] |
| 31 | 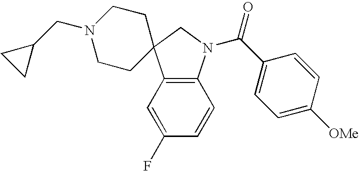 | 1′-(cyclopropylmethyl)- 1,2-dihydro-5-fluoro-1-(4- methoxy-benzoyl)- spiro[3H-indole-3,4′- piperidine] |
| 32 | 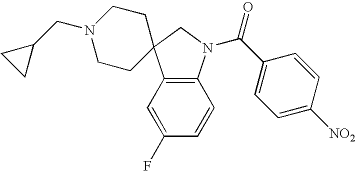 | 1′-(cyclopropylmethyl)- 1,2-dihydro-5-fluoro-1-(4- nitro-benzoyl)-spiro[3H- indole-3,4′-piperidine] |
| 33 | 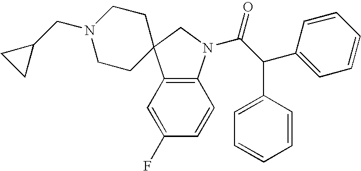 | 1′-(cyclopropylmethyl)- 1,2-dihydro-5-fluoro-1- (diphenylacetyl)-spiro[3H- indole-3,4′-piperidine] |
| 34 | 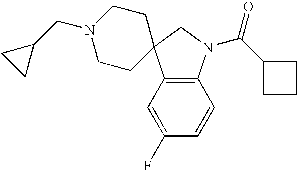 | 1′-(cyclopropylmethyl)- 1,2-dihydro-5-fluoro-1- (cyclobutylcarbonyl)- spiro[3H-indole-3,4′- piperidine] |
| 35 | 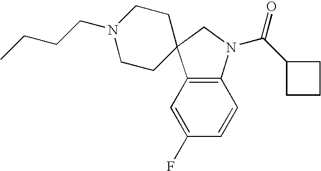 | 1′-(butyl)-1,2-dihydro-5- fluoro-1- (cyclobutylcarbonyl)- spiro[3H-indole-3,4′- piperidine] |
| 36 | 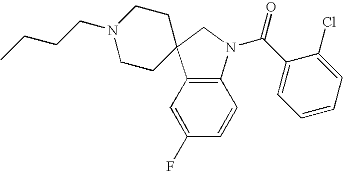 | 1′-(butyl)-1,2-dihydro-5- fluoro-1-(2-chloro- benzoyl)-spiro[3H-indole- 3,4′-piperidine] |
| 37 | 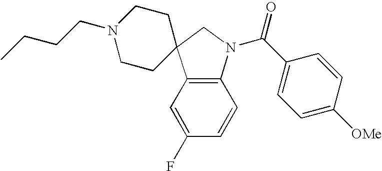 | 1′-(butyl)-1,2-dihydro-5- fluoro-1-(4-methoxy- benzoyl)-spiro[3H-indole- 3,4′-piperidine] |
| 38 | 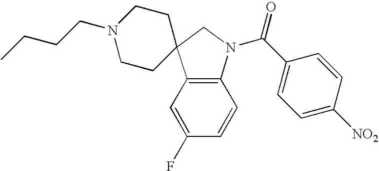 | 1′-(butyl)-1,2-dihydro-5- fluoro-1-(4-nitro-benzoyl)- spiro[3H-indole-3,4′- piperidine] |
| 39 | 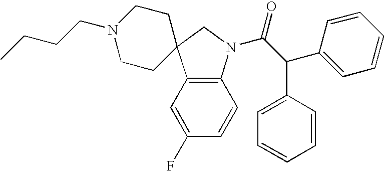 | 1′-(butyl)-1,2-dihydro-5- fluoro-1-(diphenylacetyl)- spiro[3H-indole-3,4′- piperidine] |
| 40 | 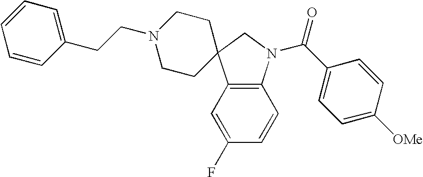 | 1′-(phenethyl)-1,2- dihydro-5-fluoro-1-(4- methoxy-benzoyl)- spiro[3H-indole-3,4′- piperidine] |
| 41 | 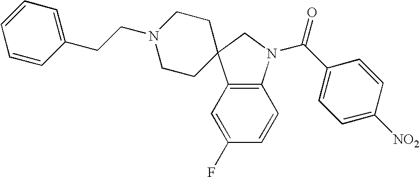 | 1′-(phenethyl)-1,2- dihydro-5-fluoro-1-(4- nitro-benzoyl)-spiro[3H- indole-3,4′-piperidine] |
| 42 | 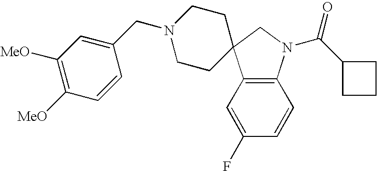 | 1′-(3,4-dimethoxy- benzyl)-1,2-dihydro-5- fluoro-1- (cyclobutylcarbonyl)- spiro[3H-indole-3,4′- piperidine] |
| 43 | 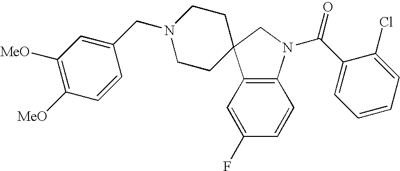 | 1′-(3,4-dimethoxy- benzyl)-1,2-dihydro-5- fluoro-1-(2-chloro- benzoyl)-spiro[3H-indole- 3,4′-piperidine] |
| 44 | 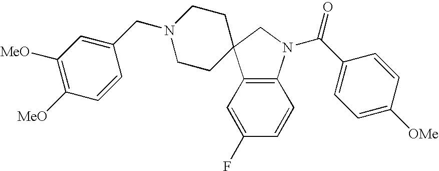 | 1′-(3,4-dimethoxy- benzyl)-1,2-dihydro-5- fluoro-1-(4-methoxy- benzoyl)-spiro[3H-indole- 3,4′-piperidine] |
| 45 | 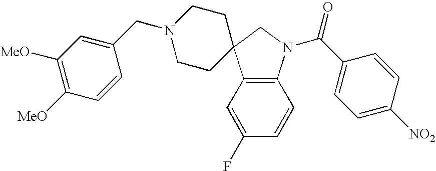 | 1′-(3,4-dimethoxy- benzyl)-1,2-dihydro-5- fluoro-1-(4-nitro-benzoyl)- spiro[3H-indole-3,4′- piperidine] |
| 46 | 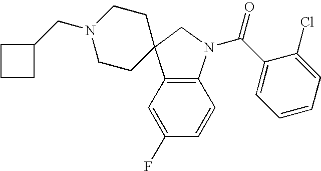 | 1′-(cyclopropylmethyl)- 1,2-dihydro-5-fluoro-1-(2- chloro-benzoyl)-spiro[3H- indole-3,4′-piperidine] |
| 47 | 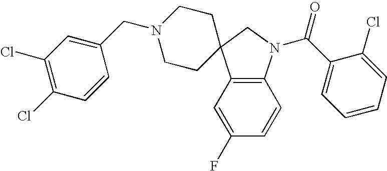 | 1′-(3,4-dichloro-benzyl)- 1,2-dihydro-5-fluoro-1-(2- chloro-benzoyl)-spiro[3H- indole-3,4′-piperidine] |
| 48 | 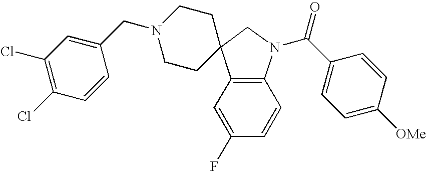 | 1′-(3,4-dichloro-benzyl)- 1,2-dihydro-5-fluoro-1-(4- methoxy-benzoyl)- spiro[3H-indole-3,4′- piperidine] |
| 49 | 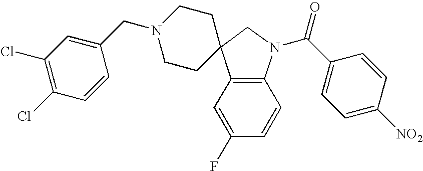 | 1′-(3,4-dichloro-benzyl)- 1,2-dihydro-5-fluoro-1-(4- nitro-benzoyl)-spiro[3H- indole-3,4′-piperidine] |
| 50 | 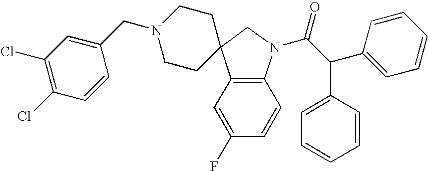 | 1′-(3,4-dichloro-benzyl)- 1,2-dihydro-5-fluoro-1- (diphenylacetyl)-spiro[3H- indole-3,4′-piperidine] |
| 51 | 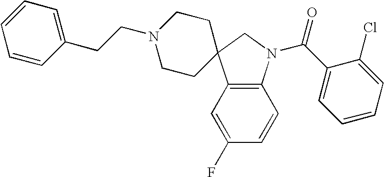 | 1′-(phenethyl)-1,2- dihydro-5-fluoro-1-(2- chloro-benzoyl)-spiro[3H- indole-3,4′-piperidine] |
| 52 | 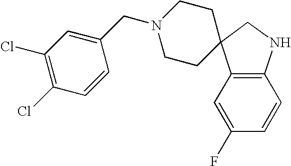 | 1′-(3,4-dichloro-benzyl)- 1,2-dihydro-5-fluoro- spiro[3H-indole-3,4′- piperidine] |
| 53 | 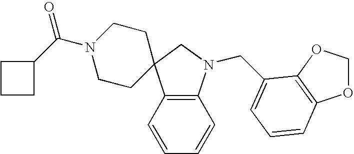 | 1′-(cyclobutylcarbonyl)- 1,2-dihydro-1- (benzo[1,3]dioxol-4- ylmethyl)-spiro[3H- indole-3,4′-piperidine] |
| 54 | 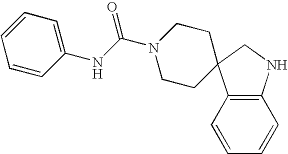 | 1′-(phenylcarbamoyl)-1,2- dihydro-spiro[3H-indole- 3,4′-piperidine] |
| 55 | 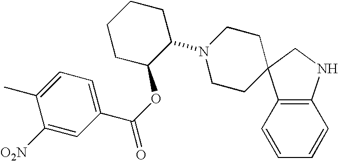 | 1′-[cis-2-(4-methyl-3- nitro-benzoyloxy)- cyclohexyl]-1,2-dihydro- spiro[3H-indole-3,4′- piperidine] |
| 56 | 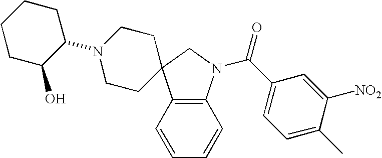 | 1′-(cis-2-hydroxy- cyclohexyl)-1,2-dihydro- 1-(3-nitro-4-methyl- benzoyl)-spiro[3H-indole- 3,4′-piperidine] |
| 57 | 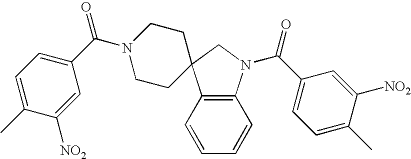 | 1′-(3-nitro-4-methyl- benzoyl)-1,2-dihydro-1-(3- nitro-4-methyl-benzoyl)- spiro[3H-indole-3,4′- piperidine] |
| 58 | 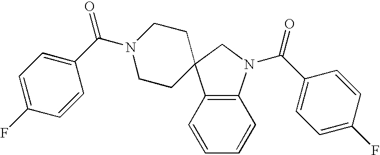 | 1′-(4-fluoro-benzoyl)-1,2- dihydro-1-(4-fluoro- benzoyl)-spiro[3H-indole- 3,4′-piperidine] |
| 59 | 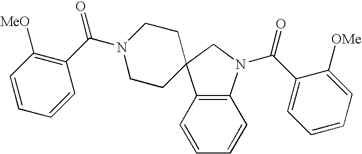 | 1′-(2-methoxy-benzoyl)- 1,2-dihydro-1-(2-methoxy- benzoyl)-spiro[3H-indole- 3,4′-piperidine] |
| 60 | 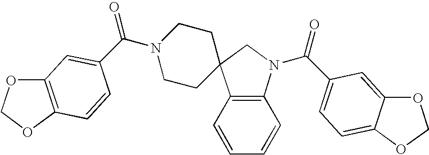 | 1′-(benzo[1,3]dioxole-5- carbonyl)-1,2-dihydro-1- (benzo[1,3]dioxole-5- carbonyl)-spiro[3H- indole-3,4′-piperidine] |
| 61 | 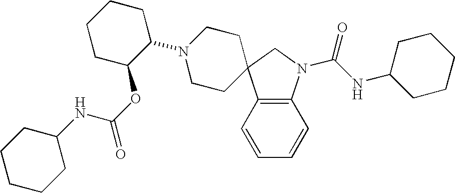 | 1′-(cis-2- cyclohexylcarbamoyloxy- cyclohexyl)-1,2-dihydro- 1-(cyclohexylcarbamoyl)- spiro[3H-indole-3,4′- piperidine] |
| 62 | 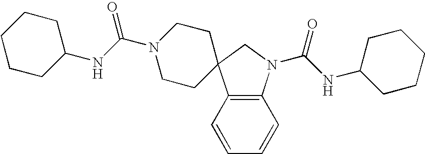 | 1′-(cyclohexylcarbamoyl)- 1,2-dihydro-1- (cyclohexylcarbamoyl)- spiro[3H-indole-3,4′- piperidine] |
| 63 | 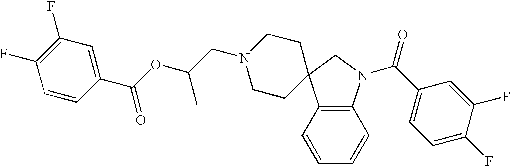 | 1′-[2-(3,4-difluoro- benzoyloxy)-propyl]-1,2- dihydro-1-(3,4-difluoro- benzoyl)-spiro[3H-indole- 3,4′-piperidine] |
| 64 | 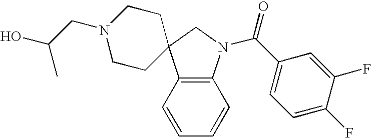 | 1′-[2-hydroxy-propyl]-1,2- dihydro-1-(3,4-difluoro- benzoyl)-spiro[3H-indole- 3,4′-piperidine] |
| 65 | 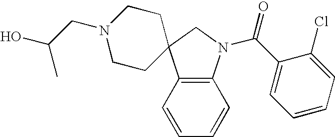 | 1′-[2-hydroxy-propyl]-1,2- dihydro-1-(2-chloro- benzoyl)-spiro[3H-indole- 3,4′-piperidine] |
| 66 | 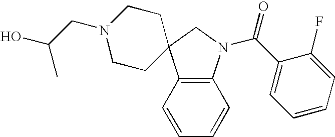 | 1′-[2-hydroxy-propyl]-1,2- dihydro-1-(2-fluoro- benzoyl)-spiro[3H-indole- 3,4′-piperidine] |
| 67 | 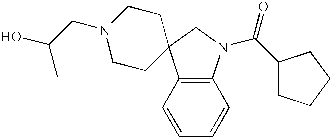 | 1′-[2-hydroxy-propyl]-1,2- dihydro-1- (cyclopentylcarbonyl)- spiro[3H-indole-3,4′- piperidine] |
| 68 | 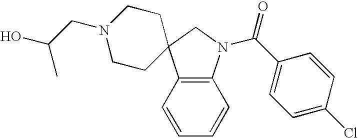 | 1′-[2-hydroxy-propyl]-1,2- dihydro-1-(4-chloro- benzoyl)-spiro[3H-indole- 3,4′-piperidine] |
| 69 | 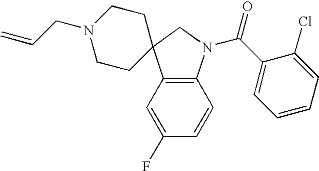 | 1′-(allyl)-1,2-dihydro-5- fluoro-1-(2-chloro- benzoyl)-spiro[3H-indole- 3,4′-piperidine] |
| 70 | 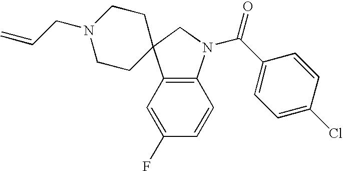 | 1′-(allyl)-1,2-dihydro-5- fluoro-1-(4-chloro- benzoyl)-spiro[3H-indole- 3,4′-piperidine] |
| 71 | 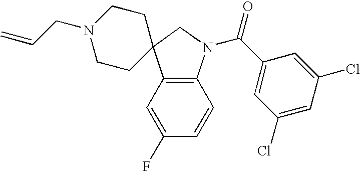 | 1′-(allyl)-1,2-dihydro-5- fluoro-1-(3,5-dichloro- benzoyl)-spiro[3H-indole- 3,4′-piperidine] |
| 72 | 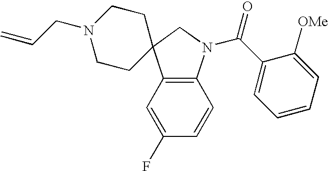 | 1′-(allyl)-1,2-dihydro-5- fluoro-1-(2-methoxy- benzoyl)-spiro[3H-indole- 3,4′-piperidine] |
| 73 | 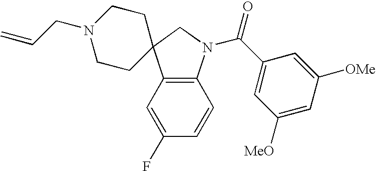 | 1′-(allyl)-1,2-dihydro-5- fluoro-1-(3,5-dimethoxy- benzoyl)-spiro[3H-indole- 3,4′-piperidine] |
| 74 | 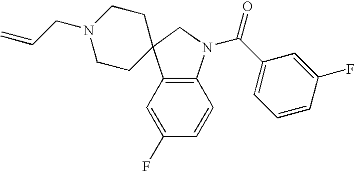 | 1′-(allyl)-1,2-dihydro-5- fluoro-1-(3-fluoro- benzoyl)-spiro[3H-indole- 3,4′-piperidine] |
| 75 | 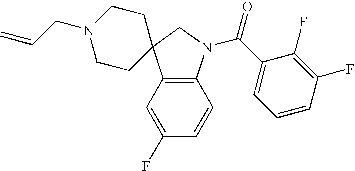 | 1′-(allyl)-1,2-dihydro-5- fluoro-1-(2,3-difluoro- benzoyl)-spiro[3H-indole- 3,4′-piperidine] |
| 76 | 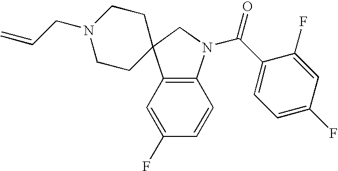 | 1′-(allyl)-1,2-dihydro-5- fluoro-1-(2,4-difluoro- benzoyl)-spiro[3H-indole- 3,4′-piperidine] |
| 77 | 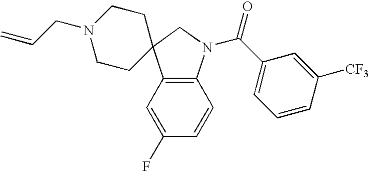 | 1′-(allyl)-1,2-dihydro-5- fluoro-1-[3- trifluoromethyl-benzoyl]- spiro[3H-indole-3,4′- piperidine] |
| 78 | 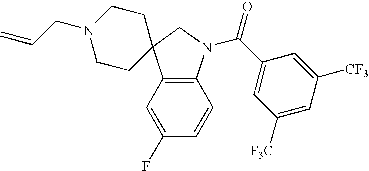 | 1′-(allyl)-1,2-dihydro-5- fluoro-1-(3,5-bis- trifluoromethyl-benzoyl)- spiro[3H-indole-3,4′- piperidine] |
| 79 | 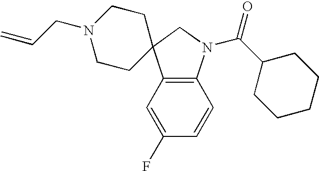 | 1′-(allyl)-1,2-dihydro-5- fluoro-1- (cyclohexylcarbonyl)- spiro[3H-indole-3,4′- piperidine] |
| 80 | 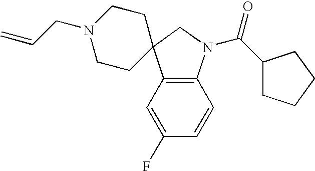 | 1′-(allyl)-1,2-dihydro-5- fluoro-1- (cyclopetnylcarbonyl)- spiro[3H-indole-3,4′- piperidine] |
| 81 | 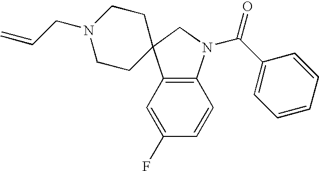 | 1′-(allyl)-1,2-dihydro-5- fluoro-1-(benzoyl)- spiro[3H-indole-3,4′- piperidine] |
| 82 | 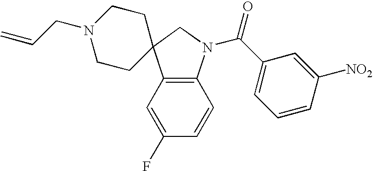 | 1′-(allyl)-1,2-dihydro-5- fluoro-1-(3-nitro-benzoyl)- spiro[3H-indole-3,4′- piperidine] |
| 83 | 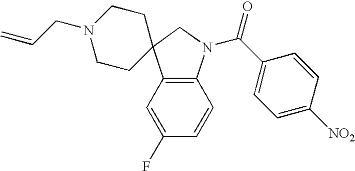 | 1′-(allyl)-1,2-dihydro-5- fluoro-1-(4-nitro-benzoyl)- spiro[3H-indole-3,4′- piperidine] |
| 84 | 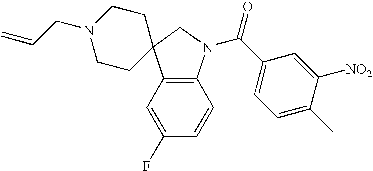 | 1′-(allyl)-1,2-dihydro-5- fluoro-1-(3-nitro-4- methyl-benzoyl)-spiro[3H- indole-3,4′-piperidine] |
| 85 | 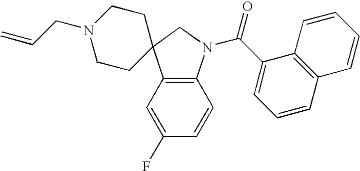 | 1′-(allyl)-1,2-dihydro-5- fluoro-1-(naphthalene-1- carbonyl)-spiro[3H- indole-3,4′-piperidine] |
| 86 | 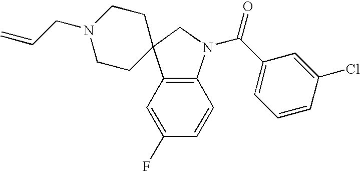 | 1′-(allyl)-1,2-dihydro-5- fluoro-1-(3-chloro- benzoyl)-spiro[3H-indole- 3,4′-piperidine] |
| 87 | 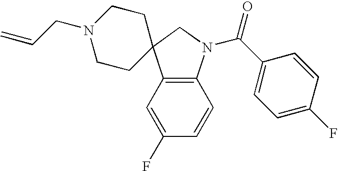 | 1′-(allyl)-1,2-dihydro-5- fluoro-1-(4-fluoro- benzoyl)-spiro[3H-indole- 3,4′-piperidine] |
| 88 | 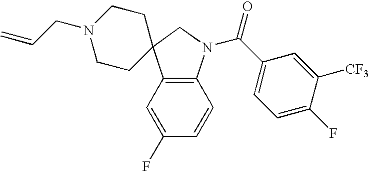 | 1′-(allyl)-1,2-dihydro-5- fluoro-1-(3- trifluoromethyl-4-fluoro- benzoyl)-spiro[3H-indole- 3,4′-piperidine] |
| 89 | 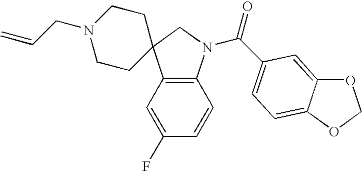 | 1′-(allyl)-1,2-dihydro-5- fluoro-1- (benzo[1,3]dioxole-5- carbonyl)-spiro[3H- indole-3,4′-piperidine] |
| 90 | 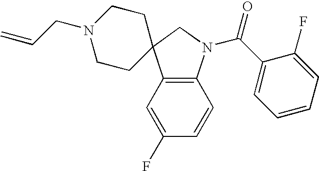 | 1′-(allyl)-1,2-dihydro-5- fluoro-1-(2-fluoro- benzoyl)-spiro[3H-indole- 3,4′-piperidine] |
| 91 | 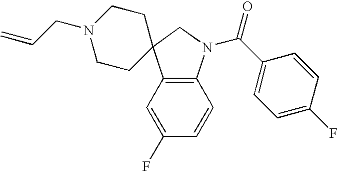 | 1′-(allyl)-1,2-dihydro-5- fluoro-1-(4-fluoro- benzoyl)-spiro[3H-indole- 3,4′-piperidine] |
| 92 | 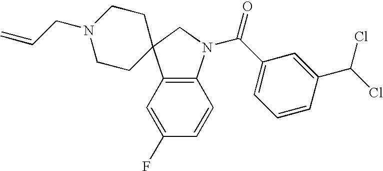 | 1′-(allyl)-1,2-dihydro-5- fluoro-1-(3- dichloromethyl-benzoyl)- spiro[3H-indole-3,4′- piperidine] |
| 93 | 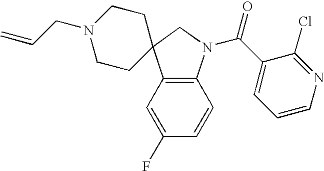 | 1′-(allyl)-1,2-dihydro-5- fluoro-1-(2-chloro- pyridine-3-carbonyl)- spiro[3H-indole-3,4′- piperidine] |
| 94 | 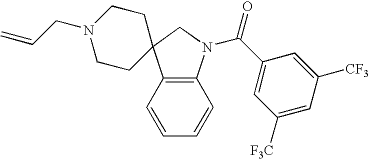 | 1′-(allyl)-1,2-dihydro-1- (3,5-bis-trifluoromethyl- benzoyl)-spiro[3H-indole- 3,4′-piperidine] |
| 95 | 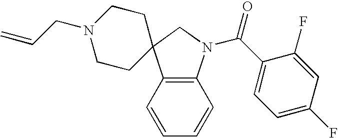 | 1′-(allyl)-1,2-dihydro-1- (2,4-difluoro-benzoyl)- spiro[3H-indole-3,4′- piperidine] |
| 96 | 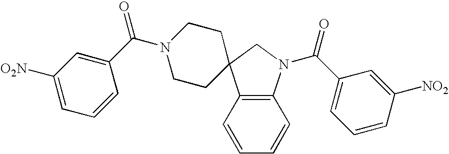 | 1′-(3-nitro-benzoyl)-1,2- dihydro-1-(3-nitro- benzoyl)-spiro[3H-indole- 3,4′-piperidine] |
| 97 | 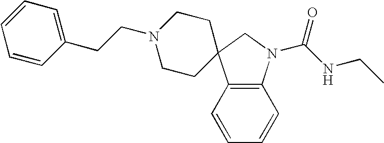 | 1′-(phenethyl)-1,2- dihydro-1- (ethylcarbamoyl)- spiro[3H-indole-3,4′- piperidine] |
| 98 | 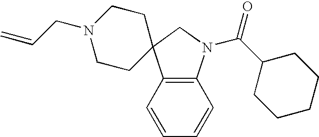 | 1′-(allyl)-1,2-dihydro-1- (cyclohexylcarbonyl)- spiro[3H-indole-3,4′- piperidine] |
| 99 | 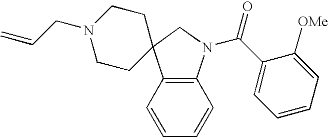 | 1′-(allyl)-1,2-dihydro-1-(2- methoxy-benzoyl)- spiro[3H-indole-3,4′- piperidine] |
| 100 | 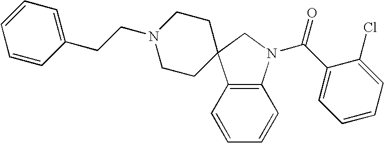 | 1′-(phenethyl)-1,2- dihydro-1-(2-chloro- benzoyl)-spiro[3H-indole- 3,4′-piperidine] |
| 101 | 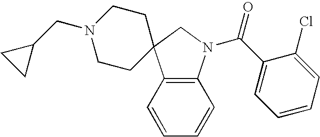 | 1′-(cyclopropylmethyl)- 1,2-dihydro-1-(2-chloro- benzoyl)-spiro[3H-indole- 3,4′-piperidine] |
| 102 | 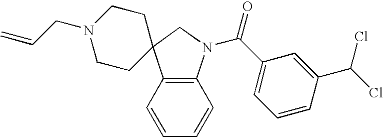 | 1′-(allyl)-1,2-dihydro-1-(3- dichloromethyl-benzoyl)- spiro[3H-indole-3,4′- piperidine] |
| 103 | 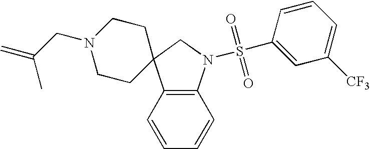 | 1′-(2-methyl-allyl)-1,2- dihydro-1-(3- trifluoromethyl- benzenesulfonyl)- spiro[3H-indole-3,4′- piperidine] |
| 104 | 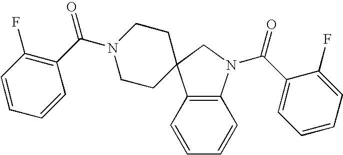 | 1′-(2-fluoro-benzoyl)-1,2- dihydro-1-(2-fluoro- benzoyl)-spiro[3H-indole- 3,4′-piperidine] |
| 105 | 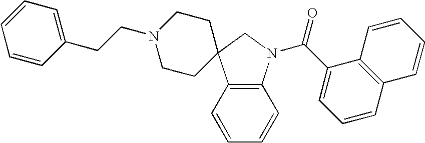 | 1′-(phenethyl)-1,2- dihydro-1-(naphthylene-1- carbonyl)-spiro[3H- indole-3,4′-piperidine] |
| 106 | 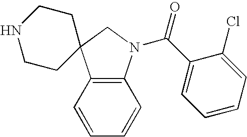 | 1,2-dihydro-1-(2-chloro- benzoyl)-spiro[3H-indole- 3,4′-piperidine] |
| 107 | 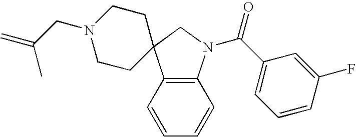 | 1′-(2-methyl-allyl)-1,2- dihydro-1-(3-fluoro- benzoyl)-spiro[3H-indole- 3,4′-piperidine] |
| 108 | 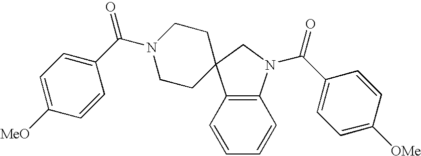 | 1′-(4-methoxy-benzoyl)- 1,2-dihydro-1-(4-methoxy- benzoyl)-spiro[3H-indole- 3,4′-piperidine] |
| 109 | 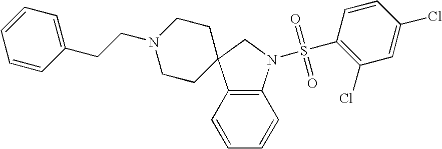 | 1′-(phenethyl)-1,2- dihydro-1-(2,4-dichloro- benzenesulfonyl)- spiro[3H-indole-3,4′- piperidine] |
| 110 | 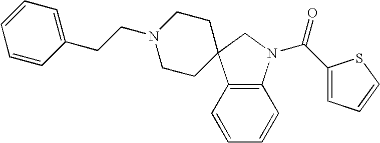 | 1′-(phenethyl)-1,2- dihydro-1-(thiophene-2- carbonyl)-spiro[3H- indole-3,4′-piperidine] |
| 111 | 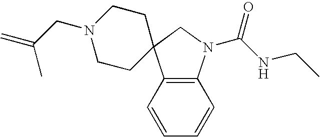 | 1′-(2-methyl-allyl)-1,2- dihydro-1- (ethylcarbamoyl)- spiro[3H-indole-3,4′- piperidine] |
| 112 | 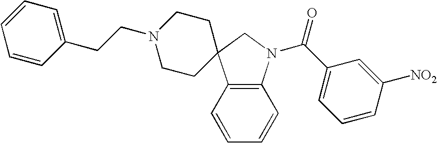 | 1′-(phenethyl)-1,2- dihydro-1-(3-nitro- benzoyl)-spiro[3H-indole- 3,4′-piperidine] |
| 113 | 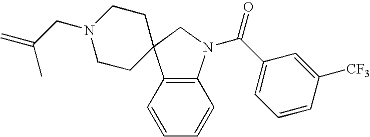 | 1′-(2-methyl-allyl)-1,2- dihydro-1-(3- trifluoromethyl-benzoyl)- spiro[3H-indole-3,4′- piperidine] |
| 114 | 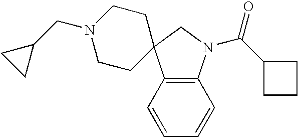 | 1′-(cyclopropylmethyl)- 1,2-dihydro-1- (cyclobutylcarbonyl)- spiro[3H-indole-3,4′- piperidine] |
| 115 | 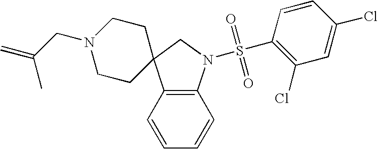 | 1′-(2-methyl-allyl)-1,2- dihydro-1-(2,4-dichloro- benzenesulfonyl)- spiro[3H-indole-3,4′- piperidine] |
| 116 | 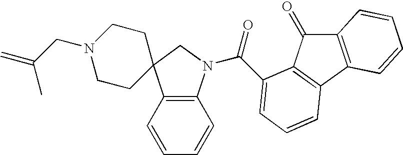 | 1′-(2-methyl-allyl)-1,2- dihydro-1-(9-oxo-9H- fluorene-1-carbonyl)- spiro[3H-indole-3,4′- piperidine] |
| 117 | 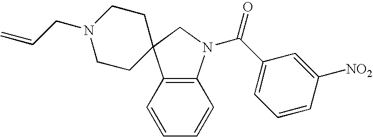 | 1′-(allyl)-1,2-dihydro-1-(3- nitro-benzoyl)-spiro[3H- indole-3,4′-piperidine] |
| 118 | 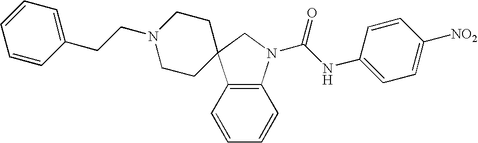 | 1′-(phenethyl)-1,2- dihydro-1-(4-nitro- phenylcarbamoyl)- spiro[3H-indole-3,4′- piperidine] |
| 119 | 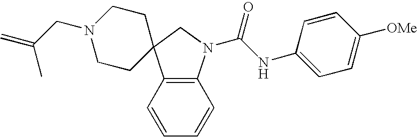 | 1′-(2-methyl-allyl)-1,2- dihydro-1-(4-methoxy- phenylcarbamoyl)- spiro[3H-indole-3,4′- piperidine] |
| 120 | 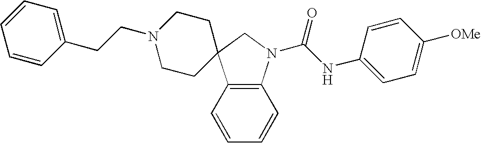 | 1′-(phenethyl)-1,2- dihydro-1-(4-methoxy- phenylcarbamoyl)- spiro[3H-indole-3,4′- piperidine] |
| 121 | 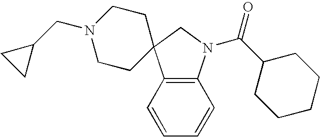 | 1′-(cyclopropylmethyl)- 1,2-dihydro-1- (cyclohexylcarbonyl)- spiro[3H-indole-3,4′- piperidine] |
| 122 | 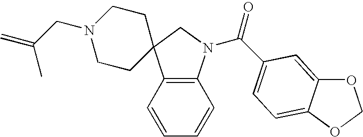 | 1′-(2-methyl-allyl)-1,2- dihydro-1- (benzo[1,3]dioxole-5- carbonyl)-spiro[3H- indole-3,4′-piperidine] |
| 123 | 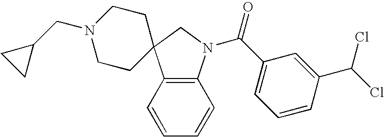 | 1′-(cyclopropylmethyl)- 1,2-dihydro-1-(3- dichloromethyl-benzoyl)- spiro[3H-indole-3,4′- piperidine] |
| 124 | 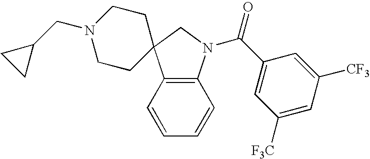 | 1′-(cyclopropylmethyl)- 1,2-dihydro-1-(3,5-bis- trifluoromethyl-benzoyl)- spiro[3H-indole-3,4′- piperidine] |
| 125 | 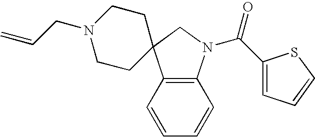 | 1′-(allyl)-1,2-dihydro-1- (thiophene-2-carbonyl)- spiro[3H-indole-3,4′- piperidine] |
| 126 | 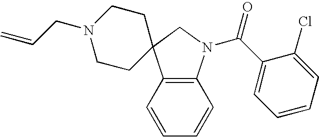 | 1′-(allyl)-1,2-dihydro-1-(2- chloro-benzoyl)-spiro[3H- indole-3,4′-piperidine] |
| 127 | 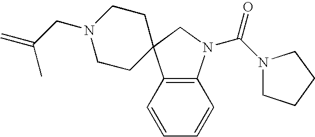 | 1′-(2-methyl-allyl)-1,2- dihydro-1-(pyrrolidine-1- carbonyl)-spiro[3H- indole-3,4′-piperidine] |
| 128 | 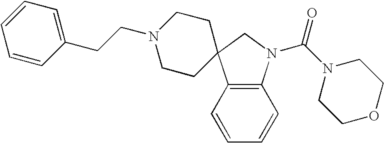 | 1′-(phenethyl)-1,2- dihydro-1-(morpholine-4- carbonyl)-spiro[3H- indole-3,4′-piperidine] |
| 129 | 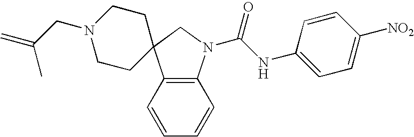 | 1′-(2-methyl-allyl)-1,2- dihydro-1-(4-nitro- phenylcarbamoyl)- spiro[3H-indole-3,4′- piperidine] |
| 130 | 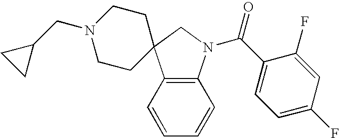 | 1′-(cyclopropylmethyl)- 1,2-dihydro-1-(2,4- difluoro-benzoyl)- spiro[3H-indole-3,4′- piperidine] |
| 131 | 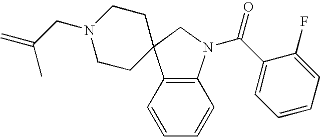 | 1′-(2-methyl-allyl)-1,2- dihydro-1-(2-fluoro- benzoyl)-spiro[3H-indole- 3,4′-piperidine] |
| 132 | 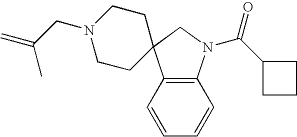 | 1′-(2-methyl-allyl)-1,2- dihydro-1- (cyclobutylcarbonyl)- spiro[3H-indole-3,4′- piperidine] |
| 133 | 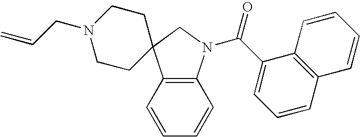 | 1′-(allyl)-1,2-dihydro-1- (naphthylene-1-carbonyl)- spiro[3H-indole-3,4′- piperidine] |
| 134 | 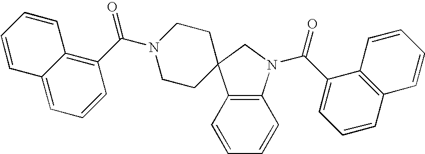 | 1′-(naphthylene-1- carbonyl)-1,2-dihydro-1- (naphthylene-1-carbonyl)- spiro[3H-indole-3,4′- piperidine] |
| 135 | 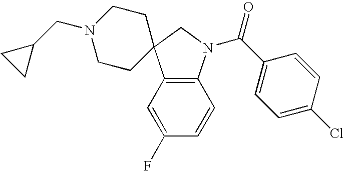 | 1′-(cyclopropylmethyl)- 1,2-dihydro-5-fluoro-1-(4- chloro-benzoyl)-spiro[3H- indole-3,4′-piperidine] |
| 136 | 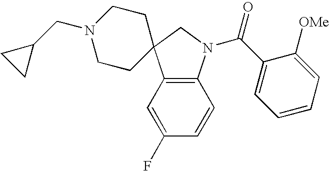 | 1′-(cyclopropylmethyl)- 1,2-dihydro-5-fluoro-1-(2- methoxy-benzoyl)- spiro[3H-indole-3,4′- piperidine] |
| 137 | 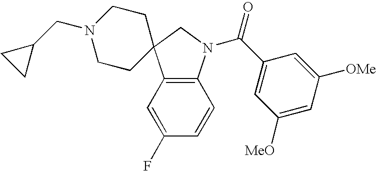 | 1′-(cyclopropylmethyl)- 1,2-dihydro-5-fluoro-1- (3,5-dimethoxy-benzoyl)- spiro[3H-indole-3,4′- piperidine] |
| 138 | 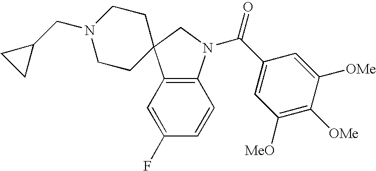 | 1′-(cyclopropylmethyl)- 1,2-dihydro-5-fluoro-1- (3,4,5-trimethoxy- benzoyl)-spiro[3H-indole- 3,4′-piperidine] |
| 139 | 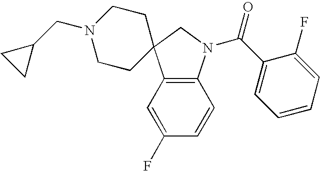 | 1′-(cyclopropylmethyl)- 1,2-dihydro-5-fluoro-1-(2- fluoro-benzoyl)-spiro[3H- indole-3,4′-piperidine] |
| 140 | 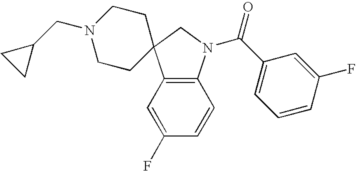 | 1′-(cyclopropylmethyl)- 1,2-dihydro-5-fluoro-1-(3- fluoro-benzoyl)-spiro[3H- indole-3,4′-piperidine] |
| 141 | 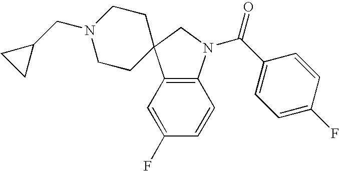 | 1′-(cyclopropylmethyl)- 1,2-dihydro-5-fluoro-1-(4- fluoro-benzoyl)-spiro[3H- indole-3,4′-piperidine] |
| 142 | 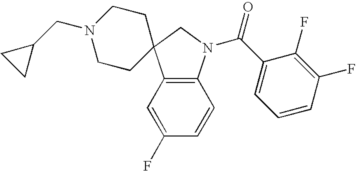 | 1′-(cyclopropylmethyl)- 1,2-dihydro-5-fluoro-1- (2,3-difluoro-benzoyl)- spiro[3H-indole-3,4′- piperidine] |
| 143 | 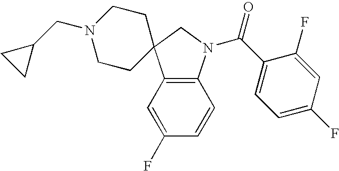 | 1′-(cyclopropylmethyl)- 1,2-dihydro-5-fluoro-1- (2,4-difluoro-benzoyl)- spiro[3H-indole-3,4′- piperidine] |
| 144 | 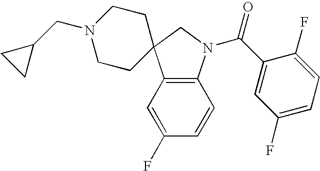 | 1′-(cyclopropylmethyl)- 1,2-dihydro-5-fluoro-1- (2,5-difluoro-benzoyl)- spiro[3H-indole-3,4′- piperidine] |
| 145 | 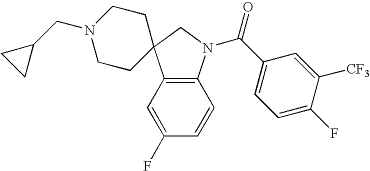 | 1′-(cyclopropylmethyl)- 1,2-dihydro-5-fluoro-1-(3- trifluoromethyl-4-fluoro- benzoyl)-spiro[3H-indole- 3,4′-piperidine] |
| 146 | 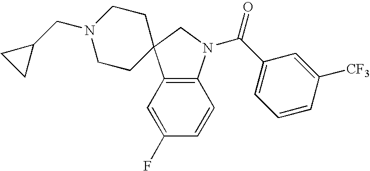 | 1′-(cyclopropylmethyl)- 1,2-dihydro-5-fluoro-1-(3- trifluoromethyl-benzoyl)- spiro[3H-indole-3,4′- piperidine] |
| 147 | 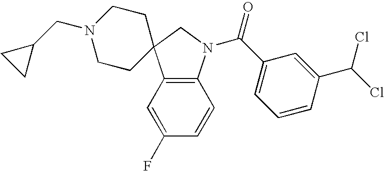 | 1′-(cyclopropylmethyl)- 1,2-dihydro-5-fluoro-1-(3- dichloromethyl-benzoyl)- spiro[3H-indole-3,4′- piperidine] |
| 148 | 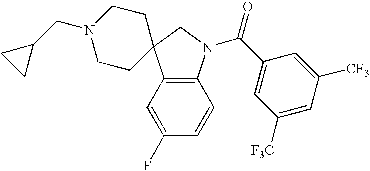 | 1′-(cyclopropylmethyl)- 1,2-dihydro-5-fluoro-1- (3,5-bis-trifluoromethyl- benzoyl)-spiro[3H-indole- 3,4′-piperidine] |
| 149 | 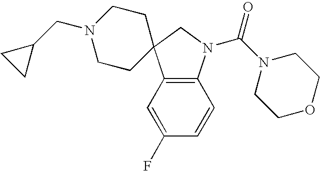 | 1′-(cyclopropylmethyl)- 1,2-dihydro-5-fluoro-1- (morpholine-4-carbonyl)- spiro[3H-indole-3,4′- piperidine] |
| 150 | 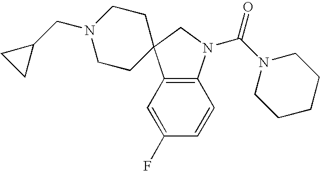 | 1-(cyclopropylmethyl)- 1,2-dihydro-5-fluoro-1- (piperidine-1-carbonyl)- spiro[3H-indole-3,4′- piperidine] |
| 151 | 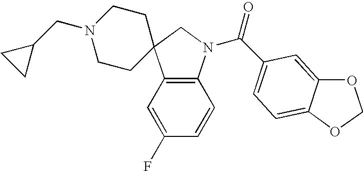 | 1′-(cyclopropylmethyl)- 1,2-dihydro-5-fluoro-1- (benzo[1,3]dioxole-5- carbonyl)-spiro[3H- indole-3,4′-piperidine] |
| 152 | 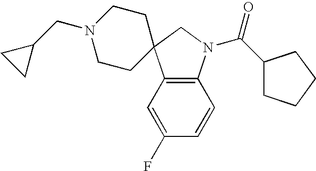 | 1′-(cyclopropylmethyl)- 1,2-dihydro-5-fluoro-1- (cyclopentylcarbonyl)- spiro[3H-indole-3,4′- piperidine] |
| 153 | 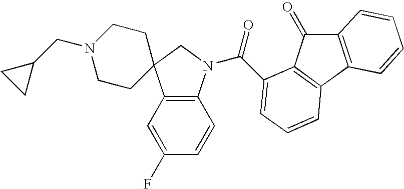 | 1′-(cyclopropylmethyl)- 1,2-dihydro-5-fluoro-1-(9- oxo-9H-fluorene-1- carbonyl)-spiro[3H- indole-3,4′-piperidine] |
| 154 | 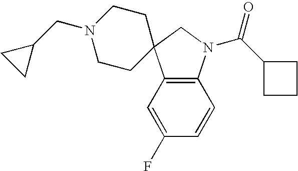 | 1′-(cyclopropylmethyl)- 1,2-dihydro-5-fluoro-1- (cyclobutylcarbonyl)- spiro[3H-indole-3,4′- piperidine] |
| 155 | 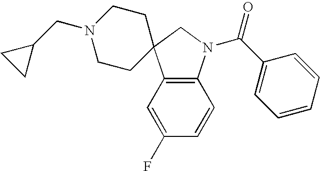 | 1′-(cyclopropylmethyl)- 1,2-dihydro-5-fluoro-1- (benzoyl)-spiro[3H- indole-3,4′-piperidine] |
| 156 | 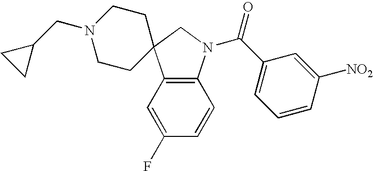 | 1′-(cyclopropylmethyl)- 1,2-dihydro-5-fluoro-1-(3- nitro-benzoyl)-spiro[3H- indole-3,4′-piperidine] |
| 157 | 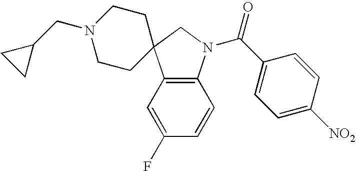 | 1′-(cyclopropylmethyl)- 1,2-dihydro-5-fluoro-1-(4- nitro-benzoyl)-spiro[3H- indole-3,4′-piperidine] |
| 158 | 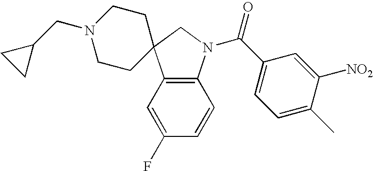 | 1′-(cyclopropylmethyl)- 1,2-dihydro-5-fluoro-1-(3- nitro-4-methyl-benzoyl)- spiro[3H-indole-3,4′- piperidine] |
| 159 | 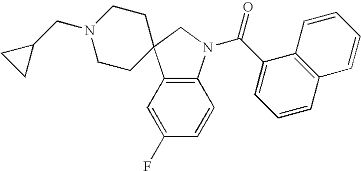 | 1′-(cyclopropylmethyl)- 1,2-dihydro-5-fluoro-1- (naphthylene-1-carbonyl)- spiro[3H-indole-3,4′- piperidine] |
| 160 | 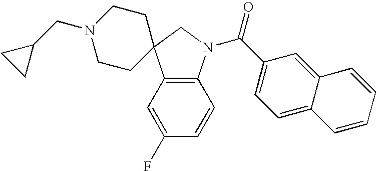 | 1′-(cyclopropylmethyl)- 1,2-dihydro-5-fluoro-1- (naphthylene-2-carbonyl)- spiro[3H-indole-3,4′- piperidine] |
| 161 | 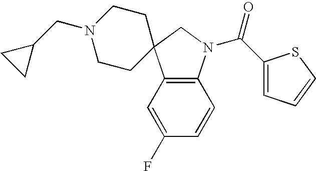 | 1′-(cyclopropylmethyl)- 1,2-dihydro-5-fluoro-1- (thiophene-2-carbonyl)- spiro[3H-indole-3,4′- piperidine] |
| 162 | 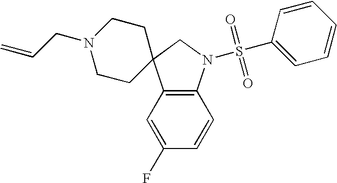 | 1′-(allyl)-1,2-dihydro-5- fluoro-1- (benzenesulfonyl)- spiro[3H-indole-3,4′- piperidine] |
| 163 | 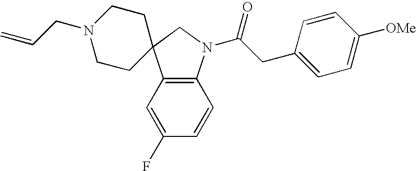 | 1′-(allyl)-1,2-dihydro-5- fluoro-1-[2-(4-methoxy- phenyl)-acetyl]-spiro[3H- indole-3,4′-piperidine] |
| 164 | 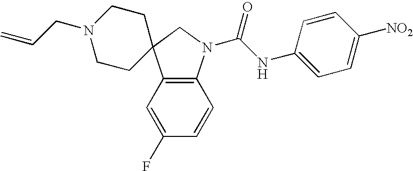 | 1′-(allyl)-1,2-dihydro-5- fluoro-1-(4-nitro- phenylcarbamoyl)- spiro[3H-indole-3,4′- piperidine] |
| 165 | 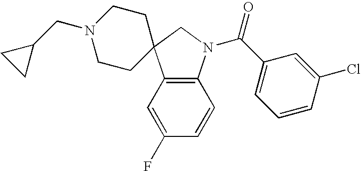 | 1′-(cyclopropylmethyl)- 1,2-dihydro-5-fluoro-1-(3- chloro-benzoyl)-spiro[3H- indole-3,4′-piperidine] |
| 166 | 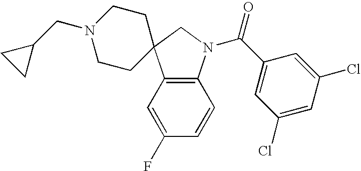 | 1′-(cyclopropylmethyl)- 1,2-dihydro-5-fluoro-1- (3,5-dichloro-benzoyl)- spiro[3H-indole-3,4′- piperidine] |
| 167 | 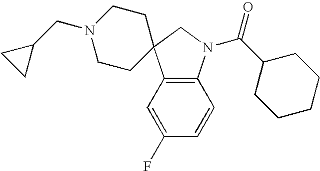 | 1′-(cyclopropylmethyl)- 1,2-dihydro-5-fluoro-1- (cyclohexylcarbonyl)- spiro[3H-indole-3,4′- piperidine] |
| 168 | 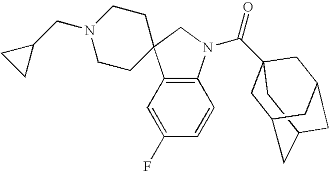 | 1′-(cyclopropylmethyl)- 1,2-dihydro-5-fluoro-1- (adamantane-1-carbonyl)- spiro[3H-indole-3,4′- piperidine] |
| 169 | 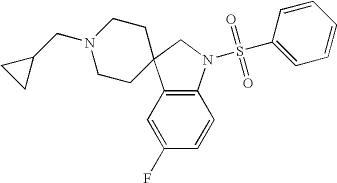 | 1′-(cyclopropylmethyl)- 1,2-dihydro-5-fluoro-1- (benzenesulfonyl)- spiro[3H-indole-3,4′- piperidine] |
| 170 | 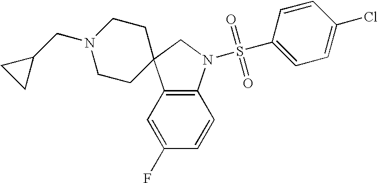 | 1′-(cyclopropylmethyl)- 1,2-dihydro-5-fluoro-1-(4- chloro-benzenesulfonyl)- spiro[3H-indole-3,4′- piperidine] |
| 171 | 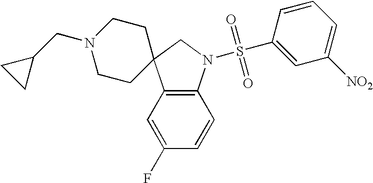 | 1′-(cyclopropylmethyl)- 1,2-dihydro-5-fluoro-1-(3- nitro-benzenesulfonyl)- spiro[3H-indole-3,4′- piperidine] |
| 172 | 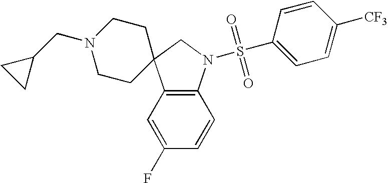 | 1′-(cyclopropylmethyl)- 1,2-dihydro-5-fluoro-1-(4- trifluoromethyl- benzenesulfonyl)- spiro[3H-indole-3,4′- piperidine] |
| 173 | 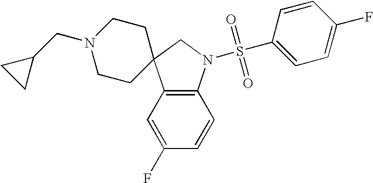 | 1′-(cyclopropylmethyl)- 1,2-dihydro-5-fluoro-1-(4- fluoro-benzenesulfonyl)- spiro[3H-indole-3,4′- piperidine] |
| 174 | 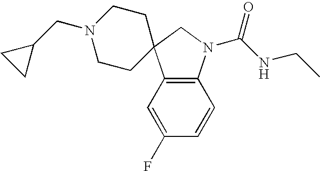 | 1′-(cyclopropylmethyl)- 1,2-dihydro-5-fluoro-1- (ethylcarbamoyl)- spiro[3H-indole-3,4′- piperidine] |
| 175 | 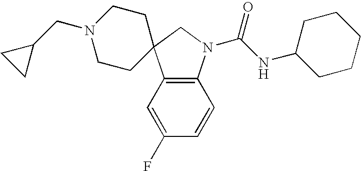 | 1′-(cyclopropylmethyl)- 1,2-dihydro-5-fluoro-1- (cyclohexylcarbamoyl)- spiro[3H-indole-3,4′- piperidine] |
| 176 | 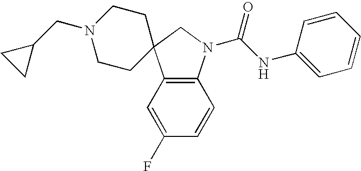 | 1′-(cyclopropylmethyl)- 1,2-dihydro-5-fluoro-1- (phenylcarbamoyl)- spiro[3H-indole-3,4′- piperidine] |
| 177 | 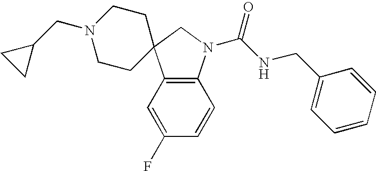 | 1′-(cyclopropylmethyl)- 1,2-dihydro-5-fluoro-1- (benzylcarbamoyl)- spiro[3H-indole-3,4′- piperidine] |
| 178 | 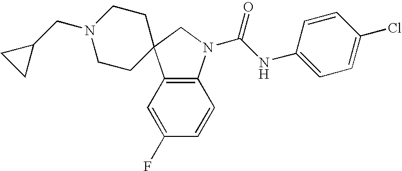 | 1′-(cyclopropylmethyl)- 1,2-dihydro-5-fluoro-1-(4- chloro-phenylcarbamoyl)- spiro[3H-indole-3,4′- piperidine] |
| 179 | 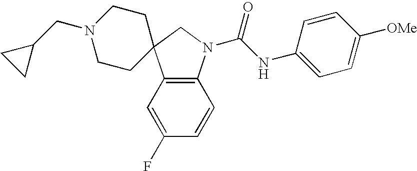 | 1′-(cyclopropylmethyl)- 1,2-dihydro-5-fluoro-1-(4- methoxy- phenylcarbamoyl)- spiro[3H-indole-3,4′- piperidine] |
| 180 | 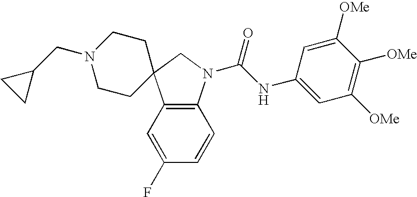 | 1′-(cyclopropylmethyl)- 1,2-dihydro-5-fluoro-1- (2,3,4-tri-methoxy- phenylcarbamoyl)- spiro[3H-indole-3,4′- piperidine] |
| 181 | 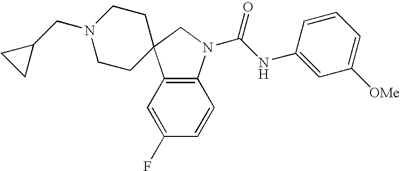 | 1′-(cyclopropylmethyl)- 1,2-dihydro-5-fluoro-1-[2- (3-methoxy-phenyl)- acetyl]-spiro[3H-indole- 3,4′-piperidine] |
| 182 | 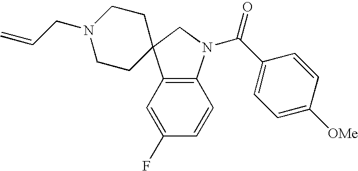 | 1′-(allyl)-1,2-dihydro-5- fluoro-1-(4-methoxy- benzoyl)-spiro[3H-indole- 3,4′-piperidine] |
| 183 | 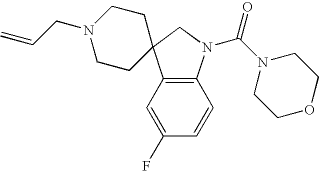 | 1′-(allyl)-1,2-dihydro-5- fluoro-1-(morpholine-4- carbonyl)-spiro[3H- indole-3,4′-piperidine] |
| 184 | 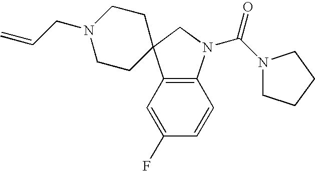 | 1′-(allyl)-1,2-dihydro-5- fluoro-1-(pyrrolidine-1- carbonyl)-spiro[3H- indole-3,4′-piperidine] |
| 185 | 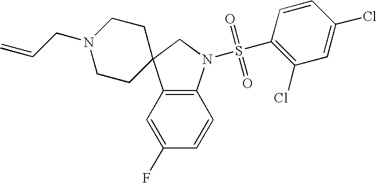 | 1′-(allyl)-1,2-dihydro-5- fluoro-1-(2,4-dichloro- benzenesulfonyl)- spiro[3H-indole-3,4′- piperidine] |
| 186 | 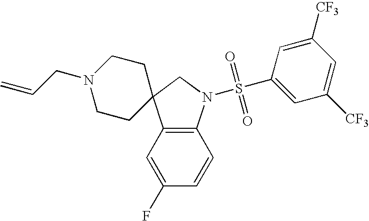 | 1′-(allyl)-1,2-dihydro-5- fluoro-1-(3,5-bis- trifluoromethyl- benzenesulfonyl)- spiro[3H-indole-3,4′- piperidine] |
| 187 | 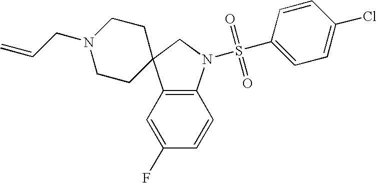 | 1′-(allyl)-1,2-dihydro-5- fluoro-1-(4-chloro- benzenesulfonyl)- spiro[3H-indole-3,4′- piperidine] |
| 188 | 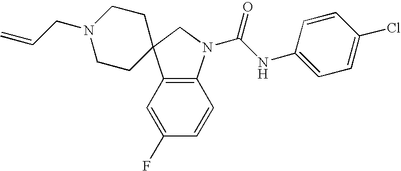 | 1′-(allyl)-1,2-dihydro-5- fluoro-1-(4-chloro- phenylcarbamoyl)- spiro[3H-indole-3,4′- piperidine] |
| 189 | 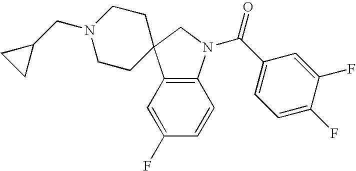 | 1′-(cyclopropylmethyl)- 1,2-dihydro-5-fluoro-1- (3,4-difluoro-benzoyl)- spiro[3H-indole-3,4′- piperidine] |
| 190 | 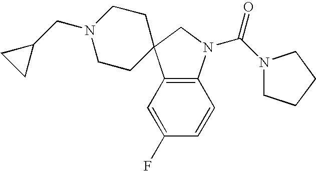 | 1′-(cyclopropylmethyl)- 1,2-dihydro-5-fluoro-1- (pyrrolidine-1-carbonyl)- spiro[3H-indole-3,4′- piperidine] |
| 191 | 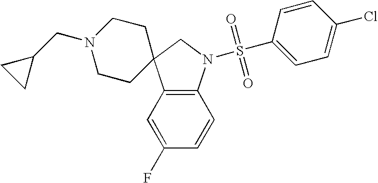 | 1′(cyclopropylmethyl)- 1,2-dihydro-5-fluoro-1-(4- chloro-benzenesulfonyl)- spiro[3H-indole-3,4′- piperidine] |
| 192 | 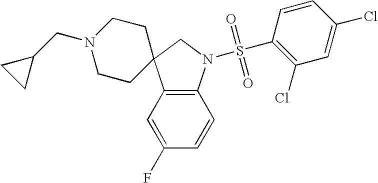 | 1′-(cyclopropylmethyl)- 1,2-dihydro-5-fluoro-1- (2,4-dichloro- benzenesulfonyl)- spiro[3H-indole-3,4′- piperidine] |
| 193 | 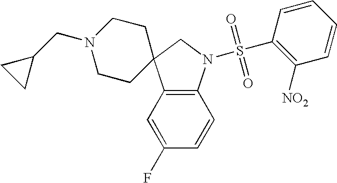 | 1′-(cyclopropylmethyl)- 1,2-dihydro-5-fluoro-1-(2- nitro-benzenesulfonyl)- spiro[3H-indole-3,4′- piperidine] |
| 194 | 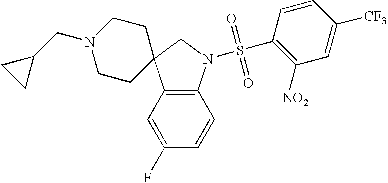 | 1′-(cyclopropylmethyl)- 1,2-dihydro-5-fluoro-1-(2- nitro-4-trifluoromethyl- benzenesulfonyl)- spiro[3H-indole-3,4′- piperidine] |
| 195 | 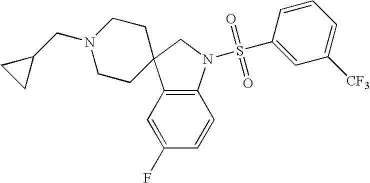 | 1′-(cyclopropylmethyl)- 1,2-dihydro-5-fluoro-1-(3- trifluoromethyl- benzenesulfonyl)- spiro[3H-indole-3,4′- piperidine] |
| 196 | 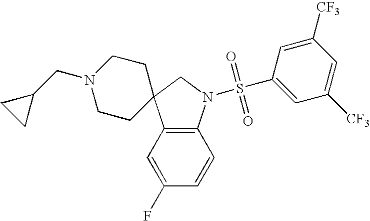 | 1′-(cyclopropylmethyl)- 1,2-dihydro-5-fluoro-1- (3,5-bis-trifluoromethyl- benzenesulfonyl)- spiro[3H-indole-3,4′- piperidine] |
| 197 | 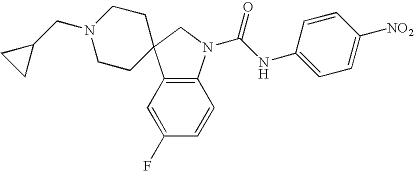 | 1′-(cyclopropylmethyl)- 1,2-dihydro-5-fluoro-1-(4- nitro-phenylcarbamoyl)- spiro[3H-indole-3,4′- piperidine] |
| 198 | 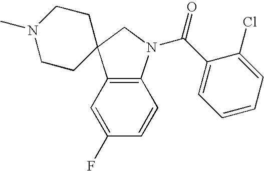 | 1′-(methyl)-1,2-dihydro-5- fluoro-1-(2-chloro- benzoyl)-spiro[3H-indole- 3,4′-piperidine] |
| 199 | 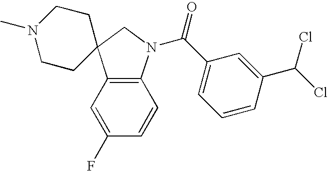 | 1′-(methyl)-1,2-dihydro-5- fluoro-1-(3- dichloromethyl-benzoyl)- spiro[3H-indole-3,4′- piperidine] |
| 200 | 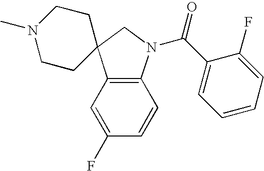 | 1′-(methyl)-1,2-dihydro-5- fluoro-1-(2-fluoro- benzoyl)-spiro[3H-indole- 3,4′-piperidine] |
| 201 | 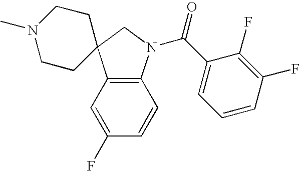 | 1′-(methyl)-1,2-dihydro-5- fluoro-1-(2,3-difluoro- benzoyl)-spiro[3H-indole- 3,4′-piperidine] |
| 202 | 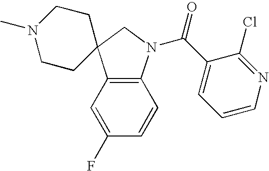 | 1′-(methyl)-1,2-dihydro-5- fluoro-1-(2-chloro- pyridine-3-carbonyl)- spiro[3H-indole-3,4′- piperidine] |
| 203 | 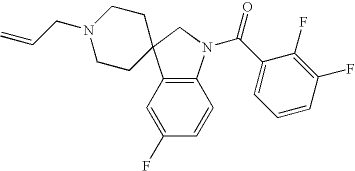 | 1′-(allyl)-1,2-dihydro-5- fluoro-1-(2,3-difluoro- benzoyl)-spiro[3H-indole- 3,4′-piperidine] |
| 204 | 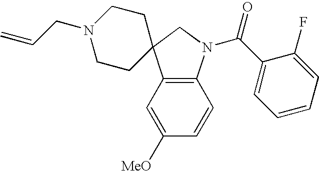 | 1′-(allyl)-1,2-dihydro-5- methoxy-1-(2-fluoro- benzoyl)-spiro[3H-indole- 3,4′-piperidine] |
| 205 | 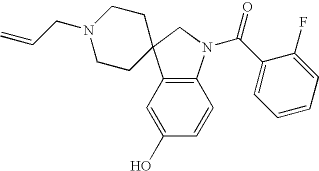 | 1′-(allyl)-1,2-dihydro-5- hydroxy-1-(2-fluoro- benzoyl)-spiro[3H-indole- 3,4′-piperidine] |
| 206 | 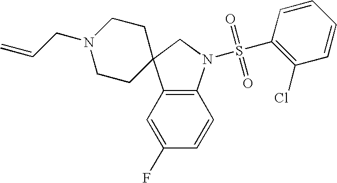 | 1′-(allyl)-1,2-dihydro-5- fluoro-1-(2-chloro- benzenesulfonyl)- spiro[3H-indole-3,4′- piperidine] |
| 207 | 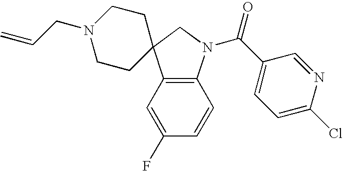 | 1′-(allyl)-1,2-dihydro-5- fluoro-1-(6-chloro- pyridine-3-carbonyl)- spiro[3H-indole-3,4′- piperidine] |
| 208 | 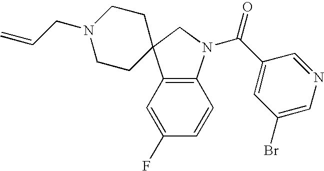 | 1′-(allyl)-1,2-dihydro-5- fluoro-1-(5-bromo- pyridine-3-carbonyl)- spiro[3H-indole-3,4′- piperidine] |
| 209 | 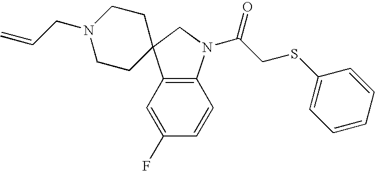 | 1′-(allyl)-1,2-dihydro-5- fluoro-1-(2- phenylsulfanyl-acetyl)- spiro[3H-indole-3,4′- piperidine] |
| 210 | 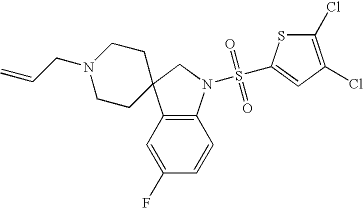 | 1′-(allyl)-1,2-dihydro-5- fluoro-1-(4,5-dichloro- thiophene-2-sulfonyl)- spiro[3H-indole-3,4′- piperidine] |
| 211 | 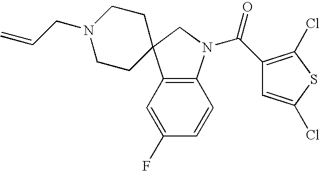 | 1′-(allyl)-1,2-dihydro-5- fluoro-1-(2,5-dichloro- thiophene-3-carbonyl)- spiro[3H-indole-3,4′- piperidine] |
| 212 | 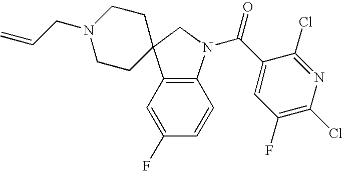 | 1′-(allyl)-1,2-dihydro-5- fluoro-1-(2,6-dichloro-5- fluoro-pyridine-3- carbonyl)-spiro[3H- indole-3,4′-piperidine] |
| 213 | 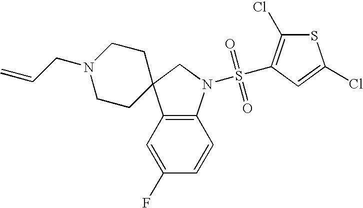 | 1′-(allyl)-1,2-dihydro-5- fluoro-1-(2,5-dichloro- thiophene-3-sulfonyl)- spiro[3H-indole-3,4′- piperidine] |
| 214 | 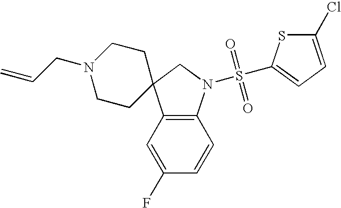 | 1′-(allyl)-1,2-dihydro-5- fluoro-1-(5-chloro- thiophene-2-sulfonyl)- spiro[3H-indole-3,4′- piperidine] |
| 215 | 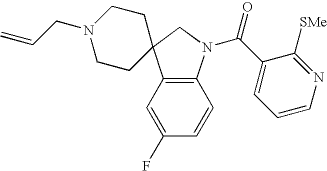 | 1′-(allyl)-1,2-dihydro-5- fluoro-1-(2- methylsulfanyl-pyridine-3- carbonyl)-spiro[3H- indole-3,4′-piperidine] |
| 216 | 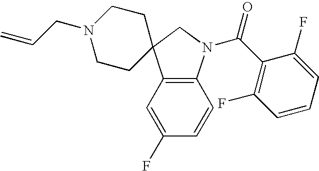 | 1′-(allyl)-1,2-dihydro-5- fluoro-1-(2,6-difluoro- benzoyl)-spiro[3H-indole- 3,4′-piperidine] |
| 217 | 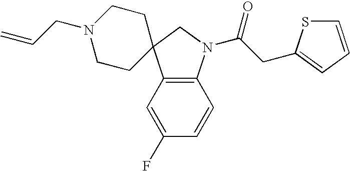 | 1′-(allyl)-1,2-dihydro-5- fluoro-1-(2-thiophen-2-yl- acetyl)-spiro[3H-indole- 3,4′-piperidine] |
| 218 | 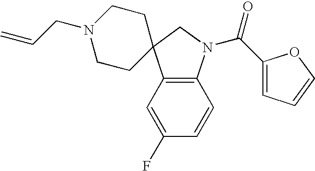 | 1′-(allyl)-1,2-dihydro-5- fluoro-1-(furan-2- carbonyl)-spiro[3H- indole-3,4′-piperidine] |
| 219 | 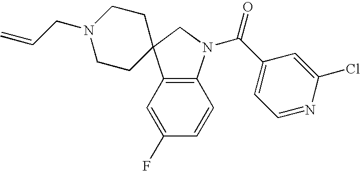 | 1′-(allyl)-1,2-dihydro-5- fluoro-1-(2-chloro- pyridine-4-carbonyl)- spiro[3H-indole-3,4′- piperidine] |
| 220 | 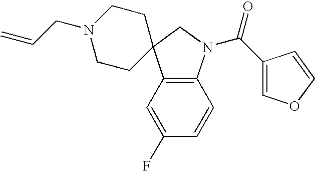 | 1′-(allyl)-1,2-dihydro-5- fluoro-1-(furan-3- carbonyl)-spiro[3H- indole-3,4′-piperidine] |
| 221 | 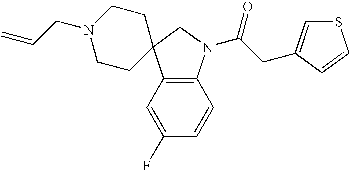 | 1′-(allyl)-1,2-dihydro-5- fluoro-1-(2-thiophen-3-yl- acetyl)-spiro[3H-indole- 3,4′-piperidine] |
| 222 | 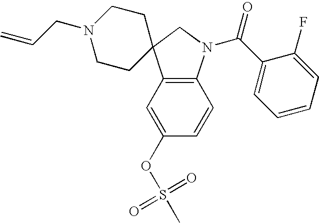 | 1′-(allyl)-1,2-dihydro-5- (methanesulfonyloxy)-1- (2-fluoro-benzoyl)- spiro[3H-indole-3,4′- piperidine] |
| 223 | 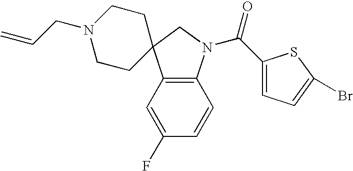 | 1′-(allyl)-1,2-dihydro-5- fluoro-1-(5-bromo- thiophene-2-carbonyl)- spiro[3H-indole-3,4′- piperidine] |
| 224 | 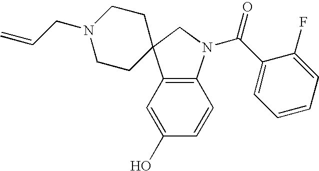 | 1′-(allyl)-1,2-dihydro-5- hydroxy-1-(2-fluoro- benzoyl)-spiro[3H-indole- 3,4′-piperidine] |
| 225 | 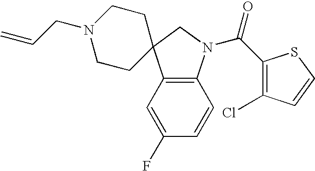 | 1′-(allyl)-1,2-dihydro-5- fluoro-1-(3-chloro- thiophene-2-carbonyl)- spiro[3H-indole-3,4′- piperidine] |
| 226 | 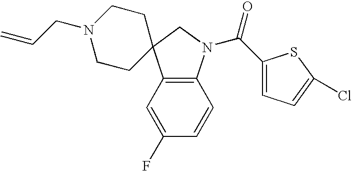 | 1′-(allyl)-1,2-dihydro-5- fluoro-1-(5-chloro- thiophene-2-carbonyl)- spiro[3H-indole-3,4′- piperidine] |
| 227 | 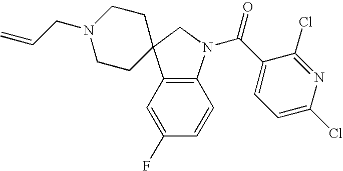 | 1′-(allyl)-1,2-dihydro-5- fluoro-1-(2,6-dichloro- pyridine-3-carbonyl)- spiro[3H-indole-3,4′- piperidine] |
| 228 | 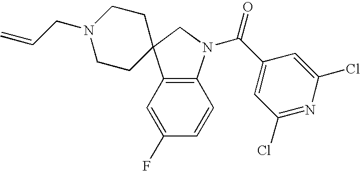 | 1′-(allyl)-1,2-dihydro-5- fluoro-1-(2,6-dichloro- pyridine-4-carbonyl)- spiro[3H-indole-3,4′- piperidine] |
| 229 | 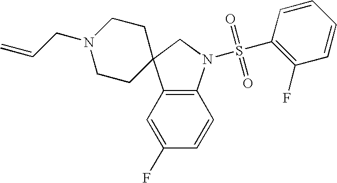 | 1′-(allyl)-1,2-dihydro-5- fluoro-1-(2-fluoro- benzenesulfonyl)- spiro[3H-indole-3,4′- piperidine] |
| 230 | 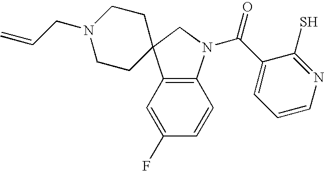 | 1′-(allyl)-1,2-dihydro-5- fluoro-1-(2-mercapto- pyridine-3-carbonyl)- spiro[3H-indole-3,4′- piperidine] |
| 231 | 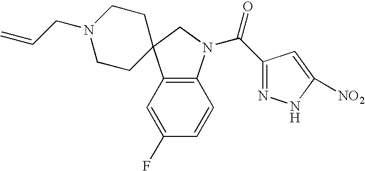 | 1′-(allyl)-1,2-dihydro-5- fluoro-1-(5-nitro-1H- pyrazole-3-carbonyl)- spiro[3H-indole-3,4′- piperidine] |
| 232 | 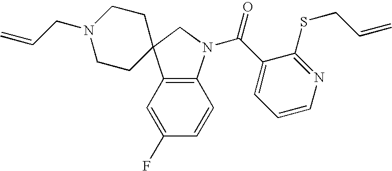 | 1′-(allyl)-1,2-dihydro-5- fluoro-1-(2-allylsulfanyl- pyridine-3-carbonyl)- spiro[3H-indole-3,4′- piperidine] |
| 233 | 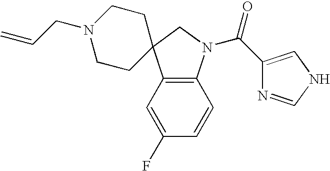 | 1′-(allyl)-1,2-dihydro-5- fluoro-1-(1H-imidazole-4- carbonyl)-spiro[3H- indole-3,4′-piperidine] |
| 234 | 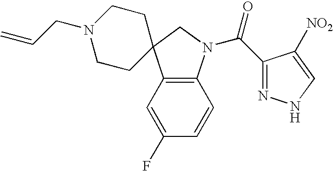 | 1′-(allyl)-1,2-dihydro-5- fluoro-1-(4-nitro-1H- pyrazole-3-carbonyl)- spiro[3H-indole-3,4′- piperidine] |
| 235 | 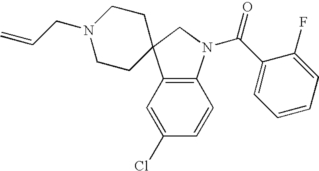 | 1′-(allyl)-1,2-dihydro-5- chloro-1-(2-fluoro- benzoyl)-spiro[3H-indole- 3,4′-piperidine] |
| 236 | 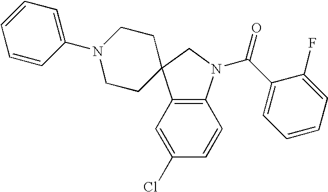 | 1′-(phenyl)-1,2-dihydro-5- chloro-1-(2-fluoro- benzoyl)-spiro[3H-indole- 3,4′-piperidine] |
| 237 | 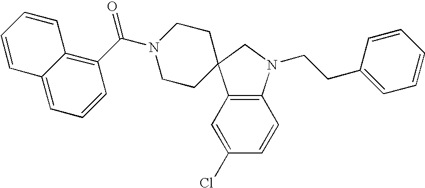 | 1′-(naphthylene-1- carbonyl)-1,2-dihydro-5- chloro-1-(phenethyl)- spiro[3H-indole-3,4′- piperidine] |
| 238 | 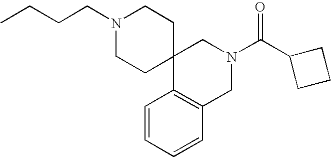 | 1′-(butyl)-2,3-dihydro-2- (cyclobutylcarbonyl)- spiro[isoquinoline- 4(1H),4′-piperidine] |
| 239 | 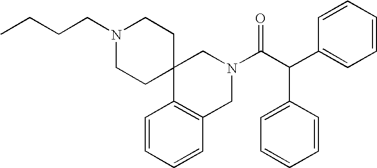 | 1′-(butyl)-2,3-dihydro-2- (diphenylacetyl)- spiro[isoquinoline- 4(1H),4′-piperidine] |
| 240 | 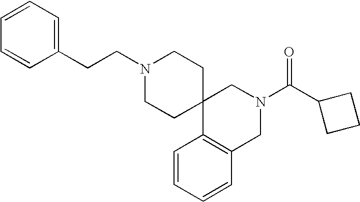 | 1′-(phenethyl)-2,3- dihydro-2- (cyclobutylcarbonyl)- spiro[isoquinoline- 4(1H),4′-piperidine] |
| 241 | 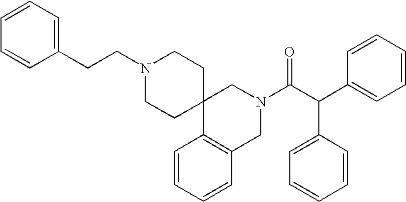 | 1′-(phenethyl)-2,3- dihydro-2- (diphenylacetyl)- spiro[isoquinoline- 4(1H),4′-piperidine] |
| 242 | 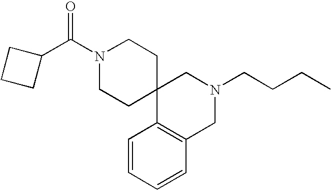 | 1′-(cyclobutylcarbonyl)- 2,3-dihydro-2-(butyl)- spiro[isoquinoline- 4(1H),4′-piperidine] |
| 243 | 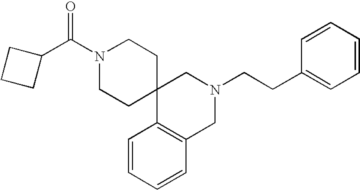 | 1′-(cyclobutylcarbonyl)- 2,3-dihydro-2-(phenethyl)- spiro[isoquinoline- 4(1H),4′-piperidine] |
| 244 | 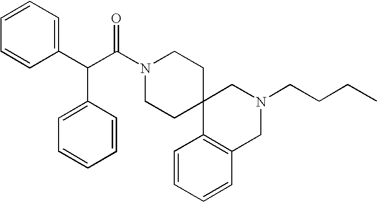 | 1′-(diphenylacetyl)-2,3- dihydro-2-(butyl)- spiro[isoquinoline- 4(1H),4′-piperidine] |
| 245 | 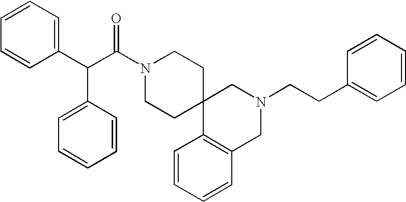 | 1′-(diphenylacetyl)-2,3- dihydro-2-(phenethyl)- spiro[isoquinoline- 4(1H),4′-piperidine] |
| 246 | 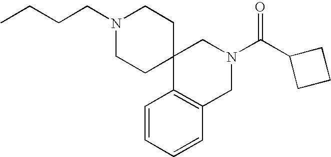 | 1′-(butyl)-2,3-dihydro-2- (cyclobutylcarbonyl)- spiro[isoquinoline- 4(1H),4′-piperidine] |
| 247 | 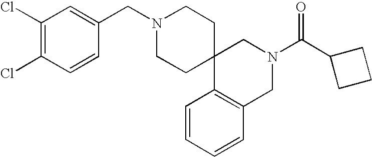 | 1′-(3,4-dichloro-benzyl)- 2,3-dihydro-2- (cyclobutylcarbonyl)- spiro[isoquinoline- 4(1H),4′-piperidine] |
| 248 | 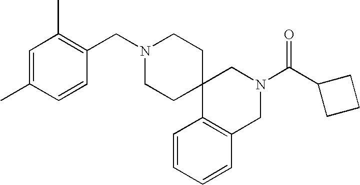 | 1′-(2,4-dimethyl-benzyl)- 2,3-dihydro-2- (cyclobutylcarbonyl)- spiro[isoquinoline- 4(1H),4′-piperidine] |
| 249 | 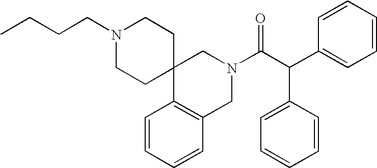 | 1′-(butyl)-2,3-dihydro-2- (diphenylacetyl)- spiro[isoquinoline- 4(1H),4′-piperidine] |
| 250 | 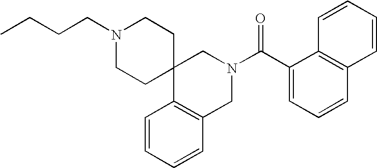 | 1′-(butyl)-2,3-dihydro-2- (naphthylene-1-carbonyl)- spiro[isoquinoline- 4(1H),4′-piperidine] |
| 251 | 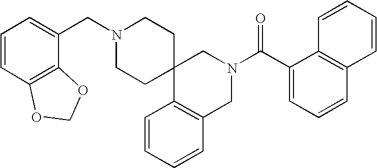 | 1′-(benzo[1,3]dioxol-4- ylmethyl)-2,3-dihydro-2- (naphthylene-1-carbonyl)- spiro[isoquinoline- 4(1H),4′-piperidine] |
| 252 | 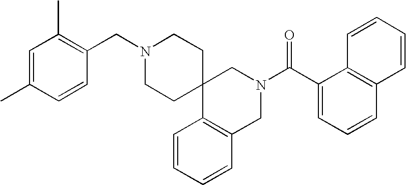 | 1′-(2,4-dimethyl-benzyl)- 2,3-dihydro-2- (naphthylene-1-carbonyl)- spiro[isoquinoline- 4(1H),4′-piperidine] |
| 253 | 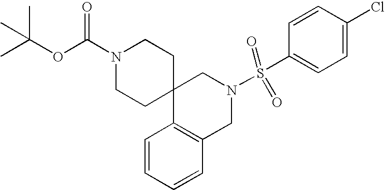 | 1′-(tert-butoxycarbonyl)- 2,3-dihydro-2-(4-chloro- benzenesulfonyl)- spiro[isoquinoline- 4(1H),4′-piperidine] |
| 254 | 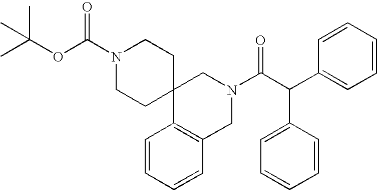 | 1′-(tert-butoxycarbonyl)- 2,3-dihydro-2- (diphenylacetyl)- spiro[isoquinoline- 4(1H),4′-piperidine] |
| 255 | 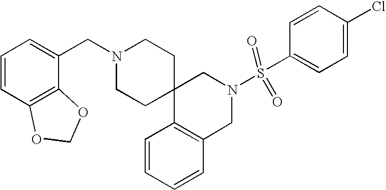 | 1′-(benzo[1,3]dioxol-4- ylmethyl)-2,3-dihydro-2- (4-chloro- benzenesulfonyl)- spiro[isoquinoline- 4(1H),4′-piperidine] |
| 256 | 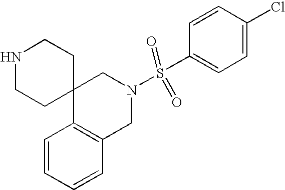 | 2,3-dihydro-2-(4-chloro- benzenesulfonyl)- spiro[isoquinoline- 4(1H),4′-piperidine] |
| 257 | 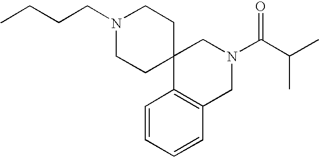 | 1′-(butyl)-2,3-dihydro-2- (isobutyryl)- spiro[isoquinoline- 4(1H),4′-piperidine] |
| 258 | 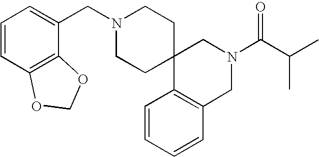 | 1′-(benzo[1,3]dioxol-4- ylmethyl)-2,3-dihydro-2- (isobutyryl)- spiro[isoquinoline- 4(1H),4′-piperidine] |
| 259 | 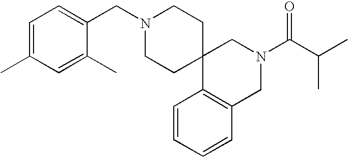 | 1′-(2,4-dimethyl-benzyl)- 2,3-dihydro-2- (isobutyryl)- spiro[isoquinoline- 4(1H),4′-piperidine] |
| 260 | 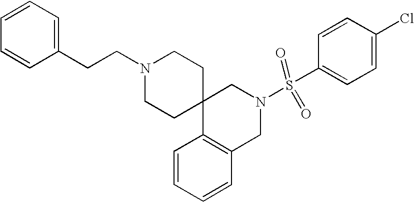 | 1′-(phenethyl)-2,3- dihydro-2-(4-chloro- benzenesulfonyl)- spiro[isoquinoline- 4(1H),4′-piperidine] |
| 261 | 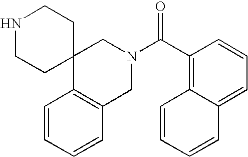 | 2,3-dihydro-2- (naphthylene-1-carbonyl)- spiro[isoquinoline- 4(1H),4′-piperidine] |
| 262 | 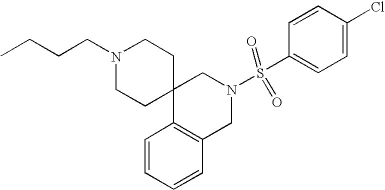 | 1′-(butyl)-2,3-dihydro-2- (4-chloro- benzenesulfonyl)- spiro[isoquinoline- 4(1H),4′-piperidine] |
| 263 | 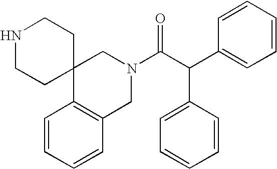 | 2,3-dihydro-2- (diphenylacetyl)- spiro[isoquinoline- 4(1H),4′-piperidine] |
| 264 | 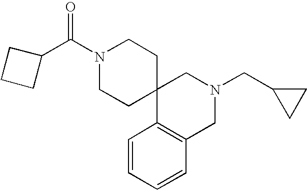 | 1′-(cyclobutylcarbonyl)- 2,3-dihydro-2- (cyclopropylmethyl)- spiro[isoquinoline- 4(1H),4′-piperidine] |
| 265 | 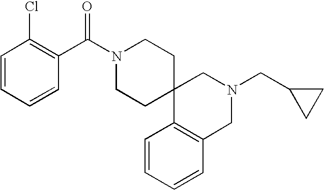 | 1′-(2-chloro-benzoyl)-2,3- dihydro-2- (cyclopropylmethyl)- spiro[isoquinoline- 4(1H),4′-piperidine] |
| 266 | 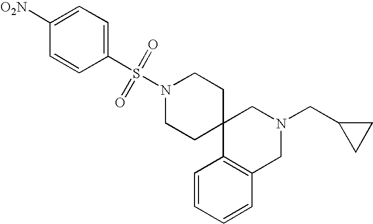 | 1′-(4-nitro- benzenesulfonyl)-2,3- dihydro-2- (cyclopropylmethyl)- spiro[isoquinoline- 4(1H),4′-piperidine] |
| 267 | 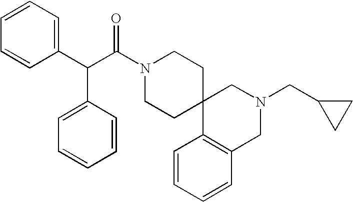 | 1′-(diphenylacetyl)-2,3- dihydro-2- (cyclopropylmethyl)- spiro[isoquinoline- 4(1H),4′-piperidine] |
| 268 | 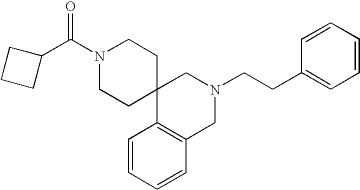 | 1′-(cyclobutylcarbonyl)- 2,3-dihydro-2-(phenethyl)- spiro[isoquinoline- 4(1H),4′-piperidine] |
| 269 | 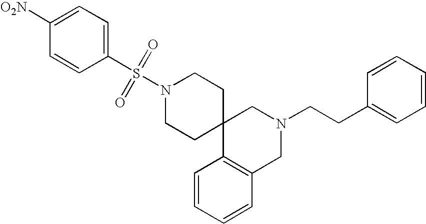 | 1′-(4-nitro- benzenesulfonyl)-2,3- dihydro-2-(phenethyl)- spiro[isoquinoline- 4(1H),4′-piperidine] |
| 270 | 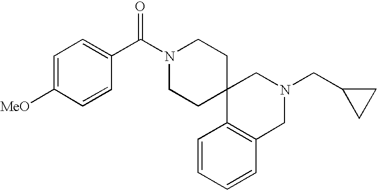 | 1′-(4-methoxy-benzoyl)- 2,3-dihydro-2- (cyclopropylmethyl)- spiro[isoquinoline- 4(1H),4′-piperidine] |
| 271 | 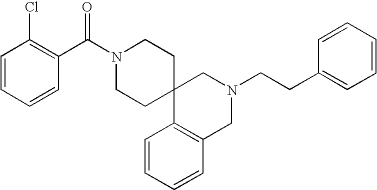 | 1′-(2-chloro-benzoyl)- 2,3-dihydro-2-(phenethyl)- spiro[isoquinoline-4(1H),4′-piperidine] |
| 272 | 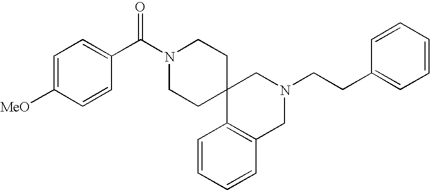 | 1′-(4-methoxy-benzoyl)- 2,3-dihydro-2-(phenethyl)- spiro[isoquinoline- 4(1H),4′-piperidine] |
| 273 | 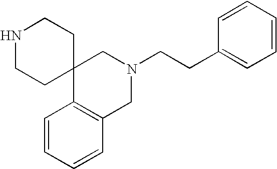 | 2,3-dihydro-2-(phenethyl)- spiro[isoquinoline- 4(1H),4′-piperidine] |
| 274 | 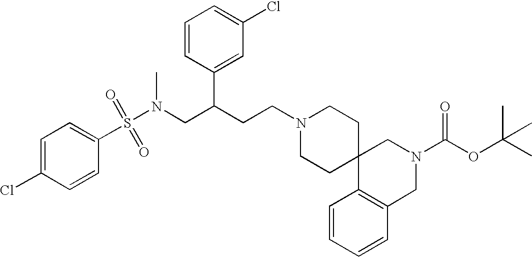 | 1′-{4-[(4-chloro- benzenesulfonyl)-methyl- amino]-3-(3-chloro- phenyl)-butyl}-2,3- dihydro-2-(tert- butoxycarbonyl)- spiro[isoquinoline- 4(1H),4′-piperidine] |
| 275 | 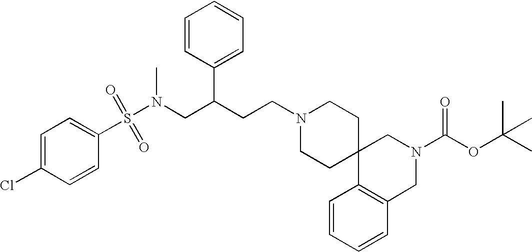 | 1′-{4-[(4-chloro- benzenesulfonyl)-methyl- amino]-3-phenyl-butyl}- 2,3-dihydro-2-(tert- butoxycarbonyl)- spiro[isoquinoline- 4(1H),4′-piperidine] |
| 276 | 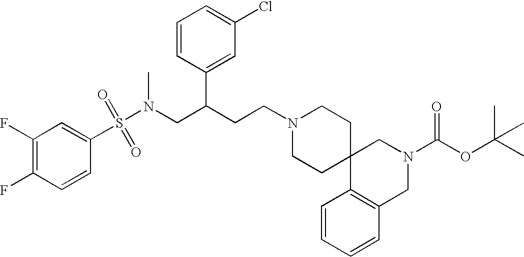 | 1′-{4-[(3,4-difluoro- benzenesulfonyl)-methyl- amino]-3-(3-chloro- phenyl)-butyl}-2,3- dihydro-2-(tert- butoxycarbonyl)- spiro[isoquinoline- 4(1H),4′-piperidine] |
| 277 | 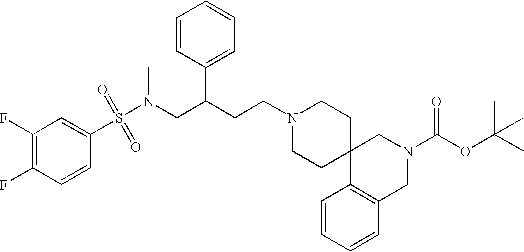 | 1′-{4-[(3,4-difluoro- benzenesulfonyl)-methyl- amino]-3-phenyl-butyl}- 2,3-dihydro-2-(tert- butoxycarbonyl)- spiro[isoquinoline- 4(1H),4′-piperidine] |
| 278 | 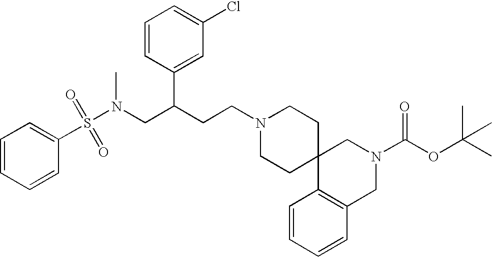 | 1′-{4-[(benzenesulfonyl)- methyl-amino]-3-(3- chloro-phenyl)-butyl}-2,3- dihydro-2-(tert- butoxycarbonyl)- spiro[isoquinoline- 4(1H),4′-piperidine] |
| 279 | 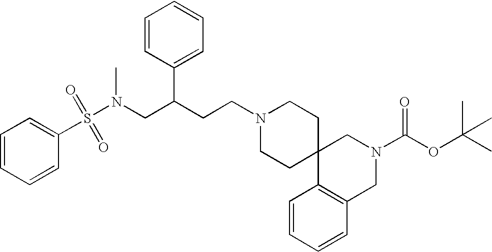 | 1′-{4-[(benzenesulfonyl)- methyl-amino]-3-phenyl- butyl}-2,3-dihydro-2-(tert- butoxycarbonyl)- spiro[isoquinoline- 4(1H),4′-piperidine] |
| 280 | 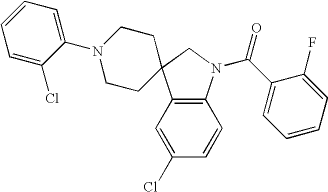 | 1′-(2-chlorophenyl)-1,2- dihydro-5-chloro-1-(2- fluoro-benzoyl)-spiro[3H- indole-3,4′-piperidine] |
| 281 | 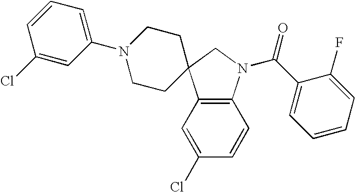 | 1′-(3-chlorophenyl)-1,2- dihydro-5-chloro-1-(2- fluoro-benzoyl)-spiro[3H- indole-3,4′-piperidine] |
| 282 | 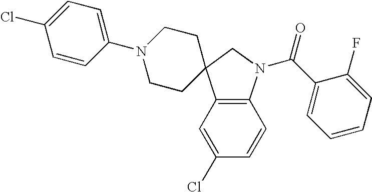 | 1′-(4-chlorophenyl)-1,2- dihydro-5-chloro-1-(2- fluoro-benzoyl)-spiro[3H- indole-3,4′-piperidine] |
| 283 | 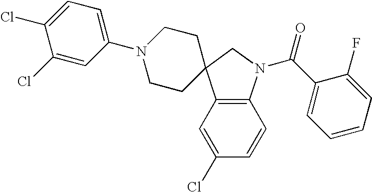 | 1′-(3,4-dichlorophenyl)- 1,2-dihydro-5-chloro-1-(2- fluoro-benzoyl)-spiro[3H- indole-3,4′-piperidine] |
| 284 | 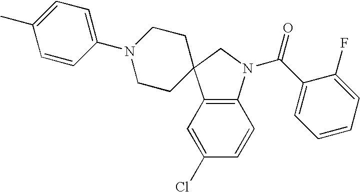 | 1′-(4-methylphenyl)-1,2- dihydro-5-chloro-1-(2- fluoro-benzoyl)-spiro[3H- indole-3,4′-piperidine] |
| 285 | 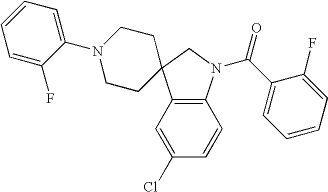 | 1′-(2-fluorophenyl)-1,2- dihydro-5-chloro-1-(2- fluoro-benzoyl)-spiro[3H- indole-3,4′-piperidine] |
| 286 | 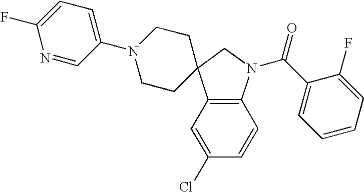 | 1′-(6-fluoro-pyridin-3-yl)- 1,2-dihydro-5-chloro-1-(2- fluoro-benzoyl)-spiro[3H- indole-3,4′-piperidine] |
| 287 | 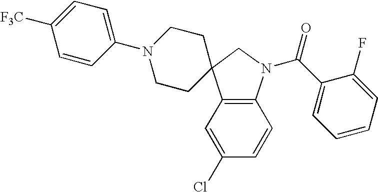 | 1′-(4-trifluoromethyl- phenyl)-1,2-dihydro-5- chloro-1-(2-fluoro- benzoyl)-spiro[3H-indole- 3,4′-piperidine] |
| 288 | 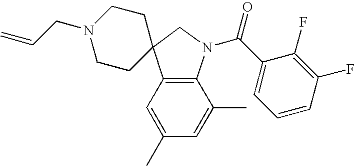 | 1′-(allyl)-1,2-dihydro-5,7- dimethyl-1-(2,3-difluoro- benzoyl)-spiro[3H-indole- 3,4′-piperidine] |
| 289 | 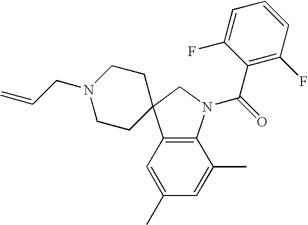 | 1′-(allyl)-1,2-dihydro-5,7- dimethyl-1-(2,6-difluoro- benzoyl)-spiro[3H-indole- 3,4′-piperidine] |
| 290 | 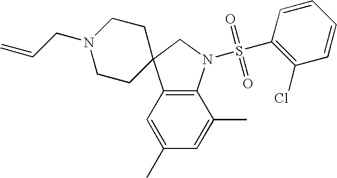 | 1′-(allyl)-1,2-dihydro-5,7- dimethyl-1-(2-chloro- benzenesulfonyl)- spiro[3H-indole-3,4′- piperidine] |
| 291 | 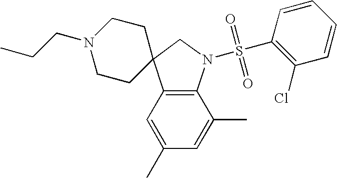 | 1′-(propyl)-1,2-dihydro- 5,7-dimethyl-1-(2-chloro- benzenesulfonyl)- spiro[3H-indole-3,4′- piperidine] |
| 292 | 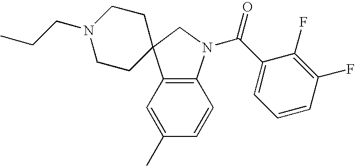 | 1′-(propyl)-1,2-dihydro-5- methyl-1-(2,3-difluoro- benzoyl)-spiro[3H-indole- 3,4′-piperidine] |
| 293 | 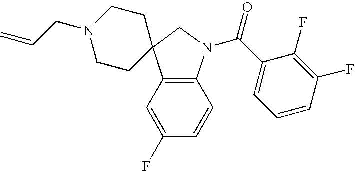 | 1′-(allyl)-1,2-dihydro-5- fluoro-1-(2,3-difluoro- benzoyl)-spiro[3H-indole- 3,4′-piperidine] |
| 294 | 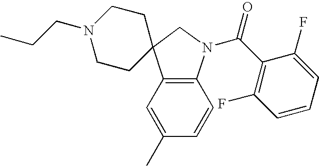 | 1′-(propyl)-1,2-dihydro-5- methyl-1-(2,6-difluoro- benzoyl)-spiro[3H-indole- 3,4′-piperidine] |
| 295 | 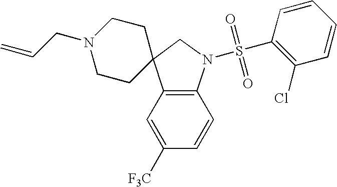 | 1′-(allyl)-1,2-dihydro-5- trifluoromethyl-1-(2- chloro-benzenesulfonyl)- spiro[3H-indole-3,4′- piperidine] |
| 296 | 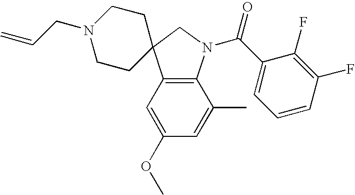 | 1′-(allyl)-1,2-dihydro-5- methoxy-7-methyl-1-(2,3- difluoro-benzoyl)- spiro[3H-indole-3,4′- piperidine] |
| 297 | 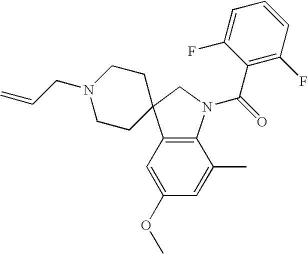 | 1′-(allyl)-1,2-dihydro-5- methoxy-7-methyl-1-(2,6- difluoro-benzoyl)- spiro[3H-indole-3,4′- piperidine] |
| 298 | 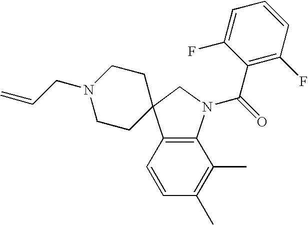 | 1′-(allyl)-1,2-dihydro-6,7- dimethyl-1-(2,6-difluoro- benzoyl)-spiro[3H-indole- 3,4′-piperidine] |
| 299 | 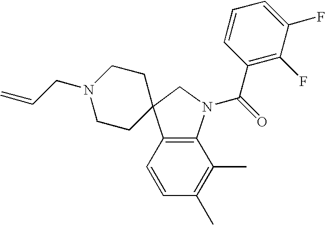 | 1′-(allyl)-1,2-dihydro-6,7- dimethyl-1-(2,3-difluoro- benzoyl)-spiro[3H-indole- 3,4′-piperidine] |
| 300 | 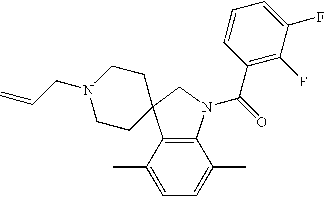 | 1′-(allyl)-1,2-dihydro-4,7- dimethyl-1-(2,3-difluoro- benzoyl)-spiro[3H-indole- 3,4′-piperidine] |
| 301 | 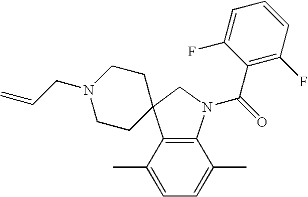 | 1′-(allyl)-1,2-dihydro-4,7- dimethyl-1-(2,6-difluoro- benzoyl)-spiro[3H-indole- 3,4′-piperidine] |
| 302 | 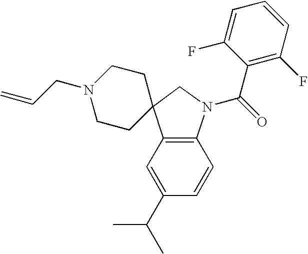 | 1′-(allyl)-1,2-dihydro-5- isopropyl-1-(2,6-difluoro- benzoyl)-spiro[3H-indole- 3,4′-piperidine] |
| 303 | 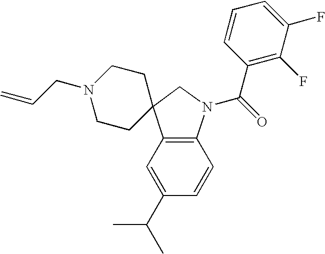 | 1′-(allyl)-1,2-dihydro-5- isopropyl-1-(2,3-difluoro- benzoyl)-spiro[3H-indole- 3,4′-piperidine] |
| 304 | 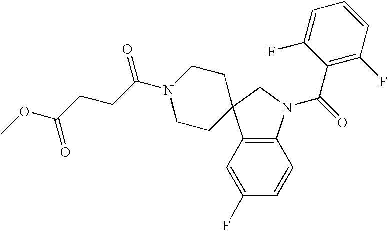 | 1′-(3-methoxycarbonyl- propionyl)-1,2-dihydro-5- fluoro-1-(2,6-difluoro- benzoyl)-spiro[3H-indole- 3,4′-piperidine] |
| 305 | 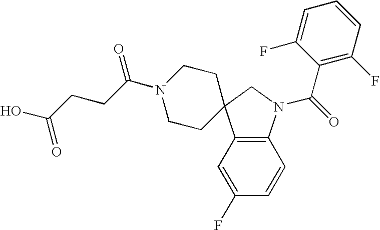 | 1′-(3-carboxy-propionyl)- 1,2-dihydro-5-fluoro-1- (2,6-difluoro-benzoyl)- spiro[3H-indole-3,4′- piperidine] |
| 306 | 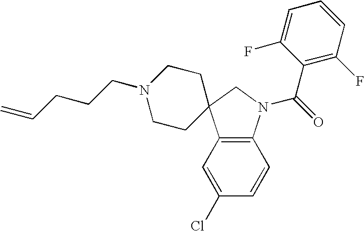 | 1′-(1-pent-4-enyl)-1,2- dihydro-5-chloro-1-(2,6- difluoro-benzoyl)- spiro[3H-indole-3,4′- piperidine] |
| 307 | 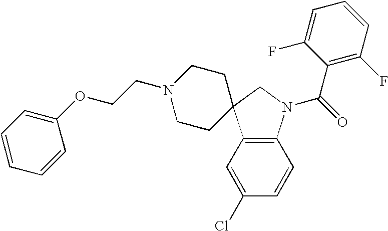 | 1′-(2-phenoxy-ethyl)-1,2- dihydro-5-chloro-1-(2,6- difluoro-benzoyl)- spiro[3H-indole-3,4′- piperidine] |
| 308 | 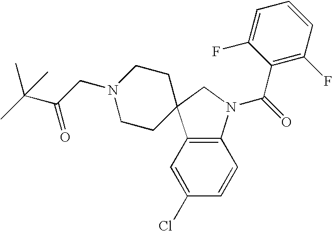 | 1′-(3,3-dimethyl-2-oxo- butyl)-1,2-dihydro-5- chloro-1-(2,6-difluoro- benzoyl)-spiro[3H-indole- 3,4′-piperidine] |
| 309 | 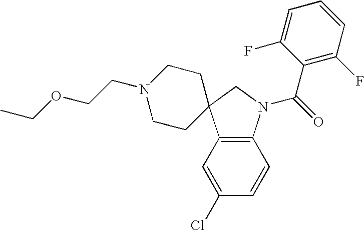 | 1′-(2-ethoxy-ethyl)-1,2- dihydro-5-chloro-1-(2,6- difluoro-benzoyl)- spiro[3H-indole-3,4′- piperidine] |
| 310 | 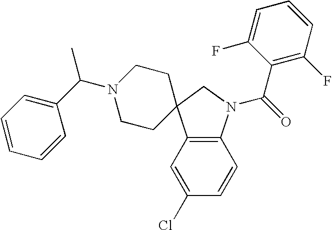 | 1′-(1-phenyl-ethyl)-1,2- dihydro-5-chloro-1-(2,6- difluoro-benzoyl)- spiro[3H-indole-3,4′- piperidine] |
| 311 | 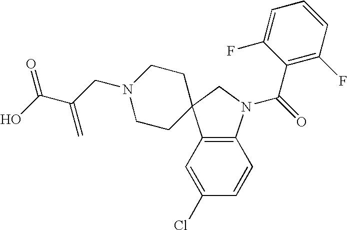 | 1′-(2-carboxy-allyl)-1,2- dihydro-5-chloro-1-(2,6- difluoro-benzoyl)- spiro[3H-indole-3,4′- piperidine] |
| 312 | 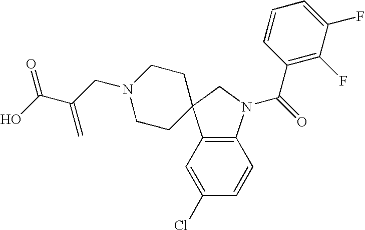 | 1′-(2-carboxy-allyl)-1,2- dihydro-5-chloro-1-(2,3- difluoro-benzoyl)- spiro[3H-indole-3,4′- piperidine] |
| 313 | 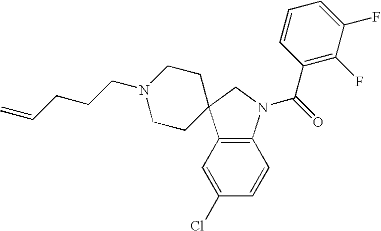 | 1′-(1-pent-4-enyl)-1,2- dihydro-5-chloro-1-(2,3- difluoro-benzoyl)- spiro[3H-indole-3,4′- piperidine] |
| 314 | 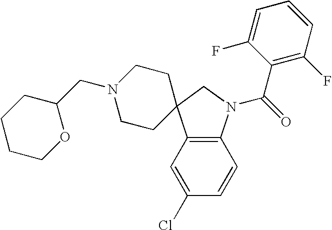 | 1′-(tetrahydro-pyran-2- ylmethyl)-1,2-dihydro-5- chloro-1-(2,6-difluoro- benzoyl)-spiro[3H-indole- 3,4′-piperidine] |
| 315 | 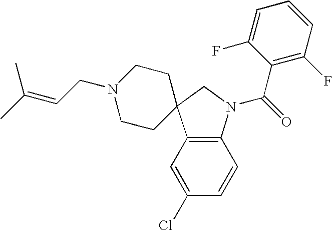 | 1′-(3-methyl-but-2-enyl)- 1,2-dihydro-5-chloro-1- (2,6-difluoro-benzoyl)- spiro[3H-indole-3,4′- piperidine] |
| 316 | 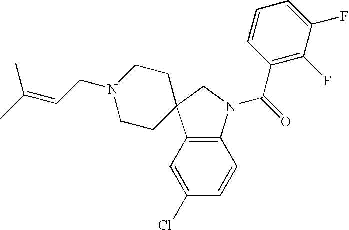 | 1′-(3-methyl-but-2-enyl)- 1,2-dihydro-5-chloro-1- (2,3-difluoro-benzoyl)- spiro[3H-indole-3,4′- piperidine] |
| 317 | 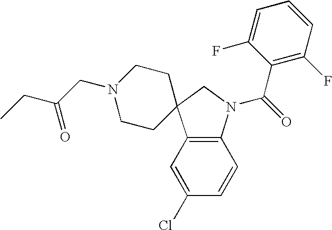 | 1′-(2-oxo-butyl)-1,2- dihydro-5-chloro-1-(2,6- difluoro-benzoyl)- spiro[3H-indole-3,4′- piperidine] |
| 318 | 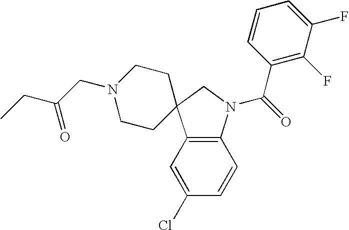 | 1′-(2-oxo-butyl)-1,2- dihydro-5-chloro-1-(2,3- difluoro-benzoyl)- spiro[3H-indole-3,4′- piperidine] |
| 319 | 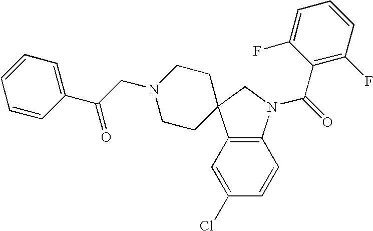 | 1′-(2-oxo-2-phenyl-ethyl)- 1,2-dihydro-5-chloro-1- (2,6-difluoro-benzoyl)- spiro[3H-indole-3,4′- piperidine] |
| 320 | 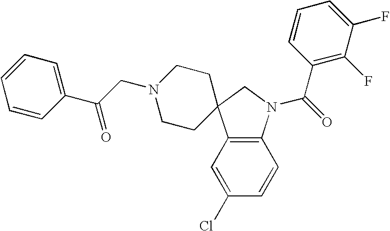 | 1′-(2-oxo-2-phenyl-ethyl)- 1,2-dihydro-5-chloro-1- (2,3-difluoro-benzoyl)- spiro[3H-indole-3,4′- piperidine] |
| 321 | 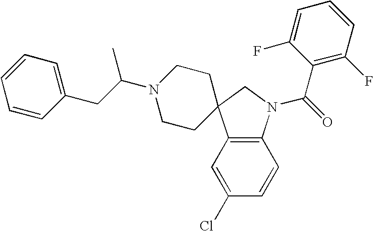 | 1′-(1-methyl-2-phenyl- ethyl)-1,2-dihydro-5- chloro-1-(2,6-difluoro- benzoyl)-spiro[3H-indole- 3,4′-piperidine] |
| 322 | 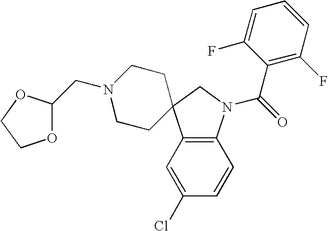 | 1′-([1,3]dioxolan-2- ylmethyl)-1,2-dihydro-5- chloro-1-(2,6-difluoro- benzoyl)-spiro[3H-indole- 3,4′-piperidine] |
| 323 | 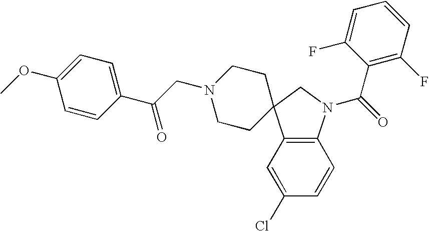 | 1′-[2-(4-methoxy-phenyl)- 2-oxo-ethyl]-1,2-dihydro- 5-chloro-1-(2,6-difluoro- benzoyl)-spiro[3H-indole- 3,4′-piperidine] |
| 324 | 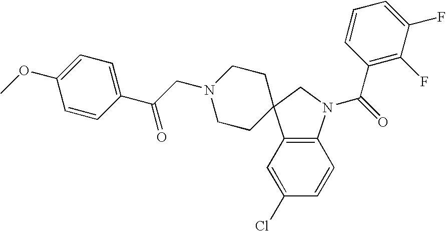 | 1′-[2-(4-methoxy-phenyl)- 2-oxo-ethyl]-1,2-dihydro- 5-chloro-1-(2,3-difluoro- benzoyl)-spiro[3H-indole- 3,4′-piperidine] |
| 325 | 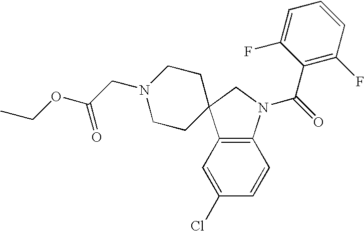 | 1′-(ethoxycarbonyl methyl)-1,2-dihydro-5- chloro-1-(2,6-difluoro- benzoyl)-spiro[3H-indole- 3,4′-piperidine] |
| 326 | 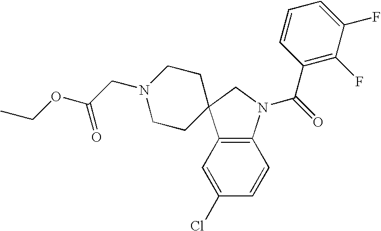 | 1′-(ethoxycarbonyl methyl)-1,2-dihydro-5- chloro-1-(2,3-difluoro- benzoyl)-spiro[3H-indole- 3,4′-piperidine] |
| 327 | 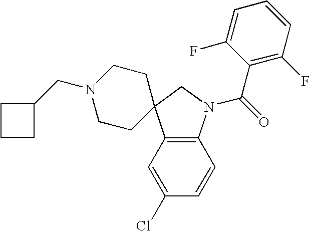 | 1′-(cyclobutylmethyl)-1,2- dihydro-5-chloro-1-(2,6- difluoro-benzoyl)- spiro[3H-indole-3,4′- piperidine] |
| 328 | 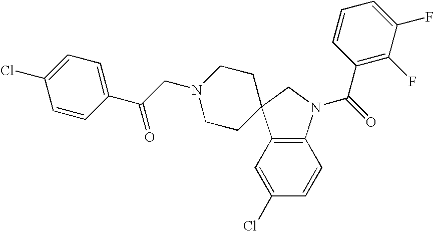 | 1′-[2-(4-chloro-phenyl)-2- oxo-ethyl]-1,2-dihydro-5- chloro-1-(2,3-difluoro- benzoyl)-spiro[3H-indole- 3,4′-piperidine] |
| 329 | 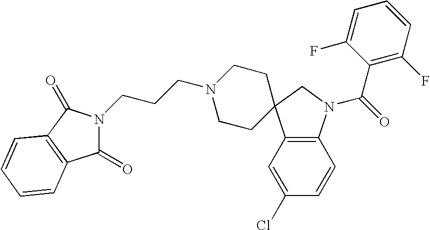 | 1′-[3-(1,3-dioxo-1,3- dihydro-isoindol-2-yl)- propyl]-1,2-dihydro-5- chloro-1-(2,6-difluoro- benzoyl)-spiro[3H-indole- 3,4′-piperidine] |
| 330 | 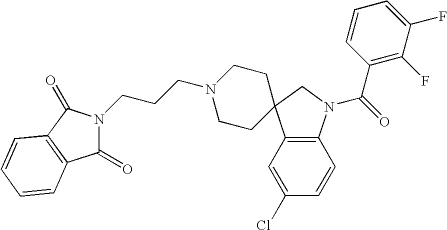 | 1′-[3-(1,3-dioxo-1,3- dihydro-isoindol-2-yl)- propyl]-1,2-dihydro-5- chloro-1-(2,3-difluoro- benzoyl)-spiro[3H-indole- 3,4′-piperidine] |
| 331 | 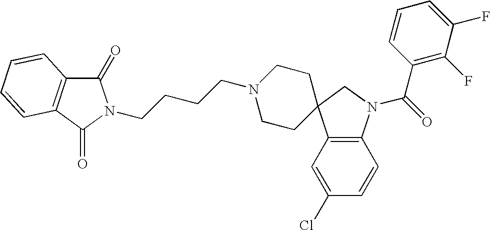 | 1′-[4-(1,3-dioxo-1,3- dihydro-isoindol-2-yl)- butyl]-1,2-dihydro-5- chloro-1-(2,3-difluoro- benzoyl)-spiro[3H-indole- 3,4′-piperidine] |
| 332 | 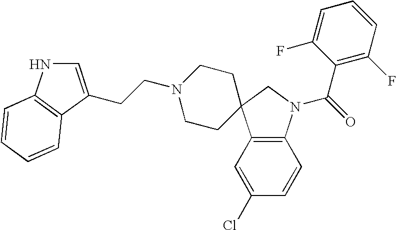 | 1′-[2-(1H-indo1-3-yl)- ethyl]-1,2-dihydro-5- chloro-1-(2,6-difluoro- benzoyl)-spiro[3H-indole- 3,4′-piperidine] |
| 333 | 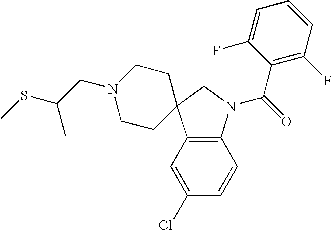 | 1′-(2-methylsulfanyl- propyl)-1,2-dihydro-5- chloro-1-(2,6-difluoro- benzoyl)-spiro[3H-indole- 3,4′-piperidine] |
| 334 | 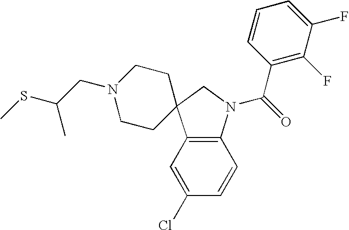 | 1′-(2-methylsulfanyl- propyl)-1,2-dihydro-5- chloro-1-(2,3-difluoro- benzoyl)-spiro[3H-indole- 3,4′-piperidine] |
| 335 | 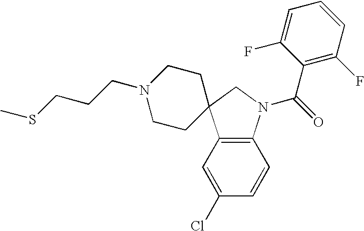 | 1′-(3-methylsulfanyl- propyl)-1,2-dihydro-5- chloro-1-(2,6-difluoro- benzoyl)-spiro[3H-indole- 3,4′-piperidine] |
| 336 | 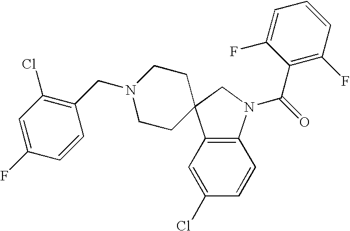 | 1′-(2-chloro-4-fluoro- benzyl)-1,2-dihydro-5- chloro-1-(2,6-difluoro- benzoyl)-spiro[3H-indole- 3,4′-piperidine] |
| 337 | 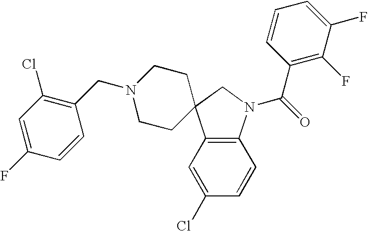 | 1′-(2-chloro-4-fluoro- benzyl)-1,2-dihydro-5- chloro-1-(2,3-difluoro- benzoyl)-spiro[3H-indole- 3,4′-piperidine] |
| 338 | 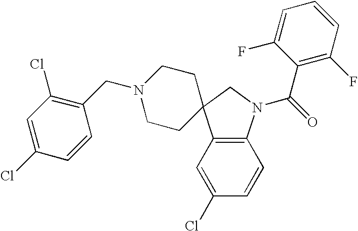 | 1′-(2,4-dichloro-benzyl)- 1,2-dihydro-5-chloro-1- (2,6-difluoro-benzoyl)- spiro[3H-indole-3,4′- piperidine] |
| 339 | 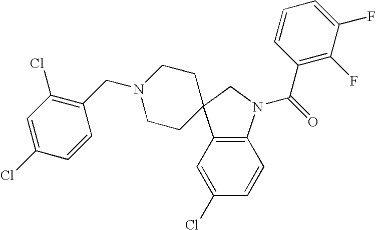 | 1′-(2,4-dichloro-benzyl)- 1,2-dihydro-5-chloro-1- (2,3-difluoro-benzoyl)- spiro[3H-indole-3,4′- piperidine] |
| 340 | 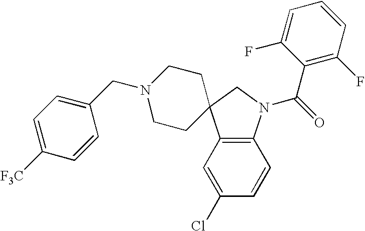 | 1′-(4-trifluoromethyl- benzyl)-1,2-dihydro-5- chloro-1-(2,6-difluoro- benzoyl)-spiro[3H-indole- 3,4′-piperidine] |
| 341 | 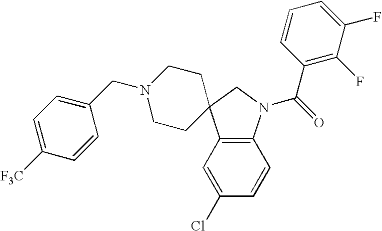 | 1′-(4-trifluoromethyl- benzyl)-1,2-dihydro-5- chloro-1-(2,3-difluoro- benzoyl)-spiro[3H-indole- 3,4′-piperidine] |
| 342 | 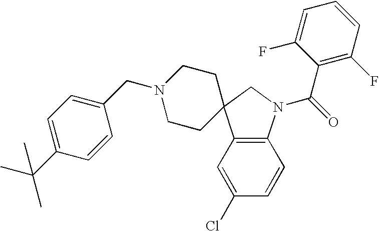 | 1′-(4-tert-butyl-benzyl)- 1,2-dihydro-5-chloro-1- (2,6-difluoro-benzoyl)- spiro[3H-indole-3,4′- piperidine] |
| 343 | 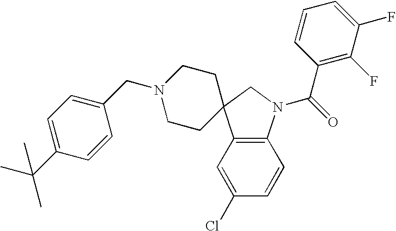 | 1′-(4-tert-butyl-benzyl)- 1,2-dihydro-5-chloro-1- (2,3-difluoro-benzoyl)- spiro[3H-indole-3,4′- piperidine] |
| 344 | 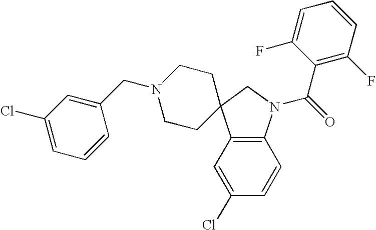 | 1′-(3-chloro-benzyl)-1,2- dihydro-5-chloro-1- (2,6-difluoro-benzoyl)- spiro[3H-indole-3,4′- piperidine] |
| 345 | 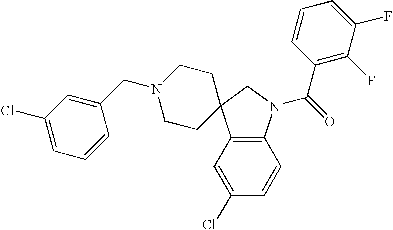 | 1′-(3-chloro-benzyl)-1,2- dihydro-5-chloro-1-(2,3- difluoro-benzoyl)- spiro[3H-indole-3,4′- piperidine] |
| 346 | 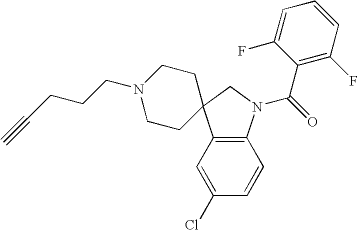 | 1′-(pent-4-ynyl)-1,2- dihydro-5-chloro-1-(2,6- difluoro-benzoyl)- spiro[3H-indole-3,4′- piperidine] |
| 347 | 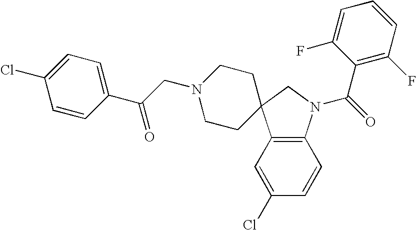 | 1′-[2-(4-chloro-phenyl)-2- oxo-ethyl]-1,2-dihydro-5- chloro-1-(2,6-difluoro- benzoyl)-spiro[3H-indole- 3,4′-piperidine] |
| 348 | 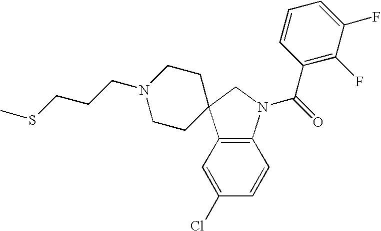 | 1′-(3-methylsulfanyl- propyl)-1,2-dihydro-5- chloro-1-(2,3-difluoro- benzoyl)-spiro[3H-indole- 3,4′-piperidine] |
| 349 | 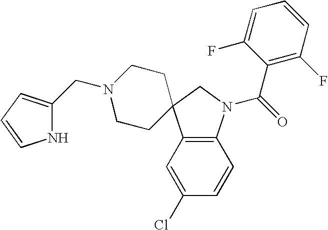 | 1′-(1H-pyrrol-2-ylmethyl)- 1,2-dihydro-5-chloro-1- (2,6-difluoro-benzoyl)- spiro[3H-indole-3,4′- piperidine] |
| 350 | 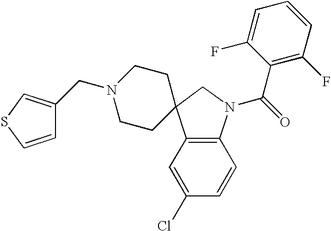 | 1′-(thiophen-3-ylmethyl)- 1,2-dihydro-5-chloro-1- (2,6-difluoro-benzoyl)- spiro[3H-indole-3,4′- piperidine] |
| 351 | 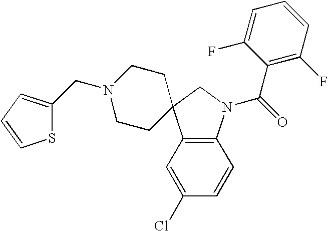 | 1′-(thiophen-2-ylmethyl)- 1,2-dihydro-5-chloro-1- (2,6-difluoro-benzoyl)- spiro[3H-indole-3,4′- piperidine] |
| 352 | 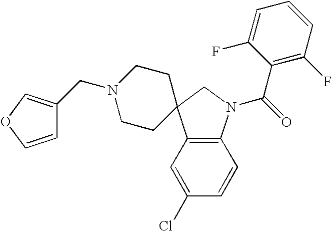 | 1′-(furan-3-ylmethyl)-1,2- dihydro-5-chloro-1-(2,6- difluoro-benzoyl)- spiro[3H-indole-3,4′- piperidine] |
| 353 | 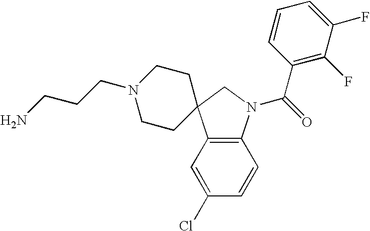 | 1′-(3-amino-propyl)-1,2- dihydro-5-chloro-1-(2,3- difluoro-benzoyl)- spiro[3H-indole-3,4′- piperidine] |
| 354 | 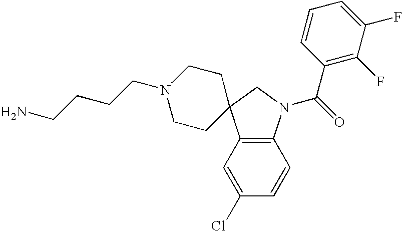 | 1′-(4-amino-butyl)-1,2- dihydro-5-chloro-1-(2,3- difluoro-benzoyl)- spiro[3H-indole-3,4′- piperidine] |
| 355 | 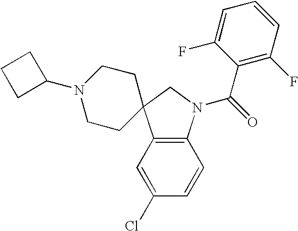 | 1′-(cyclobutyl)-1,2- dihydro-5-chloro-1-(2,6- difluoro-benzoyl)- spiro[3H-indole-3,4′- piperidine] |
| 356 | 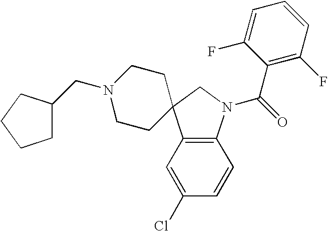 | 1′-(cyclopentylmethyl)- 1,2-dihydro-5-chloro-1- (2,6-difluoro-benzoyl)- spiro[3H-indole-3,4′- piperidine] |
| 357 | 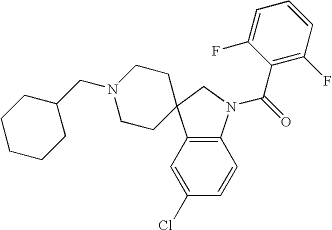 | 1′-(cyclohexylmethyl)-1,2- dihydro-5-chloro-1-(2,6- difluoro-benzoyl)- spiro[3H-indole-3,4′- piperidine] |
| 358 | 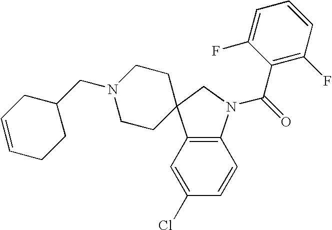 | 1′-(cyclohex-3- enylmethyl)-1,2-dihydro- 5-chloro-1-(2,6-difluoro- benzoyl)-spiro[3H-indole- 3,4′-piperidine] |
| 359 | 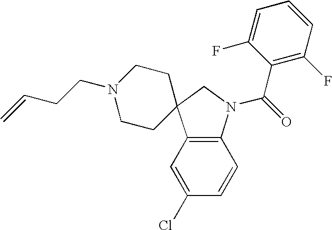 | 1′-(but-3-enyl)-1,2- dihydro-5-chloro-1-(2,6- difluoro-benzoyl)- spiro[3H-indole-3,4′- piperidine] |
| 360 | 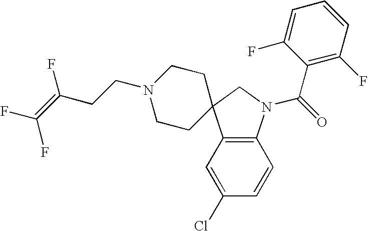 | 1′-(3,4,4-trifluoro-but-3- enyl)-1,2-dihydro-5- chloro-1-(2,6-difluoro- benzoyl)-spiro[3H-indole- 3,4′-piperidine] |
| 361 | 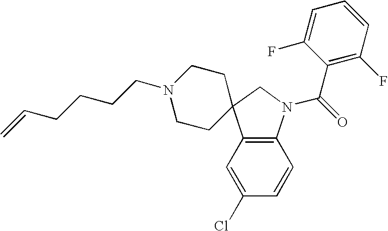 | 1′-(hex-5-enyl)-1,2- dihydro-5-chloro-1-(2,6- difluoro-benzoyl)- spiro[3H-indole-3,4′- piperidine] |
| 362 | 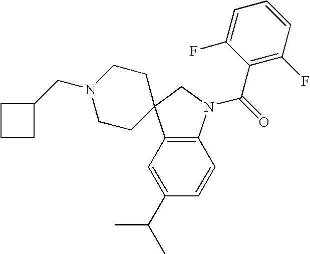 | 1′-(cyclobutylmethyl)-1,2- dihydro-5-isopropyl-1- (2,6-difluoro-benzoyl)- spiro[3H-indole-3,4′- piperidine] |
| 363 | 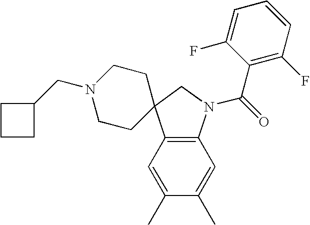 | 1′-(cyclobutylmethyl)-1,2- dihydro-5,6-dimethyl-1- (2,6-difluoro-benzoyl)- spiro[3H-indole-3,4′- piperidine] |
| 364 | 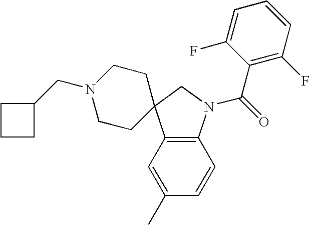 | 1′-(cyclobutylmethyl)-1,2- dihydro-5-methyl-1-(2,6- difluoro-benzoyl)- spiro[3H-indole-3,4′- piperidine] |
| 365 | 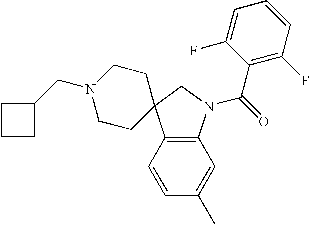 | 1′-(cyclobutylmethyl)-1,2- dihydro-6-methyl-1-(2,6- difluoro-benzoyl)- spiro[3H-indole-3,4′- piperidine] |
| 366 | 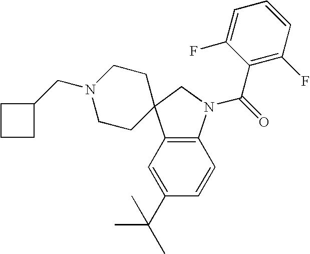 | 1′-(cyclobutylmethyl)-1,2- dihydro-5-tert-butyl-1- (2,6-difluoro-benzoyl)- spiro[3H-indole-3,4′- piperidine] |
| 367 | 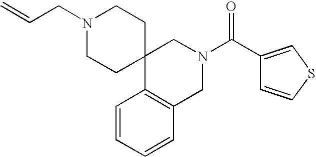 | 1′-(allyl)-2,3-dihydro-2- (thiophene-3-carbonyl)- spiro[isoquinoline- 4(1H),4′-piperidine] |
| 367 | 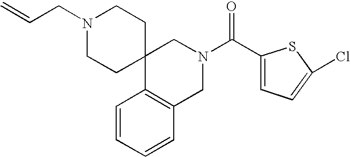 | 1′-(allyl)-2,3-dihydro-2-(5- chloro-thiophene-2- carbonyl)- spiro[isoquinoline- 4(1H),4′-piperidine] |
| 369 | 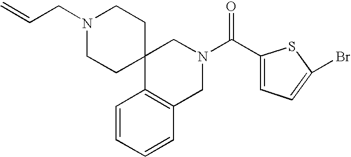 | 1′-(allyl)-2,3-dihydro-2-(5- bromo-thiophene-2- carbonyl)- spiro[isoquinoline- 4(1H),4′-piperidine] |
| 370 | 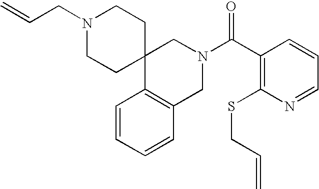 | 1′-(allyl)-2,3-dihydro-2-(2- allylsulfanyl-pyridine-3- carbonyl)- spiro[isoquinoline- 4(1H),4′-piperidine] |
| 371 | 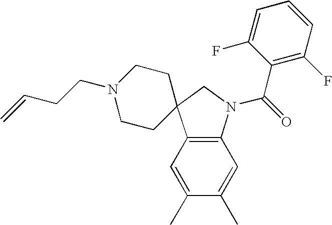 | 1′-(but-3-enyl)-1,2- dihydro-5,6-dimethyl-1- (2,6-difluoro-benzoyl)- spiro[3H-indole-3,4′- piperidine] |
| 372 | 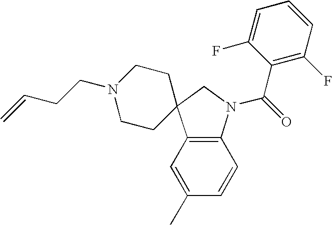 | 1′-(but-3-enyl)-1,2- dihydro-5-methyl-1-(2,6- difluoro-benzoyl)- spiro[3H-indole-3,4′- piperidine] |
| 373 | 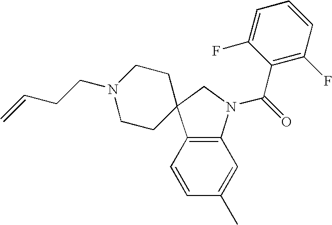 | 1′-(but-3-enyl)-1,2- dihydro-6-methyl-1-(2,6- difluoro-benzoyl)- spiro[3H-indole-3,4′- piperidine] |
| 374 | 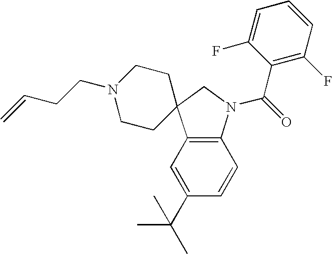 | 1′-(but-3-enyl)-1,2- dihydro-5-tert-butyl-1- (2,6-difluoro-benzoyl)- spiro[3H-indole-3,4′- piperidine] |
| 375 | 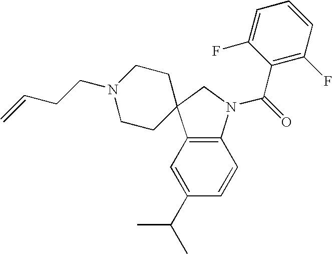 | 1′-(but-3-enyl)-1,2- dihydro-5-isopropyl-1- (2,6-difluoro-benzoyl)- spiro[3H-indole-3,4′- piperidine] |
| 376 | 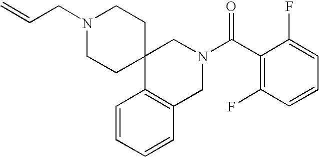 | 1′-(allyl)-2,3-dihydro-2- (2,6-difluoro-benzoyl)- spiro[isoquinoline- 4(1H),4′-piperidine] |
| 377 | 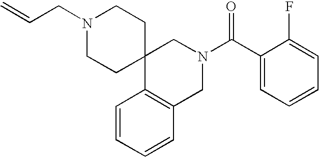 | 1′-(allyl)-2,3-dihydro-2-(2- fluoro-benzoyl)- spiro[isoquinoline- 4(1H),4′-piperidine] |
| 378 | 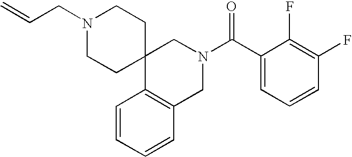 | 1′-(allyl)-2,3-dihydro-2- (2,3-difluoro-benzoyl)- spiro[isoquinoline- 4(1H),4′-piperidine] |
| 379 | 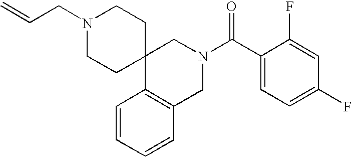 | 1′-(allyl)-2,3-dihydro-2- (2,4-difluoro-benzoyl)- spiro[isoquinoline- 4(1H),4′-piperidine] |
| 380 | 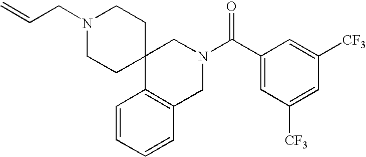 | 1′-(allyl)-2,3-dihydro-2- (3,5-bis-ditrifluoromethyl- benzoyl)- spiro[isoquinoline- 4(1H),4′-piperidine] |
| 381 | 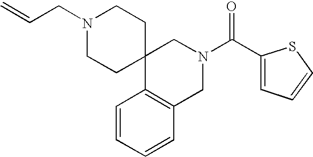 | 1′-(allyl)-2,3-dihydro-2- (thiophene-2-carbonyl)- spiro[isoquinoline- 4(1H),4′-piperidine] |
| 382 | 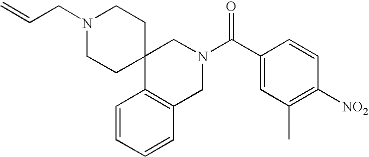 | 1′-(allyl)-2,3-dihydro-2-(3- methyl-4-nitro-benzoyl)- spiro[isoquinoline- 4(1H),4′-piperidine] |
| 383 | 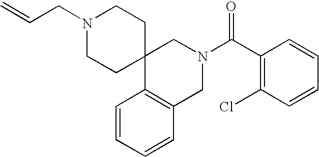 | 1′-(allyl)-2,3-dihydro-2-(2- chloro-benzoyl)- spiro[isoquinoline- 4(1H),4′-piperidine] |
| 384 | 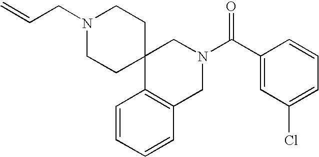 | 1′-(allyl)-2,3-dihydro-2-(3- chloro-beuzoyl)- spiro[isoquinoline- 4(1H),4′-piperidine] |
| 385 | 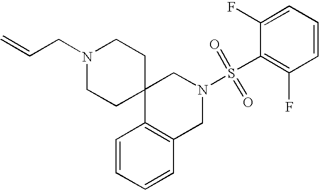 | 1′-(allyl)-2,3-dihydro-2- (2,6-difluoro- benzenesulfonyl)- spiro[isoquinoline- 4(1H),4′-piperidine] |
| 386 | 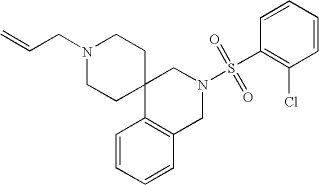 | 1′-(allyl)-2,3-dihydro-2-(2- chloro-benzenesulfonyl)- spiro[isoquinoline- 4(1H),4′-piperidine] |
| 387 | 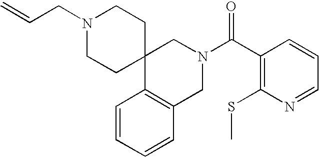 | 1′-(allyl)-2,3-dihydro-2-(2- methylsulfanyl-pyridine-3- carbonyl)- spiro[isoquinoline- 4(1H),4′-piperidine] |
| 388 | 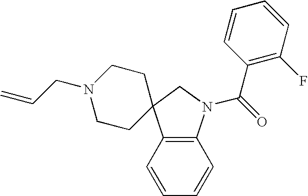 | 1′-(allyl)-1,2-dihydro-1-(2- fluoro-benzoyl)-spiro[3H- indole-3,4′-piperidine] |
| 389 | 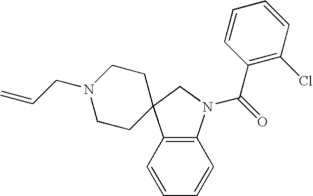 | 1′-(allyl)-1,2-dihydro-1-(2- chloro-benzoyl)-spiro[3H- indole-3,4′-piperidine] |
| 390 | 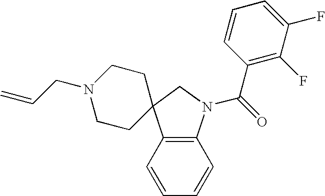 | 1′-(allyl)-1,2-dihydro-1- (2,3-difluoro-benzoyl)- spiro[3H-indole-3,4′- piperidine] |
| 391 | 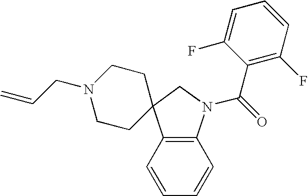 | 1′-(allyl)-1,2-dihydro-1- (2,6-difluoro-benzoyl)- spiro[3H-indole-3,4′- piperidine] |

### 5.7 CHEMICAL DEFINITIONS

As used herein, the terms used above have the following meaning:

“—(C₁₋₈)alkyl” means a saturated straight chain or branched non-cyclic hydrocarbon having from 1 to 8 carbon atoms. Representative saturated straight chain —(C₁₋₈)alkyls include -methyl, -ethyl, -n-propyl, -n-butyl, -n-pentyl, -n-hexyl, -n-heptyl and -n-octyl. Representative saturated branched —(C₁₋₈)alkyls include -isopropyl, -sec-butyl, -isobutyl, -tert-butyl, -isopentyl, -2-methylbutyl, -3-methylbutyl, -2,2-dimethylbutyl, -2,3-dimethylbutyl, -2-methylpentyl, -3-methylpentyl, -4-methylpentyl, -2,2-dimethylhexyl, -3,3-dimethylhexyl, -1-ethylhexyl and the like.

“—(C₁₋₆)alkyl” means a saturated straight chain or branched non-cyclic hydrocarbon having from 1 to 6 carbon atoms. Representative saturated straight chain —(C₁₋₆)alkyls include -methyl, -ethyl, -n-propyl, -n-butyl, -n-pentyl, and -n-hexyl. Representative saturated branched —(C₁₋₆)alkyls include -isopropyl, -sec-butyl, -isobutyl, -tert-butyl, -isopentyl, -2-methylbutyl, -3-methylbutyl, -2,2-dimethylbutyl, -2,3-dimethylbutyl, -2-methylpentyl, -3-methylpentyl, -4-methylpentyl and the like.

“—(C₁₋₄)alkyl” means a saturated straight chain or branched non-cyclic hydrocarbon having from 1 to 4 carbon atoms. Representative saturated straight chain —(C₁₋₄)alkyls include -methyl, -ethyl, -n-propyl, and -n-butyl. Representative saturated branched —(C₁₋₄)alkyls include -isopropyl, -sec-butyl, -isobutyl, and -tert-butyl.

“—(C₀₋ₓ)alkyl” means a direct bond or a saturated straight chain or branched non-cyclic hydrocarbon having up to X carbon atoms, such as those described above.

“—(C₂₋₆)alkenyl” means a straight chain or branched non-cyclic hydrocarbon having from 2 to 6 carbon atoms and including at least one carbon-carbon double bond. Representative straight chain and branched (C₂₋₆)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl and the like.

“—(C₂₋₆)alkynyl” means a straight chain or branched non-cyclic hydrocarbon having from 2 to 6 carbon atoms and including at lease one carbon-carbon triple bond. Representative straight chain and branched (C₂₋₆)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl and the like.

“Aryl” means a monocyclic, bicyclic or tricyclic carbocyclic, aromatic group containing from 6 to 14 carbon atoms in the ring. Representative examples include, but are not limited to, phenyl, anthracenyl, phenanthryl, fluorenyl, naphthyl, and the like. Additional examples include benzo-fused carbocyclic moieties, such as, 5,6,7,8-tetrahydronaphthyl, indenyl, indanyl, and the like. An aryl group can be unsubstituted or substituted. In one embodiment, the aryl group is a phenyl group.

“—(C₃₋₈) cycloalkyl” means a saturated cyclic hydrocarbon having from 3 to 8 carbon atoms. Representative (C₃₋₈)cycloalkyls include -cyclopropyl, -cyclobutyl, -cyclopentyl, -cyclohexyl, -cycloheptyl and -cyclooctyl.

“—(C₈₋₁₄) bicycloalkyl” means a bi-cyclic hydrocarbon ring system having from 8 to 14 carbon atoms and at least one saturated cyclic alkyl ring. Representative —(C₈₋₁₄)bicycloalkyls include -indanyl, -1,2,3,4-tetrahydronaphthyl, -5,6,7,8-tetrahydronaphthyl, -perhydronaphthyl and the like.

“—(C₈₋₁₄) tricycloalkyl” means a tri-cyclic hydrocarbon ring system having from 8 to 14 carbon atoms and at least one saturated cycloalkyl ring. Representative —(C₈₋₁₄)tricycloalkyls include -pyrenyl, -adamantyl, -1,2,3,4-tetrahydroanthracenyl, -perhydroanthracenyl, -aceanthreneyl, -1,2,3,4-tetrahydropenanthrenyl, -5,6,7,8-tetrahydrophenanthrenyl, -perhydrophenanthrenyl and the like.

“—(C₅₋₁₀) cycloalkenyl” means a cyclic non-aromatic hydrocarbon having at least one carbon-carbon double bond in the cyclic system and from 5 to 10 carbon atoms. Representative (C₅-C₁₀)cycloalkenyls include -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, -cyclooctatrienyl, -cyclooctatetraenyl, -cyclononenyl, -cyclononadienyl, -cyclodecenyl, -cyclodecadienyl and the like.

“-(5 to 10 membered) heteroaryl” means an aromatic heterocycle ring of 5 to 10 members, including both mono- and bicyclic ring systems, where at least one carbon atom of one or both of the rings is replaced with a heteroatom independently selected from nitrogen, oxygen, and sulfur. In one embodiment one of the -(5 to 10 membered)heteroaryl's rings contain at least one carbon atom. In another embodiment both of the -(5 to 10 membered)heteroaryl's rings contain at least one carbon atom. Representative (5 to 10 membered)heteroaryls include pyridyl, furyl, benzofuranyl, benzo(1,3)dioxole, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl and the like. An -(5 to 10 membered) heteroaryl group can be unsubstituted or substituted.

“-(3 to 7 membered)heterocycle” or “-(3 to 7 membered)heterocyclo” means a 3- to 7-membered monocyclic heterocyclic ring which is either saturated, unsaturated, non-aromatic or aromatic. A 3- or a 4-membered heterocycle can contain up to 3 heteroatoms, a 5-membered heterocycle can contain up to 4 heteroatoms, a 6-membered heterocycle can contain up to 6 heteroatoms, and a 7-membered heterocycle can contain up to 7 heteroatoms. Each heteroatom is independently selected from nitrogen, which can be quaternized with a hydrogen or alkyl group; oxygen; and sulfur, including sulfoxide and sulfone. The -(3 to 7 membered)heterocycle can be attached via any heteroatom or carbon atom. Representative -(3 to 7 membered)heterocycles include pyridyl, furyl, thiophenyl, pyrrolyl, oxazolyl, imidazolyl, thiazolyl, isoxazolyl, pyrazolyl, isothiazolyl, pyridazinyl, pyrimidinyl, pyrazinyl, triazinyl, morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl and the like.

“-(7 to 10 membered)bicycloheterocycle” or “-(7 to 10 membered) bicycloheterocyclo” means a 7 to 10 membered bicyclic, heterocyclic ring having a saturated, unsaturated, non-aromatic or aromatic group. A -(7 to 10 membered)bicycloheterocycle contains from 1 to 4 heteroatoms independently selected from nitrogen, which can be quaternized with a hydrogen or alkyl group; oxygen; and sulfur, including sulfoxide and sulfone. The (7 to 10 membered)bicycloheterocycle can be attached via any heteroatom or carbon atom. Representative -(7 to 10 membered)bicycloheterocycles include -quinolinyl, -isoquinolinyl, -chromonyl, -coumarinyl, -indolyl, -indolizinyl, -benzo[b]furanyl, -benzo[b]thiophenyl, -indazolyl, -purinyl, -4H-quinolizinyl, -isoquinolyl, -quinolyl, -phthalazinyl, -naphthyridinyl, -carbazolyl, -β-carbolinyl, -benzo(1,3)dioxole and the like. A benzo(1,3)dioxole has the structure:

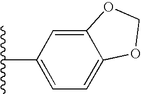

The term “C₁₋₆ acyl” means a C₁₋₆ alkyl radical attached to a carbonyl group wherein the definition of alkyl has the same definition as described herein; some examples include but not limited to, acetyl, propionyl, n-butanoyl, iso-butanoyl, sec-butanoyl, t-butanoyl (i.e., pivaloyl), pentanoyl and the like.

The term “C₁₋₆ acyloxy” means an acyl radical attached to an oxygen atom wherein acyl has the same definition has described herein; some examples include but not limited to acetyloxy, propionyloxy, butanoyloxy, iso-butanoyloxy, sec-butanoyloxy, t-butanoyloxy and the like.

The term “C₂₋₆ alkenyl” means a radical containing 2 to 6 carbons wherein at least one carbon-carbon double bond is present, some embodiments are 2 to 4 carbons, some embodiments are 2 to 3 carbons, and some embodiments have 2 carbons. Both E and Z isomers are embraced by the term “alkenyl.” Furthermore, the term “alkenyl” includes di- and tri-alkenyls. Accordingly, if more than one double bond is present then the bonds may be all E or Z or a mixture of E and Z. Examples of an alkenyl include vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2,4-hexadienyl and the like.

The term “C₂₋₆ alkenylthio” means a C₂₋₆ alkenyl radical, as defined herein, directly bonded to a sulfur atom (i.e., —S—C₂₋₆ alkenyl), wherein at least one carbon-carbon double bond is present, examples include, but not limited to, —S—CH₂CH═CH₂, —S—CH₂CH₂CH═CH₂, and the like.

The term “C₁₋₆ alkoxy” as used herein means a radical alkyl, as defined herein, attached directly to an oxygen atom. Examples include methoxy, ethoxy, n-propoxy, iso-propoxy, n-butoxy, t-butoxy, iso-butoxy, sec-butoxy and the like.

The term “C₁₋₆ alkylamino” means a C₁₋₆ alkyl group attached to the —NH group, examples include but not limited to, methylamino (—NHCH₃), ethylamino, propylamino, isopropylamino, and the like.

The term “C₁₋₆ alkylcarboxamido” or “C₁₋₆ alkylcarboxamide” means a single C₁₋₆ alkyl group attached to the nitrogen of an amide group, wherein alkyl has the same definition as found herein. The C₁₋₆ alkylcarboxamido may be represented by the following: 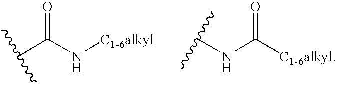
Examples include, but not limited to, N-methylcarboxamide, N-ethylcarboxamide, N-n-propylcarboxamide, N-iso-propylcarboxamide, N-n-butylcarboxamide, N-sec-butylcarboxamide, N-iso-butylcarboxamide, N-t-butylcarboxamide and the like.

The term “C₁₋₃ alkylene” refers to a C₁₋₃ divalent straight carbon group. In some embodiments C₁₋₃ alkylene refers to, for example, —CH₂—, —CH₂CH₂—, —CH₂CH₂CH₂—, and the like.

The term “C₂₋₆ alkynyl” means a radical containing 2 to 6 carbons and at least one carbon-carbon triple bond, some embodiments are 2 to 4 carbons, some embodiments are 2 to 3 carbons, and some embodiments have 2 carbons. Examples of an alkynyl include, but not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “C₁₋₆ alkylsulfonamide” refers to the groups 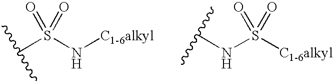
wherein C₁₋₆ alkyl has the same definition as described herein.

The term “C₁₋₆ alkylsulfinyl” means a C₁₋₆ alkyl radical attached to a sulfoxide radical of the formula: —S(O)— wherein the alkyl radical has the same definition as described herein. Examples include, but not limited to, methylsulfinyl, ethylsulfinyl, n-propylsulfinyl, iso-propylsulfonyl, n-butylsulfinyl, sec-butylsulfinyl, iso-butylsulfinyl, t-butyl, and the like.

The term “C₁₋₆ alkylsulfonyl” means a C₁₋₆ alkyl radical attached to a sulfone radical of the formula: —S(O)₂— wherein the alkyl radical has the same definition as described herein. Examples include, but not limited to, methylsulfonyl, ethylsulfonyl, n-propylsulfonyl, isopropylsulfonyl, n-butylsulfonyl, sec-butylsulfonyl, iso-butylsulfonyl, t-butyl, and the like.

The term “C₁₋₆ alkylthio” means a C₁₋₆ alkyl radical attached to a sulfide of the formula: —S— wherein the alkyl radical has the same definition as described herein. Examples include, but not limited to, methylsulfanyl (i.e., CH₃S—), ethylsulfanyl, n-propylsulfanyl, iso-propylsulfanyl, n-butylsulfanyl, sec-butylsulfanyl, iso-butylsulfanyl, t-butyl, and the like.

The term “C₁₋₆ alkylureyl” means the group of the formula: —NC(O)N— wherein one are both of the nitrogens are substituted with the same or different C₁₋₆ alkyl group wherein alkyl has the same definition as described herein. Examples of an alkylureyl include, but not limited to, CH₃NHC(O)NH—, NH₂C(O)NCH₃—, (CH₃)₂N(O)NH—, (CH₃)₂N(O)NH—, (CH₃)₂N(O)NCH₃—, CH₃CH₂NHC(O)NH—, CH₃CH₂NHC(O)NCH₃—, and the like.

The term “arylsulfonyl” means an aryl attached to a sulfone radical of the formula: —S(O)₂— wherein the aryl radical has the same definition as described herein. Examples of an arylsulfonyl include benzenesulfonyl, and the like.

The term “carbo-C₁₋₆-alkoxy” means a C₁₋₆ alkyl ester of a carboxylic acid and can be represented by the formula: —C(═O)—O—C₁₋₆ alkyl, wherein the alkyl group is as defined herein. Examples include, but not limited to, carbomethoxy (—C(═O)OCH₃), carboethoxy, carbopropoxy, carboisopropoxy, carbobutoxy, carbo-sec-butoxy, carbo-iso-butoxy, carbo-t-butoxy, carbo-n-pentoxy, carbo-iso-pentoxy, carbo-t-pentoxy, carbo-neo-pentoxy, carbo-in-hexyloxy, and the like.

The term “carboxamide” means the group —CONH₂.

The term “C₁₋₆ dialkylamino” means two C₁₋₆ alkyl radicals, that are the same or different, attached to a nitrogen atom, examples include, but not limited to, dimethylamino [—N(CH₃)₂], methylethylamino, diethylamino, methylpropylamino, and the like.

The term “C₁₋₆ dialkylcarboxamido” or “C₁₋₆ dialkylcarboxamide” means two C₁₋₆ alkyl radicals, that are the same or different, attached to an amide group, wherein alkyl has the same definition as described herein. A C₁₋₆ dialkylcarboxamido may be represented by the following groups: 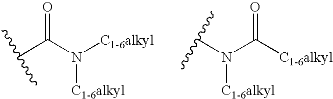
Examples of a dialkylcarboxamide include, but not limited to, N,N-dimethylcarboxamide, N-methyl-N-ethylcarboxamide, N,N-diethylcarboxamide, N-methyl-N-isopropylcarboxamide, and the like.

The term “C₁₋₆ haloalkoxy” means a haloalkyl, as defined herein, which is directly attached to an oxygen atom. Examples include, but not limited to, difluoromethoxy, trifluoromethoxy, 2,2,2-trifluoroethoxy, pentafluoroethoxy and the like.

The term “C₁₋₆ haloalkyl” means an C₁₋₆ alkyl group, defined herein, wherein the alkyl is substituted with one halogen up to fully substituted and a fully substituted C₁₋₆ haloalkyl can be represented by the formula CₙL₂ₙ₊₁ wherein L is a halogen and “n” is 1, 2, 3, 4, 5, or 6; when more than one halogen is present then they may be the same or different and selected from the group consisting of F, Cl, Br and I, preferably F. Examples of C₁₋₄ haloalkyl groups include, but not limited to, fluoromethyl, difluoromethyl, trifluoromethyl, chlorodifluoromethyl, 2,2,2-trifluoroethyl, pentafluoroethyl and the like.

The term “C₁₋₆ haloalkylsulfinyl” means a haloalkyl radical attached to a sulfoxide group of the formula: —S(═O)— wherein the haloalkyl radical has the same definition as described herein. Examples include, but not limited to, trifluoromethylsulfinyl, 2,2,2-trifluoroethylsulfinyl, 2,2-difluoroethylsulfinyl and the like.

The term “C₁₋₆ haloalkylsulfonyl” means a haloalkyl radical attached to a sulfone group of the formula: —S(═O)₂— wherein haloalkyl has the same definition as described herein. Examples include, but not limited to, trifluoromethylsulfonyl, 2,2,2-trifluoroethylsulfonyl, 2,2-difluoroethylsulfonyl and the like.

The term “C₁₋₆ haloalkylthio” means a haloalkyl radical directly attached to a sulfur wherein the haloalkyl has the same meaning as described herein. Examples include, but not limited to, trifluoromethylthio (i.e., CF3S—), 1,1-difluoroethylthio, 2,2,2-trifluoroethylthio and the like.

The term “heterocyclic” means a non-aromatic carbon ring (i.e., cycloalkyl or cycloalkenyl as defined herein) wherein one, two or three ring carbons are replaced by a heteroatom selected from, but not limited to, the group consisting of O, S, N, wherein the N can be optionally substituted with H, C₁₋₆ acyl or C₁₋₆ alkyl, and ring carbon atoms optionally substituted with oxo or a thiooxo thus forming a carbonyl or thiocarbonyl group. The heterocyclic group is a 3-, 4-, 5-, 6- or 7-membered containing ring. Examples of a heterocyclic group include but not limited to aziridin-1-yl, aziridin-2-yl, azetidin-1-yl, azetidin-2-yl, azetidin-3-yl, piperidin-1-yl, piperidin-4-yl, morpholin-4-yl, piperidin-1-yl, piperzin-4-yl, pyrrolidin-1-yl, pyrrolidin-3-yl, [1,3]-dioxolan-2-yl, and the like.

The term “heterocyclicsulfonyl” means a heterocyclic group, as defined herein, with a ring nitrogen where the ring nitrogen is bonded directly to an —S(═O)₂— group forming an sulfonamide. Examples include, but not limited to, 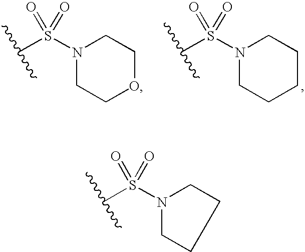
and the like.

The term “phenoxy” means the group C₆H₅O—.

The term “phenyl” means the group C₆H₅—.

The term “sulfonamide” means the group —S(═O)₂NH₂.

The term “sulfonic acid” means the group —SO₃H.

The term “thiol” means the group —SH.

The term “halogen” or “halo” mean —F, —Cl, —Br or —I.

The term “hydroxy” or “hydroxyl” mean —OH.

The term “amino” means —NH₂.

The term “cyano” means —CN.

The term “nitro” means —NO₂.

The term “carboxy” means —CO₂H or —CO₂⁻.

The phrase “pharmaceutically acceptable salt,” as used herein, is a salt formed from an acid and a basic nitrogen group of one of the Compounds of the Invention. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. The term “pharmaceutically acceptable salt” also refers to a salt prepared from a Compound of the Invention having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia and organic amines, such as unsubstituted or hydroxy-substituted mono-, di- or trialkylamines; dicyclohexylamine; tributyl amine; pyridine; N-methyl-N-ethylamine; diethylamine; triethylamine; mono-, bis- or tris-(2-hydroxy-lower alkyl amines), such as mono-, bis- or tris-(2-hydroxyethyl)amine, 2-hydroxy-tert-butylamine or tris-(hydroxymethyl)methylamine, N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine and the like.

The terms, “polymorph(s)” and “polymorphic forms” and related terms herein refer to solid forms of the Compound of the Invention having different physical properties as a result of the order of the molecules in the crystal lattice. The differences in physical properties exhibited by solid forms affect pharmaceutical parameters such as storage stability, compressibility and density (important in formulation and product manufacturing), and dissolution rates (an important factor in determining bioavailability). Differences in stability can result from changes in chemical reactivity (e.g., differential oxidation, such that a dosage form discolors more rapidly when comprised of one solid form than when comprised of another solid form) or mechanical changes (e.g., tablets crumble on storage as a kinetically favored polymorph converts to thermodynamically more stable solid form) or both (e.g., tablets of one solid form are more susceptible to breakdown at high humidity). As a result of solubility/dissolution differences, in the extreme case, some solid form transitions may result in lack of potency or, at the other extreme, toxicity. In addition, the physical properties of the crystal may be important in processing, for example, one solid form might be more likely to form solvates or might be difficult to filter and wash free of impurities (i.e., particle shape and size distribution might be different between one solid form relative to the other).

As used herein and unless otherwise indicated, the term “clathrate” means a Compound of the Invention, or a salt thereof, in the form of a crystal lattice that contains spaces (e.g., channels) that have a guest molecule (e.g., a solvent or water) trapped within.

As used herein and unless otherwise indicated, the term “hydrate” means a Compound of the Invention, or a salt thereof, that further includes a stoichiometric or non-stoichiometric amount of water bound by non-covalent intermolecular forces.

As used herein and unless otherwise indicated, the term “prodrug” means a Compound of the Invention derivative that can be hydrolyzed, oxidized, or otherwise reacted under biological conditions (in vitro or in vivo) to provide an active compound, particularly a Compound of the Invention. Examples of prodrugs include, but are not limited to, derivatives and metabolites of a Compound of the Invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

The terms “isotopically” or “radio-labeled” refer to Compounds of the Invention which are identical to the Compounds of the Invention disclosed herein, but for the fact that one or more atoms are replaced or substituted by an atom having an atomic mass or mass number different from the atomic mass or mass number typically found in nature (i.e., naturally occurring) including, but not limited to, ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I.

### 5.8 PREPARATION OF COMPOUNDS OF THE INVENTION

### General Synthetic Methods

The novel substituted spiro 3H-indole-3,4′-piperidines and spiro isoquinoline-4(1H),4′-piperidines of the present invention can be readily prepared according to a variety of synthetic manipulations, all of which would be familiar to one skilled in the art. Certain methods for the preparation of compounds of the present invention include, but are not limited to, those described in Scheme 1-20.

The common intermediate 4 used in the synthesis of novel substituted 3H-indole-3,4′-piperidines can be prepared as shown in Schemes 1 and 2, infra. The nitrogen of methanol 1, wherein A and B are as defined above, is protected with a suitable protecting group (i.e., -PG₁) and the alcohol is subsequently converted to aldehyde 2 via an oxidation step. A particularly useful alcohol 1 is piperidin-4-yl-methanol, wherein A and B are both —CH₂CH₂—. Suitable protecting groups for the nitrogen of alcohol 1 include, but are not limited to, t-butyl carbamate (Boc), benzyl carbamate (Cbz), p-methoxybenzyl carbamate (Moz), allyl carbamate (Alloc), 9-fluorenylmethyl carbamate (Fmoc), and the like. Various methods can be used to protect the nitrogen of alcohol 1. For example, the t-butyl carbamate group can be introduced using a variety of reagents, such as (Boc)₂O, with a suitable base (such as, NaOH, KOH, or Me₄NOH) in a suitable solvent(s) (THF, CH₃CN, DMF, EtOH, MeOH, H₂O, or mixtures thereof) and at a temperature of about −10° C. to about 50° C. It is understood that a protecting group can also be a soluble or insoluble resin commonly used in the art in the preparation of compound libraries. Other representative protecting groups suitable for a wide variety of synthetic transformations are disclosed in Greene and Wuts, Protective Groups in Organic Synthesis, third edition, John Wiley & Sons, New York, 1999, the disclosure of which is incorporated herein by reference in its entirety. Suitable oxidizing methods for the conversion to aldehyde 2 are known in the art and include for example, Swem or Swem-like oxidations, Corey oxidation with NCS or any other suitable procedures such as those described by Hudlicky, M. in Oxidations in Organic Chemistry, ACS Monograph 186 (1990), incorporated herein by reference in its entirety. One particularly useful oxidation procedure employs pyridine.SO₃ and a tertiary amine (such as, N,N-diisopropylethylamine, triethylamine, or N-methylmorpholine) in a suitable solvent(s) (such as, THF, CH₂Cl₂, CH₃CN, DMF, or mixtures thereof). Reaction temperature ranges from about −20° C. to about 50° C., preferably about −5° C. to about 35° C.

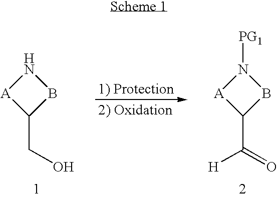

The method for the conversion of aldehyde 2 to indole 4 is shown in Scheme 2. A mixture of an appropriately substituted arylhydrazine 3 and an N-protected aldehyde 2 in the presence of an acid produces the intermediate indole (not shown). Suitable acids include, but are not limited to, trifluoroacetic acid, p-toluenesulfonic acid, and the like. Reduction of intermediate indole to the indoline 4 can be accomplished by a number of reducing agents. Suitable reducing agents include, but not limited to, alkali metal aluminum hydrides (such as, lithium aluminum hydride), alkali metal borohydrides (such as, sodium borohydride, lithium borohydride), alkali metal trialkoxyaluminum hydrides (such as, lithium tri-tert-butoxyaluminum hydride), and the like; see, e.g., Maligres, P. E.; Houpis, I.; Rossen, K.; Molina, A.; Sager, J.; Upadhyay, V.; Wells, K. M.; Reamer, R. A.; Lynch, J. E.; Askin, D.; Volante, R. P.; Reider, P. J. Tetrahedron 53:10983-10992 (1997)). The solvent includes ethereal solvents (such as, tetrahydrofuran or dioxane), aromatic solvents (such as, toluene), alcoholic solvents for use with primarily borohydride (such as, ethanol, methanol or isopropanol) or mixtures thereof. Reaction temperature ranges from about −78° C. to 120° C., preferably about −20° C. to 80° C.

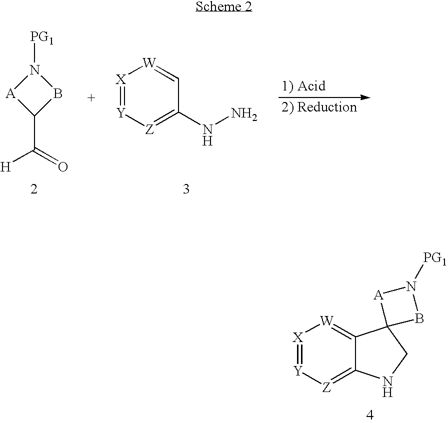

The common intermediate 10, used in the synthesis of novel substituted 3H-indole-3,4′-piperidines and spiro isoquinoline-4(1H),4′-piperidines, can be prepared as shown in Schemes 3-6, infra.

As shown in Scheme 3, the carboxylic acid 5 is converted to an acid halide [(ClCO)₂, SOCl₂, SOBr₂, and the like, optionally in the presence of DMF] and subsequently coupled with amine 6 using a base in an inert solvent to give amide 7, wherein PG, is similar as described supra. The coupling base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide preferably sodium hydroxide or potassium hydroxide, etc.), a tertiary amine (preferably N,N-diisopropylethylamine, triethylamine, or N-methylmorpholine, etc.), or an aromatic amine (preferably pyridine, imidazole, poly-(4-vinylpyridine), etc.). The inert solvent includes lower halocarbon solvents (preferably dichloromethane, dichloroethane, or chloroform, etc.), ethereal solvents (preferably tetrahydrofuran or dioxane), amide solvents (preferably N,N-dimethylformamide, etc.), or aromatic solvents (preferably toluene or pyridine, etc.). Reaction temperature ranges from about −20° C. to 50° C., preferably about 0° C. to 40° C.

Alternatively, carboxylic acid 5 is reacted with amine 6 and a dehydrating condensing agent in an inert solvent with or without a base to provide the novel amide (W) of the present invention. The dehydrating condensing agent includes dicyclohexylcarbodiimide (DCC), 1,3-diisopropylcarbodimide (DIC), 1-ethyl-3-(3-dimethylaminopropyl)carbodiimide hydrochloride (EDC.HCl), bromo-tris-pyrrolidino-phosphonium hexafluorophosphate (PyBroP), O-(7-azabenzotriazol-1-yl)-1,1,3,3-tetramethyluronium hexafluorophosphate (HATU), benzotriazoloyloxytris(dimethylamino) phosphonium hexafluorophosphate (BOP), or 1-cyclohexyl-3-methylpolystyrene-carbodiimide. The base includes a tertiary amine (preferably N,N-diisopropylethylamine or triethylamine, etc.). The inert solvent includes lower halocarbon solvents (preferably dichloromethane, dichloroethane, or chloroform, etc.), ethereal solvents (preferably tetrahydrofuran or dioxane), nitrile solvents (preferably acetonitrile, etc.), or amide solvents (preferably N,N-dimethylformamide, etc.). In case of need, 1-hydroxybenzotriazole (HOBT), HOBT-6-carboxamidomethyl polystyrene, or 1-hydroxy-7-azabenzotriazole (HOAT) can be used as a reactant agent. Reaction temperature ranges from about −20° C. to 50° C., preferably about 0° C. to 40° C.

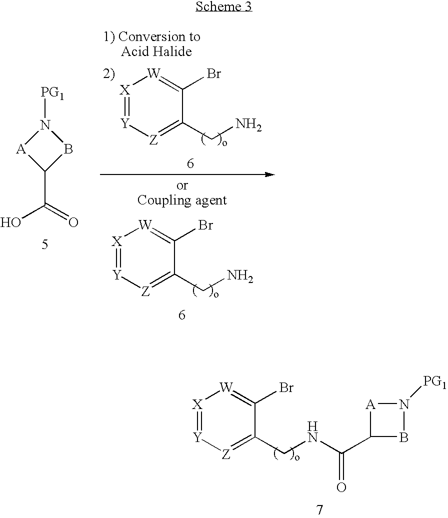

The nitrogen of amide 7 is masked with an appropriate protecting group (—PG₂) that is optionally orthogonal to other protective groups in the molecule to give protected amide 8, as illustrated in Scheme 4. Suitable groups for —PG₂ include benzyl, p-methoxybenzyl, acyl, and the like. Other suitable protecting groups are described by Greene and Wuts, in Protective Groups in Organic Synthesis, third edition, John Wiley & Sons, New York, 1999, supra. It is understood that —PG₂ can also be a soluble or insoluble resin commonly used in the preparation of the compound libraries.

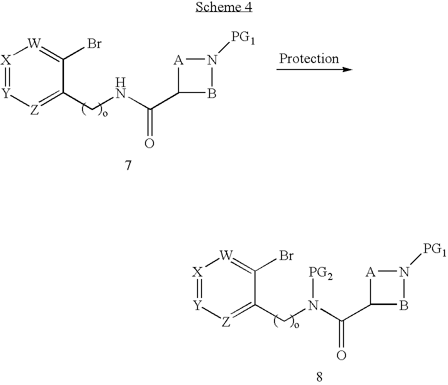

As shown in Scheme 5, the protected amine 8 is cyclized by treatment with a strong base in the presence of a metal catalyst and a suitable ligand in a suitable solvent to give the lactam as the intermediate. A similar process has been described by Lee, S.; and Hartwig, J. F. in J. Org. Chem. 2001, 66, 3402-3415, encorporated by reference in it's entirity. A suitable strong base is one that is appropriate to remove the a-hydrogen of the protected amide group, explicitly shown in Scheme 5. The strong base includes, alkali metal alkoxide (such as, potassium tert-butoxide, sodium tert-butoxide, and the like); alkyl lithiums (such as, tert-butyl lithium, and the like). A suitable metal catalyst includes palladium acetate, Pd(dbd)₃, and the like, and a suitable ligand includes tricyclohexyl phosphine (Cy₃P), BINAP, t-Bu₃P, carbene ligands known in the art, and the like. The inert solvent includes ethereal solvents (such as, tetrahydrofuran, dioxane and the like), aromatic solvents (such as, benzene, toluene, and the like), and mixtures thereof. Reaction temperature ranges from about RT to about 200° C. The carbonyl of the resulting lactam is reduced with a reducing agent in an inert solvent to give spirocyclic amine 9. The reducing agent includes alkali metal aluminum hydrides (preferably lithium aluminum hydride), alkali metal borohydrides (preferably lithium borohydride), alkali metal trialkoxyaluminum hydrides (preferably lithium tri-tert-butoxyaluminum hydride), dialkylaluminum hydrides (preferably di-isobutylaluminum hydride), borane, dialkylboranes (preferably di-isoamyl borane), alkali metal trialkylboron hydrides (preferably lithium triethylboron hydride). The inert solvent includes ethereal solvents (preferably tetrahydrofuran or dioxane) or aromatic solvents (preferably toluene, etc.). Reaction temperature ranges from about −78° C. to 200° C., preferably about 50° C. to 120° C.

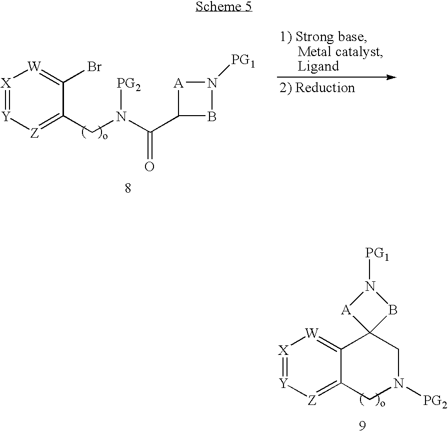

It is understood that spirocyclic amine 9 is a diprotected scaffold which is useful in the preparation of Compounds of the Invention. In general, this particular protecting strategy is illustrated Scheme 6 to give amines 10 and 11. For convenience, the two protecting groups for spirocyclic amine 9 are selected so one protecting group can be substantially removed without substantially affecting the other protecting group. This type of strategy is referred to as orthogonal protection. One example includes when —PG₁ is a Boc group and —PG₂ is a benzyl group. In this example, the Boc group can be removed under acidic conditions without substantially affecting the benzyl group. Alternatively, the benzyl group can be removed under conditions that will not substantially remove the Boc group. Many orthogonal protection schemes are known in the art.

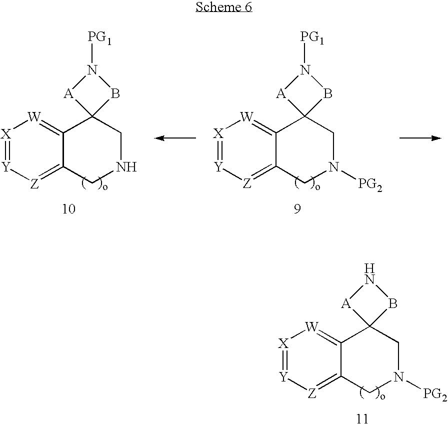

Alternatively, the common intermediate 10, used in the synthesis of novel substituted 3H-indole-3,4′-piperidines and Spiro isoquinoline-4(1H),4′-piperidines, can be prepared as shown in Scheme 7, infra.

The amine 18 is substituted via reductive amination reaction using an aldehyde (ArCHO, wherein Ar is substituted or unsubstituted) and a reducing agent in an inert solvent with or without an acid. The reducing agent includes sodium triacetoxyborohydride, sodium cyanoborohydride, sodium borohydride, or borane-pyridine complex, preferably sodium triacetoxyborohydride or sodium cyanoborohydride. The inert solvent includes lower alkyl alcohol solvents (preferably methanol or ethanol, etc.), lower halocarbon solvents (preferably dichloromethane, dichloroethane, or chloroform, etc.), ethereal solvents (preferably tetrahydrofuran or dioxane), or aromatic solvents (preferably toluene, etc.). The acid includes an inorganic acid (preferably hydrochloric acid or sulfuric acid) or an organic acid (preferably acetic acid). Reaction temperature ranges from about −20° C. to 120° C., preferably about 0° C. to 100° C. Also this reaction can be carried out under microwave conditions. The 2° amine is subsequently coupled with a 2-haloacetic acid or 2-haloacetyl halide via a coupling reaction, such as reacting with chloroacetyl chloride, bromoacetyl bromide, chloroacetic acid anhydride, and the like. The resulting amide 19 is cyclized via reaction with a suitable palladium catalyst to give the cyclic amide 20 (See, e.g., Hennessey, E. J.; Buchwald, S. L. J. Am. Chem. Soc. 125:12084-12085 (2003)). Double alkylation with a protected bis-haloalkyl amine produces the spirocyclic amide 21 which can be reduced as described herein, give Intermediate 10.

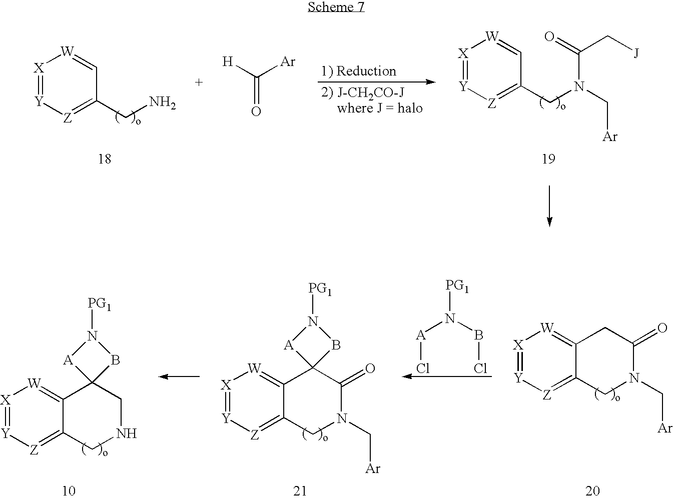

The common intermediate 10 (wherein o=1), used in the synthesis of novel substituted spiro isoquinoline-4(1H),4′-piperidines, can be prepared as shown in Scheme 8, infra.

Intermediate 10 (wherein o=1) can be synthesized by alkylation of a substituted or unsubstituted benzyl nitrile 22 by treatment with a strong base, such as, but not limited to, potassium tert-butoxide, and a protected bis-haloalkyl amine to give the resulting nitrile 23. A similar process has been described by Ong, H. H., et. al. in J. Med. Chem. 1983, 26, 981-986, incorporated herein by reference in its entirety. The nitrile can be reduced to the amine 24 with a reducing agent such as, but not limited to, lithium aluminum hydride, which is then reacted with formaldehyde to form imine 25. An intramolecular Pictet-Spengler reaction, mediated by a strong acid, such as, but not limited to, HCl, forms the monoprotected spirocyclic system (Intermediate 10, wherein o=1) which can be used to produce the Compounds of the Invention.

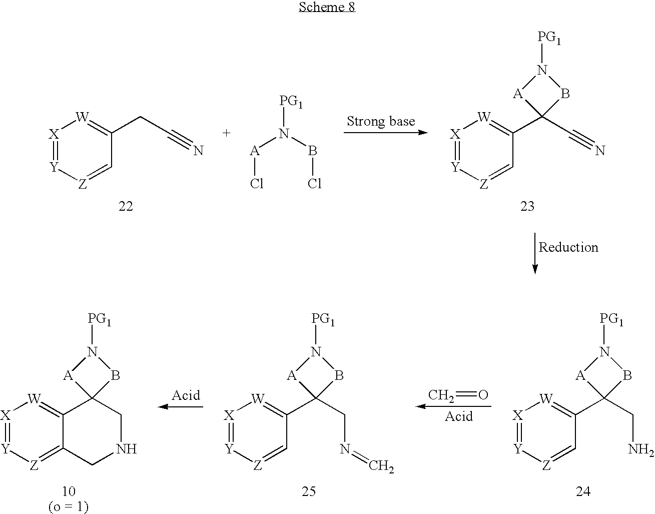

Outlined in Schemes 9-18 is the preparation of Compounds 10a-h and 11a-h that are useful in the making of novel substituted 3H-indole-3,4′-piperidines and spiro isoquinoline-4(1H),4′-piperidines.

As shown in Scheme 9, the common intermediate 10 can be functionalized while —PG₁ is still present. The common intermediate 10 is reacted with a carboxylic acid (R₁₄CO₂H, wherein as using in Scheme 9, R₁₄ is Ar, or a C₁₋₆ alkyl-Ar, and Ar has the same meaning as described herein) with a dehydrating condensing agent in an inert solvent with or without a base to provide the amide 10a of the present invention. The dehydrating condensing agent includes dicyclohexylcarbodiimide (DCC), 1,3-diisopropylcarbodimide (DIC), 1-ethyl-3-(3-dimethylaminopropyl)carbodiimide hydrochloride (EDC.HCl), bromo-tris-pyrrolidinophosphonium hexafluorophosphate (PyBroP), benzotriazoloyloxytris(dimethylamino)phosphonium hexafluorophosphate (BOP), O-(7-azabenzo triazol-1-yl)-1,1,3,3-tetramethyluronium hexafluorophosphate (HATU), or 1-cyclohexyl-3-methylpolystyrenecarbodimide. The base includes a tertiary amine (such as, N,N-diisopropylethylamine, triethylamine, and the like). The inert solvent includes lower halocarbon solvents (such as, dichloromethane, dichloroethane, chloroform, and the like), ethereal solvents (such as, tetrahydrofuran, dioxane, and the like), nitrile solvents (such as, acetonitrile, and the like), amide solvents (N,N-dimethylformamide, N,N-dimethylacetamide, and the like) and mixtures thereof. Optionally, 1-hydroxybenzotriazole (HOBT), HOBT-6-carboxaamidomethyl polystyrene, or 1-hydroxy-7-azabenzotriazole (HOAT) can be used as a reactant agent. Reaction temperature ranges from about −20° C. to 50° C., preferably about 0° C. to 40° C.

Alternatively, the amide 10a of the present invention can be obtained by an amidation reaction using an acid halide (such as, R₁₄COCl) and a base in an inert solvent. The base includes an alkali metal carbonate (such as, sodium carbonate, potassium carbonate, and the like), an alkali metal hydrogencarbonate (such as, sodium hydrogencarbonate, potassium hydrogencarbonate, and the like), an alkali hydroxide (such as, sodium hydroxide or potassium hydroxide, and like), a tertiary amine (such as, N,N-diisopropylethylamine, triethylamine, N-methylmorpholine, and the like), or an aromatic amine (such as, pyridine, imidazole, poly-(4-vinylpyridine), and the like). The inert solvent includes lower halocarbon solvents (such as, dichloromethane, dichloroethane, chloroform, and the like), ethereal solvents (such as, tetrahydrofuran, dioxane, and the like), amide solvents (such as, N,N-dimethylformamide, and the like), aromatic solvents (benzene, toluene, pyridine, and the like) and mixtures thereof Reaction temperature ranges from about −20° C. to 50° C., preferably about 0° C. to 40° C.

Also illustrated in Scheme 9, amide 10a can be reacted with a reducing agent in an inert solvent to provide the amine bob of the present invention. The reducing agent includes alkali metal aluminum hydrides (such as, lithium aluminum hydride, and the like), alkali metal borohydrides (such as, lithium borohydride, and the like), alkali metal trialkoxyaluminum hydrides (such as, lithium tri-tert-butoxyaluminum hydride, and the like), dialkylaluminum hydrides (such as, di-isobutylaluminum hydride, and the like), borane, dialkylboranes (such as, di-isoamyl borane, and the like), alkali metal trialkylboron hydrides (such as, lithium triethylboron hydride, and the like). The inert solvent includes ethereal solvents (such as, tetrahydrofuran, dioxane, and the like), aromatic solvents (such as, toluene, and the like) and mixtures thereof. Reaction temperature ranges from about −78° C. to 200° C., such as, about 50° C. to 120° C.

Alternatively, the amine 10b of the present invention can be obtained by a reductive amination reaction using with common intermediate 10 with an aldehyde (R₁₄CHO) and a reducing agent in an inert solvent with or without an acid. The reducing agent includes sodium triacetoxyborohydride, sodium cyanoborohydride, sodium borohydride, borane-pyridine complex, and the like. The inert solvent includes lower alkyl alcohol solvents (such as, methanol, ethanol, and the like), lower halocarbon solvents (such as, dichloromethane, dichloroethane, chloroform, and the like), ethereal solvents (such as, tetrahydrofuran, dioxane, and the like), aromatic solvents (such as, benzene, toluene, and the like) and mixtures thereof. The acid includes an inorganic acid (such as, hydrochloric acid, sulfuric acid, and the like) or an organic acid (such as, acetic acid, and the like). Reaction temperature ranges from about −20° C. to 120° C., preferably about 0° C. to 100° C. In addition, this reaction can optionally be carried out under microwave conditions.

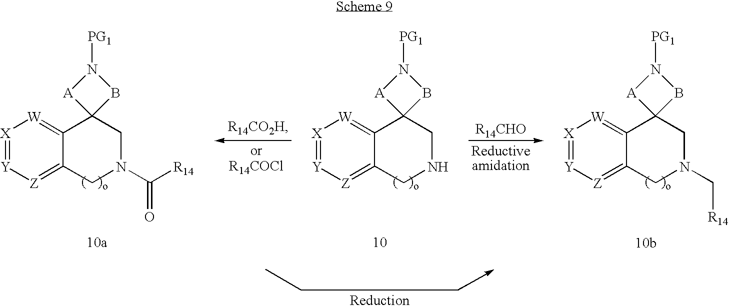

In an alternative manner, common intermediate 10 can be alkylated directly with an alkylating agent, such as R₁₅-halide (wherein R₁₅ is substituted or unsubstituted C₁₋₆ alkyl, or substituted or unsubstituted C₁₋₆ alkyl-Ar, and halide is chloro, bromo and iodo), in the presence of a base and in an inert solvent to provide amine 10c. The base includes an alkali metal carbonate (such as, sodium carbonate, potassium carbonate, and the like), an alkali metal hydride (such as, sodium hydride, potassium hydride, and the like), alkali metal alkoxide (such as, potassium tert-butoxide, sodium tert-butoxide, and the like); alkyl lithiums (such as, tert-butyl lithium, n-butyl lithium and the like). The inert solvents include, ethereal solvents (such as, tetrahydrofuran, dioxane), aromatic solvents (such as, benzene, toluene, and the like), amide solvents (such as, N,N-dimethylformamide, and the like) and mixtures thereof. Reaction temperature ranges from about −20° C. to 120° C., preferably about 0° C. to 100° C.

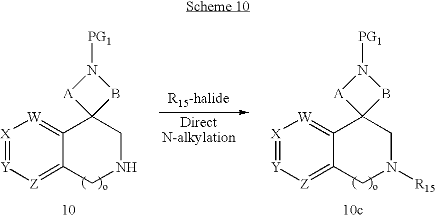

In addition, urea 10d of the present invention can be obtained by a urea reaction with common intermediate amine 10 using an isocyanate (R₇NCO, wherein R₇ has the same meaning as described herein) in an inert solvent with or without a base as shown in Scheme 11. The base includes an alkali metal carbonate (such as, sodium carbonate, potassium carbonate, and the like), an alkali metal hydrogencarbonate (such as, sodium hydrogencarbonate, potassium hydrogencarbonate, and the like), an alkali hydroxide (such as, sodium hydroxide, potassium hydroxide, and the like), a tertiary amine (such as, N,N-diisopropylethylamine, triethylamine, N-methylmorpholine, and the like), or an aromatic amine (such as, pyridine, imidazole, and the like). The inert solvent includes lower halocarbon solvents (such as, dichloromethane, dichloroethane, chloroform, and the like), ethereal solvents (such as, tetrahydrofuran, dioxane, and the like), aromatic solvents (such as, benzene, toluene, and the like), or polar solvents (such as, N,N-dimethylformamide, dimethyl sulfoxide, and the like). Reaction temperature ranges from about −20° C. to 120° C., preferably about 0° C. to 100° C.

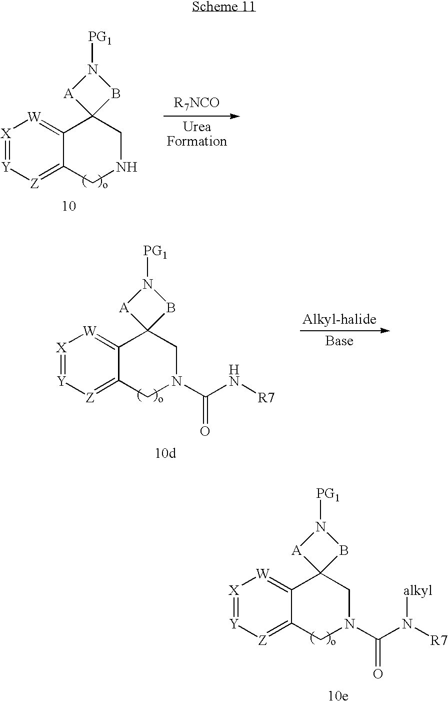

Also shown in Scheme 11 is the preparation of additional compounds of the invention via alkylating the nitrogen of urea 10d with an alkyl-halide (wherein halide is chloro, bromo and iodo) in the presence of a base in an inert solvent to provide di-substituted urea 10e. The base includes an alkali metal hydride (such as, sodium hydride, potassium hydride, and the like), alkali metal alkoxide (such as, potassium tert-butoxide, sodium tert-butoxide, and the like); alkyl lithiums (such as, tert-butyl lithium, n-butyl lithium and the like). The inert solvents include, ethereal solvents (such as, tetrahydrofuran, dioxane), aromatic solvents (such as, benzene, toluene, and the like), amide solvents (such as, N,N-dimethylformamide, and the like) and mixtures thereof. Reaction temperature ranges from about −20° C. to 120° C., preferably about 0° C. to 100° C.

In another method, common intermediate 10 is reacted with an aryl-halide (such as, Ar—Br, where Ar has the same meaning as described herein) or aryl-boronic acid with a metal catalyst and a ligand in a suitable solvent with a base to provide the N-aryl 10f as illustrated in Scheme 12. The metal catalyst includes palladium acetate, Pd₂(dba)₃, CuI, Cu(OTf)₂, Ni/C, Ni(COD)₂, Ni(acac)₂, and the like. The ligand includes 2,2′-bis(diphenyl-phosphino)-1,1′-binaphthyl (BINAP), P(o-tolyl)₃, t-Bu₃P, DPPF, carbene ligands in the art, and the like. The base includes an alkali metal carbonate (such as, sodium carbonate, potassium carbonate, cesium carbonate, and the like), an alkali metal hydrogencarbonate (such as, sodium hydrogencarbonate, potassium hydrogencarbonate, and the like), an alkali hydroxide (such as, sodium hydroxide, potassium hydroxide, and the like), an alkali metal phosphate (such as, potassium phosphate, and the like), a tertiary amine (such as, N,N-diisopropylethylamine, triethylamine, N-methylmorpholine, and the like), or an aromatic amine (such as, pyridine, imidazole, and the like). The inert solvent includes lower halocarbon solvents (such as, dichloromethane, dichloroethane, chloroform, and the like), ethereal solvents (such as, tetrahydrofuran, dioxane), aromatic solvents (such as, benzene, toluene, and the like), or amide solvents (such as, N,N-dimethylformamide, and the like). Reaction temperature ranges from about −20° C. to 120° C., preferably about 0° C. to 100° C.

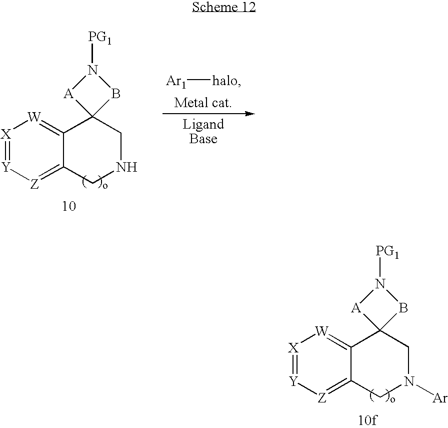

As illustrated in Scheme 13, urethane 10g of the present invention can be obtained by a urethane reaction using R₂₀OCO-halide (wherein R₂₀ is Ar or C₁₋₆ alkyl, halide is chloro, bromo, or iodo, particularly useful is chloro) in an inert solvent with or without a base. The base includes an alkali metal carbonate (such as, sodium carbonate, potassium carbonate, and the like), an alkali metal hydrogencarbonate (such as, sodium hydrogencarbonate, potassium hydrogencarbonate, and the like), an alkali hydroxide (such as, sodium hydroxide, potassium hydroxide, and the like), a tertiary amine (such as, N,N-diisopropylethylamine, triethylamine, N-methylmorpholine, and the like), or an aromatic amine (such as, pyridine, imidazole, poly-(4-vinylpyridine), and the like). The inert solvent includes lower halocarbon solvents (such as, dichloromethane, dichloroethane, chloroform, and the like), ethereal solvents (such as, tetrahydrofuran, dioxane, and the like), aromatic solvents (such as, benzene, toluene, and the like), or polar solvents (such as, N,N-dimethylformamide, dimethyl sulfoxide, and the like). Reaction temperature ranges from about −20° C. to 120° C., preferably about 0° C. to 100° C.

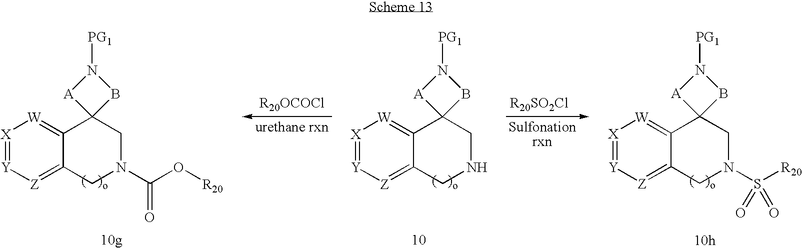

Also, shown in Scheme 13 is another method used in the preparation of compounds of the invention. The sulfonamide 10h of the present invention can be obtained from common intermediate amine 10 by a sulfonation reaction using R₁₄SO₂halo (such as a R₁₄SO₂Cl, and the like) in an inert solvent with or without a base. The base includes an alkali metal carbonate (such as, sodium carbonate, potassium carbonate, and the like), an alkali metal hydrogencarbonate (such as, sodium hydrogencarbonate, potassium hydrogencarbonate, and the like), an alkali hydroxide (such as, sodium hydroxide, potassium hydroxide, and the like), a tertiary amine (such as, N,N-diisopropylethylamine, triethylamine, N-methylmorpholine, and the like), or an aromatic amine (such as, pyridine, imidazole, poly-(4-vinylpyridine), and the like). The inert solvent includes lower halocarbon solvents (such as, dichloromethane, dichloroethane, chloroform, and the like), ethereal solvents (such as, tetrahydrofuran, dioxane, and the like), aromatic solvents (such as, benzene, toluene, and the like), or polar solvents (such as, N,N-dimethylformamide, dimethyl sulfoxide, and the like). Reaction temperature ranges from about −20° C. to 120° C., preferably about 0° C. to 100° C.

Alternatively, common intermediate 11 can be functionalized while —PG₂ is still present. In general, a similar set of methods can be utilized as described supra. These methods are represented in Schemes 14-18.

As shown in Scheme 14, the common intermediate 11 is reacted with a carboxylic acid (R₇CO₂H, wherein R₇ has the same meaning as described herein) with a dehydrating condensing agent in an inert solvent with or without a base to provide the amide 11a of the present invention. The dehydrating condensing agent, inert solvent and base are similar to those described above.

Alternatively, the amide 11a of the present invention can be obtained by an amidation reaction using an acid halide (such as, R₇COCl) and a base in an inert solvent. The base and inert solvent are similar to those described above.

Also illustrated in Scheme 14, amide 11a can be reacted with a reducing agent in an inert solvent to provide the amine 11b of the present invention. The reducing agent and inert solvent are similar to those described above.

Alternatively, the amine lib of the present invention can be obtained by a reductive amination reaction using common intermediate 10 with an aldehyde (R₁CHO or R₇CHO) and a reducing agent in an inert solvent with or without an acid. The reducing agent and inert solvent are similar to those described above.

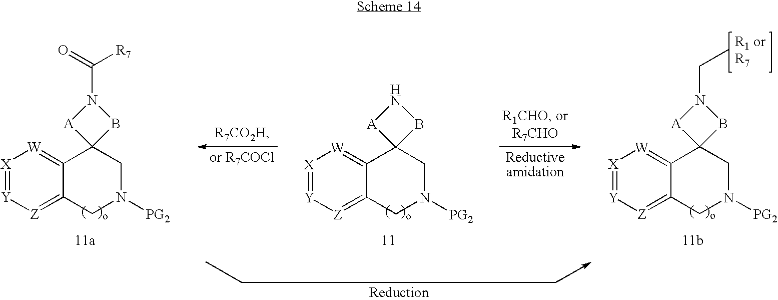

In an alternative manner, common intermediate 11 can be alkylated directly with an alkylating agent, such as R₁-E-halide (wherein R₁ and E have the same meaning as described herein, and halide is chloro, bromo and iodo), in the presence of a base and in an inert solvent to provide amine 11c. One particularly useful alkylating agent is R₁—(CH₂)ₚ—Br. The base and inert solvent are similar to those described above.

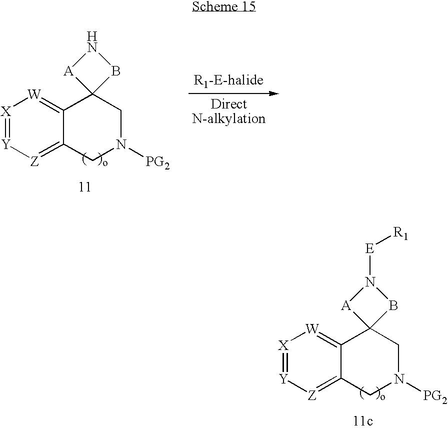

In addition, urea 11d of the present invention can be obtained by a urea reaction with common intermediate amine 11 using an isocyanate (R₇NCO) in an inert solvent with or without a base as shown in Scheme 16. The inert solvent and base are similar to those described above.

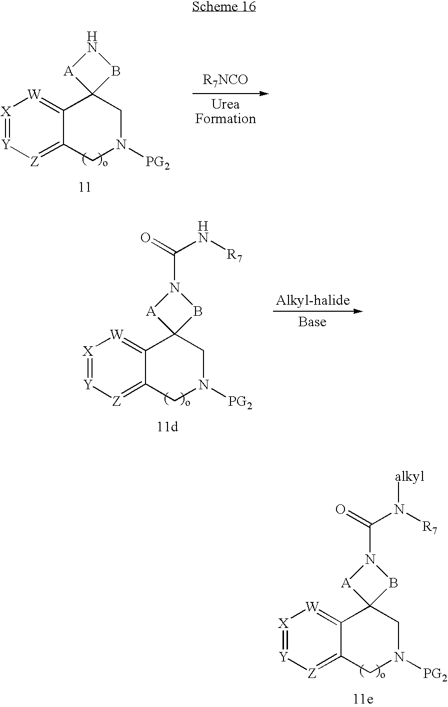

Also shown in Scheme 16 is the preparation of compounds via alkylating the nitrogen of urea 11d with an alkyl-halide (wherein halide is chloro, bromo and iodo) in the presence of a base in an inert solvent to provide di-substituted urea 11e. The base and inert solvent are similar to those described above.

In another method, common intermediate 11 is reacted with an aryl-halide (Ar₁-halide) wherein Ar₁ is a substituted or unsubstituted aryl, or substituted or unsubstituted -(5 to 10) membered heteroaryl) with a metal catalyst and ligand in an inert solvent with a base to provide the N-aryl 11f as illustrated in Scheme 17. As used in Scheme 17, Ar₁ is a substituted or unsubstituted aryl, or substituted or unsubstituted -(5 to 10) membered heteroaryl. The metal catalyst, inert solvent and base are similar to those described above.

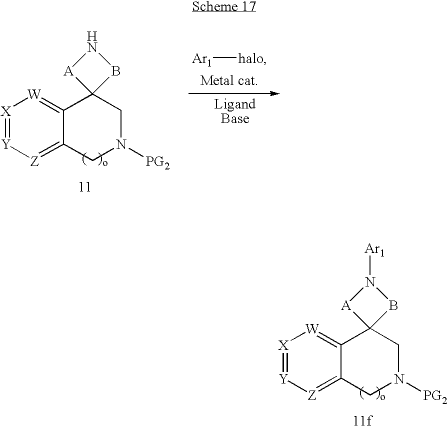

As illustrated in Scheme 18, urethane 11g of the present invention can be obtained by a urethane reaction using R₇OCO-halide (wherein R₇ has the same meaning as described herein) in an inert solvent with or without a base. The solvent and base are similar to those described above.

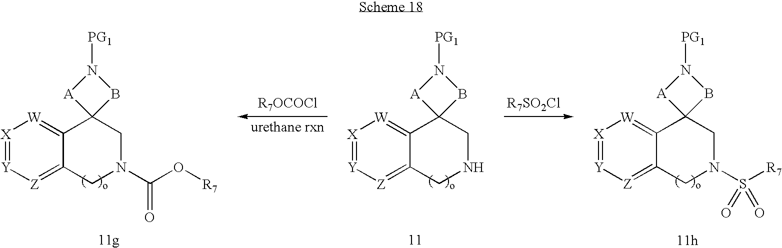

The protecting groups for monoprotected intermediates 10a to 10h (e.g., —PG₁) and 11a to 11h (e.g., —PG₂) can be removed and the resulting nitrogen modified using similar procedures as described above to provide compounds of the invention.

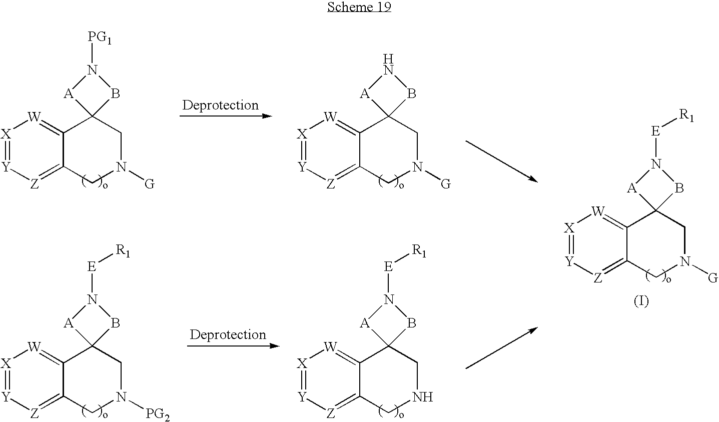

One particularly useful method for preparing compounds is the epoxide ring opening reaction as shown in Scheme 20. For example, intermediate 11 can be reacted with an optionally substituted epoxide catalyzed by a Lewis acid such as, but not limited to, lithium trifluoromethanesulfonimide to give alcohol 12.

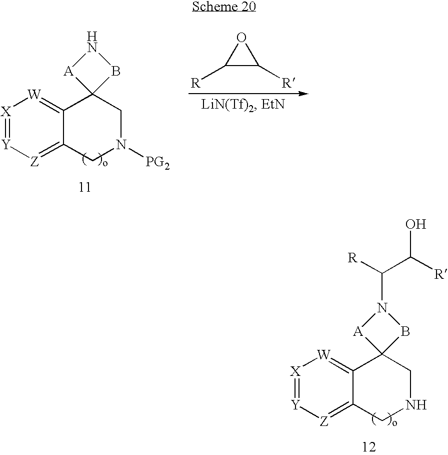

Synthetic methods for incorporating isotopes or radio-isotopes into organic compounds are applicable to the Compounds of the Invention and are well known in the art. Synthetic methods for incorporating activity levels of tritium into target molecules, are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl [³H] products by treating appropriate precursors with high specific activity methyl iodide [³H]. This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na¹²⁵I. A represented procedure is found in Zhu, D.-G. et al., J. Org. Chem. 67, 943-948 (2002).

B. Ortho ¹²⁵Iodination of phenols—This procedure allows for the incorporation of ¹²⁵I at the ortho position of a phenol as reported by Collier, T. L. et al., J. Labeled Compd Radiopharm. 42, S264-S266 (1999).

C. Aryl and heteroaryl bromide exchange with ¹²⁵I—This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. et al., J. Labeled Compd Radiopharm. 44, S280-S282 (2001).

Certain Compounds of the Invention can have asymmetric centers and therefore exist in different enantiomeric and diastereomeric forms. A Compound of the Invention can be in the form of an optical isomer or a diastereomer. Accordingly, the invention encompasses Compounds of the Invention and their uses as described herein in the form of their optical isomers, diasteriomers and mixtures thereof, including a racemic mixture. Optical isomers of the Compounds of the Invention can be obtained by known techniques such as chiral chromatography or formation of diastereomeric salts from an optically active acid or base.

In addition, one or more hydrogen, carbon or other atoms of a Compound of the Invention can be replaced by an isotope of the hydrogen, carbon or other atoms. Such compounds, which are encompassed by the present invention, are useful as research and diagnostic tools as well as in Mas receptor binding assays.

### 5.9 CARDIO-PROTECTIVE COMPOUNDS AND METHODS

The invention also provides a method for identifying a modulator of a Mas receptor comprising contacting a candidate compound with the receptor and determining whether the receptor functionality is modulated. The candidate compound would be a compound not previously known to modulate the Mas receptor. A modulator is a compound that alters the functionality of a receptor. Modulators include, for example, agonists, partial agonists, inverse agonists and antagonists.

Several assays are well known in the art for determining whether a compound alters the functionality of a receptor, for example, the ability of a receptor to bind a ligand or other compound, or the ability of a receptor to initiate a signal transduction cascade. GPCR binding assays and functional assays are well known in the art (see, for example, “From Neuron To Brain” (3^{rd} Ed.) Nichols, J. G. et al eds. Sinauer Assoicates, Inc. (1992)). For example, ligand binding assays, IP₃ assays, cAMP assays, GPCR fusion protein assays, calcium flux assays, and GTP7S binding assays are well known in the art.

The invention relates to a method for identifying a cardio-protective compound, comprising: a) contacting a candidate compound with a Mas receptor, and b) determining whether the receptor functionality is decreased, wherein a decrease in receptor functionality is indicative of the candidate compound being a cardio-protective compound. In one embodiment, the Mas receptor is human. In another embodiment, the cardio-protective compound is an inverse agonist or antagonist of the Mas receptor. In a further embodiment, the cardio-protective compound is an inverse agonist of the Mas receptor. In another embodiment, determining whether the receptor functionality is decreased comprises using an IP₃ assay. The invention further relates to a cardio-protective compound identified according to this method. In one embodiment, the cardio-protective compound is an inverse agonist. In another embodiment, the cardio-protective compound is an inverse agonist that does not significantly increase blood pressure.

As used herein, a “candidate compound” can be a molecule, for example, a chemical compound or a polypeptide, which is amenable to a screening technique. Candidate compounds can include for example, chemical or biological molecules such as simple or complex organic molecules, metal-containing compounds, carbohydrates, polypeptides, peptidomimetics and the like. Candidate compounds can be chosen randomly such as from a combinatorial chemical library or candidate compounds can be chosen based on a structural or biochemical feature. Candidate compounds exclude compounds known to bind to or modulate the Mas receptor, for example, peptide ligands of the Mas receptor that are known in the art. The term modulate means an increase or decrease in the amount, quality, or effect of a particular activity, function or molecule.

A Mas receptor refers to a polypeptide with substantially the same amino acid sequence as that shown in SEQ ID NO: 2 or referenced in GenBank as Accession No. NP_{—}002368.1. Substantially the same amino acid sequence is intended to mean an amino acid sequence contains a considerable degree of sequence identity or similarity, such as at least 80%, at least, 85%, at least 90%, at least 93%, at least 95%, at least 97%, at least 99%, or 100% sequence identity or similarity to a reference amino acid sequence. Conservative and non-conservative amino acid changes, gaps, and insertions to an amino acid sequence can be compared to a reference sequence using available algorithms and programs such as the Basic Local Alignment Search Tool (“BLAST”) using default settings [See, e.g., Karlin and Altschul, Proc Natl Acad Sci USA (1990) 87:2264-8; Altschul et al., J Mol Biol (1990) 215:403-410; Altschul et al., Nature Genetics (1993) 3:266-72; and Altschul et al., Nucleic Acids Res (1997) 25:3389-3402].

It is understood that a fragment of a Mas receptor which retains substantially a function of the entire polypeptide is included in the definition. For example, a ligand binding domain of a Mas receptor can be used in lieu of the entire polypeptide in the methods of the invention.

It is also understood that limited modifications to the Mas receptor can be made without destroying its activity. For example, Mas receptor is intended to include other Mas receptor polypeptides, for example, species homologues of the human Mas receptor polypeptide (SEQ ID NO: 2). The sequence of species homologs of the human Mas receptor are present in the database, for example, a rat homolog of the Mas receptor can be found in GenBank at Accession No. NP_{—}036889.1. In addition, a Mas receptor includes splice variants and allelic variants of Mas receptors that retain substantially a function of the entire Mas receptor polypeptide.

As used herein, “contacting” means bringing at least two moieties together, whether in an in vitro system or an in vivo system. As used herein, an in vitro system means outside of a living cell and in vivo means in a living cell or organism.

As understood by one skilled in the art, the term agonist means material (for example, a ligand or candidate compound) that activates an intracellular response when it binds to a receptor. A partial agonist is material (for example, a ligand or candidate compound) that activates an intracellular response when it binds to the receptor but to a lesser degree or extent than do fall agonists.

As used herein, “antagonist” means material (for example, a candidate compound) that competitively binds to the receptor at the same site as an agonist but which does not activate an intracellular response, and can thereby inhibit an intracellular response elicited by an agonist. An antagonist does not diminish the baseline intracellular response in the absence of an agonist. In some embodiments, an antagonist is a material not previously known to compete with an agonist to inhibit a cellular response when it binds to the receptor.

As used herein, “inverse agonist” means material (for example, a candidate compound) that binds either to an endogenous form or to a constitutively activated form of a receptor so as to reduce the baseline intracellular response of the receptor observed in the absence of an agonist.

Generally, most inverse agonists and antagonists are synthetically derived compounds with an IC₅₀ value of anywhere from about 100 μM down to 50 μM. Initial screening assays of synthetic or natural compounds generally begin by using concentrations in the range of 1 μM to 20 μM. In some embodiments, a cardio-protective compound of the invention is an inverse agonist or antagonist with an IC₅₀ of less than 100 μM, or of less than 10 μM, of less than 1 μM, of less than 0.1 μM, of less than 0.01 μM, or of less than 0.001 μM. In some embodiments said cardio-protective compound of the invention is an inverse agonist or antagonist with an IC₅₀ of less than 100 μM, or of less than 10 μM, of less than 1 [M, of less than 0.1 μM, of less than 0.01 μM, or of less than 0.001 μM in an IP₃ assay carried out with membrane from cells known to express a Mas receptor or transiently or stably transfected cells, such as HEK or CHO cells, or in pigment dispersion assay carried out in transiently transfected melanophores expressing a Mas receptor. In some embodiments, said compound is an inverse agonist or antagonist with an IC₅₀ of less than 100 μM in said assay. In some embodiments, said compound is an inverse agonist or antagonist with an IC₅₀ of less than 80 μM in said assay. In some embodiments, said compound is an inverse agonist or antagonist with an IC₅₀ of less than 60 μM in said assay. In some embodiments, said compound is an inverse agonist or antagonist with an IC₅₀ of less than 40 μM in said assay. In some embodiments, said compound is an inverse agonist or antagonist with an IC₅₀ of less than 20 μM in said assay. In some embodiments, said compound is an inverse agonist or antagonist with an IC₅₀ of less than 10 μM in said assay. In some embodiments, said compound is an inverse agonist or antagonist with an IC₅₀ of less than 1 μM in said assay. In some embodiments, said compound is an inverse agonist or antagonist with an IC₅₀ of less than 0.1 μM in said assay. In some embodiments, said compound is an inverse agonist or antagonist with an IC₅₀ of less than 0.01 μM in said assay. In some embodiments, said compound is an inverse agonist or antagonist with an IC₅₀ of less than 0.001 μM in said assay. In some embodiments, said compound is an inverse agonist or antagonist with an IC₅₀ of less than 0.0001 μM in said assay. In some embodiments, said compound is an inverse agonist or antagonist with an IC₅₀ of between 0.0001-100 μM in said assay. In some embodiments, said compound is an inverse agonist or antagonist with an IC₅₀ of between 0.001-20 μM in said assay. In some embodiments, said compound is an inverse agonist or antagonist with an IC₅₀ of between 1-20 μM in said assay. In some embodiments, said compound is an inverse agonist or antagonist with an IC₅₀ of between 0.001-1 μM in said assay. In some embodiments, said compound is an inverse agonist or antagonist with an IC₅₀ of between 0.001-0.1 μM in said assay. In some embodiments, said compound is an inverse agonist or antagonist with an IC₅₀ of between 0.001-0.01 μM in said assay.

In some embodiments, said identified compound is bioavailable. A number of computational approaches available to those of ordinary skill in the art have been developed for prediction of oral bioavailability of a drug [Ooms et al., Biochim Biophys Acta (2002) 1587:118-25; Clark & Grootenhuis, Curr OpinDrug Discov Devel (2002) 5:382-90; Cheng et al., J Comput Chem (2002) 23:172-83; Norinder & Haeberlein, Adv Drug Deliv Rev (2002) 54:291-313; Matter et al., Comb Chem High Throughput Screen (2001) 4:453-75; Podlogar & Muegge, Curr Top Med Chem (2001) 1:257-75; the disclosure of each of which is hereby incorporated by reference in its entirety]. Furthermore, positron emission tomography (PET) has been successfully used by a number of groups to obtain direct measurements of drug distribution, including an assessment of oral bioavailability, in the mammalian body following oral administration of the drug, including non-human primate and human body [Noda et al., J Nucl Med (2003) 44:105-8; Gulyas et al., Eur J Nucl Med Mol Imaging (2002) 29:1031-8; Kanervaet al., Psychopharmacology (1999) 145:76-81; the disclosure of each of which is hereby incorporated by reference in its entirety].

In some embodiments, said compound is orally bioavailable. In some embodiments, said oral bioavailability can be shown to be at least 1%, at least 5%, at least 10%, at least 15%, at least 20%, at least 25%, at least 30%, at least 35%, at least 40%, or at least 45% relative to intraperitoneal administration. In some embodiments, said oral bioavailablity can be shown to be at least 1%, at least 5%, at least 10%, or at least 15% relative to intraperitoneal administration. In some embodiments, said oral bioavailability can be shown to be at least 1%, at least 5%, at least 10%, at least 15%, at least 20%, at least 25%, at least 30%, at least 35%, at least 40%, or at least 45% relative to intravenous administration. In some embodiments, said oral bioavailablity can be shown to be at least 1%, at least 5%, at least 10%, or at least 15% relative to intravenous administration.

The invention also relates to a method for identifying a cardio-protective compound, comprising: a) contacting a candidate compound with a Mas receptor, b) determining whether the receptor functionality is decreased, and c) determining the effect of the compound on blood pressure, wherein a decrease in receptor functionality and no significant increase in blood pressure is indicative of the candidate compound being a cardio-protective compound.

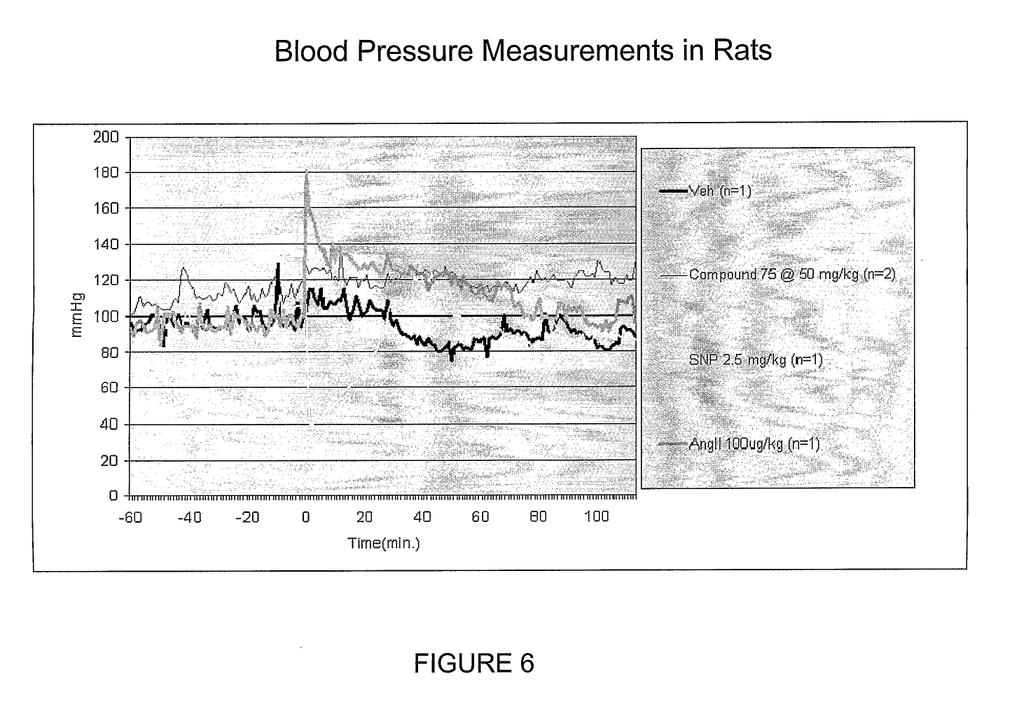

A significant increase in blood pressure is the increase in blood pressure that would be observed after treatment with a known vasoconstrictor compound. An example of a significant increase in blood pressure is shown in FIG. 6. In FIG. 6, the known vasoconstrictor angiotensin II was administered to rats and a significant increase in blood pressure was recorded after administration. A significant increase in blood pressure can be, for example, an increase in blood pressure of 10% or more, 15% or more, 20% or more, 30% or more, 40% or more, 50% or more, 60% or more, 70% or more, 80% or more, 90% or more, or 100% or more. As understood by one skilled in the art, blood pressure readings can be increased in response to factors other than administration of a compound, such as stress. Therefore, care should be taken to control for these other factors.

The invention further relates to a method for inhibiting Mas receptor function in a cell, comprising contacting a cell capable of expressing Mas with an effective amount of the cardio-protective compound identified by a method comprising: a) contacting a candidate compound with a Mas receptor, and b) determining whether the receptor functionality is decreased, wherein a decrease in receptor functionality is indicative of the candidate compound being a cardio-protective compound.

The invention also relates to a method for inhibiting Mas receptor activity in a human host, comprising administering a compound that inhibits activity of the Mas receptor gene product to a human host in need of such treatment. For example, the invention relates to a method for selectively inhibiting Mas receptor activity in a human host, comprising administering a compound of the invention that selectively inhibits activity of the Mas receptor gene product to a human host in need of such treatment. Further, for example, the invention relates to a method for selectively inhibiting Mas receptor activity in a human host, comprising administering an inverse agonist of the Mas receptor that selectively inhibits activity of the Mas receptor gene product to a human host in need of such treatment. Selectively inhibiting Mas receptor activity means significantly inhibiting Mas receptor activity while not significantly inhibiting the activity of, for example, one or more other GPCR, a majority of other GPCRs, or any other GPCR.

The invention further relates to a method for selectively inhibiting Mas receptor activity in a human host, comprising administering a compound of Formula (I) or a pharmaceutically acceptable salt, free base, solvate, hydrate or stereoisomer thereof, as described herein, that selectively inhibits activity of the Mas receptor gene product to a human host in need of such treatment.

For example, a compound of Formula (I) consists of: 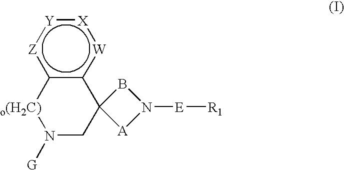
and pharmaceutically acceptable salts, free bases, solvates, hydrates, stereoisomers, clathrates or prodrugs thereof, wherein:

R₁ is H, halogen, hydroxy, nitro, cyano, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, substituted or unsubstituted -(3 to 7) membered heterocycle, substituted or unsubstituted -(7 to 10) membered bicycloheterocycle, substituted or unsubstituted -(5 to 10) membered heteroaryl, —NR₂R₂′, —C(═O)—R₇, —S(═O)₂—R₇, —C(═O)O—R₇, or —C(═O)N(R₇)(C₁₋₆ alkyl);

A is a substituted or unsubstituted C₁-C₃ alkylene;

B is a substituted or unsubstituted C₁-C₃ alkylene;

E is a bond, or a substituted or unsubstituted C₁-C₃ alkylene;

G is H, —Ar, —C(═O)—Ar, —C(═O)O—Ar, substituted or unsubstituted —C(═O)O—C₁₋₆ alkyl, —C(═O)N(R₇)(Ar), substituted or unsubstituted —C(═O)N(R₇)(C₁₋₆ alkyl), —S(═O)₂—Ar, substituted or unsubstituted —S(═O)₂—C₁₋₆ alkyl, substituted or unsubstituted C₁₋₆ alkyl, substituted or unsubstituted C₁₋₆ alkyl-Ar, substituted or unsubstituted —C(═O)C₁₋₆ alkyl-Ar, or substituted or unsubstituted —C(═O)C₁₋₆ alkyl;

W is N or —CR₃—;

X is N or —CR₄—;

Y is N or —CR₅—;

Z is N or —CR₆—;

R₂, R₂′, R₃, R₄, R₅, R₆ and R₇ are at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, —C(═O)—O—C₁₋₆ alkyl, —O—C₁₋₆ alkyl, —C₁₋₆ alkyl-O—C₁₋₆ alkyl, —C₁₋₆ alkyl-NH₂, —C₀₋₆ alkyl-C(═O)—NH(C₁₋₆ alkyl), —C₀₋₆ alkyl-C(═O)—N(C₁₋₆ alkyl)(C₁₋₆ alkyl), —C₁₋₆ alkyl-NH—C(═O)—C₁₋₆ alkyl, —C₁₋₆ alkyl-S(═O)—C₁₋₆ alkyl, —C₀₋₆ alkyl-O—S(═O)₂—C₁₋₆ alkyl, —C₁₋₆ alkyl-S(═O)₂—C₁₋₆ alkyl, —C₁₋₆ alkyl-NR′—S(═O)₂—R′, —C₁₋₆ alkyl-SH, —C₁₋₆ alkyl-S—C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —(C(R′)₂)₀₋₆——(C(R′)₂)₁₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂—(C(R′)₂)₁₋₅C(R′)₃;

o is 0 or 1;

R′ is at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted aryl, or substituted or unsubstituted C₃₋₈ cycloalkyl; and

Ar is substituted or unsubstituted aryl, substituted or unsubstituted C₃₋₇ cycloalkyl, substituted or unsubstituted C₈₋₁₄ bicycloalkyl, substituted or unsubstituted C₈₋₁₄ tricycloalkyl, substituted or unsubstituted -(3 to 7) membered heterocycle, substituted or unsubstituted -(7 to 10) membered bicycloheterocycle, or substituted or unsubstituted -(5 to 10 membered)heteroaryl. The compounds of Formula (I) are further described herein.

The invention also relates to a method for preparing a composition which comprises identifying a cardio-protective compound and then admixing said modulator and carrier, wherein the modulator is identified by a method comprising: a) contacting a candidate compound with a Mas receptor, and b) determining whether the receptor functionality is decreased, wherein a decrease in receptor functionality is indicative of the candidate compound being a cardio-protective compound.

The invention also relates to a pharmaceutical composition comprising, consisting essentially of, or consisting of an inverse agonist identified by a method comprising: a) contacting a candidate compound with a Mas receptor, and b) determining whether the receptor functionality is decreased, wherein a decrease in receptor functionality is indicative of the candidate compound being a cardio-protective compound. A pharmaceutical composition is a composition comprising at least one active ingredient, whereby the composition is amenable to investigation for a specified, efficacious outcome in a mammal (for example, a human). Those of ordinary skill in the art will understand and appreciate the techniques appropriate for determining whether an active ingredient has a desired efficacious outcome based upon the needs of the artisan.

The invention further relates to a method for effecting cardio protection in an individual in need of said cardio protection, comprising administering to said individual an effective amount of the pharmaceutical composition comprising, consisting essentially of, or consisting of an inverse agonist identified according to the method of a) contacting a candidate compound with a Mas receptor, and b) determining whether the receptor functionality is decreased, wherein a decrease in receptor functionality is indicative of the candidate compound being a cardio-protective compound.

The invention also relates to a method for treating or preventing a vascular or cardiovascular disease or disorder in an individual in need of said treating or preventing, comprising administering to said individual an effective amount of the pharmaceutical composition comprising, consisting essentially of, or consisting of an inverse agonist identified according to the method of a) contacting a candidate compound with a Mas receptor, and b) determining whether the receptor functionality is decreased, wherein a decrease in receptor functionality is indicative of the candidate compound being a vascular- or cardio-protective compound.

In one embodiment, the pharmaceutical compositions of the invention are used alone for treating or preventing a disease or disorder. In another embodiment, the pharmaceutical compositions of the invention are used in combination with another compound or therapy for treating or preventing a disease or disorder.

An “individual” or “patient” is defined herein to include any animal (e.g., cow, horse, sheep, pig, chicken, turkey, quail, cat, dog, mouse, rat, rabbit or guinea pig), in one embodiment a mammal such as a non-primate or a primate (e.g., monkey or human), and in another embodiment a human. In certain embodiments, the human is an infant, child, adolescent or adult. In a particular embodiment, the patient is at risk for a vascular, cardiovascular or neurological disease or disorder. Patients who are at risk include, but are not limited to, those with hereditary history of a vascular, cardiovascular or neurological disease or disorder, or in a state of physical health which puts them at risk for a vascular, cardiovascular or neurological disease or disorder. In another embodiment, the patient has previously had a stroke or is at risk to have a stroke.

The phrase “effective amount” when used in connection with a Compound of the Invention means an amount effective for: (a) treating, preventing or managing a vascular or cardiovascular disease or disorder or a neurological disease or disorder; (b) preventing or reducing damage caused by a vascular or cardiovascular disease or disorder or a neurological disease or disorder; (c) inhibiting Mas receptor function in a cell capable of expressing Mas; or (d) detection by an instrument useful for detecting and/or measuring radioactivity (e.g. a liquid scintillation counter).

The phrase “effective amount” when used in connection with another active agent means an amount for treating, preventing or managing a vascular or cardiovascular disease or disorder or a neurological disease or disorder while the Compound of the Invention is exerting its effect.

The phrases “treatment of,” “treating” and the like include the amelioration or cessation of a vascular or cardiovascular disease or disorder or a neurological disease or disorder. In one embodiment, treating includes inhibiting, for example, decreasing the overall frequency of episodes of a cardiovascular disease or disorder or a neurological disease or disorder.

The phrases “prevention of,” “preventing” and the like include the avoidance of the onset of a vascular or cardiovascular disease or disorder or a neurological disease or disorder. In one embodiment, neurological or vascular damage caused by stroke is prevented.

The phrases “management of”, “managing” and the like include the prevention of worsening of a vascular or cardiovascular disease or disorder or a neurological disease or disorder, or a symptom thereof.

As understood by one skilled in the art, a vascular disease or disorder is a disease or disorder related to blood vessels in an animal and a cardiovascular disease or disorder is a disease or disorder related to the heart or blood vessels. Thus, a cardiovascular disease can be considered as a subset of vascular diseases. A neurological disease or disorder is a disease or disorder related to the nervous system in an animal. Some diseases such as stroke and migraine can be considered as both a neurological disease and as a vascular disease.

In one embodiment, said vascular or cardiovascular disease or disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine. In another embodiment, said vascular or cardiovascular disease or disorder is reperfusion injury, acute myocardial infarction, acute or chronic congestive heart failure, left ventricular hypertrophy or vascular hypertrophy.

The invention also relates to a method of effecting a needed change in cardiovascular function in an individual in need of said change, comprising administering an effective amount of a pharmaceutical composition comprising, consisting essentially of, or consisting of an inverse agonist identified by a method comprising: a) contacting a candidate compound with a Mas receptor, and b) determining whether the receptor functionality is decreased, wherein a decrease in receptor functionality is indicative of the candidate compound being a cardio-protective compound, and wherein said needed change in cardiovascular function is an increase in ventricular contractile function. In one embodiment the ventricle is the left ventricle of the heart.

The invention also relates to a method for the manufacture of a medicament comprising this pharmaceutical composition, for use in the treatment of a vascular or cardiovascular disease. The invention further relates to a method for the manufacture of a medicament comprising this pharmaceutical composition, for use as a cardio-protective agent.

The invention further relates to a pharmaceutical composition comprising, consisting essentially of, or consisting of an inverse agonist identified by a method comprising: a) contacting a candidate compound with a Mas receptor, and b) determining whether the receptor functionality is decreased, wherein a decrease in receptor functionality is indicative of the candidate compound being a cardio-protective compound, for use in a method of treatment of the human or animal body by therapy.

### 5.10 THERAPEUTIC USES OF THE COMPOUNDS OF THE INVENTION

In accordance with the invention, the Compounds of the Invention are useful as cardio-protective and/or neuro-protective agents. The Compounds of the Invention can also be administered to a patient in need of treatment, prevention and/or management of a vascular or cardiovascular or neurological disease or disorder.

In one embodiment, the vascular or cardiovascular disease or disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic neuropathy, glomerulonephritis, scleroderma, glomerular sclerosis, renal failure, renal transplant therapy, diabetic retinopathy, or another vascular disorders such as migraine.

In another embodiment, the neurological disease or disorder is diabetic peripheral neuropathy, pain, stroke, cerebral ischemia or Parkinson's disease.

In another embodiment, the Compounds of the Invention are useful as neuro-protective and/or cardio-protective agents and have the ability to prevent or lessen the severity of cerebral ischemia. In a certain embodiment, the cerebral ischemia results from stroke. Without being bound by any particular theory, it is thought that the Compounds of the Invention can prevent or lessen the severity of cerebral ischemia by preventing or lessening acute injury to ischemic neurons.

In another embodiment, the Compounds of the Invention are used in combination with, or in place of, angiotensin-converting enzyme (ACE) inhibitors to treat the diseases or disorders for which such ACE inhibitors are conventionally used. Such diseases or disorders include, but are not limited to, refractory hypertension, congestive heart failure, myocardial infarction, diabetes mellitus, chronic renal insufficiency, atherosclerotic cardiovascular disease, reinfarction, angina, end-stage renal disease, left ventricular dysfunction, or any disease or disorder associated with the renin-angiotensin system.

In one embodiment, an effective amount of a Compound of the Invention can be used to treat, prevent and/or manage any disease or disorder treatable, preventable and/or manageable by binding to the Mas receptor. Examples of diseases or disorders that are treatable or preventable by inhibiting binding to the Mas receptor include, but are not limited to, vascular, cardiovascular or neurological diseases or disorders. In a particular embodiment, an effective amount of a Compound of the Invention can be used to treat, prevent and/or manage any disease or disorder treatable, preventable and/or manageable by inhibiting Mas receptor function.

The invention further relates to methods for inhibiting Mas function in a cell comprising contacting a cell capable of expressing Mas with an amount of a Compound of the Invention effective to inhibit Mas function in the cell. This method can be used in vitro, for example, as an assay to select cells that express Mas and, accordingly, is useful as part of an assay to select compounds useful for treating, preventing and/or managing a vascular or cardiovascular disease or disorder or a neurological disease or disorder. The method is also useful for inhibiting Mas function in a cell in vivo, such as in a patient, in a human in one embodiment, by contacting a cell, in a patient, with an amount of a Compound of the Invention effective to inhibit Mas function in the cell.

Preferred Compounds of the Invention for use in the methods described herein are those wherein G is —C(═O)—Ar. Still further preferred Compounds of the Invention for use in the methods described herein are those wherein G is —C(═O)—NH—Ar. Still further preferred Compounds of the Invention for use in the methods described herein are those wherein A and B are both —(CH₂)₂—. Still further preferred Compounds of the Invention for use in the methods described herein are those wherein Ar is substituted phenyl, preferable halogenated phenyl. Still further preferred Compounds of the Invention for use in the methods described herein are those wherein W, X, Y and Z are —CR₃—, —CR₄—, —CR₅— and —CR₆—, respectively. Still further preferred Compounds of the Invention for use in the methods described herein are those wherein W, X and Y are —CH—, and Z is —CF—. Still further preferred Compounds of the Invention for use in the methods described herein are those wherein p is 1 and R₁ is cyclopropyl. Still further preferred Compounds of the Invention for use in the methods described herein are those wherein p is 1 and R₁ is —CH═CH₂.

### 5.11 THERAPEUTIC/PROPHYLACTIC ADMINISTRATION AND COMPOSITIONS OF THE INVENTION

Due to their activity, the Compounds of the Invention are advantageously useful in veterinary and human medicine. As described above, the Compounds of the Invention are useful for treating, preventing and/or managing a vascular or cardiovascular or neurological disease or disorder in a patient in need thereof. Accordingly, in one embodiment, the present invention relates to a method for manufacturing a medicament comprising one or more Compounds of the Invention and a pharmaceutically acceptable vehicle or excipient. In another embodiment, the medicament can further comprise another active agent.

When administered to a patient, the Compounds of the Invention can be administered as a component of a composition, such as a pharmaceutical composition, that comprises a pharmaceutically acceptable vehicle or excipient. The present compositions, which comprise a Compound of the Invention, can be administered intradermally, intramuscularly, intraperitoneally, intravenously, subcutaneously, intranasally, epidurally, orally, sublingually, intracerebrally, intravaginally, transdermally, rectally, by inhalation, topically (particularly to the ears, nose, eyes, or skin), by infusion or bolus injection, or by absorption through epithelial or mucocutaneous linings (e.g., oral, rectal, or intestinal mucosa) and can optionally be administered together with another active agent. Administration can be systemic or local. Various delivery systems are known, e.g., encapsulation in liposomes, microparticles, microcapsules or capsules, and can be used to administer the Compound of the Invention.

In specific embodiments, it can be desirable to administer the Compounds of the Invention locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Compounds of the Invention into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the Compounds of the Invention can be formulated as a suppository, with traditional binders and excipients such as triglycerides.

In another embodiment, the Compounds of the Invention can be delivered in a vesicle, in particular a liposome (See Langer, Science 249:1527-1533 (1990) and Treat et al., Liposomes in the Therapy of Infectious Disease and Cancer 317-327 and 353-365 (1989).

In yet another embodiment, the Compounds of the Invention can be delivered in a controlled-release system or sustained-release system (See, e.g., Goodson, in Medical Applications of Controlled Release, supra, vol. 2, pp. 115-138 (1984)). Other controlled- or sustained-release systems discussed in the review by Langer, Science 249:1527-1533 (1990) can be used. In one embodiment, a pump can be used (Langer, Science 249:1527-1533 (1990); Sefton, CRC Crit. Ref Biomed. Eng. 14:201 (1987); Buchwald et al., Surgery; 88:507 (1980); and Saudek et al., N. Engl. J. Med. 321:574 (1989)). In another embodiment, polymeric materials can be used (See Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)). In yet another embodiment, a controlled- or sustained-release system can be placed in proximity of a target of the Compounds of the Invention, e.g., the spinal column, brain, or gastrointestinal tract, thus requiring only a fraction of the systemic dose.

The present pharmaceutical compositions can optionally comprise a suitable amount of a pharmaceutically acceptable excipient so as to provide the form for proper administration to the patient.

The pharmaceutical compositions can be for a single, one-time use or can contain antimicrobial excipients, as described herein, rendering the pharmaceutical compositions suitable for multiple uses, for example a multi-use vial. In another embodiment, the pharmaceutical compositions can be in unit dose or unit-of-use packages. As is known to those of skill in the art, a unit dose package provides delivery of a single dose of a drug to a subject. The methods of the invention provide for a unit dose package of a pharmaceutical composition comprising, for example, 700 mcg of a Compound of the Invention per unit. The 700 mcg of a Compound of the Invention, is an amount that administers 10 mcg/kg to a 70 kg subject, for example. The unit can be, for example, a single use vial, a pre-filled syringe, a single transdermal patch and the like.

As is known to those of skill in the art, a unit-of-use package is a convenient, prescription size, patient ready unit labeled for direct distribution by health care providers. A unit-of-use package contains a pharmaceutical composition in an amount necessary for a typical treatment interval and duration for a given indication. The methods of the invention provide for a unit-of-use package of a pharmaceutical composition comprising, for example, a Compound of the Invention in an effective amount for treating an average sized adult male or female. It will be apparent to those of skill in the art that the doses described herein are based on the subject's body weight.

The pharmaceutical compositions can be labeled and have accompanying labeling to identify the composition contained therein and other information useful to health care providers and subjects in the treatment of a vascular or cardiovascular or neurological disorder, including, but not limited to, instructions for use, dose, dosing interval, duration, indication, contraindications, warnings, precautions, handling and storage instructions and the like.

The term “label” refers to a display of written, printed or graphic matter upon the immediate container of an article, for example the written material displayed on a vial containing a pharmaceutically active agent.

The term “labeling” refers to all labels and other written, printed or graphic matter upon any article or any of its containers or wrappers or accompanying such article, for example, a package insert or instructional videotapes or DVDs accompanying or associated with a container of a pharmaceutically active agent.

Pharmaceutical excipients for use in the present pharmaceutical compositions can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to an animal. Water, and in one embodiment physiological saline, is a particularly useful excipient when the Piperazine Compound is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsions, tablets, pills, pellets, capsules, capsules containing liquids, powders, sustained-release formulations, suppositories, aerosols, sprays, suspensions, or any other form suitable for use. In one embodiment, the composition is in the form of a capsule (See, e.g., U.S. Pat. No. 5,698,155). Other examples of suitable pharmaceutical excipients are described in Remington's Pharmaceutical Sciences 1447-1676 (Alfonso R. Gennaro ed., 19th ed. 1995), incorporated herein by reference.

In one embodiment, the Compounds of the Invention are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the Compounds of the Invention can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lidocaine to lessen pain at the site of the injection. The ingredients can be supplied either separately or mixed together in unit dosage form, for example, as a dry lyophilized powder or water free concentrate in a hermetically sealed container such as an ampoule or sachette indicating the quantity of active agent. Where the Compounds of the Invention are to be administered by infusion, they can be dispensed, for example, with an infusion bottle containing sterile pharmaceutical grade water or saline. Where the Compounds of the Invention are administered by injection, an ampoule of sterile water for injection or saline can be provided so that the ingredients can be mixed prior to administration.

The Compounds of the Invention can be administered by controlled-release or sustained-release means or by delivery devices that are known to those skilled in the art. Examples include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference. Such dosage forms can be used to provide controlled- or sustained-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

Controlled- or sustained-release pharmaceutical compositions can have a common goal of improving drug therapy over that achieved by their non-controlled or non-sustained counterparts. In one embodiment, a controlled- or sustained-release composition comprises a minimal amount of a Compound of the Invention to treat or prevent a disease or disorder in a minimal amount of time. Advantages of controlled- or sustained-release compositions include extended activity of the drug, reduced dosage frequency, and increased patient compliance. In addition, controlled- or sustained-release compositions can favorably affect the time of onset of action or other characteristics, such as blood levels of the Compound of the Invention, and can thus reduce the occurrence of adverse side effects.

Controlled- or sustained-release compositions can initially release an amount of a Compound of the Invention that promptly produces the desired therapeutic or prophylactic effect, and gradually and continually release other amounts of the Compound of the Invention to maintain this level of therapeutic or prophylactic effect over an extended period of time. To maintain a constant level of the Compound of the Invention in the body, the Compound of the Invention can be released from the dosage form at a rate that will replace the amount of the Compound of the Invention being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of the Compound of the Invention that is effective in the treatment or prevention of a disease or disorder can be determined by standard clinical techniques. In addition, in vitro or in vivo assays can optionally be employed to help identify optimal dosage ranges. The precise dose to be employed will also depend on the route of administration, and the seriousness of the disorder and can be decided according to the judgment of a practitioner and/or each patient's circumstances. Suitable effective dosage amounts, however, range from about 0.01 mg/kg of body weight to about 2500 mg/kg of body weight about every 4 h, although they are typically about 100 mg/kg of body weight or less. In one embodiment, the effective dosage amount ranges from about 0.01 milligrams to about 100 milligrams of a Compound of the Invention, in another embodiment, about 0.02 mg/kg of body weight to about 50 mg/kg of body weight, and in another embodiment, about 0.025 mg/kg of body weight to about 20 mg/kg of body weight. In one embodiment, an effective dosage amount is administered about every 12 h. In another embodiment, an effective dosage amount is administered about every 24 h. In another embodiment, an effective dosage amount is administered about every two days. In another embodiment, an effective dosage amount is administered twice a week. In another embodiment, an effective dosage amount is administered about once a week. In another embodiment, an effective dosage amount is administered about once every two weeks. In another embodiment, an effective dosage amount is administered about once per month.

Where a cell capable of expressing Mas is contacted with a Compound of the Invention in vitro, the amount effective for inhibiting the Mas receptor function in a cell will typically range from about 0.01 μg/L to about 5 mg/L, in one embodiment, from about 0.01 μg/L to about 2.5 mg/L, in another embodiment, from about 0.01 μg/L to about 0.5 mg/L, and in another embodiment, from about 0.01 μg/L to about 0.25 mg/L of a solution or suspension of a pharmaceutically acceptable carrier or excipient. In one embodiment, the volume of solution or suspension comprising the Compound of the Invention is from about 0.01 μL to about 1 mL. In another embodiment, the volume of solution or suspension is about 200 μL.

Where a cell capable of expressing Mas is contacted with a Compound of the Invention in vivo, the amount effective for inhibiting the receptor function in a cell will typically range from about 0.01 mg/kg of body weight to about 2500 mg/kg of body weight, although it typically ranges from about 100 mg/kg of body weight or less. In one embodiment, the effective dosage amount ranges from about 0.01 mg/kg of body weight to about 100 mg/kg of body weight of a Compound of the Invention, in another embodiment, about 0.02 mg/kg of body weight to about 50 mg/kg of body weight and in another embodiment, about 0.025 mg/kg of body weight to about 20 mg/kg of body weight. In one embodiment, an effective dosage amount is administered about every 24 h. In another embodiment, an effective dosage amount is administered about every 12 h. In another embodiment, an effective dosage amount is administered about every 8 h. In another embodiment, an effective dosage amount is administered about every 6 h. In another embodiment, an effective dosage amount is administered about every 4 h.

The Compounds of the Invention can be assayed in vitro or in vivo for the desired therapeutic or prophylactic activity prior to use in a humans. Animal model systems can be used to demonstrate safety and efficacy in humans.

The present methods for treating or preventing a disease or disorder in a patient in need thereof can further comprise administering another therapeutic agent to a patient being administered a Compound of the Invention. In one embodiment, the other therapeutic agent is administered in an effective amount.

The present methods for inhibiting Mas receptor function in a cell capable of expressing a Mas receptor can further comprise contacting the cell with an effective amount of another therapeutic agent.

Effective amounts of the other therapeutic agents are known to those skilled in the art. However, it is within the skilled artisan's purview to determine the other therapeutic agent's optimal effective-amount range. In one embodiment of the invention, where another therapeutic agent is administered to an animal, the effective amount of the Compound of the Invention is less than its effective amount would be where the other therapeutic agent is not administered. In this case, without being bound by theory, it is believed that the Compounds of the Invention and the other therapeutic agent act synergistically to treat or prevent a vascular or cardiovascular or neurological disease or disorder.

The other therapeutic agents can be, but is not limited to, aspirin, nitrates (e.g. nitroglycerin), ACE inhibitors, beta-blockers, calcium channel blockers, statins, N-methyl-D-aspartate FODA) receptor antagonists, non-NMDA neuroprotective agents, free-radical scavengers, or any other agent useful for treating, preventing and/or managing a vascular or cardiovascular or neurological disorder or useful as a neuroprotective agent.

Examples of ACE inhibitors include, but are not limited to, trandolapril, benazepril, captopril, enalapril, fosinopril, lisinopril, moexipril, quinapril and ramipril.

Examples of beta-blockers include, but are not limited to, propranolol, verapamil, and divalproex.

Examples of calcium channel blockers include, but are not limited to, bepridil, clentiazem, diltiazem, fendiline, gallopamil, mibefradil, prenylamine, semotiadil, terodiline, verapamil, amlodipine, aranidipine, bamnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, fantofarone, and perhexiline.

Examples of NMDA receptor antagonists include, but are not limited to, selfotel, aptiganel and magnesium.

Examples of non-NMDA neuroprotective agents include, but are not limited to, nalmefene, lubeluzole and clomethiazole.

An example of a free-radical scavenger includes, but is not limited to, tirilizad.

Examples of useful therapeutic agents for treating or preventing Parkinson's disease include, but are not limited to, carbidopa/levodopa, pergolide, bromocriptine, ropinirole, pramipexole, entacapone, tolcapone, selegiline, amantadine, and trihexyphenidyl hydrochloride.

Examples of useful therapeutic agents for treating or preventing stroke include, but are not limited to, anticoagulants such as heparin, agents that break up clots such as streptokinase or tissue plasminogen activator, agents that reduce swelling such as mannitol or corticosteroids, and acetylsalicylic acid.

Examples of useful therapeutic agents for treating or preventing a migraine include, but are not limited to, sumatriptan, methysergide, ergotamine, caffeine and beta-blockers.

A Compound of the Invention and the other therapeutic agent(s) can act additively or, in one embodiment, synergistically. In one embodiment, a Compound of the Invention is administered concurrently with another therapeutic agent; for example, a composition comprising an effective amount of a Compound of the Invention, an effective amount of another therapeutic agent can be administered. Alternatively, a composition comprising an effective amount of a Compound of the Invention and a different composition comprising an effective amount of another therapeutic agent can be concurrently administered. In another embodiment, an effective amount of a Compound of the Invention is administered prior or subsequent to administration of an effective amount of another therapeutic agent. In this embodiment, the Compound of the Invention is administered while the other therapeutic agent exerts its therapeutic effect, or the other therapeutic agent is administered while the Compound of the Invention exerts its preventative or therapeutic effect for treating or preventing a vascular or cardiovascular or neurological disorder.

In another embodiment, the Compound of the Invention is administered in combination with surgery associated with a vascular or cardiovascular or neurological disorder. Examples of surgery associated with a vascular or cardiovascular disorder include, but are not limited to, open-heart surgery, closed-heart surgery, coronary artery bypass surgery, heart valve surgery or angioplasty.

### 5.12 DIAGNOSTIC USES OF THE COMPOUNDS OF THE INVENTION

The invention further relates to methods for assaying the ability of a Compound of the Invention to bind to a Mas receptor, comprising contacting a radio-labeled Compound of the Invention with a cell or tissue capable of expressing a Mas receptor.

Radio-labeled Compounds of the Invention including, but not limited to, those containing one or more ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I or ¹³¹I atoms. The radionuclide that is incorporated in the radio-labeled Compound of the Invention will depend on the specific application of that radio-labeled compound. For example, for in vitro Mas receptor labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

Certain isotopically-labeled Compounds of the Invention are useful in compound and/or substrate tissue distribution assays. In certain embodiments, the Compounds of the Invention containing a ³H and/or ¹⁴C isotopes are useful in these studies. In other embodiments, substitution with heavier isotopes such as deuterium (ie., ²H) can afford certain therapeutic advantages resulting from greater metabolic stability including, but not limited to, increased in vivo half-life or reduced dosage requirements. Isotopically labeled Compounds of the Invention can generally be prepared by synthetic procedures analogous to those disclosed herein, by substituting an isotopically labeled reagent for a non-isotopically labeled reagent. It should be understood that all of the atoms represented in the compounds of the invention can be either the most commonly occurring isotope of such atoms or the more scarce radio-isotope or non-radioactive isotope.

In one embodiment, the invention relates to screening assays useful for identifying and/or evaluating Mas receptor binding ability of test compounds comprising the use of a radio-labeled Compound of the Invention. In general terms, a test compound can be evaluated for its ability to reduce binding of the radio-labeled Compound of the Invention to a Mas receptor. Accordingly, the ability of a test compound to compete with the radio-labeled Compound of the Invention for the binding to the Mas receptor directly correlates to its Mas receptor binding affinity.

In another embodiment, the invention relates to assays useful for locating or quantitating Mas receptor in a tissue sample, comprising contacting the tissue sample with an effective amount of a radio-labeled Compound of the Invention.

The radio-labeled Compounds of the Invention bind to the Mas receptor. In one embodiment the radio-labeled Compound of the Invention has an IC₅₀ less than about 500 μM, in another embodiment the radio-labeled Compound of the Invention has an IC₅₀ less than about 100 μM, in yet another embodiment the radio-labeled Compound of the Invention has an IC₅₀ less than about 10 μM, in yet another embodiment the radio-labeled Compound of the Invention has an IC₅₀ less than about 1 μM, in yet another embodiment the radio-labeled Compound of the Invention has an IC₅₀ less than about 0.1 μM, in yet another embodiment the radio-labeled Compound of the Invention has an IC₅₀ less than about 10 nM, and in still yet another embodiment the radio-labeled Compound of the Invention has an IC₅₀ less than about 1 nM.

Other uses of the disclosed radio-labeled Compounds of the Invention and methods will become apparent to those in the art based upon, inter alia, a review of this disclosure.

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative and not intended to be limiting.

### 5.13 KITS

The invention encompasses kits that can simplify the administration of a Compound of the Invention to a patient.

A typical kit of the invention comprises a unit dosage form of a Compound of the Invention. In one embodiment, the unit dosage form is a container, which can be sterile, containing an effective amount of a Compound of the Invention and a pharmaceutically acceptable vehicle or excipient. The kit can further comprise a label or printed instructions instructing the use of the Compound of the Invention. The kit can also further comprise a unit dosage form of another therapeutic agent, for example, a second container containing an effective amount of the other therapeutic agent and a pharmaceutically acceptable vehicle or excipient. In another embodiment, the kit comprises a container containing an effective amount of a Compound of the Invention, an effective amount of another therapeutic agent and a pharmaceutically acceptable vehicle or excipient. Examples of other therapeutic agents include, but are not limited to, those listed above.

Kits of the invention can further comprise a device that is useful for administering the unit dosage forms. Examples of such a device include but are not limited to a syringe, a drip bag, a patch, an inhaler, and an enema bag.

### 6. EXAMPLES

The following examples are set forth to assist in understanding the invention and should not be construed as specifically limiting the invention described and claimed herein.

### 6.1 ILLUSTRATIVE COMPOUNDS OF THE INVENTION

Examples 1-34 are illustrative Compounds of the Invention which were prepared using similar methods as set forth in Section 5.8 supra.

### Example 1

### Preparation of 1′-(allyl)-1,2-dihydro-5-methoxy-spiro[3H-indole-3,4′-piperidine]

### Step 1: 1-Allyl-piperidine-4-carbaldehyde

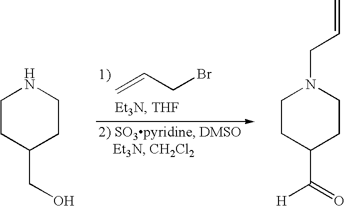

To a stirring solution of 4-piperidinemethanol (3.62 g, 31.4 mmol) and Et₃N (6.0 mL, 44.0 mmol) in THF (50 mL) was added allyl bromide (3.19 mL, 37.7 mmol). The reaction was stirred for about 5 h at ambient temperature, diluted with EtOAc (100 mL) and washed with H₂O (2×100 mL). NaOH (5N aq., 50 mL) was added to the aqueous phase followed by back-extraction of the aqueous phase with CH₂Cl₂ (2×100 mL). The combined organics were dried over MgSO₄, filtered and concentrated. The resulting oil was dissolved in CH₂Cl₂ (83 mL) followed by the addition of Et₃N (6.8 mL, 50.13 mmol), DMSO (16 mL, 225 mmol), and SO₃.pyridine (5.32 g, 33.4 mmol). The mixture was stirred at room temperature for 15 h and washed with H₂O (2×100 mL). The aqueous phase was back extracted with CH₂Cl₂ (100 mL) and the combined organics were dried over Na₂SO₄, filtered, and concentrated to give the resulting compound (2.08 g, 13.6 mmol, 43% overall yield) as a yellow oil.

¹H NMR (CDCl₃, 400 MHz): δ 9.64 (1H, s), 5.85 (1H, m), 5.18 (1H, d, J=16.8 Hz), 5.14 (1H, d, J=8.4 Hz), 3.00 (2H, d, J =6.4 Hz),2.84 (2H, m), 2.24 (1H, m), 2.10 (2H, m), 1.90 (2H, m), 1.72 (2H, m).

### Step 2: 1′-(Allyl)-1,2-dihydro-5-methoxy-spiro[3H-indole-3,4′-piperidine]

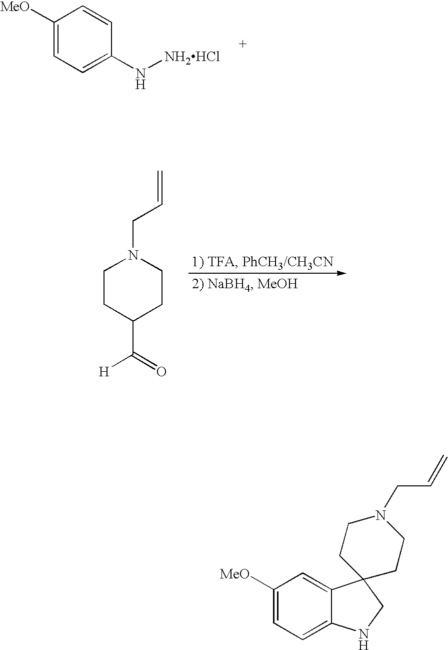

To a flask under N₂ containing the above hydrazine (629 mg, 3.60 mmol) in degassed PhCH₃/CH₃CN (50:1, v/v, 16 mL) and TFA (0.75 mL, 9.74 mmol), was added the above aldehyde (500 mg, 3.26 mmol) at room temperature. After stirring for 15 min at room temperature the reaction was heated to 37° C. and stirred for 20 h. The reaction was cooled to −5° C. (ice/salt bath) and MeOH (20 mL) was added followed by the slow addition of NaBH₄ (185 mg, 4.89 mmol, added over 5 min). The reaction was stirred for 1 h, diluted with EtOAc (50 mL) and washed with NaOH (1M aq., 2×50 mL) and brine (50 mL). The organics were dried over MgSO₄, filtered, and concentrated. The material was purified by reverse-phase HPLC: Phenomenex® Luna C18 column (10 μ, 250×50 min), 5% (v/v) CH₃CN (containing 1% v/v TFA) in H₂O (containing 1% v/v TFA) gradient to 95% H₂O, 60 ml/min, λ=214 nm. Products were isolated as mono-TFA salts after lyophilization to give the resulting compound as the bis-TFA salt (740 mg, 1.52 mmol, 47% overall yield).

¹H NMR (CDCl₃, 400 MHz): δ 6.72 (1H, d, J=2.0 Hz), 6.60 (1H, d, J=2.0 Hz), 6.59 (1H, s), 5.92 (1H, ddt, J=16.8, 10.0, 6.4 Hz), 5.20 (1H, d, J=17.2 Hz), 5.16 (1H, d, J=10.4 Hz), 3.73 (3H, s), 3.42 (2H, s), 3.04 (2H, d, J=6.4 Hz), 2.91 (2H, d, J=12.0 Hz), 2.06 (2H, t, J=13.6 Hz), 1.94 (2H, td, J=13.2, 3.6 Hz), 1.75 (2H, d, J=13.2 Hz). HPLCIMS: Discovery®C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tr =0.92 min, ESI⁺=259.2 (M+H).

### Example 2

### Preparation of 1′-(tert-butoxycarbonyl)-1-benzyl-5,7-dimethyl-spiro[3H-indole-3,4′-piperidin]-2(1H)-one

### Step 1: Preparation of 4-(2-bromo-4-methyl-phenylcarbamoyl)-piperidine-1-carboxylic acid tert-butyl ester

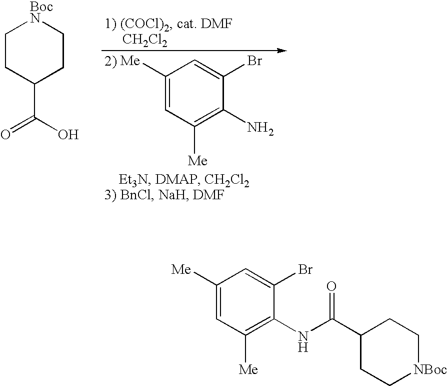

To a solution of the N-Boc-piperidine-4-carboxylic acid (4.00 g, 17.5 mmol) in CH₂Cl₂ (80 mL) stirred under N₂ at room temperature was added oxalyl chloride (1.50 mL, 17.2 mmol) followed by DMF (68 uL, 0.88 mmol). The reaction was stirred for 1 h and Et₃N (5.5 mL, 40 mmol) was added followed by the addition of 2-bromo-4,6-dimethyl aniline (2.60 mL, 20.8 mmol) and 4-(dimethylamino) pyridine (210 mg, 1.72 mmol). After stirring for 18 h at room temperature, the reaction mixture was diluted with CH₂Cl₂ (100 mL) and washed sequentially with HCl (1N aq., 3×100 mL) and NaHCO₃ (sat. aq., 100 mL). The organic layer was dried with MgSO₄, filtered, and concentrated. Purification by silica gel chromatography (15% ethyl acetate in hexanes) gave 4-(2-bromo-4,6-dimethyl-phenylcarbamoyl)-piperidine-1-carboxylic acid tert-butyl ester (2.75 g, 6.94 mmol, 40% yield) as a white powder.

### Step 2: Preparation of 4-[benzyl-(2-bromo-4,6-dimethyl-phenyl)-carbamoyl]-piperidine-1-carboxylic acid tert-butyl ester

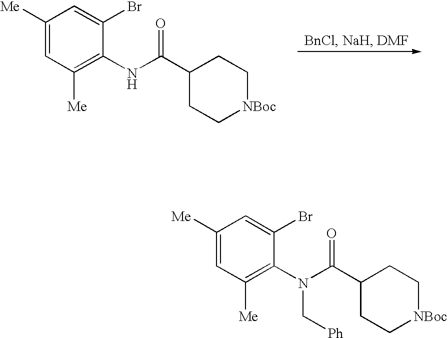

To a solution of NaH (118 mg, 4.91 mmol) in anhydrous DMF (1.9 mL) at 0° C. was added 4-(2-bromo-4,6-dimethyl-phenylcarbamoyl)-piperidine-1-carboxylic acid tert-butyl ester (1.50 g, 3.79 mmol) as a solution in anhydrous DMF (2.3 mL added drop-wise). The resulting solution was stirred for 30 min while warming to room temperature. The reaction was cooled to 0° C. and benzyl chloride (0.45 mL, 3.78 mmol) was added. The reaction was warmed slowly to room temperature and stirred under N₂ for 18 h. The reaction was quenched by the addition of NH₄Cl (sat. aq., 20 mL) and the mixture was extracted with ethyl acetate (3×20 mL). The organic layer was washed with brine (30 mL) and dried over MgSO4. The solvent was evaporated under reduced pressure and the crude product was purified by flash chromatography using 20% ethyl acetate in hexanes to give 4-[benzyl-(2-bromo-4,6-dimethyl-phenyl)-carbamoyl]-piperidine-1-carboxylic acid tert-butyl ester (1.65 g, 3.39 mmol, 89% yield) as a white powder.

¹H NMR (400 MHz, CDCl₃): δ 7.3 (s, 1H), 7.15 (m, 5H), 6.9 (s, 1H), 5.4 (d, J=13.9, 1H), 4.2 (d, J=13.8, 1H), 4.0 (m, 2H), 2.45 (t, J=12.1, 2H), 2.3 (s, 3H), 2.0 (m, 1H), 1.75 (m, 2H), 1.6 (s, 3H), 1.4 (s, 1 1H).

### Step 3: Preparation of 1′-(tert-butoxycarbonyl)-1-benzyl-5-methyl-spiro[3H-indole-3,4′-piperidin]-2(1H)-one

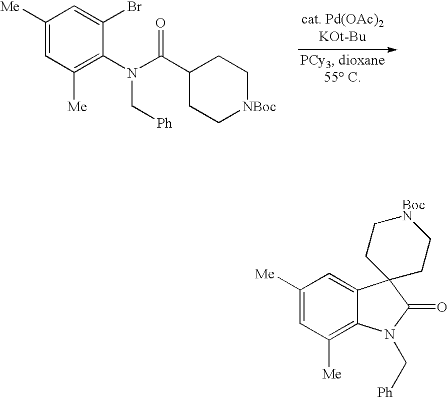

To a 250 mL Schlenck flask (w/injection port) containing Pd(OAc)₂ (270 mg, 1.2 mmol) was added PCy₃ (336 mg, 1.2 mmol) as a solution in dioxane (70 mL). To the same flask was then added KOtBu as a 1M solution in THF (24 mL,24 mmol). 4-[Benzyl-(2-bromo-2,4-dimethyl-phenyl)-carbamoyl]-piperidine-1-carboxylic acid tert-butyl ester (6.05g, 12 mmol) in dioxane (16 mL) was then added and the resulting solution was stirred under nitrogen, at 55° C. for 18 h. After cooling to room temperature the reaction was diluted with ethyl acetate (200 mL) and washed with NH₄Cl (sat. aq., 3×100 mL and brine (100 mL). The organic layer was dried over MgSO₄ and concentrated. Purification by silica gel chromatography (15% ethyl acetate in hexanes) gave the resulting spiroindoline (2.4 g, 5.7 mmol, 48% yield).

¹H NMR (400 MHz, CD₃CN): δ7.33 (m, 2H), 7.26 (m, 1H), 7.13 (d, J=7.1, 2H), 7.06 (s, 1H), 6.78 (s, 1H), 5.15 (s, 2H), 3.85 (m, 2H1), 3.7 (m, 2H), 2.25 (s, 3H), 2.18 (s, 3H), 1.8 (m, 4H), 1.5 (s,9H).

### Example 3

### General Method—Preparation of 1′-(tert-butoxycarbonyl)-1,2-dihydro-spiro-3H-indole-3,4′-piperidines

### Example 3.1

### Preparation of 1′-(tert-Butoxycarbonyl)-1,2-dihydro-5,7-dimethyl-spiro-3H-indole-3,4′-piperidine.

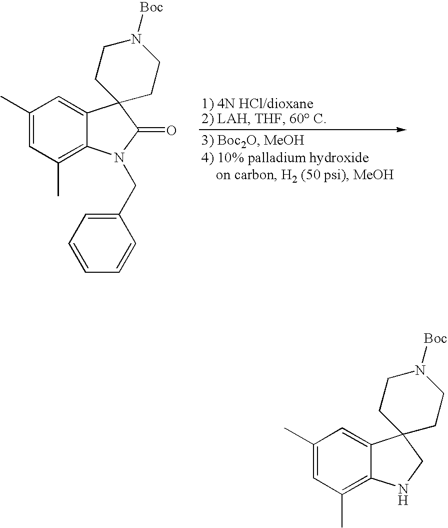

The above spiroindoline (prepared in a similar manner as described in Example 2, above) (1.52 mmol, 1.0 equiv.) was treated with 4N HCl/dioxane (11 mL) for 2 h at room temperature. The volatiles were removed in vacuo and the residue was dissolved in EtOAc (25 mL) and washed with NaOH (1M aq., 25 mL). The organics were dried over MgSO₄, filtered, and concentrated. The concentrate was dissolved in THF (1.4 mL) and cooled to 0° C. A solution of LAH (1M in THF, 4 mL, 2.6 equiv.) was added and the mixture was warmed slowly to room temperature. A reflux condenser was attached and the reaction was heated to 60° C. under N₂ for 16 h. The reaction was monitored by LC/MS and, if necessary, additional LAH was added until the reaction was complete. After cooling to room temperature, the reaction was quenched by the addition of H₂O (0.5 mL). The mixture was diluted with EtOAc (25 mL), washed sequentially with NaOH (1M aq., 25 mL) and brine (25 mL). The organics were dried over MgSO₄, filtered, and concentrated. The concentrate was dissolved in MeOH (4 mL) and treated with Boc₂O (1.3 equiv. based on mass of mono-benzylated product). The reaction was stirred for 20 hours at room temperature, diluted with EtOAc (25 mL), and washed with NaOH (1M aq., 25 mL). The organics were dried over MgSO₄, filtered, and concentrated. The crude mono-Boc/benzyl-spiroindole was added to a 27 mL reaction vessel containing 10% palladium hydroxide on carbon (32 mg) and methanol (20 mL). The solution was placed under H₂ atmosphere at 50 psi, and shaken for 18 h. The solution was filtered and concentrated in vacuo. Purification by silica gel chromatography (5% methanol in CH₂Cl₂) gave the mono-Boc spiroindole product.

NMR and LC/MS characterization for 1′-(tert-butoxycarbonyl)-1,2-dihydro-5,7-dimethyl-spiro-3H-indole-3,4′-piperidine

¹H NMR (400 MHz, CDCl₃): δ 6.75 (s, 1H) 6.68 (s, 1H) 4.15-3.95 (d, J=13.4, 2H), 3.4 (s, 2H) 3.0-2.85 (m, 2H) 2.2 (s, 3H) 2.05 (s, 311) 1.75-1.65 (m, 2H) 1.65-1.55 (m, 2H), 1.48 (s, 9H).

HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tᵣ=1.81 min, ESI⁺=317.2 (M+H).

Other exemplary compounds are shown below and were prepared using essentially the same procedure and methodology as described for 1′-(tert-butoxycarbonyl)-1,2-dihydro-5,7-dimethyl-spiro-3H-indole-3,4′-piperidine, supra.

### Example 3.2

### NMR and LC/MS Characterization for 1′-(tert-butoxycarbonyl)-1,2-dihydro-6-fluoro-spiro-3H-indole-3,4′-piperidine.

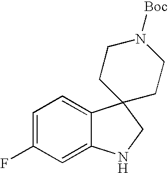

HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tᵣ=2.43 min, ESI⁺=306.4 (M+H).

### Example 3.3

### LC/MS characterization for 1′-(tert-butoxycarbonyl)-1,2-dihydro-6-methoxy-spiro-3H-indole-3,4′-piperidine

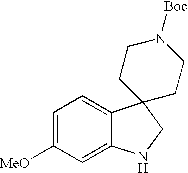

HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tᵣ=1.76 min, ESI⁺=319.2 (M+H).

### Example 3.4

### LC/MS Characterization for 1′-(tert-butoxycarbonyl)-1,2-dihydro-6-trifluoromethyl-spiro-3H-indole-3,4′-piperidine

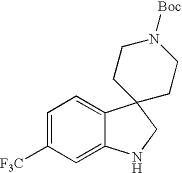

HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tᵣ=2.81 min, ESI⁺=357.1 (M+H).

### Example 4

### Preparation of Compounds of the Invention Via Parallel Synthesis

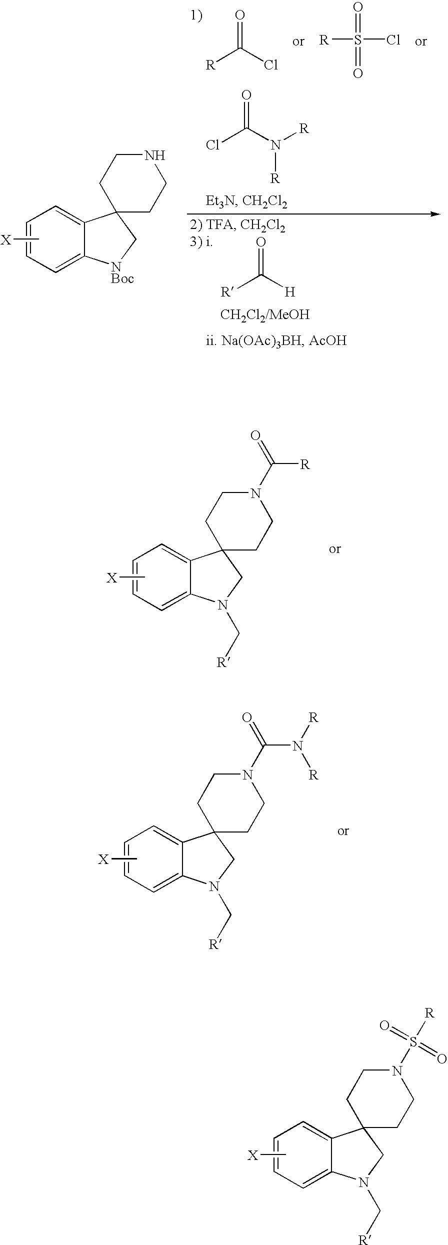

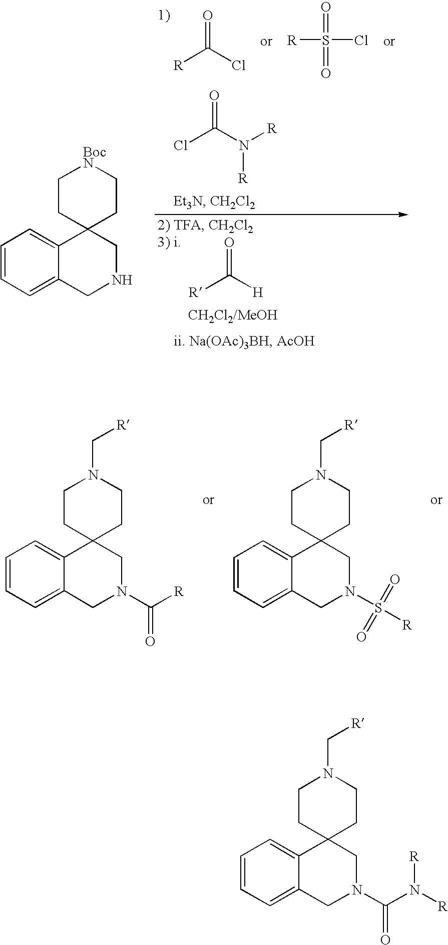

To a solution of Boc-spirocycle (Boc-spm rocycles are commercially available from WuXi PharmaTech Co., Ltd., Shanghai 200131, China) (2.0 mmol, 1.0 equiv.) and Et₃N (3.0 mmol, 1.5 equiv.) in CH₂Cl₂ (3.5 mL) at room temperature was added acid/carbamoyl/sulfthionyl chloride (2.0 mmol, 1.0 equiv.) as a solution in CH₂Ch₂ (4 mL). Reactions were stirred for 4 h and washed with HCl (1M aq., 5 mL) and NaOH C₃ (sat. aq., 5 mL). Organics were dried over Na₂SO₄, filtered, and concentrated. To the concentrate was added 20% TFA/DCM (v/v, 6 mL) and the reaction was stirred for 20 h at ambient temperature at which time NaOH (2.5 N aq., 10 mL) was added. The organic phase was separated, dried over Na₂SO₄, filtered, and concentrated.

The reductive aminations were performed on split portions of the deprotected products as described: To the amine (0.4 mmol, 1.0 equiv.) in CH₂Cl₂/MeOH (4:1, v/v, 5 mL) was added aldehyde (0.4 mmol, 1.0 equiv.) at room temperature. The reaction was stirred for 5 h at room temperature at which time AcOH (0.8 mmol, 2.0 equiv.) and Na(OAC)₃BH (0.8 mmol, 2.0 equiv.) were added. The reactions were stirred for an additional 20 h, diluted with CH₂Cl₂ (5 mL), and washed with NaOH (1M aq., 8 mL). The reactions were concentrated and purified by reverse-phase HPLC: Phenomenex® Luna C18column (10 μ, 250×21.2 mm), 5% (v/v) CH₃CN (containing 1% v/v TFA) in H₂O (containing 1% v/v TFA) gradient to 95% H₂O, 20 ml/min, λ=214 nm. Products were isolated as mono-TFA salts after lyophilization.

### Example 5

### Preparation of Compounds of the Invention Via Parallel Synthesis.

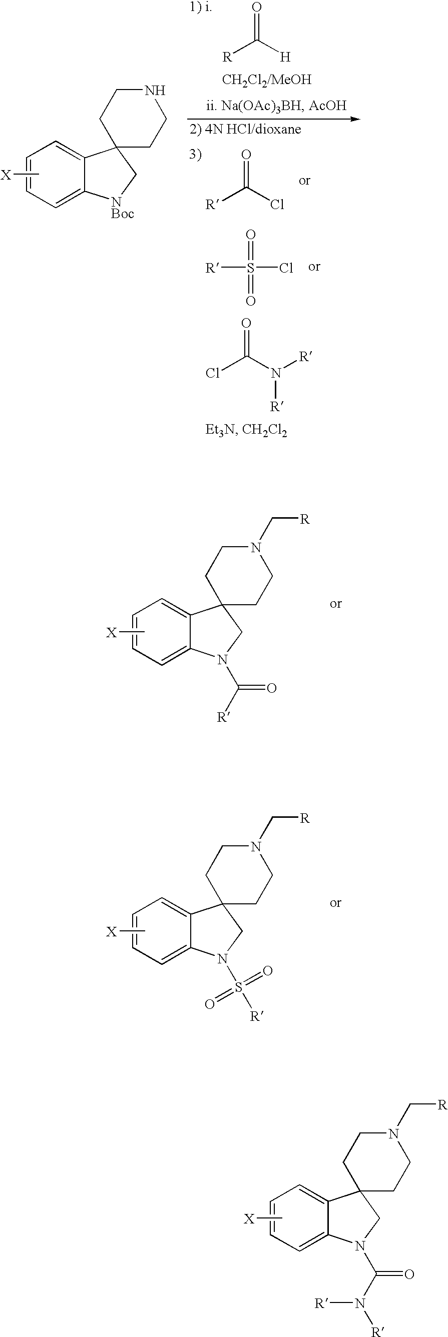

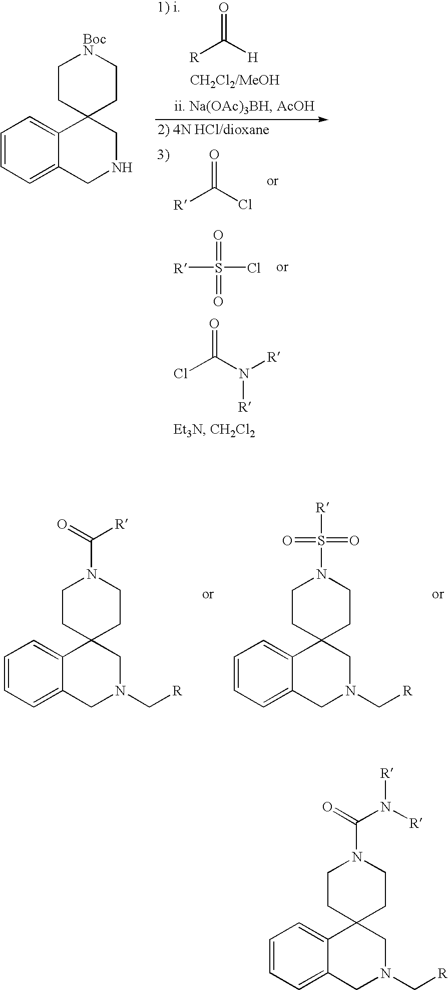

To a solution of Boc-spirocycle (0.86 mmol, 1.0 equiv.) in DCM/MeOH (4: 1, v/v, 3.5 mL) was added aldehyde (1.7 mmol, 2.0 equiv.) at room temperature . After stirring for 5 h, AcOH (2.58 mmol, 3.0 equiv.) and Na(OAc)₃BH (1.72 mmol, 2.0 equiv.) were added. Reactions were stirred for 20 h, diluted with CH₂Cl₂ (5 mL) and washed with NaOH (1M aq., 6 mL). The organics were dried over Na₂SO₄, filtered, and concentrated. The Boc-group was removed by stirring in 4N HCl/dioxane for 4 h at room temperature followed by removal of volatiles in vacuo.

The acylation/sulphonylation/carbamoylations were performed on split portions of the deprotected spirocycles as described herein. To the amine (˜0.11 mmol, 1.0 equiv.) in DCM (5 mL) containing Et3N (0.37 mmol) at room temperature was added acid/sulphonyl/carbamoyl chloride (0.22 mmol, 2.0 equiv.). After stirring for 48 h at ambient temperature the reactions were washed with NaHCO3 (sat. aq., 5 mL) and H2O (2×5 mL). The organics were dried over Na2SO4 and loaded on Silacycle® 12 mL-2 g Si-Tosic Acid SPE cartridges. MeOH (10 mL) was passed through the column to remove unbound impurities. The product was then eluted by passing a solution of 2N NH3 in MeOH (10 mL) through the column. The fractions were concentrated and, if necessary, purified by reverse-phase HPLC: Phenomenex® Luna C18 column (10 μ, 250×21.2 mm), 5% (v/v) CH3CN (containing 1% v/v TFA) in H2O (containing 1% v/v TFA) gradient to 95% H2O, 20 ml/min, λ=214 nm. Products were isolated as mono-TFA salts after lyophilization.

Certains compounds of the present invention have been characterized by high-performance liquid chromatography-mass spectrometry (HPLC/MS) analysis. Accordingly, the retention time (RT) and mass to charge ratio (m/z) by HPLC/MS is shown in TABLE 2.

TABLE 2

| | HPLC/MS | | |
|---|---|---|---|
| Cmpd No. | Method | RT (min) | m/z |
| 1 | A | 3.08 | 327.5 |
| 2 | A | 3.42 | 439.4 |
| 3 | A | 3.37 | 375.2 |
| 4 | A | 3.57 | 487.2 |
| 5 | A | 2.03 | 327.3 |
| 6 | A | 2.29 | 375.2 |
| 7 | A | 2.63 | 439.5 |
| 8 | A | 2.81 | 487.4 |
| 9 | C | 2.83 | 642.4 |
| 10 | C | 2.73 | 653.5 |
| 11 | A | 3.53 | 483.2 |
| 12 | A | 3.02 | 359.1 |
| 13 | A | 3.15 | 371.1 |
| 14 | A | 1.44 | 271.1 |
| 15 | B | 3.63 | 389.5 |
| 16 | A | 2.03 | 363.3 |
| 17 | A | 3.45 | 517.4 |
| 18 | A | 3.8 | 521.1 |
| 19 | B | 3.79 | 501.5 |
| 20 | A | 2.13 | 383.3 |
| 21 | B | 3.43 | 497.2 |
| 22 | B | 3.77 | 481.4 |
| 23 | B | 3.4 | 477.4 |
| 24 | B | 3.79 | 461.1 |
| 25 | A | 3.25 | 363.3 |
| 26 | B | 2.91 | 315.3 |
| 27 | B | 3.08 | 393.2 |
| 28 | B | 3.53 | 377.4 |
| 29 | A | 3.7 | 429.3 |
| 30 | C | 1.83 | 399.2 |
| 31 | C | 1.81 | 395.3 |
| 32 | C | 2.09 | 446.5 |
| 33 | B | 2.53 | 455.4 |
| 34 | B | 1.88 | 343.2 |
| 35 | C | 1.79 | 345.2 |
| 36 | C | 1.94 | 401.2 |
| 37 | C | 1.88 | 397.2 |
| 38 | C | 2.19 | 448.4 |
| 39 | C | 2.4 | 457.1 |
| 40 | C | 2.09 | 445.3 |
| 41 | C | 2.36 | 496.2 |
| 42 | C | 1.93 | 439.3 |
| 43 | C | 2.06 | 495.3 |
| 44 | C | 1.99 | 491.3 |
| 45 | C | 2.26 | 542.1 |
| 46 | C | 2.23 | 447.4 |
| 47 | C | 2.29 | 503.2 |
| 48 | C | 2.26 | 499.4 |
| 49 | C | 2.48 | 550.3 |
| 50 | C | 2.63 | 559.3 |
| 51 | C | 2.18 | 449.2 |
| 52 | C | 1.49 | 365.1 |
| 53 | A | 3.18 | 405.5 |
| 54 | C | 2.6 | 615.4 |
| 55 | C | 1.93 | 450.4 |
| 56 | C | 1.91 | 450.3 |
| 57 | C | 2.85 | 515.3 |
| 58 | C | 2.66 | 433.1 |
| 59 | C | 2.51 | 457.3 |
| 60 | C | 2.5 | 485.2 |
| 61 | C | 2.48 | 537.3 |
| 62 | C | 2.76 | 439.5 |
| 63 | C | 2.36 | 527.5 |
| 64 | C | 1.61 | 358.2 |
| 65 | C | 1.64 | 385.2 |
| 66 | C | 1.56 | 369.1 |
| 67 | C | 1.59 | 343.2 |
| 68 | C | 1.78 | 385.2 |
| 69 | C | 1.81 | 385.2 |
| 70 | C | 1.88 | 385.2 |
| 71 | C | 2.14 | 419.3 |
| 72 | C | 1.69 | 381.2 |
| 73 | C | 1.83 | 411.4 |
| 74 | C | 1.79 | 369.1 |
| 75 | C | 1.86 | 387.3 |
| 76 | C | 1.81 | 387.3 |
| 77 | C | 2.04 | 419.4 |
| 78 | C | 2.33 | 487.3 |
| 79 | C | 1.91 | 357.3 |
| 80 | C | 1.78 | 343.2 |
| 81 | C | 1.68 | 351.1 |
| 82 | C | 1.81 | 396.2 |
| 83 | C | 1.83 | 396.2 |
| 84 | C | 1.91 | 410.3 |
| 85 | C | 1.96 | 401.2 |
| 86 | C | 1.93 | 385.2 |
| 87 | C | 1.73 | 369.1 |
| 88 | C | 2.11 | 437.2 |
| 89 | C | 1.73 | 395.1 |
| 90 | C | 1.71 | 369.2 |
| 91 | C | 1.71 | 369.1 |
| 92 | C | 2.06 | 433.3 |
| 93 | C | 1.57 | 386.2 |
| 94 | C | 2.31 | 469.3 |
| 95 | C | 1.74 | 369.3 |
| 96 | C | 2.63 | 487.2 |
| 97 | C | 1.66 | 364.2 |
| 98 | C | 1.83 | 339.4 |
| 99 | C | 1.64 | 363.4 |
| 100 | C | 2.03 | 445.4 |
| 101 | C | 1.81 | 381.2 |
| 102 | C | 2.03 | 415.2 |
| 103 | C | 2.26 | 451.3 |
| 104 | C | 2.61 | 433.4 |
| 105 | C | 2.23 | 447.6 |
| 106 | C | 1.54 | 341.3 |
| 107 | C | 1.83 | 365.3 |
| 108 | C | 2.55 | 487.3 |
| 109 | C | 2.5 | 501.3 |
| 110 | C | 1.98 | 403.2 |
| 111 | C | 1.32 | 314.2 |
| 112 | C | 2.08 | 442.3 |
| 113 | C | 2.09 | 415.4 |
| 114 | C | 1.61 | 325.3 |
| 115 | C | 2.31 | 451.3 |
| 116 | C | 2.04 | 449.4 |
| 117 | C | 1.73 | 378.1 |
| 118 | C | 2.28 | 457.2 |
| 119 | C | 1.78 | 392.4 |
| 120 | C | 2.04 | 442.5 |
| 121 | C | 1.91 | 353.2 |
| 122 | C | 1.74 | 391.5 |
| 123 | C | 2.06 | 429.1 |
| 124 | C | 2.33 | 483.2 |
| 125 | C | 1.59 | 339.4 |
| 126 | C | 1.74 | 367.3 |
| 127 | C | 1.56 | 340.3 |
| 128 | C | 1.66 | 406.3 |
| 129 | C | 2.06 | 407.2 |
| 130 | C | 1.79 | 383.3 |
| 131 | C | 1.66 | 351.1 |
| 132 | C | 1.57 | 311.3 |
| 133 | C | 1.88 | 383.3 |
| 134 | C | 3.05 | 497.4 |
| 135 | C | 1.98 | 399.2 |
| 136 | C | 1.78 | 395.3 |
| 137 | C | 1.88 | 425.2 |
| 138 | C | 1.78 | 455.3 |
| 139 | C | 1.79 | 383.3 |
| 140 | C | 1.83 | 383.2 |
| 141 | C | 1.81 | 383.2 |
| 142 | C | 1.89 | 401.2 |
| 143 | C | 1.88 | 401.2 |
| 144 | C | 1.89 | 401.2 |
| 145 | C | 2.16 | 451.3 |
| 146 | C | 2.09 | 433.3 |
| 147 | C | 2.13 | 447.4 |
| 148 | C | 2.38 | 501.5 |
| 149 | C | 1.41 | 374.2 |
| 150 | C | 1.79 | 372.3 |
| 151 | C | 1.78 | 409.3 |
| 152 | C | 1.84 | 357.3 |
| 153 | C | 2.06 | 467.3 |
| 154 | C | 1.71 | 343.2 |
| 155 | C | 1.74 | 365.4 |
| 156 | C | 1.86 | 410.3 |
| 157 | C | 1.88 | 410.3 |
| 158 | C | 1.96 | 424.3 |
| 159 | C | 2.03 | 415.5 |
| 160 | C | 2.08 | 415.5 |
| 161 | C | 1.76 | 371.1 |
| 162 | C | 1.91 | 387.1 |
| 163 | C | 1.83 | 395.3 |
| 164 | C | 2.01 | 411.4 |
| 165 | C | 1.98 | 399.3 |
| 166 | C | 2.21 | 433.1 |
| 167 | C | 1.96 | 371.1 |
| 168 | C | 2.38 | 423.4 |
| 169 | C | 1.99 | 401.1 |
| 170 | C | 2.21 | 435.1 |
| 171 | C | 2.06 | 446.4 |
| 172 | C | 2.28 | 469.3 |
| 173 | C | 2.01 | 419.3 |
| 174 | C | 1.31 | 332.1 |
| 175 | C | 1.91 | 386.2 |
| 176 | C | 1.88 | 380.3 |
| 177 | C | 1.86 | 394.3 |
| 178 | C | 2.16 | 414.3 |
| 179 | C | 1.86 | 410.4 |
| 180 | C | 1.81 | 470.4 |
| 181 | C | 1.89 | 409.3 |
| 182 | C | 1.76 | 381.2 |
| 183 | C | 1.37 | 360.3 |
| 184 | C | 1.52 | 344 |
| 185 | C | 2.26 | 455.1 |
| 186 | C | 2.48 | 523.1 |
| 187 | C | 2.13 | 421.3 |
| 188 | C | 2.11 | 400.2 |
| 189 | C | 1.93 | 401.2 |
| 190 | C | 1.59 | 358.1 |
| 191 | C | 2.11 | 435.3 |
| 192 | C | 2.29 | 469.3 |
| 193 | C | 2.03 | 446.5 |
| 194 | C | 2.33 | 514.3 |
| 195 | C | 2.23 | 469.1 |
| 196 | C | 2.48 | 537.1 |
| 197 | C | 2.04 | 425.2 |
| 198 | C | 1.66 | 359.1 |
| 199 | C | 1.94 | 407.3 |
| 200 | C | 1.57 | 343.1 |
| 201 | C | 1.71 | 361.1 |
| 202 | C | 1.42 | 360.1 |
| 203 | C | 1.83 | 387.3 |
| 204 | A | 1.71 | 381.3 |
| 205 | A | 1.57 | 367.3 |
| 206 | C | 1.98 | 421.3 |
| 207 | C | 1.59 | 386.2 |
| 208 | C | 1.69 | 430.2 |
| 209 | C | 1.94 | 397.2 |
| 210 | C | 2.31 | 461.1 |
| 211 | C | 2.04 | 425.1 |
| 212 | C | 2.01 | 438.3 |
| 213 | C | 2.21 | 461 |
| 214 | C | 2.13 | 427.2 |
| 215 | C | 1.66 | 398.1 |
| 216 | C | 1.78 | 387.3 |
| 217 | C | 1.76 | 371.1 |
| 218 | C | 1.52 | 341.3 |
| 219 | C | 1.66 | 386.2 |
| 220 | C | 1.52 | 341.2 |
| 221 | C | 1.73 | 371.1 |
| 222 | A | 1.88 | 445.4 |
| 223 | C | 2.04 | 437.0 |
| 224 | A | 1.57 | 367.3 |
| 225 | C | 1.79 | 391.2 |
| 226 | C | 2.03 | 391.2 |
| 111 | C | 1.84 | 420.3 |
| 228 | C | 1.98 | 420.5 |
| 229 | C | 1.91 | 405.2 |
| 230 | C | 1.27 | 384.2 |
| 231 | C | 1.56 | 386.2 |
| 232 | C | 1.88 | 424.3 |
| 233 | C | 1.02 | 341.2 |
| 234 | B | 1.71 | 386.3 |
| 235 | C | 1.81 | 385.2 |
| 236 | C | 2.46 | 421.3 |
| 237 | A | 3.53 | 447.5 |
| 238 | A | 2.09 | 341.4 |
| 239 | A | 2.68 | 453.4 |
| 240 | A | 2.33 | 389.4 |
| 241 | A | 2.85 | 501.6 |
| 242 | A | 1.89 | 341.3 |
| 243 | A | 2.16 | 389.3 |
| 244 | A | 2.55 | 453.4 |
| 245 | A | 2.7 | 501.6 |
| 246 | C | 1.74 | 341.4 |
| 247 | C | 2.18 | 443.3 |
| 248 | C | 2.11 | 403.4 |
| 249 | C | 2.35 | 453.4 |
| 250 | A | 2.36 | 413.3 |
| 251 | A | 2.45 | 491.2 |
| 252 | A | 2.68 | 475.5 |
| 253 | A | 3.43 | 477.4 |
| 254 | A | 3.42 | 497.6 |
| 255 | A | 2.7 | 511.3 |
| 256 | A | 2.18 | 377 |
| 257 | B | 1.91 | 329.2 |
| 258 | B | 2.13 | 407.1 |
| 259 | B | 2.36 | 391.3 |
| 260 | B | 2.7 | 481.2 |
| 261 | A | 2.01 | 357.2 |
| 262 | B | 2.56 | 433.4 |
| 263 | A | 2.24 | 397.3 |
| 264 | C | 1.52 | 339.4 |
| 265 | C | 1.69 | 395.3 |
| 266 | B | 2.21 | 442.5 |
| 267 | B | 2.35 | 451.2 |
| 268 | C | 1.89 | 389.4 |
| 269 | C | 2.29 | 492.4 |
| 270 | C | 1.64 | 391.3 |
| 271 | C | 2.03 | 445.4 |
| 272 | C | 1.98 | 441.2 |
| 273 | C | 1.17 | 307.4 |
| 274 | B | 3.12 | 672.2 |
| 275 | B | 2.98 | 638.3 |
| 276 | B | 3.03 | 674.5 |
| 277 | B | 2.91 | 640.5 |
| 278 | B | 2.96 | 638.3 |
| 279 | B | 2.85 | 604.5 |
| 280 | C | 3.65 | 455.1 |
| 281 | C | 3.45 | 455.2 |
| 282 | C | 3.25 | 455.2 |
| 283 | C | 3.72 | 489.2 |
| 284 | C | 2.36 | 435.3 |
| 285 | C | 3.33 | 439.2 |
| 286 | C | 2.95 | 440.3 |
| 287 | C | 3.55 | 489.3 |
| 288 | B | 2.28 | 397.1 |
| 289 | B | 2.23 | 397.2 |
| 290 | B | 2.5 | 431.1 |
| 291 | C | 2.06 | 419.5 |
| 292 | B | 2.16 | 385.1 |
| 293 | C | 1.88 | 387.1 |
| 294 | B | 2.13 | 385.3 |
| 295 | C | 2.28 | 471.3 |
| 296 | C | 1.91 | 413.3 |
| 297 | C | 1.91 | 413.2 |
| 298 | B | 2.24 | 397.3 |
| 299 | C | 2.01 | 397.2 |
| 300 | C | 2.01 | 397.1 |
| 301 | C | 1.93 | 397.3 |
| 302 | C | 2.09 | 411.3 |
| 303 | B | 2.46 | 411.1 |
| 304 | B | 1.16 | 321.0 |
| 305 | A | 1.42 | 447.4 |
| 306 | B | 2.38 | 431.2 |
| 307 | B | 2.51 | 483.3 |
| 308 | B | 2.41 | 461.2 |
| 309 | C | 2.26 | 435.1 |
| 310 | B | 2.46 | 467.3 |
| 311 | B | 2.08 | 446.9 |
| 312 | C | 2.09 | 447.3 |
| 313 | B | 2.43 | 431.2 |
| 314 | B | 2.36 | 461.2 |
| 315 | B | 2.4 | 431.1 |
| 316 | C | 2.55 | 431.1 |
| 317 | B | 2.18 | 433.3 |
| 318 | B | 2.18 | 433.1 |
| 319 | B | 2.41 | 481.2 |
| 320 | C | 2.56 | 481.3 |
| 321 | B | 2.58 | 481.1 |
| 322 | B | 2.16 | 449.1 |
| 323 | C | 2.56 | 511.3 |
| 324 | B | 2.5 | 511.2 |
| 325 | B | 2.19 | 449.2 |
| 326 | B | 2.19 | 449.1 |
| 327 | B | 2.33 | 431.2 |
| 328 | C | 2.7 | 515.3 |
| 329 | B | 2.43 | 550.2 |
| 330 | B | 2.43 | 550.2 |
| 331 | C | 2.46 | 564.1 |
| 332 | B | 2.53 | 506.1 |
| 333 | B | 2.4 | 465.5 |
| 334 | C | 2.45 | 465.3 |
| 335 | B | 2.29 | 451.1 |
| 336 | B | 2.51 | 505.1 |
| 337 | B | 2.55 | 505.1 |
| 338 | C | 2.76 | 523.2 |
| 339 | B | 2.66 | 521.4 |
| 340 | C | 2.76 | 521.3 |
| 341 | B | 2.63 | 521.3 |
| 342 | C | 2.98 | 509.2 |
| 343 | B | 2.83 | 509.5 |
| 344 | C | 2.68 | 487.2 |
| 345 | C | 2.7 | 487.2 |
| 346 | C | 2.29 | 429.1 |
| 347 | B | 2.56 | 515.3 |
| 348 | C | 2.48 | 451.2 |
| 349 | B | 2.29 | 442.2 |
| 350 | B | 2.31 | 459.2 |
| 351 | B | 2.38 | 459.3 |
| 352 | C | 2.43 | 443.2 |
| 353 | C | 2.14 | 420.4 |
| 354 | C | 1.89 | 434.1 |
| 355 | C | 2.4 | 417.2 |
| 356 | B | 2.43 | 445.2 |
| 357 | B | 2.55 | 459.3 |
| 358 | B | 2.46 | 457.3 |
| 359 | B | 2.24 | 417.3 |
| 360 | B | 2.36 | 471.2 |
| 361 | B | 2.48 | 445.3 |
| 362 | B | 2.48 | 439.4 |
| 363 | B | 2.37 | 425.4 |
| 364 | B | 2.26 | 411.2 |
| 365 | B | 2.28 | 411.2 |
| 366 | B | 2.6 | 453.4 |
| 367 | B | 1.85 | 353.3 |
| 367 | B | 2.09 | 387.1 |
| 369 | B | 2.3 | 433.3 |
| 370 | B | 2.16 | 420.2 |
| 371 | C | 2.38 | 411.5 |
| 372 | C | 2.19 | 397.3 |
| 373 | C | 2.24 | 397.1 |
| 374 | C | 2.56 | 439.5 |
| 375 | C | 2.5 | 425.2 |
| 376 | B | 2.01 | 383.2 |
| 377 | B | 1.98 | 365.3 |
| 378 | B | 2.11 | 383.4 |
| 379 | B | 2.01 | 383.2 |
| 380 | B | 2.6 | 483.3 |
| 381 | B | 1.98 | 353.3 |
| 382 | B | 2.16 | 406.4 |
| 383 | B | 2.09 | 381 |
| 384 | B | 2.14 | 381.4 |
| 385 | B | 2.18 | 419.6 |
| 386 | B | 2.23 | 417.3 |
| 387 | B | 1.91 | 394.5 |
| 388 | B | 2.03 | 351.4 |
| 389 | B | 2.08 | 367.4 |
| 390 | B | 2.14 | 369.4 |
| 391 | B | 2.08 | 369.4 |

The HPLC/MS data for the compounds of TABLE 2 were obtained as follows:

Method A:

HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, ESI⁺.

Method B:

HPLC/MS: Alltech® Prevail C18 column (5μ, 50×4.6 mm), 5% v/v CH₃CN (containing 1% v/v TFA) in H₂O (containing 1% v/v TFA) gradient to 99% v/v CH₃CN in H₂O, 3.5 mL/min, ESI⁺.

Method C:

HPLC/MS: Waters® YMC™ ODS-A C18 column (5 μ, 50×4.6 mm), 5% v/v CH₃CN (containing 1% v/v TFA) in H₂O (containing 1% v/v TFA) gradient to 99% v/v CH₃CN in H₂O, 3.5 mL/min, ESI⁺.

### Example 6

### General Method—Preparation of Amides

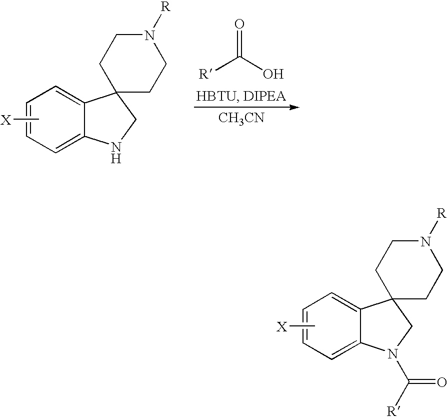

To a solution of a spiroindoline (0.124 mmol, 1.0 equiv.) in CH₃CN (1.5 mL) at room temperature was added sequentially DIPEA (0.248 mmol, 2.0 equiv.), carboxylic acid (0.173 mmol, 1.4 equiv.), and HBTU (0.173 mmol, 1.4 equiv.). Reactions were stirred for 48 h at room temperature and diluted with CH₂Cl₂ (5 mL) and washed sequentially with NaHCO₃ (sat. aq., 5 mL), HCl (1M aq., 5 mL), and water (5 mL). Organics were dried over Na₂SO₄, filtered, and concentrated. Products were purified by ‘trap and release’ on Silacycle® 12 mL-2 g Si-Tosic Acid SPE cartridges as described previously (see: parallel synthesis of spiroindole/spiropiperidines). If necessary, samples were further purified by reverse-phase HPLC: Phenomenex® Luna C18 column (10 μ, 250×21.2 mm), 5% (v/v) CH₃CN (containing 1% v/v TFA) in H₂O (containing 1% v/v TFA) gradient to 95% H₂O, 20 ml/min, λ=214 nm.

### Example 7

### General Method—Preparation of Ureas

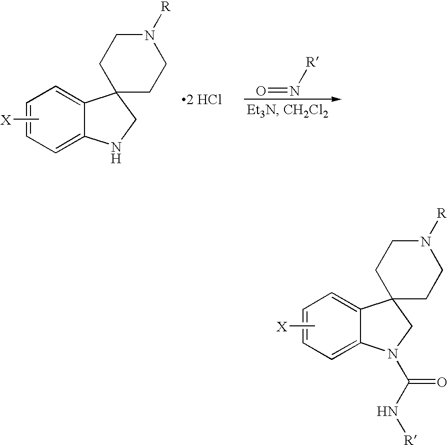

To a stirring solution of a spirocycle (0.11 mmol, 1.0 equiv.) in CH₂Cl₂ (4 mL) containing Et₃N (0.37 mmol, 3.4 equiv.) at room temperature was added isocyanate (0.22 mmol, 2.0 equiv.). After stirring for 48 h the reactions were washed with NaHCO₃ (sat. aq., 4 mL) and H₂O (2×4 mL). The organics were dried over Na₂SO₄ and concentrated. Products were purified by ‘trap and release’ on Silacycle® 12 mL-2 g Si-Tosic Acid SPE cartridges as described previously (see: parallel synthesis of spiroindole/spiropiperidines). If necessary, samples were further purified by reverse-phase HPLC: Phenomenex® Luna C18 column (10μ, 250×21.2 mm), 5% (v/v) CH₃CN (containing 1% v/v TFA) in H₂O (containing 1% v/v TFA) gradient to 95% H₂O, 20 ml/min, λ=214 nm.

### Example 8

### General Method—Alkylation using Epoxides

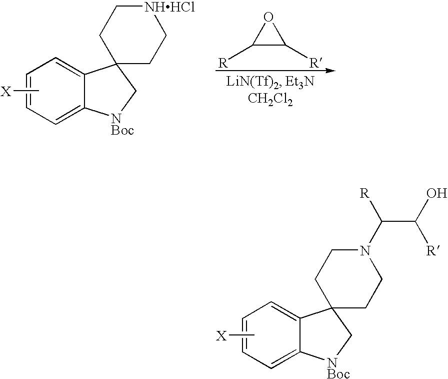

A solution of the above amine (0.46 mmol, 1.0 equiv.) in CH₂Cl₂ (2 mL) at room temperature was treated sequentially with Et₃N (0.69 mmol, 1.5 equiv.), LiN(Tf)₂ (0.92 mmol, 2.0 equiv.), and epoxide (0.92 mmol, 2.0 equiv.). After stirring for 20 h the reactions were diluted with CH₂Cl₂ (5 mL), washed with NaHCO₃ (sat. aq., 2×5 mL), dried over Na₂SO₄, filtered, and concentrated. Material obtained was deprotected (as described previously) and reacted with various electrophiles (as described previously).

### Example 9

### General Method—Nitrogen Arylation

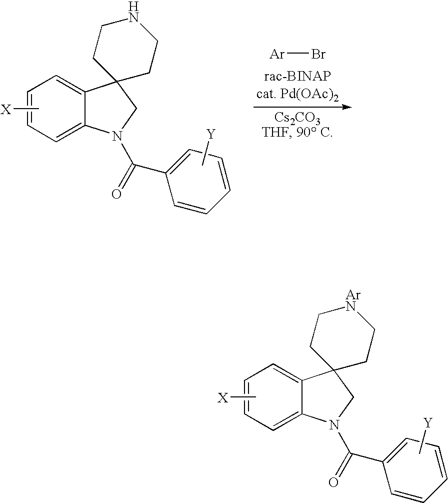

To a 4 mL vial containing Cs₂CO₃ (0.17 mmol, 1.9 equiv.) was added a solution of Pd(OAc)₂ (4.5 μmol, 0.05 equiv.) and rac-BINAP (7.2 μmol, 0.08 equiv.) in anhydrous THF (1.0 mL). The aryl bromide (0.126 mmol, 1.40 equiv.) was added followed by the addition of piperidene/spiroindoline (0.09 mmol, 1.0 equiv.) as a solution anhydrous THF (2.0 mL). The vial was capped and heated with stirring to 90° C. for 4 to 8 hours (as monitored by HPLC/MS). The reaction mixture was transferred to a 40 mL vial and diluted with MTBE (8 mL). The organic layer was washed with HCl (1M aq., 2×3 mL) water (3 mL). The organic layer was concentrated and the residue was diluted with CH₂Cl₂ (8 mL) and dried over Na₂SO₄. Products were purified by ‘trap and release’ on Silacycle® 12 mL-2 g Si-Tosic Acid SPE cartridges as described previously (see: parallel synthesis of spiroindole/spiropiperidines). If necessary, sample was further purified by reverse-phase HPLC: Phenomenex® Luna C18 Column (10 μL, 250×21.2 mm), 5% (v/v) CH₃CN (containing 1% v/v TFA) in H₂O (containing 1% v/v TFA) gradient to 95% CH₃CN, 20 mL/min, λ=214 nm.

### Example 10

### 1′-(Allyl)-1,2-dihydro-5-fluoro-1-(tert-Butoxycarbonyl)-spiro[3H-indole-3,4′-piperidine]

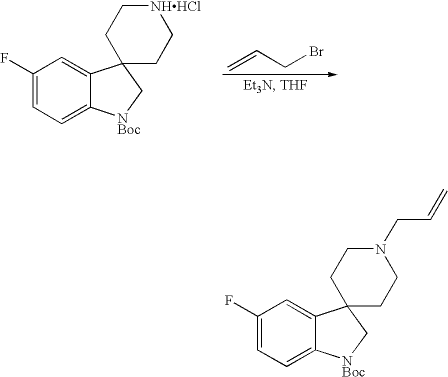

To a stirring solution of the hydrochloride salt of the above spirocyle (1.50 g, 4.37 mmol) in THF (85 mL) at 0° C. was added Et₃N (1.52 mL, 10.9 mmol) and allyl bromide (0.69 g, 5.70 mmol). The reaction was slowly warmed to room temperature and stirred for 72 h. The mixture was filtered and concentrated. The concentrate was dissolved in EtOAc (50 mL), washed with H₂O (2×50 mL), dried over MgSO₄, filtered, and concentrated to give the resulting compound (1.48 g, 4.32 mmol, 99% yield) as a white solid.

¹H NMR (CDCl₃, 400 MHz): δ 6.81 (3H, m), 5.88 (1H, ddt. J=17.6, 9.6, 6.4 Hz), 5.20 (1H, d, J=17.6 Hz), 5.17 (1H, d, J=9.6 Hz), 3.75 (2H, m), 3.03 (2H, d, J=6.4 Hz), 2.93 (2H, d, J=11.6 Hz), 2.05 (2H, m), 1.90 (2H, td, J=13.2, 3.6 Hz), 1.66 (2H, dd, J=12.8, 1.6 Hz), 1.56 (9H, s). HPLC/MS: Waters® YMC™ ODS-A C18 column (5 μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=1.93 min, ESI⁺=347.3 (M+H).

### Example 11

### 1′-(Allyl)-1,2-dihydro-5-fluoro-spiro[3H-indole-3,4′-piperidine]

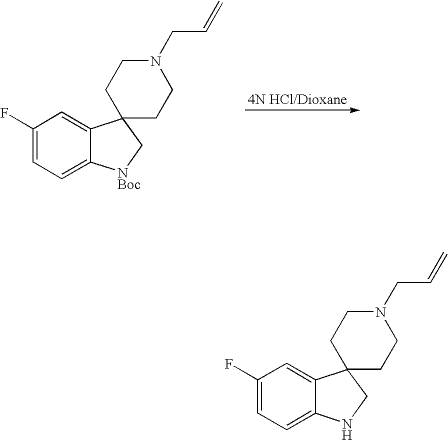

The above spiroindoline (797 mg, 2.33 mmol) was treated with 4N HCl in dioxane (5 mL) for 3 h at room temperature. The volatiles were removed in vacuo and the crude residue was washed with hexanes (2×10 mL) to give the bis-HCl salt of the resulting compound as a white solid. In order to prepare the free base of the resulting compound, the white solid was dissolved in CH₂Cl₂, washed with NaOH (1N aq.), dried over Na₂SO₄, filtered, and concentrated to give the resulting compound as a white solid.

¹H NMR (CDCl₃, 400 MHz): δ 6.78 (1H, dd, J=8.4, 2.4Hz), 6.72 (1H, td, J=8.8, 2.8 Hz), 6.53 (1H, dd, J=8.4, 4.4Hz), 5.92 (1H, ddt, J=18.0, 10.0, 6.4 Hz),5.20 (1H, dd, J=18.0, 1.6 Hz), 5.17 (1H, dd, J=10.0, 0.8 Hz), 3.44 (2H, s), 3.03 (2H, d, J=6.4 Hz), 2.90 (2H, dd, J=9.2, 2.8 Hz), 2.06 (2H, td, J=12.4, 2.4 Hz), 1.90 (2H, td, J=13.2, 4.0 Hz), 1.75 (2H, dd, J=13.2, 2.0 Hz), 1.73 (1H, bs). HPLC/MS: Waters® YMC™ ODS-A C18 column (5 μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=0.67 min. ESI⁺=247.2 (M+H).

### Example 12

### 1′-(Cyclopropylmethyl)-1,2-dihydro-5-fluoro-spiro[3H-indole-3,4′-piperidine]

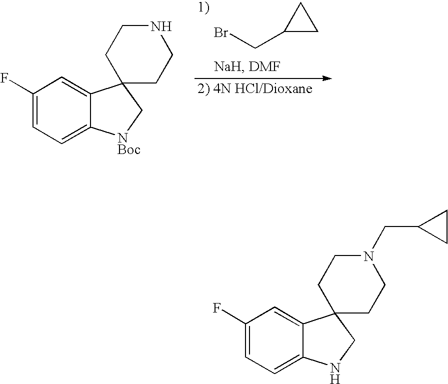

To a flask containing NaH (30.0 mg, 1.25 mmol) in DMF (10 mL) under N₂ at room temperature was added compound the above spiroindoline compound (256 mg, 0.84 mmol) as a solution in DMF (3 mL). The flask was brought to 0° C. and (bromomethyl)-cyclopropane (121 μL, 1.25 mmol) was added via syringe. The reaction was slowly warmed to room temperature and stirred for 96 h under N₂. The reaction was quenched with NH₄Cl (sat. aq., 1 mL) and the mixture was diluted with EtOAc/hexanes (1:1, v/v, 25 mL) and washed with H₂O (2×25 mL). The organics were dried over MgSO4, filtered, and concentrated. The product was treated with 4N HCl/Dioxane (5 mL) and stirred for 4 h at room temperature followed by removal of the volatiles in vacuo to give the resulting compound as the bis-HCl salt. In order to prepare the resulting compound as the free base, the white solid was dissolved in CH₂Cl₂, washed with NaOH (1N aq.), dried over Na₂SO₄, filtered, and concentrated.

¹H NMR (CDCl₃, 400 MHz): δ 6.78 (1H, m), 6.71 (1H, m), 6.53 (1H, m), 3.40 (2H, s), 3.02 (2H, m), 3.36 (2H, d, J=9.6 Hz), 2.08 (2H, td, J=12.0, 2.0 Hz), 1.93 (2H, td, J=13.6, 4.0 Hz), 1.74 (2H, m), 0.89 (1H, m), 0.53 (2H, m), 0.11 (2H, m). HPLC/MS: Waters® YMC™ ODS-A C18 column (5 μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=0.74 min, ESI⁺=261.1 (M+H).

### Example 13

### 1′-(Methyl)-1,2-dihydro-5-fluoro-spiro[3H-indole-3,4′-piperidine]

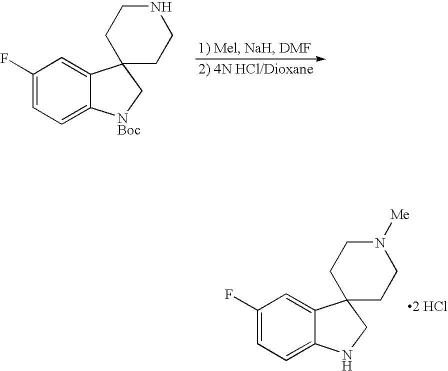

To a flask containing NaH (19.0 mg, 0.47 mmol) in DMF (2.5 mL) under N₂ at room temperature was added the above spiroindoline compound (96 mg, 0.31 mmol) as a solution in DMF (2.5 mL). The flask was brought to 0° C. and methyl iodide (29 μL, 0.47 mmol) was added via syringe. The reaction was stirred at 0° C. for 30 min at which time NH₄Cl (sat. aq., 1 mL) was added to quench remaining hydride. The mixture was diluted with EtOAc/hexanes (1:1, v/v, 15 mL) and washed with H₂O (4×10 mL). The product was treated with 4N HCl/dioxane (5 mL) for 5 h and concentrated in vacuo to give the bis-HCl salt of the resulting compound.

¹H NMR (DMSO-d_{6,} 400 MHz): δ 10.40 (1H, bs), 7.12 (2H, m), 7.02 (1H, d, J=8.0 Hz), 4.05-3.60 (2H, bs), 3.67 (2H, s), 3.43 (2H, d, J=12.0 Hz), 3.10 (2H, q, J=10.0 Hz), 2.77 (3H, d, J=4.8 Hz), 2.17 (2H, td, J=13.6, 3.6 Hz), 1.94 (2H, d, J=14.0 Hz). HPLC/MS: Alltech® Prevail C18 column (5μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=0.70 min, ESI⁺=221.0 (M+H).

Examples 14-34, below, were made using the methodology set forth herein.

### Example 14

### 1′-(3,4-Dichlorobenzyl)-2,3-dihydro-2-(cyclobutylmethyl)-spiro[isoquinoline-4(1H),4′-piperidine]

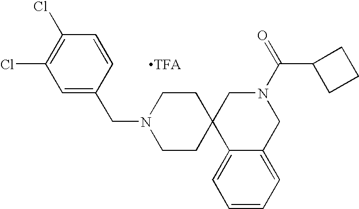

¹H NMR (CDCl₃, 400 MHz): δ 7.60 (1H, d, J=2.0 Hz), 7.52 (1H, d, J=8.4 Hz), 7.54 (2H, m), 7.30 (1H, t, J=7.6 Hz), 7.22 (1H, td, J=7.6, 1.2 Hz), 7.05 (1H, d, J=7.2 Hz), 4.56 (2H, s), 4.16 (2H, s), 3.87 (2H, s), 3.48 (2H, d, J=11.6 Hz), 3,37 (1H, q, J=8.4 Hz), 3.11 (2H, t, J=12.8 Hz), 2.57 (2H, td, J=14.4, 2.0 Hz), 2.33-2.18 (4H, m), 2.03 (1H, m), 1.90 (1H, m) 1.80-1.60 (2H, m). HPLC/MS: Waters® YMC™ ODS-A C18 column (5 μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=2.18 min, ESI⁺=443.3 (M+H).

### Example 15

### 1′-(Butyl)-1,2-dihydro-1-(diphenylacetyl)-spiro[3H-indole-3,4′-piperidine]

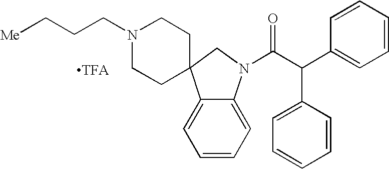

¹H NMR (CDCl₃, 400 MHz), A mixture of conformational isomers was evident: δ 8.30 (0.1H, d, J=8.4 Hz), 8.18 (0.9H, d, J=8.0 Hz), 7.33-7.16 (11H, m), 7.11-7.01 (2H, m), 5.14 (0.1H, s), 5.10 (0.9 H, s), 4.07 (0.2H, s), 3.84 (1.8H, s), 3.55 (0.2H, d, J=8.0 Hz), 3.42 (1.8H, d, J=12.0 Hz), 3.03-2.75 (2H, m), 2.42 (0.2H, m), 2.29 (1.8H, t, J=13.6 Hz), 2.19 (2H, m), 1.90-1.56 (4H, m), 1.33 (2H, m), 0.91 (3H, t, J=7.2 Hz). HPLC/MS: Discovery® C18 column (5μ, 50×2.1 mm), 5% v/v CH₃CN (containing 1% v/v TFA) in H₂O (containing 1% v/v TFA) gradient to 99% V/V CH₃CN in H₂O, 0.75 mL/min, tᵣ=2.63 min, ESI⁺=439.5 (M+H).

### Example 16

### 1′-(Isobutyryl)-1,2-dihydro-1-(phenethyl)-spiro[3H-indole-3,4′-piperidine]

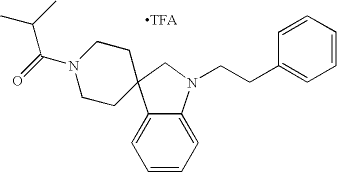

¹H NMR (CDCl₃, 400 MHz): δ 7.32 (2H, m), 7.23 (3H, m), 7.13 (1H, td, J=7.6, 1.2 Hz), 7.01 (1H, d, J=6.8 Hz), 6.74 (1H, t, J=7.2 Hz), 6.56 (1H, d, J=7.6 Hz), 4.57 (1H, d, J=13.2 Hz), 3.90 (1H, d, J=12.0 Hz), 3.40 (3H, m), 3.29 (1H, m), 3.19 1H, m), 2.92 (2H, t, J=7.6 Hz), 2.84 (1H, sept, J=6.4 Hz), 2.7 (1H, m), 1.76 (4H, m), 1.16 (6H, m). HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tᵣ=3.25 min, ESI⁺=363.3 (M+H).

### Example 17

### 1′-(Cyclobutylcarbonyl)-1,2-dihydro-1-(2,4-dimethylbenzyl)-spiro[3H-indole-3,4′-piperidine]

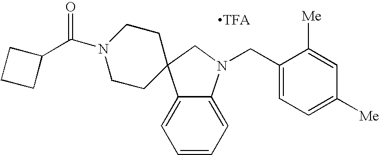

¹H NMR (CDCl₃, 400 MHz): δ 7.15 (1H, d, J=8.0 Hz), 7.10 (1H, td, J=7.6, 1.2 Hz), 7.00 (3H, m), 6.70 (1H, td, J=7.6, 1.0 Hz), 4.48 (1H, d, J=13.6 Hz), 3.63 (1H, d, J=13.2 Hz), 3.25 (1H, m), 3.19 (2H, m), 3.02 (1H, m), 2.72 (1H, m), 2.40 (2H, m), 2.33 (3H, s), 2.30 (3H, s), 2.13 (2H, m), 2.10-1.65 (8H, m). 13C NMR (CDCl₃, 100 MHz): 173.1, 151.4, 136.9, 136.5, 136.4, 132.7, 131.3, 128.4, 128.3, 126.5, 122.3, 117.8, 107.1, 62.4, 51.1, 43.2, 39.1, 37.4, 25.2, 25.1, 21.0, 18.9, 17.9. HPLC/MS: Alltech® Prevail C18 column (5μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=3.63 min, ESI⁺=389.5 (M+H).

### Example 18

### 1′-(Cyclopropylmethyl)-1,2-dihydro-5-fluoro-1-(2,4,6-trichlorobenzoyl)-spiro[3H-indole-3,4′-piperidine]

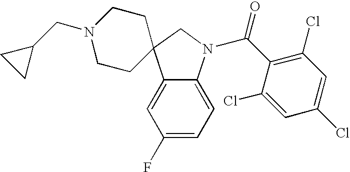

¹H NMR (CDCl₃, 400 MHz): δ 8.29 (1H, dd, J=8.8,4.8 Hz), 7.44 (2H, s), 6.99 (1H, td, J=8.8, 2.6 Hz), 6.94 (1H, dd, J=8.2, 2.6 Hz), 3.60 (2H, s), 3.09 (2H, d, J=11.8 Hz), 2.26 (2H, d, J=6.5 Hz), 2.02 (2H, dt, J=13.1, 3.3 Hz), 1.90 (2H, t, J=12.1 Hz), 1.73 (2H, d, J=12.0 Hz), 0.87 (1H, m), 0.54 (2H, m), 0.10 (2H, m). HPLC/MS: Waters® YMC™ ODS-A C18 column (5 μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=2.21 min, ESI⁺=469.3 (M+H).

### Example 19

### 1′-(Allyl)-1,2-dihydro-5-fluoro-1-(thiophene-2-carbonyl)-spiro[3H-indole-3,4′-piperidine]

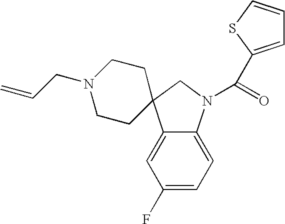

¹H NMR (CDCl₃, 400 MHz): δ 8.04 (1H, m), 7.61 (1H, d, J=3.4 Hz), 7.58 (1H, d, J=5.0 Hz), 7.16=5 (1H, dd, J=4.8, 3.9 Hz), 6.92 (2H, m), 5.90 (1H, ddt, J=16.9, 13.2, 6.6 Hz), 5.22-5.16 (2H, m), 4.21 (2H, s), 3.04 (2H, d, J=6.6 Hz), 2.97 (2H, m), 2.05-1.94 (4H, m), 1.73 (2H, m).

HPLC/MS: Waters® YMC™ ODS-A C18 column (5 μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=1.66 min, ESI⁺=357.2 (M+H).

### Example 20

### 1′-(Allyl)-1,2-dihydro-5-fluoro-1-(5-nitro-furan-2-carbonyl)-spiro[3H-indole-3,4′-piperidine]

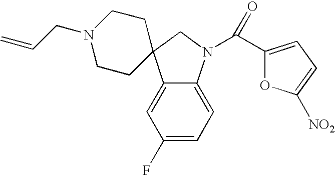

¹H NMR (CDCl₃, 400 MHz): δ 8.25 (1H, m), 7.42 (2H, s), 6.97 (2H, m), 5.92 (1H, ddt, J=16.7, 13.1, 6.6 Hz), 5.25 (2H, m), 4.44 (2H, s), 3.10-3.04 (4H, m), 2.06 (4H, m), 1.76 (2H, d, J=12.7 Hz). HPLC/MS: Waters® YMC™ ODS-A C18 column (5 μ, 50×4.6 mm), 5% v/v CH3CN (containing 1% v/v TFA) in H₂O (containing 1% v/v TFA) gradient to 99% v/v CH₃CN in H₂O, 3.5 mL/min, tᵣ=1.74 min, ESI⁺=386.1 (M+H).

### Example 21

### 1′-(Allyl)-1,2-dihydro-5-fluoro-1-(5-bromo-thiophene-2-carbonyl)-spiro[3H-indole-3,4′-piperidine]

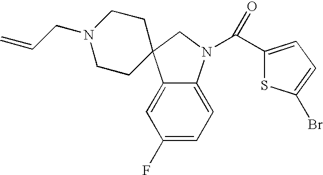

¹H NMR (CDCl₃, 400 MHz): δ 8.03 (1H, m), 7.37 (1H, d, J=4.0 Hz), 7.12 (1H, d, J=4.0 Hz), 6.93 (2H, m), 5.91 (1H, ddt, J=16.9, 13.3, 6.6 Hz), 5.25 (2H, m), 4.17 (2H, s), 3.09-3.02 (4H, m), 2.03 (4H, m), 1.74 (2H, d, J=10.8 Hz). HPLC/MS: Waters® YMC™ ODS-A C18 column (5 μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=2.04 min, ESI⁺=437.0 (M+H).

### Example 22

### 1′-(Allyl)-1,2-dihydro-5-fluoro-1-(2,3-diflurobenzoyl)-spiro[3H-indole-3,4′-piperidine]

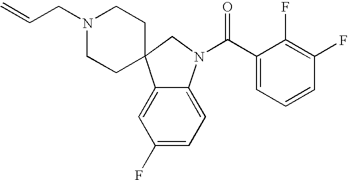

¹H NMR (CDCl₃, 400 MHz), A mixture of conformational isomers was evident: 8 8.25 (0.75H, dd, J=8.8, 4.8 Hz), 7.35-7.21 (3H, m), 6.98 (0.75H, td, J=8.8, 2.3 Hz), 6.91 (1H, dd, J=8.2, 2.6 Hz), 6.59 (0.25H, m), 5.92-5.80 (1.25 H, m), 5.30-5.14 (2H, m), 4.38 (0.25H, d, J=10.7 Hz), 3.95 (0.25H, m), 3.75 (1.5H, s), 3.08 (0.5H, d, J=6.3Hz), 2.98 (2H, d, J=6.5 Hz), 2.92 (1.5H, d, J=11.8 Hz), 2.20-1.80 (4H, m), 1.69 (2H, d, J=12.5 Hz). HPLC/MS: Waters® YMC™ ODS-A C18 column (5 μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=1.79 min, ESI⁺=387.3 (M+H).

### Example 23

### 1′-(Allyl)-1,2-dihydro-5-fluoro-1-(2-fluorobenzenesulfonyl)-spiro[3H-indole-3,4′-piperidine]

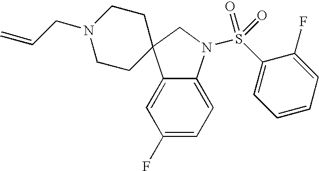

¹H NMR (CDCl₃, 400 MHz): δ 7.97 (1H, m), 7.54 (1H, m), 7.34 (1H, dd, J=8.8, 4.4 Hz), 7.28 (1H, td, J=7.8, 1.0 Hz), 7.15 (1H, m), 6.81 (2H, m), 5.87 (1H, ddt, J=16.6, 10.2, 6.6 Hz), 5.19 (2H, m), 4.0 (2H, s), 3.03 (2H, d, J=6.6 Hz), 2.89 (2H, d, J=12.0 Hz), 2.0 (2H, td, J=12.5, 2.0 Hz), 1.85 (2H, td, J=13.2, 3.8 Hz), 1.48 (2H, dd, J=13.3, 2.0 Hz).

### Example 24

### 1′-(Allyl)-1,2-dihydro-5-chloro-1-(2-fluorobenzoyl)-spiro[3H-indole-3,4′-piperidine]

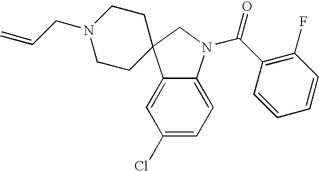

¹H NMR (CDCl₃, 400 MHz), A mixture of confirmational isomers was evident: a 8.24 (0.7H, d, J=8.6 Hz), 7.48 (2H, m), 7.30-7.24 (2.OH, m), 7.21-7.16 (1.7H, m), 6.81 (0.3H, d, J=5.9 Hz), 5.84 (1.3H, m), 5.25-5.13 (2H, m), 4.34 (0.3H, m), 3.93 (0.3H, m), 3.74 (1.4H, s), 3.07-2.84 (4H, m), 2.2-1.81 (4H, m), 1.68 (2H, d, J=12.7 Hz).

### Example 25

### 1′-(Cyclopropylmethyl)-1,2-dihydro-5-fluoro-1-(2-chlorobenzenesulfonyl)-spiro[3H-indole-3,4′-piperidine]

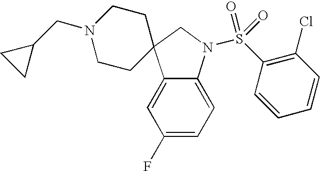

¹H NMR (CDCl₃, 400 MHz): δ 8.19 (1H, m), 7.51 (2H, m), 7.43 (1H, m), 7.19 (1H, dd, J=8.6, 4.4 Hz), 6.84-6.78 (2H, m), 4.05 (2H, s), 3.04 (2H, d, J=11.8 Hz), 2.28 (2H, d, J=6.6 Hz), 2.0 (2H, m), 1.9 (2H, td, J=13.0, 3.3 Hz), 0.87 (1H, m), 0.54 (2H, m), 0.12 (2H, m).

### Example 26

### 1′-(Allyl)-1,2-dihydro-5-fluoro-1-(2-chlorobenzoyl)-spiro[3H-indole-3,4′-piperidine]

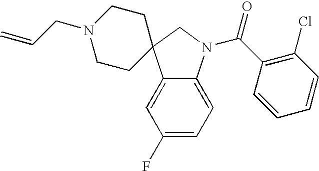

¹H NMR (CDCl₃, 400 MHz), A mixture of conformational isomers was evident: 6 8.30 (0.6H, dd, J=8.8, 4.8Hz), 7.49 (0.6H, m), 7.43 (2H, m), 7.39 (1.4H, m), 6.98 (0.6H, dt, J=8.9, 2.7 Hz), 6.90 (1H, m), 6.53 (0.4H, dt, J=8.8, 2.7 Hz), 5.95-5.80 (1H, m), 5.62 (0.4H, dd, J=8.9, 4.3 Hz), 5.26-5.14 (2H, m), 4.36 (0.4H, d, J=12.2 Hz), 4.02 (0.6 H, d, J=11.9 Hz), 3.69 (0.4H, d, J=14.9 Hz), 3.56 (0.6H, m), 3.08 (0.6H, d, J=6.6 Hz), 3.03-2.95 (2H, m), 2.90 (1.4H, d, J=10.9 Hz), 2.23-1.62 (6H, m).

### Example 27

### 1′-(Cyclopropylmethyl)-1,2-dihydro-5-fluoro-1-(2,3-difluorobenzoyl)-spiro[3H-indole-3,4′-piperidine]

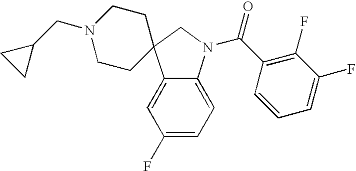

¹H NMR (CDCl₃, 400 MHz), A mixture of confirmational isomers was evident. Data reported for major isomer: δ 8.25 (1H, dd, J=8.8, 4.8 Hz), 7.30 (1H, m), 7.24 (2H, m), 6.98 (1H, td, J=6.98, 2.3 Hz), 6.92 (1H, m), 3.75 (2H, s), 3.06 (2H, d, J=11.8 Hz), 2.23 (2H, d, J=6.5 Hz) 1.98 (2H, dt, J=13.2, 3.8 Hz), 1.86 (2H, m), 1.70 (2H, d, J=12.8 Hz), 0.88 (1H, m), 0.52 (2H, m), 0.08 (2H, m).

### Example 28

### 1′-(Allyl)-1,2-dihydro-4,7-dimethyl-1-(2,3-difluorobenzoyl)-spiro[3H-indole-3,4′-piperidine]

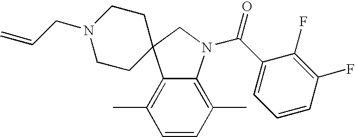

¹H NMR (MeOD): 7.4 (m, 1H), 7.25 (m, 2H), 7.0-6.8 (m, 2H), 5.75 (m, 1H), 5.1 (m, 2H), 3.75 (bs, 2H), 2.85 (m, 4H), 2.3 (m, 4H), 2.1 (bs, 2H), 1.79 (m, 2H), 1.43 (d, J=13.5, 2H), 1.2 (m, 2H).

### Example 29

### 1′-(Allyl)-1,2-dihydro-6,7-dimethyl-1-(2,3-difluorobenzoyl)-spiro[3H-indole-3,4′-piperidine]

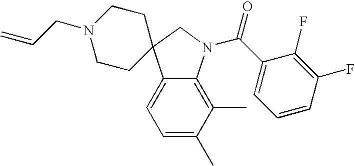

¹H NMR (MeOD) A mixture of conformational isomers was evident: 7.74 (m, 0.5H) 7.6-7.3 (m, 2H) 7.25 (m, 0.5H) 7.15 (d, J=7.4, 1H) 7.05 (d, J=7.2, 1H) 6.0 (m, 1H) 5.6 (m, 2H) 4.05 (bs, 2H) 3.75 (d, J=4.3, 2H) 3.58 (d, J=11.5, 2H) 2.95 (m, 2H) 2.35 (bs, 3H) 2.18 (m, 5H) 1.9 (d, J=14.8, 2H).

### Example 30

### 1′-(Allyl)-1,2-dihydro-5-methoxy-7-methyl-1-(2,3-difluorobenzoyl)-spiro[3H-indole-3,4′-piperidine]

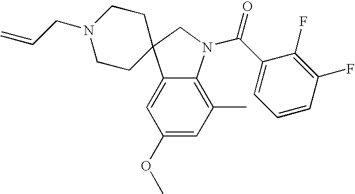

¹HNMR (MeOD): 7.4 (m, 2H) 7.25 (m, 1H) 6.65 (m, 2H) 5.85 (m, 1H) 5.55 (m, 2H) 3.95 (bs, 2H) 3.71 (s, 3H) 3.61 (d, J=4.9, 2H) 3.45 (d, J=11.9, 2H) 2.85 (m, 2H) 2.1 (m, 5H) 1.8 (d, J=13.8, 2H).

### Example 31

### 1′-(Allyl)-,2-dihydro-5-isopropyl-1-(2,3-difluorobenzoyl)-spiro[3H-indole-3,4′-piperidine]

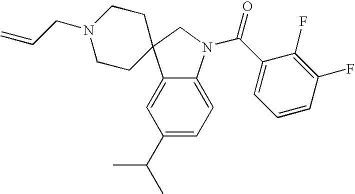

¹H NMR (MeOD) A mixture of conformational isomers was evident: 8.1 (d, J=8.9, 1H) 7.5 (m, 1H) 7.35 (m, 2H) 7.2 (m, 2H) 5.9 (m, 1H) 5.25 (m, 2H) 3.8 (s, 2H) 3.16 (m, 1H) 3.05 (d, J=6.5, 2H) 2.95 (m, 2H) 2.9 (m, 1H) 2.0 (m, 3H) 1.75 (m, 2H) 1.3 (d, J=6.9, 6H).

### Example 32

### 1′-(Cyclobutylmethyl)-1,2-dihydro-5-chloro-1-(2,6-diflurobenzoyl)-spiro[3H-indole-3,4′-piperidine]

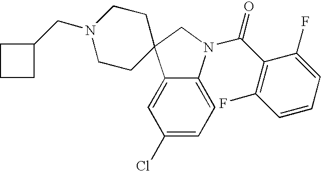

¹H NMR (CDCl₃) A mixture of conformational isomers was evident: 8.26 (d, J=8.6, 1H) 7.45 (m, 1H) 7.26 (m, 1H) 7.18 (d, J=2.1, 1H) 7.05 (m, 2H) 3.7 (s, 2H) 2.8 (d, J=11.7, 2H) 2.5 (m, 2H) 2.35 (d, J=6.8, 2H) 2.2-2.0 (m, 3H) 1.98-1.73 (m, 5H) 1.72-1.62 (m, 3H).

### Example 33

### 1′-(Allyl)-1,2-dihydro-5-fluoro-1-(benzo[1,3]dioxole-5-carbonyl)-spiro[3H-indole-3,4′-piperidine]

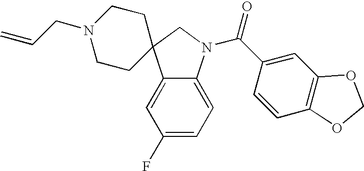

¹H NMR (CDCl₃, 400 MHz): δ 7.26 (1H, s), 7.09 (1H, dd, J=8.0, 1.5 Hz), 7.04 (1H, d, J=1.1 Hz), 6.89 (2H, m), 6.06 (2H, s), 5.88 (1H, ddt, J=17.0, 10.2, 6.6 Hz), 5.21-5.16 (2H, m), 3.95 (2H, s), 3.02 (2H, d, J=6.6 Hz), 2.93 (2H, m), 1.95, (4H, m), 1.70 (2H, m).

### Example 34

### 1′-(Cyclopropylmethyl)-1,2-dihydro-5-fluoro-1-(3-chlorobenzoyl)-spiro [3H-indole-3,4′-piperidine]

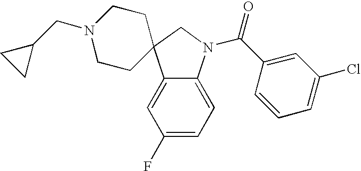

¹H NMR (CDCl₃, 400 MHz): δ 8.17 (1H, m), 7.54 (1H, s), 7.49 (1H, m), 7.42, (2H, d, J=4.9 Hz), 6.92 (2H, dd, J=8.3, 2.6 Hz), 3.87 (2H, m), 3.08 (2H, d, J=6.6 Hz), 2.28 (2H, m), 2.00 (3H, m), 1.71 (3H, m), 0.86 (1H, m), 0.53 (2H, m), 0.10 (2H, m).

### 6.2 BIOLOGICAL ASSAYS

### Example 35

### Mas Receptor IP₃ Assay

The Mas receptor IP₃ assay was performed using a mammalian cell line (HEK293) which was transfected with a plasmid containing the human Mas receptor and selected for stable expression of the receptor. For the inverse agonist assay, higher levels of Mas receptor constitutive activity were desired. To achieve this, Mas receptor expression levels were increased by transiently transfecting the same Mas receptor stable cell line with additional human Mas receptor plasmid DNA following standard procedures. These cells were used in the Mas receptor IP₃ assay approximately 24 hours post-transfection.

Cells were split into 96-well plates (50,000 cells/well) and allowed to attach for a period of 6 hours. The growth medium was then replaced with medium supplemented with 4 μCi/ml [³H]myo-inositol (100 μl; Perkin Elmer Life Sciences) and the cells were allowed to incubate for approximately 20 hours. Test compounds were serially diluted in inositol-free media containing 10 mM LiCl. The media in the plates was removed by aspiration, replaced with these test compound solutions and incubated at 37° C. for 1 hour. Following this incubation, the media was removed by aspiration and replaced with buffer containing 0.1M formic acid. The plates were then frozen overnight at −80° C. to achieve complete cell lysis.

The following day, the assay plates were thawed at room temperature. The thawed contents were then transfered to 96-well filter plates (Millipore, Multiscreen) pre-loaded with resin (Biorad, AG1-X8 100-200 mesh, formate form). The plate was filtered using a vacuum manifold and the resin was washed multiple times with water. An elution buffer was then applied (200 μl, 0.2M Ammonium formate/0.1M formic acid) and the resulting eluent was collected, under vacuum, in a 96-well collection plate. Aliquots of the eluent (80 μl) were transferred to filter plates (Whatman, Unifilter GF/C) and dried in a 45° C. oven overnight. Dried plates were counted on a scintillation counter following the addition of an appropriate scintillant (Perkin Elmer Life Sciences, Optiphase Supermix or Hi-Safe 3).

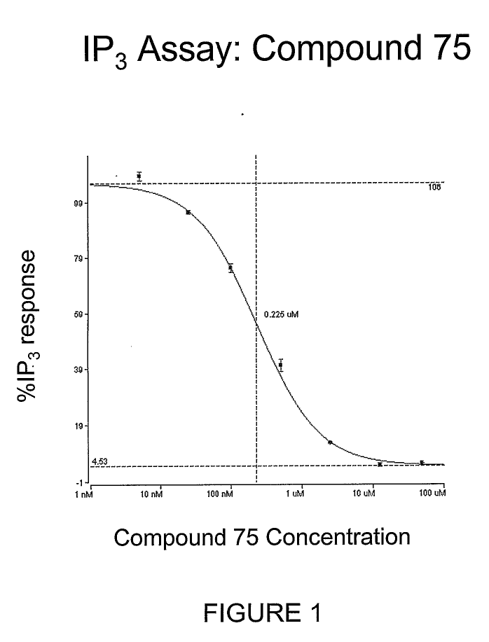

A representative experiment showing the results of an IP₃ assay for Compound 75 is shown in FIG. 1. In this particular experiment, the IC₅₀ value for Compound 75 was 225 nM. The average IC₅₀ value for Compound 75 obtained from several experiments was 297.67 nM (see Table 3).

The IC₅₀ values of several Compounds of the Invention are listed in TABLE 3.

TABLE 3

| Compound Number | IC₅₀ (nM) |
|---|---|
| 31 | 900.00 |
| 69 | 378.33 |
| 73 | 867.00 |
| 75 | 297.67 |
| 76 | 936.00 |
| 83 | 757.00 |
| 85 | 380.67 |
| 86 | 704.67 |
| 88 | 896.00 |
| 89 | 989.00 |
| 92 | 592.33 |
| 93 | 841.00 |
| 101 | 791.50 |
| 102 | 939.00 |
| 133 | 534.67 |
| 139 | 728.00 |
| 142 | 480.00 |
| 147 | 734.67 |
| 153 | 774.00 |
| 159 | 505.00 |
| 160 | 721.00 |
| 165 | 757.00 |
| 168 | 502.00 |
| 185 | 956.00 |
| 192 | 890.67 |
| 199 | 381.50 |
| 204 | 554.33 |
| 206 | 302.50 |
| 210 | 890.00 |
| 211 | 855.00 |
| 214 | 455.00 |
| 215 | 607.00 |
| 223 | 307.00 |
| 229 | 846.00 |

### Example 36

### Receptor Binding Assay

Several assays are well known in the art for identifying compounds that can bind to GPCRs. An example of a Mas receptor binding assay is described below.

Mas Receptor Preparation

293 cells (human kidney, ATCC) are transiently transfected with 10 μg human Mas receptor plasmid and 60 μl Lipofectamine (per 15-cm dish), grown in the dish for 24 hours (75% confluency (with a media change and removed with 10 ml/dish of Hepes-EDTA buffer (20 mM Hepes+10 mM EDTA, pH 7.4). The cells are then centrifuged in a Beckman Coulter centrifuge for 20 minutes, 17,000 rmp (JA-25.50 rotor). Subsequently, the pellet is resuspended in 20 mM Hepes+1 mM EDTA, pH 7.4 and homogenized with a 50-ml Dounce homogenizer and again centrifuged. After removing the supernatant, the pellets are stored at −80° C., until used in binding assay. When used in the binding assay, membranes are thawed on ice for about 20 minutes and then 10 mL of incubation buffer (20 mM Hepes, 1 mM MgCl₂, 100 mM NaCl, pH 7.4) is added. The membranes are then vortexed to resuspend the crude membrane pellet and homogenized with a Brinkmann PT-3100 Polytron homogenizer for about 15 seconds at setting 6. The concentration of membrane protein is determined using the BRL Bradford protein assay.

Binding Assay

For total binding, a total volume of 50 μl of appropriately diluted membranes (diluted in assay buffer containing 50 mM Tris HCl (pH 7.4), 10 mM MgCl₂, and 1 mM EDTA; 5-50 μg protein) is added to 96-well polypropylene microtiter plates followed by addition of 100 μl of assay buffer and 50 μl of a solution of a radiolabeled Compound of the Invention wherein the radiolabeled Compound of the Invention is present at a concentration of about 1 nM to 1 mM, preferably 1 nM to 500 μM, more preferably 1 nM to 100 μM, more preferably 10 nM to 100 μM, more preferably 100 nM to 100 μM, more preferably 1 μM to 100 μM and most preferably 10 μM to 100 μM. For nonspecific binding, 50 μl of assay buffer is added instead of 100 μl and an additional 50 μl of 10 μM cold Mas is added before 50 μl of a radiolabeled Compound of the Invention is added. Plates are then incubated at room temperature for about 60-120 minutes. The binding reaction is terminated by filtering assay plates through a Microplate Devices GF/C Unifilter filtration plate with a Brandell 96-well plate harvestor followed by washing with cold 50 mM Tris HCl, pH 7.4 containing 0.9% NaCl. The bottom of the filtration plate is then sealed, 50 μl of Optiphase Supermix is added to each well, the top of the filtration plates are sealed, and the filtration plates are counted in a Trilux MicroBeta scintillation counter. For compound competition studies, instead of adding 100 μl of assay buffer, 100 μl of appropriately diluted test compound is added to appropriate wells followed by addition of 50 μl of a radiolabeled Compound of the Invention.

### Example 37

### Ischemia-Reperfusion Injury in Isolated Adult Rat Hearts

Compounds of the invention can be characterized in several biological assays known in the art. For example, assays which analyze the effect of Compounds of the invention on the vascular, cardiovascular or nervous system can be performed. This example shows the results of assays which determine the effect of Compound 75 on ischemia-reperfusion injury in isolated adult rat hearts.

A. Ischemia-Reperfusion Assay 1 (Langendorff Apparatus):

Male Sprague-Dawley rats (300-350 g body weight) were anesthetized with pentobarbital sodium (50 mg/kg IP) then heparin (400 IU IP) was administered 10 minutes prior to surgery. The chest wall was opened and the heart was rapidly excised and immediately placed into ice-cold Krebs-Henseleit (KH) buffer (118 mM NaCl, 4.7 mM KCl, 1.2 mM MgSO₄, 1.2 mM KH₂PO₄, 1.5 mM CaCl₂, 25 mM NaHCO₃, 11 mM glucose, 1 mM pyruvate, and 0.005 mM EDTA) to produce cardiac arrest. The aorta was then cannulated and the heart retrogradely perfused with KH buffer maintained at 37° C. in a reservoir bubbled with 95% O₂/5% CO₂ (pH7.4) on the Langendorff apparatus at a constant pressure of 80 mmHg. Myocardial temperature was maintained at 37° C. by partially submerging the heart into a water-jacketed chamber filled with KH buffer. A water filled latex balloon attached to a metal cannula and inserted into the left ventricle via the mitral valve and connected to a pressure transducer (Powerlab, ADInstruments, Inc) was used for measurement of left ventricular pressure. The balloon was initially inflated to an end-diastolic pressure of 10 mmHg. After allowing 15 minutes for equilibration, rat hearts were subjected to 15 minutes of KH buffer containing drug or vehicle followed by 30 minutes of ischemia followed by 30 minutes of reperfusion. The difference between peak-systolic and end diastolic pressures, or left ventricular developed pressure (LVDP) was calculated as an index of contractile function and measured just prior to ischemia and at the end of reperfusion. Percent recovery of LV function [(LVDP post reperfusion/LVDP pre-ischemia)/100] was averaged across 8 vehicle and 8 drug treated hearts and a students t-test was used to analyze for a significant difference between the means.

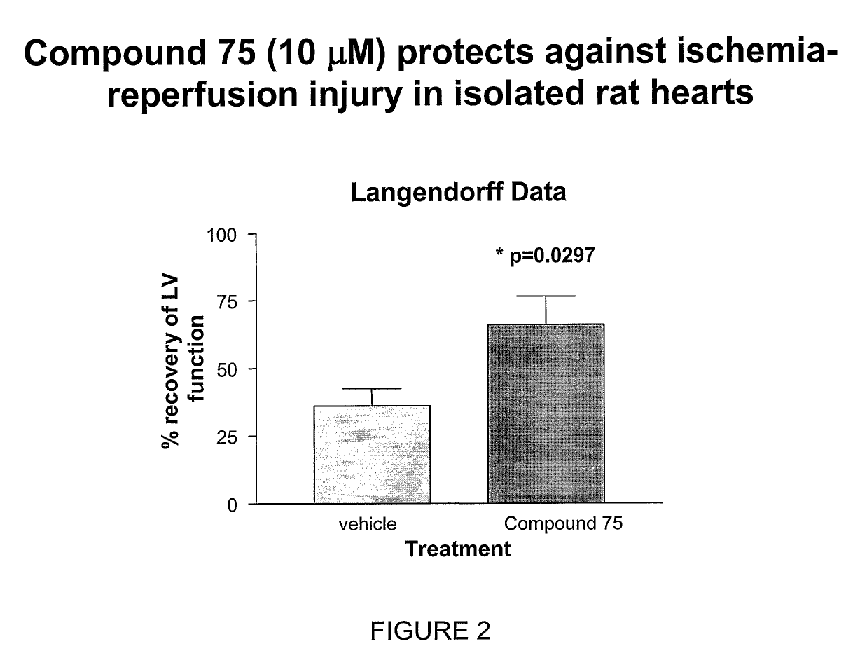

An example of a compound of the invention tested in this assay is shown in FIG. 2. In this example, Compound 75 at a concentration of 10 μM was found to provide protection against ischemia-reperfusion injury in isolated rat hearts as shown by a significant increase in percent recovery of left ventricle function compared to vehicle treatment.

After the filing of US provisional patent application No. 60/539,554 on Jan. 26, 2004, subsequent experiments using this protocol showed less consistent results with less statistical significance. Therefore, modifications were made to the protocol as shown below in section B.

The results of experiments performed using the protocol of section B have been highly reproducible and consistently show statistical significance.

B. Ischemia-Reperfusion Assay 2 (Langendorff Apparatus):

Male Sprague-Dawley rats (300-350 g body weight) were anesthetized with Inactin (100 mg/kg IP) 20 minutes prior to surgery. The chest wall was opened and the heart was rapidly excised and immediately placed into ice-cold Krebs-Henseleit (KH) buffer (118 mM NaCl, 4.7 mM KCl, 1.2 mM MgSO₄, 1.2 mM KH₂PO₄, 1.5 mM CaCl₂, 25 mM NaHCO₃, 11 mM glucose mM pyruvate, and 0.005 mM EDTA) to produce cardiac arrest. The aorta was then cannulated and the heart retrogradely perfused with KH buffer maintained at 37° C. in a reservoir bubbled with 95% O₂/5% CO₂ (pH7.4) on the Langendorff apparatus at a constant pressure of 70 mmHg. Myocardial temperature was maintained at 37° C. by partially submerging the heart into a water-jacketed chamber filled with KH buffer. A water filled latex balloon attached to a metal cannula and inserted into the left ventricle via the mitral valve and connected to a pressure transducer (Powerlab, ADInstruments, Inc) was used for measurement of left ventricular pressure. The balloon was initially inflated to an end-diastolic pressure of 10 mmHg. After allowing 20 minutes for equilibration, rat hearts were subjected to 10 minutes of KH buffer containing drug or vehicle followed by 30 minutes of ischemia followed by 30 minutes of reperfusion. The difference between peak-systolic and end diastolic pressures, or left ventricular developed pressure (LVDP) was calculated as an index of contractile function and measured just prior to ischemia and at 10, 20 and 30 minutes of reperfusion. Percent recovery of LV function [(LVDP post reperfusion/LVDP pre-ischemia)/100] was averaged across 5 vehicle and 4 drug treated hearts and one-way anova with Newman-Keuls Multiple Comparison Test was used to determine statistical significance.

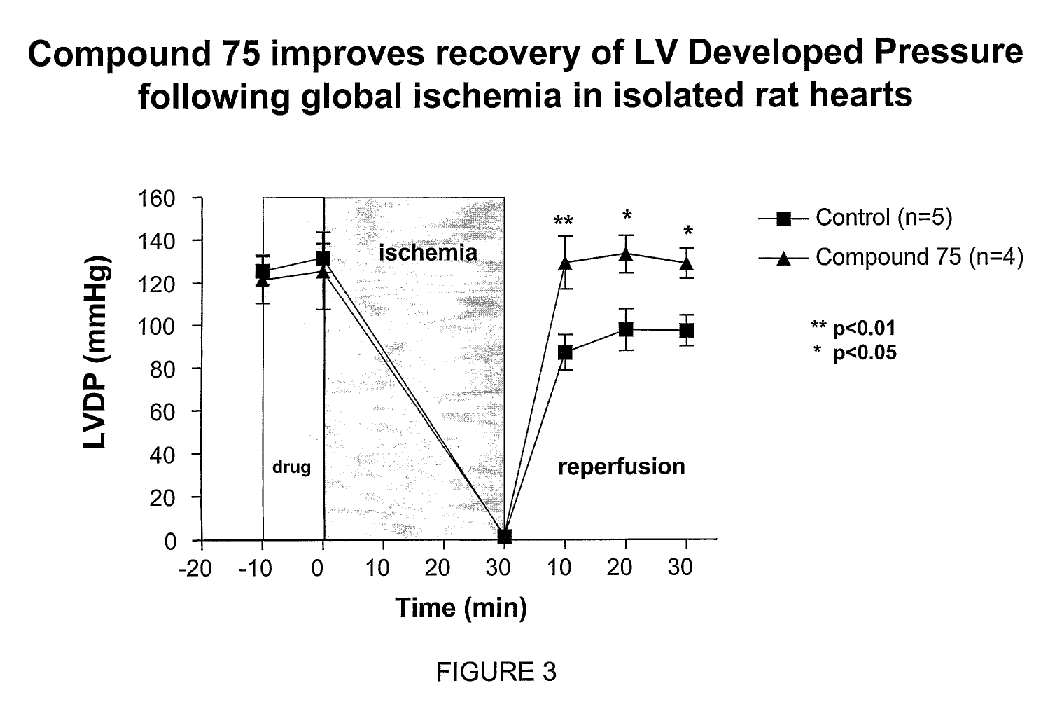

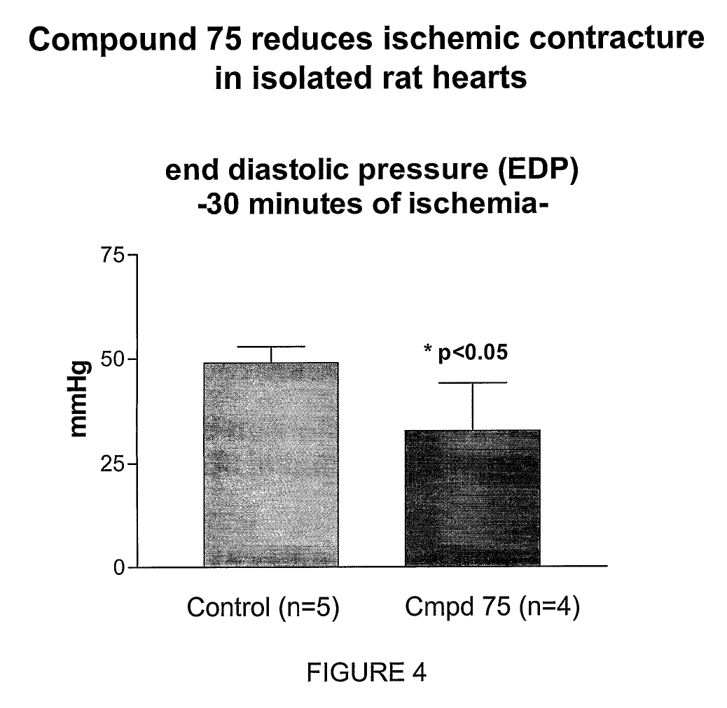

An example of a compound of the invention tested in this assay is shown in FIG. 3. In this example, Compound 75 at a concentration of 30 μM was found to provide protection against ischemia-reperfusion injury in isolated rat hearts as shown by a significant increase in left ventricle function after reperfusion compared to vehicle treatment. As shown in FIG. 3, the level of left ventricle function after reperfusion in Compound 75 treated hearts was comparable to the level before ischemia. In addition, as shown in FIG. 4, Compound 75 at a concentration of 30 μM was found to reduce ischemic contracture in isolated rat hearts as shown by a significant decrease in end diastolic pressure (EDP) compared to vehicle treatment.

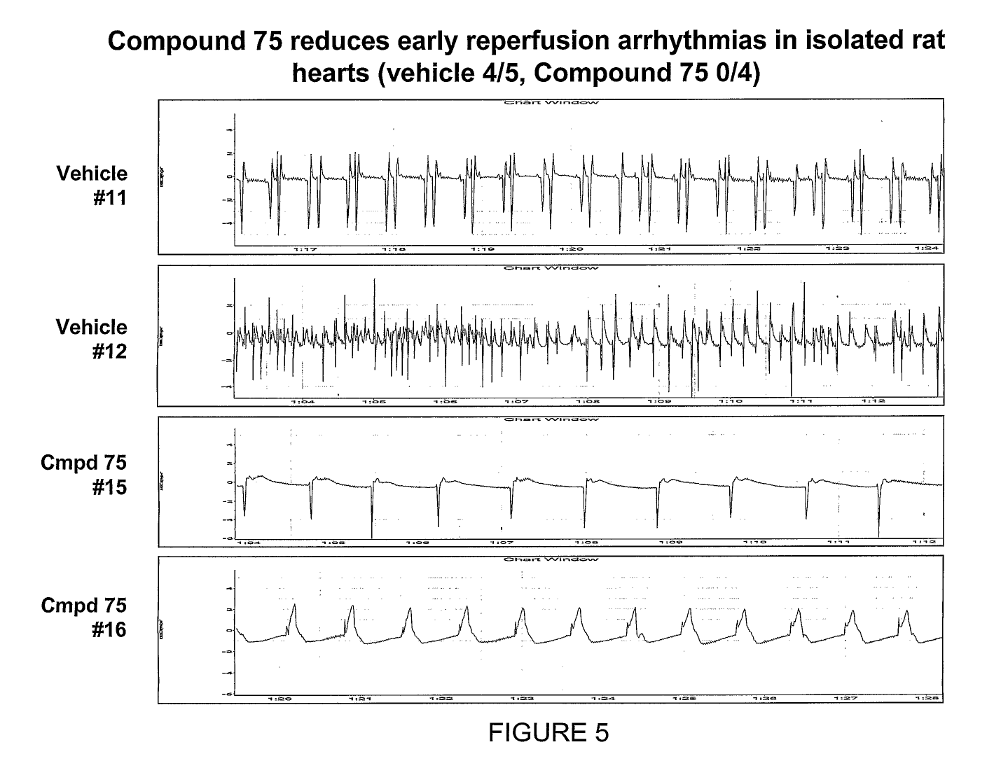

Epicardial electrogram recordings were also taken from the isolated rat hearts used in FIGS. 3 and 4. Briefly, silver wire electrodes were placed on the right atrium and the apex of the left ventricle, allowing an epicardial electrogram to be recorded. Premature ventricular contraction (PVC), ventricular tachycardia and ventricular fibrillation were common arrhythmias observed during reperfusion following 30 minutes of global ischemia. Hearts were considered positive for reperfusion arrhythmias if ventricular arrhythmias were sustained for greater than 30 seconds during the first 5 minutes of reperfusion. As shown in FIG. 5, early reperfusion arrhythmias were observed in vehicle treated isolated hearts but not in hearts treated Compound 75. In this experiment, none of the four hearts treated with Compound 75 showed early reperfusion arrhythmias while 4 of the 5 vehicle treated hearts showed early reperfusion arrhythmias.

### Example 38

### Measurement of Blood Pressure in Rats Exposed to Compound 75

Telemetry Studies:

Cardiac parameters were measured by small transmitting devices, (Data Sciences PhysioTel Telemetry devices), implanted in rats. The implanted transmitting devices were used to measure blood pressure in freely moving conscious animals. There are no external connections or tethering devices that can inhibit animal movement and induce unnecessary stress, which can affect the outcome of a study.

Transmitter Implantation:

This procedure was performed under modified aseptic conditions. Rats were anesthetized with Isoflurane gas that ranged in concentration from 1.5-2.0%. A cardiac telemetry device was implanted into the peritoneal cavity with a pressure sensing catheter situated no more than 2 cm inside the descending aorta. This was accomplished as follows: The rat was shaved and the incision site was prepared with an iodine solution. The rat was then placed on a heating pad to maintain a constant body temp of 38±0.5° C., and covered with a sterile drape. A 6 cm midline abdominal incision was made to provide access to the implantation area. Then the stomach muscle was cut with sharp scissors. The contents of the abdomen were exposed with retractors and the intestines were rearranged with wet gauze to expose the aorta. The aorta was separated from the vena cava. The aorta was then punctured just cranial to the aortic bifurcation with a bent 21 gauge needle. Immediately the pressure sensing catheter was inserted no more than 2 cm into the aorta. The site was thoroughly dried and 1-2 drops of Vet bond adhesive was applied. The site was checked to ensure there was no bleeding. Also, the signal from the transmitter was checked to verify that there was a sufficient signal from the transmitter. The gauze and retractors were then removed and the abdominal area was rinsed with sterile saline. These animals also have biopotential leads which were channeled through the stomach muscle with a sterile 16 gauge needle. Biopotential leads, which are used to measure an electrical signal generated by the contraction of the ventricles of the heart, were implanted into the muscle in order to obtain electrocardiogram (ECG) output, if desired. The skin incision sites for the biopotential leads and abdomen were closed with sterile incision staples. Antibiotic ointment was applied to the incision areas. Post operative antibiotics, (Sulfatrim-sulfamethoxazole+trimethoprim), were mixed with their drinking water, (20 mil/quart H₂O), for 5 days after surgery. The rats were monitored for 7 days to ensure proper recovery.

On the test day, injections with either vehicle or with test compound were administered via IP injection in volumes of ˜250 μl. Animals were monitored with a resolution of approximately one measurement/min for 60 minutes before injection of vehicle or compound and for about 120 minutes after injection.

FIG. 6 shows blood pressure readings obtained using the protocol described above. As expected, the vasoconstrictor angiotensin II (angII) resulted in a significant increase in blood pressure while the vasodilator sodium nitroprusside (snp) resulted in a significant decrease in blood pressure in treated rats. Treatment of rats with Compound 75 did not result in a significant change in blood pressure compared to the blood pressure readings recorded in these rats before treatment with the compound.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which are incorporated herein by reference.

1. A compound of Formula (I): 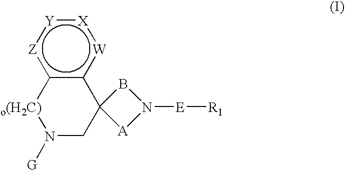
or a pharmaceutically acceptable salt, free base, solvate, hydrate or stereoisomer, thereof, wherein:
R₁ is H, halogen, hydroxy, nitro, cyano, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, substituted or unsubstituted -(3 to 7) membered heterocycle, substituted or unsubstituted -(7 to 10) membered bicycloheterocycle, substituted or unsubstituted -(5 to 10) membered heteroaryl, —NR₂R₂′, —C(═O)—R₇, —S(═O)₂—R₇, —C(═O)O—R₇, or —C(═O)N(R₇)(C₁₋₆ alkyl);
A is a substituted or unsubstituted C₁-C₃ alkylene;
B is a substituted or unsubstituted C₁-C₃ alkylene;
E is a bond, or a substituted or unsubstituted C₁-C₃ alkylene;
G is H, —Ar, —C(═O)—Ar, —C(═O)O—Ar, substituted or unsubstituted —C(═O)O—C₁₋₆ alkyl, —C(═O)N(R₇)(Ar), substituted or unsubstituted —C(═O)N(R₇)(C₁₋₆ alkyl), —S(═O)₂—Ar, substituted or unsubstituted —S(═O)₂—C₁₋₆ alkyl, substituted or unsubstituted C₁₋₆ alkyl, substituted or unsubstituted C₁₋₆ alkyl-Ar, substituted or unsubstituted —C(═O)C₁₋₆ alkyl-Ar, or substituted or unsubstituted —C(═O)C₁₋₆ alkyl;
W is N or —CR₃—;
X is N or —CR₄—;
Y is N or —CR₅—;
Z is N or —CR₆—;
R₂, R₂′, R₃, R₄, R₅, R₆ and R₇ are at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, —C(═O)—O—C₁₋₆ alkyl, —O—C₁₋₆ alkyl, —C₁₋₆ alkyl-O—C₁₋₄ alkyl, —C₁₋₆ alkyl-NH₂, —C₀₋₆ alkyl-C(═O)—NH(C₁₋₆ alkyl), —C₀₋₆ alkyl-C(═O)—N(C₁₋₆ alkyl)(C₁₋₆ alkyl), —C₁₋₆ alkyl-NH—C(═O)—C₁₋₄ alkyl, —C₁₋₆ alkyl-S(═O)—C₁₋₆ alkyl, —C₀₋₆ alkyl-O—S(═O)₂—C₁₋₆ alkyl, —C₁₋₆ alkyl-S(═O)₂—C₁₋₆ alkyl, —C₁₋₆ alkyl-NR′—S(═O)₂—R′, —C₁₋₆ alkyl-SH, —C₁₋₆ alkyl-S—C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —(C(R′)₂)₀₋₆—O—(C(R′)₂)₁₋₅C(R′)₃, —(C(R′)₂)₁₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂—(C(R′)₂)₁₋₅C(R′)₃;
o is 0 or 1;
R′ is at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted aryl, or substituted or unsubstituted C₃₋₈ cycloalkyl; and
Ar is substituted or unsubstituted aryl, substituted or unsubstituted C₃₋₇ cycloalkyl, substituted or unsubstituted C₈₋₁₄ bicycloalkyl, substituted or unsubstituted C₈₋₁₄ tricycloalkyl, substituted or unsubstituted -(3 to 7) membered heterocycle, substituted or unsubstituted -(7 to 10) membered bicycloheterocycle, or substituted or unsubstituted -(5 to 10 membered)heteroaryl.

2. The compound of claim 1, wherein E is —(CH₂)ₚ— and p is 0, 1 or 2.

3. A compound of claim 2, wherein W is —CR₃—, X is —CR₄—, Y is —CR₅— and Z is —CR₆—.

4. A compound of claim 2, wherein A and B are both —(CH₂)₂—.

5. A compound of claim 2, wherein p is 1 and R₁ is —CH═CH₂.

6. A compound of claim 2, wherein p is 1 and R₁ is -cyclopropyl.

7. A compound of claim 2, wherein R₁ is phenyl.

8. A compound of claim 2, wherein G is —C(═O)—Ar.

9. A compound of claim 2, wherein G is —C(═O)NH—Ar.

10. A compound of claim 2, wherein G is —S(═O)₂—Ar.

11. A compound of claim 2, wherein Ar is phenyl.

12. A compound of claim 2, wherein o is 0.

13. A compound of claim 1, selected from the group consisting of:
1′-(cyclobutylcarbonyl)-1,2-dihydro-1-butyl-spiro[3H-indole-3,4′-piperidine];
1′-(diphenylacetyl)-1,2-dihydro-1-butyl-spiro[3H-indole-3,4′-piperidine];
1′-(cyclobutylcarbonyl)-1,2-dihydro-1-phenethyl-spiro[3H-indole-3,4′-piperidine];
1′-(diphenylacetyl)-1,2-dihydro-1-phenethyl-spiro[3H-indole-3,4′-piperidine];
1′-(butyl)-1,2-dihydro-1-(cyclobutylcarbonyl)-spiro[3H-indole-3,4′-piperidine];
1′-(phenethyl)-1,2-dihydro-1-(cyclobutylcarbonyl)-spiro[3H-indole-3,4′-piperidine];
1′-(butyl)-1,2-dihydro-1-(diphenylacetyl)-spiro[3H-indole-3,4′-piperidine];
1′-(phenethyl)-1,2-dihydro-1-(diphenylacetyl)-spiro[3H-indole-3,4′-piperidine];
1′-{4-[(4-chloro-benzenesulfonyl)-methyl-amino]-3-phenyl-butyl}-1,2-dihydro-1-(tert-butoxycarbonyl)-5-fluoro-spiro[3H-indole-3,4′-piperidine];
1′-{4-[(4-nitro-benzenesulfonyl)-methyl-amino]-3-phenyl-butyl}-1,2-dihydro-1-(tert-butoxycarbonyl)-5-fluoro-spiro[3H-indole-3,4′-piperidine];
1′-(diphenylacetyl)-1,2-dihydro-1-(tert-butoxycarbonyl)-spiro[3H-indole-3,4′-piperidine];
1′-(isobutyryl)-1,2-dihydro-1-(tert-butoxycarbonyl)-spiro[3H-indole-3,4′-piperidine];
1′-(cyclobutylcarbonyl)-1,2-dihydro-1-(tert-butoxycarbonyl)-spiro[3H-indole-3,4′-piperidine];
1′-(cyclobutylcarbonyl)-1,2-dihydro-spiro[3H-indole-3,4′-piperidine];
1′-(cyclobutylcarbonyl)-1,2-dihydro-1-(2,4-dimethyl-benzyl)-spiro[3H-indole-3,4′-piperidine];
1′-(4-chloro-benzenesulfonyl)-1,2-dihydro-spiro[3H-indole-3,4′-piperidine];
1′-diphenylacetyl)-1,2-dihydro-1-(benzo[1,3]dioxol4-ylmethyl)-spiro[3H-indole-3,4′-piperidine];
1′-(4-chloro-benzenesulfonyl)-1,2-dihydro-1-3,4-dichloro-benzyl)-spiro[3H-indole-3,4′-piperidine];
1′-diphenylacetyl)-1,2dihydro-1-(2,4-dimethyl-benzyl)-spiro[3H-indole-3,4′-piperidine];
1′-(diphenylacetyl)-1,2-dihydro-spiro[3H-indole-3,4′-piperidine];
1′-(4-chloro-benzenesulfonyl)-1,2-dihydro-1-(benzo[1,3]dioxol4-ylmethyl)-spiro[3H-indole-3,4′-piperidine];
1′-(4chloro-benzenesulfonyl)-1,2-dihydro-1-(2,4-dimethyl-benzyl)-spiro[3H-indole-3,4′-piperidine];
1′-(naphthalene-1-carbonyl)-1,2-dihydro-1-(benzo[1,3]dioxol4-ylmethyl)-spiro[3H-indole-3,4′-piperidine];
1′-(naphthalene-1-carbonyl)-1,2-dihydro-1-(2,4-dimethyl-benzyl)-spiro[3H-indole-3,4′-piperidine];
1′-(isobutyryl)-1,2-dihydro-1-(phenethyl)-spiro[3H-indole-3,4′-piperidine];
1′-(isobutyryl)-1,2-dihydro-1-(butyl)-spiro[3H-indole-3,4′-piperidine];
1′-(isobutyryl)-1,2-dihydro-1-(benzo[1,3]dioxol4-ylmethyl)-spiro[3H-indole-3,4′-piperidine];
1′-(isobutyryl)-1,2-dihydro-1-(2,4-dimethyl-benzyl)-spiro[3H-indole-3,4′-piperidine];
1′-(cyclobutylcarbonyl)-1,2-dihydro-1-(3,4-dichloro-benzyl)-spiro[3H-indole-3,4′-piperidine];
1′-(cyclopropylmethyl)-1,2-dihydro-5-fluoro-1-(2-chloro-benzoyl)-spiro[3H-indole-3,4′-piperidine];
1′-(cyclopropylmethyl)-1,2-dihydro-5-fluoro-1-(4-methoxy-benzoyl)-spiro[3H-indole-3,4′-piperidine];
1′-(cyclopropylmethyl)-1,2-dihydro-5-fluoro-1-(4-nitro-benzoyl)-spiro[3H-indole-3,4′-piperidine];
1′-(cyclopropylmethyl)-1,2-dihydro-5-fluoro-1-(diphenylacetyl)-spiro[3H-indole-3,4′-piperidine];
1′-(cyclopropylmethyl)-1,2-dihydro-5-fluoro-1-(cyclobutylcarbonyl)-spiro[3H-indole-3,4′-piperidine];
1′-butyl)-1,2-dihydro-5-fluoro-1-cyclobutylcarbonyl)-spiro[3H-indole-3,4′-piperidine];
1′-(butyl)-1,2-dihydro-5-fluoro-1-2-chloro-benzoyl)-spiro[3H-indole-3,4′-piperidine];
1′-(butyl)-1,2-dihydro-5-fluoro-1-4-methoxy-benzoyl)-spiro[3H-indole-3,4′-piperidine];
1′-(butyl)-1,2-dihydro-5-fluoro-1-(4-nitro-benzoyl)-spiro[3H-indole-3,4′-piperidine];
1′-(butyl)-1,2-dihydro-5-fluoro-1-(diphenylacetyl)-spiro[3H-indole-3,4′-piperidine];
1′-(phenethyl)-1,2-dihydro-5-fluoro-1-(4-methoxy-benzoyl)-spiro[3H-indole-3,4′-piperidine];
1′-(phenethyl)-1,2-dihydro-5-fluoro-1-(4-nitro-benzoyl)-spiro[3H-indole-3,4′-piperidine];
1′-(3,4-dimethoxy-benzyl)-1,2-dihydro-5-fluoro-1-(cyclobutylcarbonyl)-spiro[3H-indole-3,4′-piperidine];
1′-(3,4-dimethoxy-benzyl)-1,2-dihydro-5-fluoro-1-(2-chloro-benzoyl)-spiro[3H-indole-3,4′-piperidine];
1′-(3,4-dimethoxy-benzyl)-1,2-dihydro-5-fluoro-1-(4-methoxy-benzoyl)-spiro[3H-indole-3,4′-piperidine];
1′-(3,4-dimethoxy-benzyl)-1,2-dihydro-5-fluoro-1-(4-nitro-benzoyl)-spiro[3H-indole-3,4′-piperidine];
1′-(cyclopropylmethyl)-1,2-dihydro-5-fluoro-1-(2-chloro-benzoyl)-spiro[3H-indole-3,4′-piperidine];
1′-(3,4-dichloro-benzyl)-1,2-dihydro-5-fluoro-1-(2-chloro-benzoyl)-spiro[3H-indole-3,4′-piperidine];
1′-(3,4-dichloro-benzyl)-1,2-dihydro-5-fluoro-1-(4-methoxy-benzoyl)-spiro[3H-indole-3,4′-piperidine];
1′-(3,4-dichloro-benzyl)-1,2-dihydro-5-fluoro-1-(4-nitro-benzoyl)-spiro[3H-indole-3,4′-piperidine];
1′-(3,4-dichloro-benzyl)-1,2-dihydro-5-fluoro-1-(diphenylacetyl)-spiro[3H-indole-3,4′-piperidine];
1′-(phenethyl)-1,2-dihydro-5-fluoro-1-(2-chloro-benzoyl)-spiro[3H-indole-3,4′-piperidine];
1′-(3,4-dichloro-benzyl)-1,2-dihydro-5-fluoro-spiro[3H-indole-3,4′-piperidine];
1′-cyclobutylcarbonyl)-1,2-dihydro-1-benzo[1,3]dioxol4-ylmethyl)-spiro[3H-indole-3,4′-piperidine];
1′-(phenylcarbamoyl)-1,2-dihydro-spiro[3H-indole-3,4′-piperidine];
1′-[cis-2-(4-methyl-3-nitro-benzoyloxy)-cyclohexyl]-1,2-dihydro-spiro[3H-indole-3,4′-piperidine];
1′-(cis-2-hydroxy-cyclohexyl)-1,2-dihydro-1-(3-nitro-4-methyl-benzoyl)-spiro[3H-indole-3,4′-piperidine];
1′-(3-nitro-4-methyl-benzoyl)-1,2-dihydro-1-(3-nitro-4-methyl-benzoyl)-spiro[3H-indole-3,4′-piperidine];
1′-(4-fluoro-benzoyl)-1,2-dihydro-1-(4-fluoro-benzoyl)-spiro[3H-indole-3,4′-piperidine];
1′-(2-methoxy-benzoyl)-1,2-dihydro-1-(2-methoxy-benzoyl)-spiro[3H-indole-3,4′-piperidine];
1′-(benzo[1,3]dioxole-5-carbonyl)-1,2-dihydro-1-(benzo[1,3]dioxole-5-carbonyl)-spiro[3H-indole-3,4′-piperidine];
1′-(cis-2-cyclohexylcarbamoyloxy-cyclohexyl)-1,2-dihydro-1-(cyclohexylcarbamoyl)-spiro[3H-indole-3,4′-piperidine];
1′-(cyclohexylcarbamoyl)-1,2-dihydro-1-(cyclohexylcarbamoyl)-spiro[3H-indole-3,4′-piperidine];
1′-[2-(3,4-difluoro-benzoyloxy)-propyl]-1,2-dihydro-1-(3,4-difluoro-benzoyl)-spiro[3H-indole-3,4′-piperidine];
1′-[2-hydroxy-propyl]-1,2-dihydro-1-(3,4-difluoro-benzoyl)-spiro[3H-indole-3,4′-piperidine];
1′-[2-hydroxy-propyl]-1,2-dihydro-1-(2-chloro-benzoyl)-spiro[3H-indole-3,4′-piperidine];
1′-[2-hydroxy-propyl]-1,2-dihydro-1-(2-fluoro-benzoyl)-spiro[3H-indole-3,4′-piperidine];
1′-[2-hydroxy-propyl]-1,2-dihydro-1-(cyclopentylcarbonyl)-spiro[3H-indole-3,4′-piperidine];
1′-[2-hydroxy-propyl]-1,2-dihydro-1-(4-chloro-benzoyl)-spiro[3H-indole-3,4′-piperidine];
1′-(allyl)-1,2-dihydro-5-fluoro-1-(2-chloro-benzoyl)-spiro[3H-indole-3,4′-piperidine];
1′-allyl)-1,2-dihydro-5-fluoro-1-4-chloro-benzoyl)-spiro[3H-indole-3,4′-piperidine];
1′-(allyl)-1,2-dihydro-5-fluoro-1-3,5-dichloro-benzoyl)-spiro[3H-indole-3,4′-piperidine];
1′-(allyl)-1,2-dihydro-5-fluoro-1-(2-methoxy-benzoyl)-spiro[3H-indole-3,4′-piperidine];
1′-(allyl)-1,2-dihydro-5-fluoro-1-(3,5-dimethoxy-benzoyl)-spiro[3H-indole-3,4′-piperidine];
1′-(allyl)-1,2-dihydro-5-fluoro-1-(3-fluoro-benzoyl)-spiro[3H-indole-3,4′-piperidine];
1′-(allyl)-1,2-dihydro-5-fluoro-1-(2,3-difluoro-benzoyl)-spiro[3H-indole-3,4′-piperidine];
1′-(allyl)-1,2-dihydro-5-fluoro-1-(2,4-difluoro-benzoyl)-spiro[3H-indole-3,4′-piperidine];
1′-(allyl)-1,2-dihydro-5-fluoro-1-[3-trifluoromethyl-benzoyl]-spiro[3H-indole-3,4′-piperidine];
1′-(allyl)-1,2-dihydro-5-fluoro-1-(3,5-bis-trifluoromethyl-benzoyl)-spiro[3H-indole-3,4′-piperidine];
1′-(allyl)-1,2-dihydro-5-fluoro-1-(cyclohexylcarbonyl)-spiro[3H-indole-3,4′-piperidine];
1′-(allyl)-1,2-dihydro-5-fluoro-1-(cyclopetnylcarbonyl)-spiro[3H-indole-3,4′-piperidine];
1′-(allyl)-1,2-dihydro-5-fluoro-1-(benzoyl)-spiro[3H-indole-3,4′-piperidine];
1′-(allyl)-1,2-dihydro-5-fluoro-1-(3-nitro-benzoyl)-spiro[3H-indole-3,4′-piperidine];
1′-(allyl)-1,2-dihydro-5-fluoro-1-(4-nitro-benzoyl)-spiro[3H-indole-3,4′-piperidine];
1′-(allyl)-1,2-dihydro-5-fluoro-1-(3-nitro4-methyl-benzoyl)-spiro[3H-indole-3,4′-piperidine];
1′-(allyl)-1,2-dihydro-5-fluoro-1-(naphthalene-I -carbonyl)-spiro[3H-indole-3,4′-piperidine];
1′-(allyl)-1,2-dihydro-5-fluoro-1-(3-chloro-benzoyl)-spiro[3H-indole-3,4′-piperidine];
1′-(allyl)-1,2-dihydro-5-fluoro-1-(4-fluoro-benzoyl)-spiro[3H-indole-3,4′-piperidine];
1′-(allyl)-1,2-dihydro-5-fluoro-1-(3-trifluoromethyl-4-fluoro-benzoyl)-spiro[3H-indole-3,4′-piperidine];
1′-(allyl)-1,2-dihydro-5-fluoro-1-(benzo[1,3]dioxole-5-carbonyl)-spiro[3H-indole-3,4′-piperidine];
1′-(allyl)-1,2-dihydro-5-fluoro-1-2-fluoro-benzoyl)-spiro[3H-indole-3,4′-piperidine];
1′-(allyl)-1,2-dihydro-5-fluoro-1-(3-dichloromethyl-benzoyl)-spiro[3H-indole-3,4′-piperidine];
1′-(allyl)-1,2-dihydro-5-fluoro-1-(2-chloro-pyridine-3-carbonyl)-spiro[3H-indole-3,4′-piperidine];
1′-(allyl)-1,2-dihydro-1-(3,5-bis-trifluoromethyl-benzoyl)-spiro[3H-indole-3,4′-piperidine];
1′-(allyl)-1,2-dihydro-1-(2,4-difluoro-benzoyl)-spiro[3H-indole-3,4′-piperidine];
1′-(3-nitro-benzoyl)-1,2-dihydro-1-(3-nitro-benzoyl)-spiro[3H-indole-3,4′-piperidine];
1′-(phenethyl)-1,2-dihydro-1-(ethylcarbamoyl)-spiro[3H-indole-3,4′-piperidine];
1′-(allyl)-1,2-dihydro-1-(cyclohexylcarbonyl)-spiro[3H-indole-3,4′-piperidine];
1′-(allyl)-1,2-dihydro-1-(2-methoxy-benzoyl)-spiro[3H-indole-3,4′-piperidine];
1′-(phenethyl)-1,2-dihydro-1-(2-chloro-benzoyl)-spiro[3H-indole-3,4′-piperidine];
1′-(cyclopropylmethyl)-1,2-dihydro-1-(2-chloro-benzoyl)-spiro[3H-indole-3,4′-piperidine];
1′-(allyl)-1,2-dihydro-1-(3-dichloromethyl-benzoyl)-spiro[3H-indole-3,4′-piperidine];
1′-(2-methyl-allyl)-1,2-dihydro-1-(3-trifluoromethyl-benzenesulfonyl)-spiro[3H-indole-3,4′-piperidine];
1′-(2-fluoro-benzoyl)-1,2-dihydro-1-(2-fluoro-benzoyl)-spiro[3H-indole-3,4′-piperidine];
1′-(phenethyl)-1,2-dihydro-1-(naphthylene-1-carbonyl)-spiro[3H-indole-3,4′-piperidine];
1,2-dihydro-1-(2-chloro-benzoyl)-spiro[3H-indole-3,4′-piperidine];
1′-(2-methyl-allyl)-1,2-dihydro-1-(3-fluoro-benzoyl)-spiro[3H-indole-3,4′-piperidine];
1′-(4-methoxy-benzoyl)-1,2-dihydro-1-(4-methoxy-benzoyl)-spiro[3H-indole-3,4′-piperidine];
1′-(phenethyl)-1,2-dihydro-1-2,4-dichloro-benzenesulfonyl)-spiro[3H-indole-3,4′-piperidine];
1′-(phenethyl)-1,2-dihydro-1-thiophene-2-carbonyl)-spiro[3H-indole-3,4′-piperidine];
1′-2-methyl-allyl)-1,2-dihydro-1-ethylcarbamoyl)-spiro[3H-indole-3,4′-piperidine];
1′-(phenethyl)-1,2-dihydro-1-(3-nitro-benzoyl)-spiro[3H-indole-3,4′-piperidine];
1′-2-methyl-allyl)-1,2-dihydro-1-(3-trifluoromethyl-benzoyl)-spiro[3H-indole-3,4′-piperidine];
1′-cyclopropylmethyl)-1,2-dihydro-1-cyclobutylcarbonyl)-spiro[3H-indole-3,4′-piperidine];
1′-2-methyl-allyl)-1,2-dihydro-1-(2,4-dichloro-benzenesulfonyl)-spiro[3H-indole-3,4′-piperidine];
1′-(2-methyl-allyl)-1,2-dihydro-1-(9-oxo-9H-fluorene-1-carbonyl)-spiro[3H-indole-3,4′-piperidine];
1′-(allyl)-1,2-dihydro-1-(3-nitro-benzoyl)-spiro[3H-indole-3,4′-piperidine];
1′-(phenethyl)-1,2-dihydro-1-(4-nitro-phenylcarbamoyl)-spiro[3H-indole-3,4′-piperidine];
1′-(2-methyl-allyl)-1,2-dihydro-1-(4-methoxy-phenylcarbamoyl)-spiro[3H-indole-3,4′-piperidine];
1′-(phenethyl)-1,2-dihydro-1-(4-methoxy-phenylcarbamoyl)-spiro[3H-indole-3,4′-piperidine];
1′-(cyclopropylmethyl)-1,2-dihydro-1-(cyclohexylcarbonyl)-spiro[3H-indole-3,4′-piperidine];
1′-(2-methyl-allyl)-1,2-dihydro-1-(benzo[1,3]dioxole-5-carbonyl)-spiro[3H-indole-3,4′-piperidine];
1′-(cyclopropylmethyl)-1,2-dihydro-1-(3-dichloromethyl-benzoyl)-spiro[3H-indole-3,4′-piperidine];
1′-(cyclopropylmethyl)-1,2-dihydro-1-(3,5-bis-trifluoromethyl-benzoyl)-spiro[3H-indole-3,4′-piperidine];
1′-(allyl)-1,2-dihydro-1-(thiophene-2-carbonyl)-spiro[3H-indole-3,4′-piperidine];
1′-(allyl)-1,2-dihydro-1-(2-chloro-benzoyl)-spiro[3H-indole-3,4′-piperidine];
1′-(2-methyl-allyl)-1,2-dihydro-1-(pyrrolidine-1-carbonyl)-spiro[3H-indole-3,4′-piperidine];
1′-phenethyl)-1,2-dihydro-1-(morpholine-4-carbonyl)-spiro[3H-indole-3,4′-piperidine];
1′-(2-methyl-allyl)-1,2-dihydro-1-4-nitro-phenylcarbamoyl)-spiro[3H-indole-3,4′-piperidine];
1′-(cyclopropylmethyl)-1,2-dihydro-1-(2,4-difluoro-benzoyl)-spiro[3H-indole-3,4′-piperidine];
1′-(2-methyl-allyl)-1,2-dihydro-1-2-fluoro-benzoyl)-spiro[3H-indole-3,4′-piperidine];
1′-(2-methyl-allyl)-1,2-dihydro-1-(cyclobutylcarbonyl)-spiro[3H-indole-3,4′-piperidine];
1′-(allyl)-1,2-dihydro-1-(naphthylene-1-carbonyl)-spiro[3H-indole-3,4′-piperidine];
1′-(naphthylene-1-carbonyl)-1,2-dihydro-1-(naphthylene-1-carbonyl)-spiro[3H-indole-3,4′-piperidine];
1′-(cyclopropylmethyl)-1,2-dihydro-5-fluoro-1-(4-chloro-benzoyl)-spiro[3H-indole-3,4′-piperidine];
1′-(cyclopropylmethyl)-1,2-dihydro-5-fluoro-1-(2-methoxy-benzoyl)-spiro[3H-indole-3,4′-piperidine];
1′-(cyclopropylmethyl)-1,2-dihydro-5-fluoro-1-(3,5-dimethoxy-benzoyl)-spiro[3H-indole-3,4′-piperidine];
1′-(cyclopropylmethyl)-1,2-dihydro-5-fluoro-1-(3,4,5-trimethoxy-benzoyl)-spiro[3H-indole-3,4′-piperidine];
1′-(cyclopropylmethyl)-1,2dihydro-5-fluoro-1-(2-fluoro-benzoyl)-spiro[3H-indole-3,4′-piperidine];
1′-(cyclopropylmethyl)-1,2-dihydro-5-fluoro-1-(3-fluoro-benzoyl)-spiro[3H-indole-3,4′-piperidine];
1′-(cyclopropylmethyl)-1,2-dihydro-5-fluoro-1-(4-fluoro-benzoyl)-spiro[3H-indole-3,4′-piperidine];
1′-(cyclopropylmethyl)-1,2-dihydro-5-fluoro-1-(2,3-difluoro-benzoyl)-spiro[3H-indole-3,4′-piperidine];
1′-(cyclopropylmethyl)-1,2-dihydro-5-fluoro-1-(2,4-difluoro-benzoyl)-spiro[3H-indole-3,4′-piperidine];
1′-(cyclopropylmethyl)-1,2-dihydro-5-fluoro-1-(2,5-difluoro-benzoyl)-spiro[3H-indole-3,4′-piperidine];
1′-cyclopropylmethyl)-1,2-dihydro-5-fluoro-1-(3-trifluoromethyl4-fluoro-benzoyl)-spiro[3H-indole-3,4′-piperidine];
1′-cyclopropylmethyl)-1,2-dihydro-5-fluoro-1-(3-trifluoromethyl-benzoyl)-spiro[3H-indole-3,4′-piperidine];
1′-cyclopropylmethyl)-1,2-dihydro-5-fluoro-1-(3-dichloromethyl-benzoyl)-spiro[3H-indole-3,4′-piperidine];
1′-cyclopropylmethyl)-1,2-dihydro-5-fluoro-1-(3,5-bis-trifluoromethyl-benzoyl)-spiro[3H-indole-3,4′-piperidine];
1′-(cyclopropylmethyl)-1,2-dihydro-5-fluoro-1-(morpholine-4-carbonyl)-spiro[3H-indole-3,4′-piperidine];
1′-(cyclopropylmethyl)-1,2-dihydro-5-fluoro-1-(piperidine-1-carbonyl)-spiro[3H-indole-3,4′-piperidine];
1′-(cyclopropylmethyl)-1,2-dihydro-5-fluoro-1-(benzo[1,3]dioxole-5-carbonyl)-spiro[3H-indole-3,4′-piperidine];
1′-(cyclopropylmethyl)-1,2-dihydro-5-fluoro-1-cyclopentylcarbonyl)-spiro[3H-indole-3,4′-piperidine];
1′-(cyclopropylmethyl)-1,2-dihydro-5-fluoro-1-(9-oxo-9H-fluorene-1-carbonyl)-spiro[3H-indole-3,4′-piperidine];
1′-(cyclopropylmethyl)-1,2-dihydro-5-fluoro-1-(cyclobutylcarbonyl)-spiro[3H-indole-3,4′-piperidine];
1′-(cyclopropylmethyl)-1,2-dihydro-5-fluoro-1-(benzoyl)-spiro[3H-indole-3,4′-piperidine];
1′-(cyclopropylmethyl)-1,2-dihydro-5-fluoro-1-(3-nitro-benzoyl)-spiro[3H-indole-3,4′-piperidine];
1′-(cyclopropylmethyl)-1,2-dihydro-5-fluoro-1-(4-nitro-benzoyl)-spiro[3H-indole-3,4′-piperidine];
1′-(cyclopropylmethyl)-1,2-dihydro-5-fluoro-1-(3-nitro4-methyl-benzoyl)-spiro[3H-indole-3,4′-piperidine];
1′-(cyclopropylmethyl)-1,2-dihydro-5-fluoro-1-(naphthylene-1-carbonyl)-spiro[3H-indole-3,4′-piperidine];
1′-(cyclopropylmethyl)-1,2-dihydro-5-fluoro-1-(naphthylene-2-carbonyl)-spiro[3H-indole-3,4′-piperidine];
1′-(cyclopropylmethyl)-1,2-dihydro-5-fluoro-1-(thiophene-2-carbonyl)-spiro[3H-indole-3,4′-piperidine];
1′-(allyl)-1,2-dihydro-5-fluoro-1-(benzenesulfonyl)-spiro[3H-indole-3,4′-piperidine];
1′-(allyl)-1,2-dihydro-5-fluoro-1-[2-(4-methoxy-phenyl)-acetyl]-spiro[3H-indole-3,4′-piperidine];
1′-(allyl)-1,2-dihydro-5-fluoro-1-(4-nitro-phenylcarbamoyl)-spiro[3H-indole-3,4′-piperidine];
1′-(cyclopropylmethyl)-1,2-dihydro-5-fluoro-1-(3-chloro-benzoyl)-spiro[3H-indole-3,4′-piperidine];
1′-(cyclopropylmethyl)-1,2-dihydro-5-fluoro-1-3,5-dichloro-benzoyl)-spiro[3H-indole-3,4′-piperidine];
1′-(cyclopropylmethyl)-1,2-dihydro-5-fluoro-1-(cyclohexylcarbonyl)-spiro[3H-indole-3,4′-piperidine];
1′-(cyclopropylmethyl)-1,2-dihydro-5-fluoro-1-(adamantane-1-carbonyl)-spiro [3H-indole-3,4′-piperidine];
1′-(cyclopropylmethyl)-1,2-dihydro-5-fluoro-1-(benzenesulfonyl)-spiro[3H-indole-3,4′-piperidine];
1′-(cyclopropylmethyl)-1,2-dihydro-5-fluoro-1-(4-chloro-benzenesulfonyl)-spiro[3H-indole-3,4′-piperidine];
1′-(cyclopropylmethyl)-1,2-dihydro-5-fluoro-1-(3-nitro-benzenesulfonyl)-spiro[3H-indole-3,4′-piperidine];
1′-(cyclopropylmethyl)-1,2-dihydro-5-fluoro-1-(4-trifluoromethyl-benzenesulfonyl)-spiro[3H-indole-3,4′-piperidine];
1′-(cyclopropylmethyl)-1,2-dihydro-5-fluoro-1-(4-fluoro-benzenesulfonyl)-spiro[3H-indole-3,4′-piperidine];
1′-(cyclopropylmethyl)-1,2-dihydro-5-fluoro-1-(ethylcarbamoyl)-spiro[3H-indole-3,4′-piperidine];
1′-(cyclopropylmethyl)-1,2-dihydro-5-fluoro-1-(cyclohexylcarbamoyl)-spiro[3H-indole-3,4′-piperidine];
1′-(cyclopropylmethyl)-1,2-dihydro-5-fluoro-1-(phenylcarbamoyl)-spiro[3H-indole-3,4′-piperidine];
1′-(cyclopropylmethyl)-1,2-dihydro-5-fluoro-1-(benzylcarbamoyl)-spiro[3H-indole-3,4′-piperidine];
1′-(cyclopropylmethyl)-1,2-dihydro-5-fluoro-1-(4-chloro-phenylcarbamoyl)-spiro[3H-indole-3,4′-piperidine];
1′-cyclopropylmethyl)-1,2-dihydro-5-fluoro-1-(4-methoxy-phenylcarbamoyl)-spiro[3H-indole-3,4′-piperidine];
1′-cyclopropylmethyl)-1,2-dihydro-5-fluoro-1-(2,3,4-tri-methoxy-phenylcarbamoyl)-spiro[3H-indole-3,4′-piperidine];
1′-cyclopropylmethyl)-1,2-dihydro-5-fluoro-1-[2-3-methoxy-phenyl)-acetyl]-spiro[3H-indole-3,4′-piperidine];
1′-(allyl)-1,2-dihydro-5-fluoro-1-(4-methoxy-benzoyl)-spiro[3H-indole-3,4′-piperidine];
1′-(allyl)-1,2-dihydro-5-fluoro-1 morpholine-4-carbonyl)-spiro[3H-indole-3,4′-piperidine];
1′-(allyl)-1,2-dihydro-5-fluoro-1-(pyrrolidine-1-carbonyl)-spiro[3H-indole-3,4′-piperidine];
1′-(allyl)-1,2-dihydro-5-fluoro-1-(2,4-dichloro-benzenesulfonyl)-spiro[3H-indole-3,4′-piperidine];
1′-(allyl)-1,2-dihydro-5-fluoro-1-(3,5-bis-trifluoromethyl-benzenesulfonyl)-spiro[3H-indole-3,4′-piperidine];
1′-(allyl)-1,2-dihydro-5-fluoro-1-(4-chloro-benzenesulfonyl)-spiro[3H-indole-3,4′-piperidine];
1′-(allyl)-1,2-dihydro-5-fluoro-1-(4-chloro-phenylcarbamoyl)-spiro[3H-indole-3,4′-piperidine];
1′-(cyclopropylmethyl)-1,2-dihydro-5-fluoro-1-(3,4-difluoro-benzoyl)-spiro[3H-indole-3,4′-piperidine];
1′-(cyclopropylmethyl)-1,2-dihydro-5-fluoro-1-(pyrrolidine-l -carbonyl)-spiro[3H-indole-3,4′-piperidine];
1′-(cyclopropylmethyl)-1,2-dihydro-5-fluoro-1-(4-chloro-benzenesulfonyl)-spiro[3H-indole-3,4′-piperidine];
1′-(cyclopropylmethyl)-1,2-dihydro-5-fluoro-1-(2,4-dichloro-benzenesulfonyl)-spiro[3H-indole-3,4′-piperidine];
1′-(cyclopropylmethyl)-1,2-dihydro-5-fluoro-1-(2-nitro-benzenesulfonyl)-spiro[3H-indole-3,4′-piperidine];
1′-(cyclopropylmethyl)-1,2-dihydro-5-fluoro-1-(2-nitro-4-trifluoromethyl-benzenesulfonyl)-spiro[3H-indole-3,4′-piperidine];
1′-(cyclopropylmethyl)-1,2-dihydro-5-fluoro-1-(3-trifluoromethyl-benzenesulfonyl)-spiro[3H-indole-3,4′-piperidine];
1′-cyclopropylmethyl)-1,2-dihydro-5-fluoro-1-(3,5-bis-trifluoromethyl-benzenesulfonyl)-spiro[3H-indole-3,4′-piperidine];
1′-(cyclopropylmethyl)-1,2-dihydro-5-fluoro-1-(4-nitro-phenylcarbamoyl)-spiro[3H-indole-3,4′-piperidine];
1′-methyl)-1,2-dihydro-5-fluoro-1-(2chloro-benzoyl)-spiro[3H-indole-3,4′-piperidine];
1′-methyl)-1,2-dihydro-5-fluoro-1-(3-dichloromethyl-benzoyl)-spiro[3H-indole-3,4′-piperidine];
1′-methyl)-1,2-dihydro-5-fluoro-1-(2-fluoro-benzoyl)-spiro[3H-indole-3,4′-piperidine];
1′-(methyl)-1,2-dihydro-5-fluoro-1-(2,3-difluoro-benzoyl)-spiro[3H-indole-3,4′-piperidine];
1′-(methyl)-1,2-dihydro-5-fluoro-1-(2-chloro-pyridine-3-carbonyl)-spiro[3H-indole-3,4′-piperidine];
1′-(allyl)-1,2-dihydro-5-methoxy-1-(2-fluoro-benzoyl)-spiro[3H-indole-3,4′-piperidine];
1′-(allyl)-1,2-dihydro-5-hydroxy-1-(2-fluoro-benzoyl)-spiro[3H-indole-3,4′-piperidine];
1′-(allyl)-1,2-dihydro-5-fluoro-1-(2-chloro-benzenesulfonyl)-spiro[3H-indole-3,4′-piperidine];
1′-(allyl)-1,2-dihydro-5-fluoro-1-(6-chloro-pyridine-3-carbonyl)-spiro[3H-indole-3,4′-piperidine];
1′-(allyl)-1,2-dihydro-5-fluoro-1-(5-bromo-pyridine-3-carbonyl)-spiro[3H-indole-3,4′-piperidine];
1′-(allyl)-1,2-dihydro-5-fluoro-1-(2-phenylsulfanyl-acetyl)-spiro[3H-indole-3,4′-piperidine];
1′-(allyl)-1,2-dihydro-5-fluoro-1-(4,5-dichloro-thiophene-2-sulfonyl)-spiro[3H-indole-3,4′-piperidine];
1′-(allyl)-1,2-dihydro-5-fluoro-1-(2,5-dichloro-thiophene-3-carbonyl)-spiro[3H-indole-3,4′-piperidine];
1′-(allyl)-1,2-dihydro-5-fluoro-1-(2,6-dichloro-5-fluoro-pyridine-3-carbonyl)-spiro[3H-indole-3,4′-piperidine];
1′-(allyl)-1,2-dihydro-5-fluoro-1-(2,5-dichloro-thiophene-3-sulfonyl)-spiro[3H-indole-3,4′-piperidine];
1′-(allyl)-1,2-dihydro-5-fluoro-1-(5-chloro-thiophene-2-sulfonyl)-spiro[3H-indole-3,4′-piperidine];
1′-(allyl)-1,2-dihydro-5-fluoro-1-(2-methylsulfanyl-pyridine-3-carbonyl)-spiro[3H-indole-3,4′-piperidine];
1′-(allyl)-1,2-dihydro-5-fluoro-1-(2,6-difluoro-benzoyl)-spiro[3H-indole-3,4′-piperidine];
1′-(allyl)-1,2-dihydro-5-fluoro-1-(2-thiophen-2-yl-acetyl)-spiro[3H-indole-3,4′-piperidine];
1′-(allyl)-1,2-dihydro-5-fluoro-1-(furan-2-carbonyl)-spiro[3H-indole-3,4′-piperidine];
1′-(allyl)-1,2-dihydro-5-fluoro-1-(2-chloro-pyridine-4-carbonyl)-spiro[3H-indole-3,4′-piperidine];
1′-(allyl)-1,2-dihydro-5-fluoro-1-(furan-3-carbonyl)-spiro[3H-indole-3,4′-piperidine];
1′-(allyl)-1,2-dihydro-5-fluoro-1-(2-thiophen-3-yl-acetyl)-spiro[3H-indole-3,4′-piperidine];
1′-(allyl)-1,2-dihydro-5-(methanesulfonyloxy)-1-(2-fluoro-benzoyl)-spiro[3H-indole-3,4′-piperidine];
1′-(allyl)-1,2-dihydro-5-fluoro-1-(5-bromo-thiophene-2-carbonyl)-spiro[3H-indole-3,4′-piperidine];
1′-(allyl)-1,2-dihydro-5-hydroxy-1-(2-fluoro-benzoyl)-spiro[3H-indole-3,4′-piperidine];
1′-(allyl)-1,2-dihydro-5-fluoro-1-(3-chloro-thiophene-2-carbonyl)-spiro[3H-indole-3,4′-piperidine];
1′-(allyl)-1,2-dihydro-5-fluoro-1-(5-chloro-thiophene-2-carbonyl)-spiro[3H-indole-3,4′-piperidine];
1′-(allyl)-1,2-dihydro-5-fluoro-1-(2,6-dichloro-pyridine-3-carbonyl)-spiro[3H-indole-3,4′-piperidine];
1′-(allyl)-1,2-dihydro-5-fluoro-1-(2,6-dichloro-pyridine-4-carbonyl)-spiro[3H-indole-3,4′-piperidine];
1′-(allyl)-1,2-dihydro-5-fluoro-1-(2-fluoro-benzenesulfonyl)-spiro[3H-indole-3,4′-piperidine];
1′-(allyl)-1,2-dihydro-5-fluoro-1-(2-mercapto-pyridine-3-carbonyl)-spiro[3H-indole-3,4′-piperidine];
1′-(allyl)-1,2-dihydro-5-fluoro-1-(5-nitro-1H-pyrazole-3-carbonyl)-spiro[3H-indole-3,4′-piperidine];
1′-(allyl)-1,2dihydro-5-fluoro-1-(2-allylsulfanyl-pyridine-3-carbonyl)-spiro[3H-indole-3,4′-piperidine];
1′-(allyl)-1,2-dihydro-5-fluoro-1-(1H-imidazole-4-carbonyl)-spiro[3H-indole-3,4′-piperidine];
1′-(allyl)-1,2-dihydro-5-fluoro-1-(4-nitro-1H-pyrazole-3-carbonyl)-spiro[3H-indole-3,4′-piperidine];
1′-(allyl)-1,2-dihydro-5-chloro-1-(2-fluoro-benzoyl)-spiro[3H-indole-3,4′-piperidine];
1′-(phenyl)-1,2-dihydro-5-chloro-1-(2-fluoro-benzoyl)-spiro[3H-indole-3,4′-piperidine];
1′-(naphthylene-1-carbonyl)-1,2-dihydro-5-chloro-1-(phenethyl)-spiro[3H-indole-3,4′-piperidine];
1′-(2-chlorophenyl)-1,2-dihydro-5-chloro-1-(2-fluoro-benzoyl)-spiro[3H-indole-3,4′-piperidine];
1′-(3-chlorophenyl)-1,2-dihydro-5-chloro-1-(2-fluoro-benzoyl)-spiro[3H-indole-3,4′-piperidine];
1′-(4-chlorophenyl)-1,2-dihydro-5-chloro-1-(2-fluoro-benzoyl)-spiro[3H-indole-3,4′-piperidine];
1′-(3,4-dichlorophenyl)-1,2-dihydro-5-chloro-1-(2-fluoro-benzoyl)-spiro[3H-indole-3,4′-piperidine];
1′-(4-methylphenyl)-1,2-dihydro-5-chloro-1-(2-fluoro-benzoyl)-spiro[3H-indole-3,4′-piperidine];
1′-(2-fluorophenyl)-1,2-dihydro-5-chloro-1-(2-fluoro-benzoyl)-spiro[3H-indole-3,4′-piperidine];
1′-(6-fluoro-pyridin-3-yl)-1,2-dihydro-5-chloro-1-(2-fluoro-benzoyl)-spiro[3H-indole-3,4′-piperidine];
1′-(4-trifluoromethyl-phenyl)-1,2dihydro-5-chloro-1-2-fluoro-benzoyl)-spiro[3H-indole-3,4′-piperidine];
1′-(allyl)-1,2-dihydro-5,7-dimethyl-1-(2,3-difluoro-benzoyl)-spiro[3H-indole-3,4′-piperidine];
1′-(allyl)-1,2-dihydro-5,7-dimethyl-1-(2,6difluoro-benzoyl)-spiro[3H-indole-3,4′-piperidine];
1′-(allyl)-1,2-dihydro-5,7-dimethyl-1-(2-chloro-benzenesulfonyl)-spiro[3H-indole-3,4′-piperidine];
1′-propyl)-1,2-dihydro-5,7-dimethyl-1-(2-chloro-benzenesulfonyl)-spiro[3H-indole-3,4′-piperidine];
1′-(propyl)-1,2-dihydro-5-methyl-1-(2,3-difluoro-benzoyl)-spiro[3H-indole-3,4′-piperidine];
1′-(allyl)-1,2-dihydro-5-fluoro-1-(2,3-difluoro-benzoyl)-spiro[3H-indole-3,4′-piperidine];
1′-(propyl)-1,2-dihydro-5-methyl-1-(2,6-difluoro-benzoyl)-spiro[3H-indole-3,4′-piperidine];
1′-(allyl)-1,2-dihydro-5-trifluoromethyl-1-(2-chloro-benzenesulfonyl)-spiro[3H-indole-3,4′-piperidine];
1′-(allyl)-1,2-dihydro-5-methoxy-7-methyl-1-(2,3-difluoro-benzoyl)-spiro[3H-indole-3,4′-piperidine];
1′-(allyl)-1,2-dihydro-5-methoxy-7-methyl-1-(2,6-difluoro-benzoyl)-spiro[3H-indole-3,4′-piperidine];
1′-(allyl)-1,2-dihydro-6,7-dimethyl-1-(2,6-difluoro-benzoyl)-spiro[3H-indole-3,4′-piperidine];
1′-(allyl)-1,2-dihydro-6,7-dimethyl-1-(2,3-difluoro-benzoyl)-spiro[3H-indole-3,4′-piperidine];
1′-(allyl)-1,2-dihydro-4,7-dimethyl-1-(2,3-difluoro-benzoyl)-spiro[3H-indole-3,4′-piperidine];
1′-(allyl)-1,2-dihydro-4,7-dimethyl-1-(2,6-difluoro-benzoyl)-spiro[3H-indole-3,4′-piperidine];
1′-(allyl)-1,2-dihydro-5-isopropyl-1-(2,6-difluoro-benzoyl)-spiro[3H-indole-3,4′-piperidine];
1′-(allyl)-1,2-dihydro-5-isopropyl-1-(2,3-difluoro-benzoyl)-spiro[3H-indole-3,4′-piperidine];
1′-(3-methoxycarbonyl-propionyl)-1,2-dihydro-5-fluoro-1-(2,6-fluoro-benzoyl)-spiro[3H-indole-3,4′-piperidine];
1′-(3-carboxy-propionyl)-1,2-dihydro-5-fluoro-1-2,6-difluoro-benzoyl)-spiro[3H-indole-3,4′-piperidine];
1′-(1-pent-4-enyl)-1,2-dihydro-5-chloro-1-(2,6-difluoro-benzoyl)-spiro[3H-indole-3,4′-piperidine];
1′-2-phenoxy-ethyl)-1,2-dihydro-5-chloro-1-2,6-difluoro-benzoyl)-spiro[3H-indole-3,4′-piperidine];
1′-(3,3-dimethyl-2-oxo-butyl)-1,2-dihydro-5-chloro-1-(2,6-difluoro-benzoyl)-spiro[3H-indole-3,4′-piperidine];
1′-2-ethoxy-ethyl)-1,2-dihydro-5-chloro-1-(2,6-difluoro-benzoyl)-spiro[3H-indole-3,4′-piperidine];
1′-(1-phenyl-ethyl)-1,2-dihydro-5-chloro-1-(2,6-difluoro-benzoyl)-spiro[3H-indole-3,4′-piperidine];
1′-(2-carboxy-allyl)-1,2-dihydro-5-chloro-1-(2,6-difluoro-benzoyl)-spiro[3H-indole-3,4′-piperidine];
1′-(2-carboxy-allyl)-1,2-dihydro-5-chloro-1-(2,3-difluoro-benzoyl)-spiro[3H-indole-3,4′-piperidine];
1′-(1-pent4-enyl)-1,2-dihydro-5-chloro-1-(2,3-difluoro-benzoyl)-spiro[3H-indole-3,4′-piperidine];
1′-(tetrahydro-pyran-2-ylmethyl)-1,2-dihydro-5-chloro-1-(2,6-difluoro-benzoyl)-spiro[3H-indole-3,4′-piperidine];
1′-(3-methyl-but-2-enyl)-1,2-dihydro-5-chloro-1-(2,6-difluoro-benzoyl)-spiro[3H-indole-3,4′-piperidine];
1′-(3-methyl-but-2-enyl)-1,2-dihydro-5-chloro-1-(2,3-difluoro-benzoyl)-spiro[3H-indole-3,4′-piperidine];
1′-(2-oxo-butyl)-1,2-dihydro-5-chloro-1-(2,6-difluoro-benzoyl)-spiro[3H-indole-3,4′-piperidine];
1′-(2-oxo-butyl)-1,2-dihydro-5-chloro-1-(2,3-difluoro-benzoyl)-spiro[3H-indole-3,4′-piperidine];
1′-(2-oxo-2-phenyl-ethyl)-1,2-dihydro-5chloro-1-(2,6-difluoro-benzoyl)-spiro[3H-indole-3,4′-piperidine];
1′-(2-oxo-2-phenyl-ethyl)-1,2-dihydro-5-chloro-1-(2,3-difluoro-benzoyl)-spiro[3H-indole-3,4′-piperidine];
1′-(1-methyl-2-phenyl-ethyl)-1,2-dihydro-5-chloro-1-(2,6-difluoro-benzoyl)-spiro[3H-indole-3,4′-piperidine];
1′-([1,3]dioxolan-2-ylmethyl)-1,2-dihydro-5-chloro-1-(2,6-difluoro-benzoyl)-spiro[3H-indole-3,4′-piperidine];
1′-[2-(4-methoxy-phenyl)-2-oxo-ethyl]-1,2-dihydro-5-chloro-1-(2,6-difluoro-benzoyl)-spiro[3H-indole-3,4′-piperidine];
1′-[2-(4-methoxy-phenyl)-2-oxo-ethyl]-1,2-dihydro-5-chloro-1-2,3-difluoro-benzoyl)-spiro[3H-indole-3,4′-piperidine];
1′-ethoxycarbonyl methyl)-1,2-dihydro-5-chloro-1-(2,6-difluoro-benzoyl)-spiro[3H-indole-3,4′-piperidine];
1′-ethoxycarbonyl methyl)-1,2-dihydro-5-chloro-1-(2,3-difluoro-benzoyl)-spiro[3H-indole-3,4′-piperidine];
1′-(cyclobutylmethyl)-1,2-dihydro-5-chloro-1-(2,6-difluoro-benzoyl)-spiro[3H-indole-3,4′-piperidine];
1′-[2-(4-chloro-phenyl)-2-oxo-ethyl]-1,2-dihydro-5-chloro-1-(2,3-difluoro-benzoyl)-spiro[3H-indole-3,4′-piperidine];
1′-[3-(1,3-dioxo-1,3-dihydro-isoindol-2-yl)-propyl]-1,2-dihydro-5-chloro-1-(2,6-difluoro-benzoyl)-spiro[3H-indole-3,4′-piperidine];
1′-[3-(1,3-dioxo-1,3-dihydro-isoindol-2-yl)-propyl]-1,2-dihydro-5-chloro-1-(2,3-difluoro-benzoyl)-spiro[3H-indole-3,4′-piperidine];
1′-[4-(1,3-dioxo-1,3-dihydro-isoindol-2-yl)-butyl]-1,2-dihydro-5-chloro-1-(2,3-difluoro-benzoyl)-spiro[3H-indole-3,4′-piperidine];
1′-[2-(1H-indol-3-yl)-ethyl]-1,2-dihydro-5-chloro-1-(2,6-difluoro-benzoyl)-spiro[3H-indole-3,4′-piperidine];
1′-(2-methylsulfanyl-propyl)-1,2-dihydro-5-chloro-1-(2,6-difluoro-benzoyl)-spiro[3H-indole-3,4′-piperidine];
1′-(2-methylsulfanyl-propyl)-1,2-dihydro-5-chloro-1-(2,3-difluoro-benzoyl)-spiro[3H-indole-3,4′-piperidine];
1′-(3-methylsulfanyl-propyl)-1,2-dihydro-5-chloro-1-(2,6-difluoro-benzoyl)-spiro[3H-indole-3,4′-piperidine];
1′-(2-chloro4-fluoro-benzyl)-1,2-dihydro-5-chloro-1-(2,6-difluoro-benzoyl)-spiro[3 H-indole-3,4′-piperidine];
1′-(2-chloro-4-fluoro-benzyl)-1,2-dihydro-5-chloro-1-(2,3-difluoro-benzoyl)-spiro[3H-indole-3,4′-piperidine];
1′-(2,4-dichloro-benzyl)-1,2-dihydro-5-chloro-1-(2,6-difluoro-benzoyl)-spiro[3H-indole-3,4′-piperidine];
1′-(2,4-dichloro-benzyl)-1,2-dihydro-5-chloro-1-(2,3-difluoro-benzoyl)-spiro[3H-indole-3,4′-piperidine];
1′-4-trifluoromethyl-benzyl)-1,2-dihydro-5-chloro-1-(2,6-difluoro-benzoyl)-spiro[3H-indole-3,4′-piperidine];
1′-4-trifluoromethyl-benzyl)-1,2-dihydro-5-chloro-1-(2,3-difluoro-benzoyl)-spiro[3H-indole-3,4′-piperidine];
1′-(4-tert-butyl-benzyl)-1,2-dihydro-5-chloro-1-(2,6-difluoro-benzoyl)-spiro[3H-indole-3,4′-piperidine];
1′-4-tert-butyl-benzyl)-1,2-dihydro-5-chloro-1-(2,3-difluoro-benzoyl)-spiro[3H-indole-3,4′-piperidine];
1′-(3-chloro-benzyl)-1,2-dihydro-5-chloro-1-(2,6-difluoro-benzoyl)-spiro[3H-indole-3,4′-piperidine];
1′-(3-chloro-benzyl)-1,2-dihydro-5-chloro-1-(2,3-difluoro-benzoyl)-spiro[3H-indole-3,4′-piperidine];
1′-(pent-4-ynyl)-1,2-dihydro-5-chloro-1-(2,6-difluoro-benzoyl)-spiro[3H-indole-3,4′-piperidine];
1′-[2-(4-chloro-phenyl)-2-oxo-ethyl]-1,2-dihydro-5-chloro-1-(2,6-difluoro-benzoyl)-spiro[3H-indole-3,4′-piperidine];
1′-(3-methylsulfanyl-propyl)-1,2-dihydro-5-chloro-1-(2,3-difluoro-benzoyl)-spiro[3H-indole-3,4′-piperidine];
1′-(1H-pyrrol-2-ylmethyl)-1,2-dihydro-5-chloro-1-(2,6-difluoro-benzoyl)-spiro[3H-indole-3,4′-piperidine];
1′-(thiophen-3-ylmethyl)-1,2-dihydro-5-chloro-1-(2,6-difluoro-benzoyl)-spiro[3H-indole-3,4′-piperidine];
1′-(thiophen-2-ylmethyl)-1,2-dihydro-5-chloro-1-(2,6-difluoro-benzoyl)-spiro[3H-indole-3,4′-piperidine];
1′-(furan-3-ylmethyl)-1,2-dihydro-5-chloro-1-(2,6-difluoro-benzoyl)-spiro[3H-indole-3,4′-piperidine];
1′-(3-amino-propyl)-1,2-dihydro-5-chloro-1-(2,3-difluoro-benzoyl)-spiro[3H-indole-3,4′-piperidine];
1′-(4-amino-butyl)-1,2-dihydro-5-chloro-1-(2,3-difluoro-benzoyl)-spiro[3H-indole-3,4′-piperidine];
1′-(cyclobutyl)-1,2-dihydro-5-chloro-1-(2,6-difluoro-benzoyl)-spiro[3H-indole-3,4′-piperidine];
1′-(cyclopentylmethyl)-1,2-dihydro-5-chloro-1-(2,6-difluoro-benzoyl)-spiro[3H-indole-3,4′-piperidine];
1′-(cyclohexylmethyl)-1,2-dihydro-5-chloro-1-(2,6-difluoro-benzoyl)-spiro[3H-indole-3,4′-piperidine];
1′-(cyclohex-3-enylmethyl)-1,2-dihydro-5-chloro-1-(2,6-difluoro-benzoyl)-spiro[3H-indole-3,4′-piperidine];
1′-(but-3-enyl)-1,2-dihydro-5-chloro-1-(2,6-difluoro-benzoyl)-spiro[3H-indole-3,4′-piperidine];
1′-(3,4,4-trifluoro-but-3-enyl)-1,2-dihydro-5-chloro-1-2,6-difluoro-benzoyl)-spiro[3H-indole-3,4′-piperidine];
1′-(hex-5-enyl)-1,2-dihydro-5-chloro-1-(2,6-difluoro-benzoyl)-spiro[3H-indole-3,4′-piperidine];
1′-(cyclobutylmethyl)-1,2-dihydro-5-isopropyl-1-(2,6-difluoro-benzoyl)-spiro[3H-indole-3,4′-piperidine];
1′-(cyclobutylmethyl)-1,2-dihydro-5,6-dimethyl-1-(2,6-difluoro-benzoyl)-spiro[3H-indole-3,4′-piperidine];
1′-(cyclobutylmethyl)-1,2-dihydro-5-methyl-1-(2,6-difluoro-benzoyl)-spiro[3H-indole-3,4′-piperidine];
1′-(cyclobutylmethyl)-1,2-dihydro-6-methyl-1-(2,6-difluoro-benzoyl)-spiro[3H-indole-3,4′-piperidine];
1′-(cyclobutylmethyl)-1,2-dihydro-5-tert-butyl-1-(2,6-difluoro-benzoyl)-spiro[3H-indole-3,4′-piperidine];
1′-(but-3-enyl)-1,2-dihydro-5,6-dimethyl-1-(2,6-difluoro-benzoyl)-spiro[3H-indole-3,4′-piperidine];
1′-(but-3-enyl)-1,2-dihydro-5-methyl-1-(2,6-difluoro-benzoyl)-spiro[3H-indole-3,4′-piperidine];
1′-(but-3-enyl)-1,2-dihydro-6-methyl-1-(2,6-difluoro-benzoyl)-spiro[3H-indole-3,4′-piperidine];
1′-(but-3-enyl)-1,2-dihydro-5-tert-butyl-1-(2,6-difluoro-benzoyl)-spiro[3H-indole-3,4′-piperidine];
1′-(but-3-enyl)-1,2-dihydro-5-isopropyl-1-(2,6-difluoro-benzoyl)-spiro[3H-indole-3,4′-piperidine];
1′-(allyl)-1,2-dihydro-1-(2-fluoro-benzoyl)-spiro[3H-indole-3,4′-piperidine];
1′-(allyl)-1,2-dihydro-1-(2-chloro-benzoyl)-spiro[3H-indole-3,4′-piperidine];
1′-(allyl)-1,2-dihydro-1-(2,3-difluoro-benzoyl)-spiro[3H-indole-3,4′-piperidine]; and
1′-(allyl)-1,2-dihydro-1-(2,6-difluoro-benzoyl)-spiro[3H-indole-3,4′-piperidine];
or a pharmaceutically acceptable salt thereof.

14. A compound of claim 1, selected from the group consisting of:
1′-(butyl)-2,3-dihydro-2-(cyclobutylcarbonyl)-spiro[isoquinoline-4(1H),4′-piperidine];
1′-butyl)-2,3-dihydro-2-diphenylacetyl)-spiro[isoquinoline-4(1H),4′-piperidine];
1′-(phenethyl)-2,3-dihydro-2-cyclobutylcarbonyl)-spiro[isoquinoline-4(1H),4′-piperidine];
1′-(phenethyl)-2,3-dihydro-2-(diphenylacetyl)-spiro[isoquinoline-4(1H),4′-piperidine];
1′-(cyclobutylcarbonyl)-2,3-dihydro-2-(butyl)-spiro[isoquinoline-4(1H),4′-piperidine];
1′-(cyclobutylcarbonyl)-2,3-dihydro-2-(phenethyl)-spiro[isoquinoline4(1H),4′-piperidine];
1′-(dipenylacetyl)-2,3-dihydro-2-(butyl)-spiro[isoquinoline-4(1H),4′-piperidine];
1′-(diphenylacetyl)-2,3-dihydro-2-(phenethyl)-spiro[isoquinoline-4(1H),4′-piperidine];
1′-(butyl)-2,3-dihydro-2-(cyclobutylcarbonyl)-spiro[isoquinoline4(1H),4′-piperidine];
1′-(3,4-dichloro-benzyl)-2,3-dihydro-2-(cyclobutylcarbonyl)-spiro[isoquinoline-4(1H),4′-piperidine];
1′-(2,4-dimethyl-benzyl)-2,3-dihydro-2-(cyclobutylcarbonyl)-spiro[isoquinole-4(1H),4′-piperidine];
1′-(butyl)-2,3-dihydro-2-(diphenylacetyl)-spiro[isoquinoline4(1H),4′-piperidine];
1′-(butyl)-2,3-dihydro-2-(naphthylene-1-carbonyl)-spiro[isoquinoline4(1H),4′-piperidine];
1′-(benzo[1,3]dioxol-4-ylmethyl)-2,3-dihydro-2-(naphthylene-1-carbonyl)-spiro[isoquinoline4(1H),4′-piperidine];
1′-(2,4-dimethyl-benzyl)-2,3-dihydro-2-(naphthylene-1-carbonyl)-spiro[isoquinoline4(1H),4′-piperidine];
1′-(tert-butoxycarbonyl)-2,3-dihydro-2-(4-chloro-benzenesulfonyl)-spiro[isoquinoline4(1H),4′-piperidine];
1′-(tert-butoxycarbonyl)-2,3-dihydro-2-(diphenylacetyl)-spiro[isoquinoline-4(1H),4′-piperidine];
1′-(benzo[1,3]dioxol4-ylmethyl)-2,3-dihydro-2-(4-chloro-benzenesulfonyl)-spiro[isoquinoline4(1H),4′-piperidine];
2,3-dihydro-2-(4-chloro-benzenesulfonyl)-spiro[isoquinoline4(1H),4′-piperidine];
1′-(butyl)-2,3-dihydro-2-isobutyryl)-spiro[isoquinoline4(1 H),4′-piperidine];
1′-(benzo[1,3]dioxol-4-ylmethyl)-2,3-dihydro-2-isobutyryl)-spiro[isoquinoline-4(1H),4′-piperidine];
1′-2,4-dimethyl-benzyl)-2,3-dihydro-2′-isobutyryl)-spiro[isoquinoline4(1H),4′-piperidine];
1′-(phenethyl)-2,3dihydro-2-(4-chloro-benzenesulfonyl)-spiro[isoquinoline-4(1H),4′-piperidine];
2,3-dihydro-2-(naphthylene-1-carbonyl)-spiro[isoquinoline4(1H),4′-piperidine];
1′-(butyl)-2,3-dihydro-2-(4-chloro-benzenesulfonyl)-spiro[isoquinoline-4(H),4′-piperidine];
2,3-dihydro-2-(diphenylacetyl)-spiro[isoquinoline4(1H),4′-piperidine];
1′-(cyclobutylcarbonyl)-2,3-dihydro-2-(cyclopropylmethyl)-spiro[isoquinoline-4(1H),4′-piperidine];
1′-(2-chloro-benzoyl)-2,3-dihydro-2-(cyclopropylmethyl)-spiro[isoquinoline-4(1H),4′-piperidine];
1′-(4-nitro-benzenesulfonyl)-2,3-dihydro-2-(cyclopropylmethyl)-spiro[isoquinoline-4(1H),4′-piperidine];
1′-(diphenylacetyl)-2,3-dihydro-2-(cyclopropylmethyl)-spiro[isoquinoline-4(1H),4′-piperidine];
1′-(cyclobutylcarbonyl)-2,3-dihydro-2-(phenethyl)-spiro[isoquinoline-4(1H),4′-piperidine];
1′-(4-nitro-benzenesulfonyl)-2,3-dihydro-2-(phenethyl)-spiro[isoquinoline-4(1H),4′-piperidine];
1′-(4-methoxy-benzoyl)-2,3-dihydro-2-(cyclopropylmethyl)-spiro[isoquinoline-4(1H),4′-piperidine];
1′-(2-chloro-benzoyl)-2,3-dihydro-2-(phenethyl)-spiro[isoquinoline-4(1H),4′-piperidine];
1′-(4-methoxy-benzoyl)-2,3-dihydro-2-(phenethyl)-spiro[isoquinoline-4(1H),4′-piperidine];
2,3-dihydro-2-(phenethyl)-spiro[isoquinoline-4(1 H),4′-piperidine];
1′-{4-[(4-chloro-benzenesulfonyl)-methyl-amino]-3-(3-chloro-phenyl)-butyl}-2,3-dihydro-2-(tert-butoxycarbonyl)-spiro[isoquinoline-4(1H),4′-piperidine];
1∝-{4-[(4-chloro-benzenesulfonyl)-methyl-amino]-3-phenyl-butyl}-2,3-dihydro-2-(tert-butoxycarbonyl)-spiro[isoquinoline-4(1H),4′-piperidine];
1′-{4-[(3,4-difluoro-benzenesulfonyl)-methyl-amino]-3-3-chloro-phenyl)-butyl}-2,3-dihydro-2-(tert-butoxycarbonyl)-spiro[isoquinoline-4(1H),4′-piperidine];
1′-{4-[(3,4-difluoro-benzenesulfonyl)-methyl-amino]-3-phenyl-butyl}-2,3-dihydro-2-tert-butoxycarbonyl)-spiro[isoquinoline-4(1H),4′-piperidine];
1′-{4-[(benzenesulfonyl)-methyl-amino]-3-3-chloro-phenyl)-butyl}-2,3-dihydro-2-(tert-butoxycarbonyl)-spiro[isoquinoline-4(1H),4′-piperidine];
1′-{4-[(benzenesulfonyl)-methyl-amino]-3-phenyl-butyl}-2,3-dihydro-2-(tert-butoxycarbonyl)-spiro[isoquinoline-4(1H),4′-piperidine];
1′-(allyl)-2,3-dihydro-2-thiophene-3-carbonyl)-spiro[isoquinoline-4(1H),4′-piperidine];
1′-(allyl)-2,3-dihydro-2-(5-chloro-thiophene-2-carbonyl)-spiro[isoquinoline-4(1H),4′-piperidine];
1′-(allyl)-2,3-dihydro-2-(5-bromo-thiophene-2-carbonyl)-spiro[isoquinoline-4(1H),4′-piperidine];
1′-(allyl)-2,3-dihydro-2-(2-allylsulfanyl-pyridine-3-carbonyl)-spiro[isoquinoline-4(1H),4′-piperidine];
1′-(allyl)-2,3-dihydro-2-(2,6-difluoro-benzoyl)-spiro[isoquinoline-4(1H),4′-piperidine];
1′-(allyl)-2,3-dihydro-2-(2-fluoro-benzoyl)-spiro[isoquinoline-4(1H),4′-piperidine];
1′-(allyl)-2,3-dihydro-2-(2,3-difluoro-benzoyl)-spiro[isoquinoline-4(1H),4′-piperidine];
1′-(allyl)-2,3-dihydro-2-(2,4-difluoro-benzoyl)-spiro[isoquinoline-4(1H),4′-piperidine];
1′-(allyl)-2,3-dihydro-2-(3,5-bis-ditrifluoromethyl-benzoyl)-spiro[isoquinoline-4(1H),4′-piperidine];
1′-(allyl)-2,3-dihydro-2-(thiophene-2-carbonyl)-spiro[isoquinoline-4(1,4′-piperidine];
1′-(allyl)-2,3-dihydro-2-(3-methyl4-nitro-benzoyl)-spiro[isoquinoline-4(1H),4′-piperidine];
1′-(allyl)-2,3-dihydro-2-(2-chloro-benzoyl)-spiro[isoquinoline-4(1H),4′-piperidine];
1′-(allyl)-2,3-dihydro-2-(3-chloro-benzoyl)-spiro[isoquinoline-4(1H),4′-piperidine];
1′-(allyl)-2,3-dihydro-2-(2,6-difluoro-benzenesulfonyl)-spiro[isoquinoline-4(1H),4′-piperidine];
1′-(allyl)-2,3-dihydro-2-2-chloro-benzenesulfonyl)-spiro[isoquinoline-4(1H),4′-piperidine]; and
1′-(allyl)-2,3-dihydro-2-(2-methylsulfanyl-pyridine-3-carbonyl)-spiro[isoquinoline-4(1H),4′-piperidine];
or a pharmaceutically acceptable salt thereof.

15. A compound of claim 1 or 2, wherein said compound is cardio-protective.

16. The compound of claim 15, wherein said compound does not significantly increase blood pressure.

17. A compound of claim 1 or 2, wherein said compound is neuro-protective.

18. (canceled)

19. A method for treating or preventing a vascular or cardiovascular disease or disorder comprising administering to a patient in need thereof an effective amount of a compound of Formula (I): 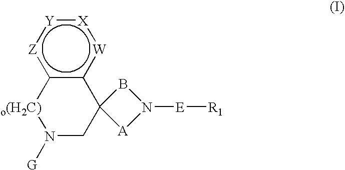
or a pharmaceutically acceptable salt, free base, solvate, hydrate or stereoisomer, thereof, wherein:
R₁ is H, halogen, hydroxy, nitro, cyano, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, substituted or unsubstituted -(3 to 7) membered heterocycle, substituted or unsubstituted -(7 to 10) membered bicycloheterocycle, substituted or unsubstituted -(5 to 10) membered heteroaryl, —NR₂R₂′, —C(═O)—R₇, —S(═O)₂—R₇, —C(═O)O—R₇, or —C(═O)N(R₇)(C₁₋₆ alkyl);
A is a substituted or unsubstituted C₁-C₃ alkylene;
B is a substituted or unsubstituted C₁-C₃ alkylene;
E is a bond, or a substituted or unsubstituted C₁-C₃ alkylene;
G is H, —Ar, —C(═O)—Ar, —C(═O)O—Ar, substituted or unsubstituted —C(═O)O—C₁₋₄ alkyl, —C(═O)N(R₇)(Ar), substituted or unsubstituted —C(═O)N(R₇)(C₁₋₆ alkyl), —S(═O)₂—Ar, substituted or unsubstituted —S(═O)₂—C₁₋₆ alkyl, substituted or unsubstituted C₁₋₆ alkyl, substituted or unsubstituted C₁₋₆ alkyl-Ar, substituted or unsubstituted —C(═O)C₁₋₆ alkyl-Ar, or substituted or unsubstituted —C(═O)C₁₋₆ alkyl;
W is N or —CR₃—;
X is N or —CR₄—;
Y is N or —CR₅—;
Z is N or —CR₆—;
R₂, R₂′, R₃, R₄, R₅, R₆ and R₇ are at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, —C(═O)—O—C₁₋₆ alkyl, —O—C₁₋₆ alkyl, —C₁₋₆ alkyl-O—C₁₋₆ alkyl, —C₁₋₆ alkyl-NH₂, —C₀₋₆ alkyl-C(═O)—NH(C₁₋₆ alkyl), —C₀₋₆ alkyl-C(═O)—N(C₁₋₆ alkyl)(C₁₋₆ alkyl), —C₁₋₆ alkyl-NH—C(═O)—C₁₋₆ alkyl, —C₁₋₆ alkyl-S(═O)—C₁₋₆ alkyl, —C₀₋₆ alkyl-O—S(═O)₂—C₁₋₆ alkyl, —C₁₋₆ alkyl-S(═O)₂—C₁₋₆ alkyl, —C₁₋₆ alkyl-NR′—S(═O)₂—R′, —C₁₋₆ alkyl-SH, —C₁₋₆ alkyl-S—C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —(C(R′)₂)₀₋₆—O—(C(R′)₂)₁₋₅C(R′)₃, —(C(R′)₂)₁₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂—(C(R′)₂)₁₋₅C(R′)₃;
o is 0 or 1;
R′ is at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted aryl, or substituted or unsubstituted C₃₋₈ cycloalkyl; and
Ar is substituted or unsubstituted aryl, substituted or unsubstituted C₃₋₇ cycloalkyl, substituted or unsubstituted C₈₋₁₄ bicycloalkyl, substituted or unsubstituted C₈₋₁₄ tricycloalkyl, substituted or unsubstituted -(3 to 7) membered heterocycle, substituted or unsubstituted -(7 to 10) membered bicycloheterocycle, or substituted or unsubstituted -(5 to 10 membered)heteroaryl.

20. The method of claim 19, wherein E is —(CH₂)ₚ— and p is 0, 1 or 2.

21. The method of claim 20, wherein W is —CR₃—, X is —CR₄—, Y is —CR₅— and Z is —CR₆.

22. The method of claim 20, wherein A and B are both —(CH₂)₂—.

23. The method of claim 20, wherein p is 1 and R₁ is —CH═CH₂.

24. The method of claim 20, wherein p is 1 and R₁ is -cyclopropyl.

25. The method of claim 20, wherein R₁ is phenyl.

26. The method of claim 20, wherein G is —C(═O)—Ar.

27. The method of claim 20, wherein G is —C(═O)NH—Ar.

28. The method of claim 20, wherein G is —S(═O)₂—Ar.

29. The method of claim 20, wherein Ar is phenyl.

30. The method of claim 20, wherein o is 0.

31. The method of claim 20, wherein said compound is cardio-protective.

32. The method of claim 31, wherein said compound does not significantly increase blood pressure.

33. The method of claim 19 or 20, wherein the vascular or cardiovascular disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine.

34. A method for treating or preventing a neurological disease or disorder comprising administering to a patient in need thereof an effective amount of a compound of Formula (I): 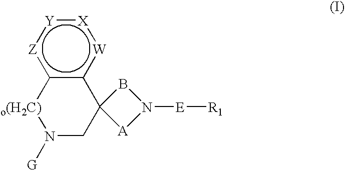
or a pharmaceutically acceptable salt, free base, solvate, hydrate or stereoisomer, thereof, wherein:
R₁ is H, halogen, hydroxy, nitro, cyano, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, substituted or unsubstituted -(3 to 7) membered heterocycle, substituted or unsubstituted -(7 to 10) membered bicycloheterocycle, substituted or unsubstituted -(5 to 10) membered heteroaryl, —NR₂R₂′, —C(═O)—R₇, —S(═O)₂—R₇, —C(═O)O—R₇, or —C(═O)N(R₇)(C₁₋₆ alkyl);
A is a substituted or unsubstituted C₁-C₃ alkylene;
B is a substituted or unsubstituted C₁-C₃ alkylene;
E is a bond, or a substituted or unsubstituted C₁-C₃ alkylene;
G is H, —Ar, —C(═O)—Ar, —C(═O)O—Ar, substituted or unsubstituted —C(═O)O—C₁₋₆ alkyl, —C(═O)N(R₇)(Ar), substituted or unsubstituted —C(═O)N(R₇)(C₁₋₆ alkyl), —S(═O)₂—Ar, substituted or unsubstituted —S(═O)₂—C₁₋₆ alkyl, substituted or unsubstituted C₁₋₆ alkyl, substituted or unsubstituted C₁₋₆ alkyl-Ar, substituted or unsubstituted —C(═O)C₁₋₆ alkyl-Ar, or substituted or unsubstituted —C(═O)C₁₋₆ alkyl;
W is N or —CR₃—;
X is N or —CR₄—;
Y is N or —CR₅—;
Z is N or —CR₆—;
R₂, R₂′, R₃, R₄, R₅, R₆ and R₇ are at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, —C(═O)—O—C₁₋₆ alkyl, —O—C₁₋₆ alkyl, —C₁₋₆ alkyl-O—C₁₋₆ alkyl, —C₁₋₆ alkyl-NH₂, —C₀₋₆ alkyl-C(═O)—NH(C₁₋₆ alkyl), —C₀₋₆ alkyl-C(═O)—N(C₁₋₆ alkyl)(C₁₋₆ alkyl), —C₁₋₆ alkyl-NH—C(═O)—C₁₋₆ alkyl, —C₁₋₆ alkyl-S(═O)—C₁₋₆ alkyl, —C₀₋₆ alkyl-O—S(═O)₂—C₁₋₆ alkyl, —C₁₋₆ alkyl-S(═O)₂—C₁₋₆ alkyl, —C₁₋₆ alkyl-NR′—S(═O)₂—R′, —C₁₋₆ alkyl-SH, —C₁₋₆ alkyl-S—C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —(C(R′)₂)₀₋₆—O—(C(R′)₂)₁₋₅C(R′)₃, —(C(R′)₂)₁₋₅C(R′)₃, —(C(R′)₂)₀₋₆—S—(C(R′)₂)₁₋₅C(R′)₃, —(C(R′)₂)₀₋₆—S(═O)—(C(R′)₂₁₋₅C(R′)₃ or —(C(R′)₂)₀₋₆—S(═O)₂—(C(R′)₂)₁₋₅C(R′)₃;
o is 0 or 1;
R′ is at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted aryl, or substituted or unsubstituted C₃₋₈ cycloalkyl; and
Ar is substituted or unsubstituted aryl, substituted or unsubstituted C₃₋₇ cycloalkyl, substituted or unsubstituted C₈₋₁₄ bicycloalkyl, substituted or unsubstituted C₈₋₁₄ tricycloalkyl, substituted or unsubstituted -(3 to 7) membered heterocycle, substituted or unsubstituted -(7 to 10) membered bicycloheterocycle, or substituted or unsubstituted -(5 to 10 membered)heteroaryl.

35. The method of claim 34, wherein E is —(CH₂)ₚ— and p is 0, 1 or 2.

36. The method of claim 35, wherein W is —CR₃—, X is —CR₄—, Y is —CR₅— and Z is —CR₆.

37. The method of claim 35, wherein A and B are both —(CH₂)₂—.

38. The method of claim 35, wherein p is 1 and R₁ is —CH═CH₂—.

39. The method of claim 35, wherein p is 1 and R₁ is -cyclopropyl.

40. The method of claim 35, wherein R₁ is phenyl.

41. The method of claim 35, wherein G is —C(═O)—Ar.

42. The method of claim 35, wherein G is —C(═O)NH—Ar.

43. The method of claim 35, wherein G is —S(═O)₂—Ar.

44. The method of claim 35, wherein Ar is phenyl.

45. The method of claim 35, wherein o is 0.

46. The method of claim 34 or 35, wherein the neurological disease or disorder is diabetic peripheral neuropathy, pain, stroke, cerebral ischemia or Parkinson's disease.

47. A method for treating or preventing a disorder treatable or preventable by inhibiting Mas receptor function, comprising administering to a patient in need thereof an effective amount of a compound of Formula (I): 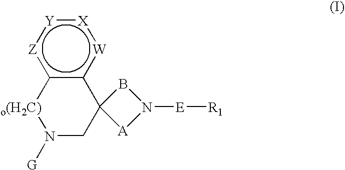
or a pharmaceutically acceptable salt, free base, solvate, hydrate or stereoisomer, thereof, wherein:
R₁ is H, halogen, hydroxy, nitro, cyano, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, substituted or unsubstituted -(3 to 7) membered heterocycle, substituted or unsubstituted -(7 to 10) membered bicycloheterocycle, substituted or unsubstituted -(5 to 10) membered heteroaryl, —NR₂R₂′, —C(═O)—R₇, —S(═O)₂—R₇, —C(═O)O—R₇, or —C(═O)N(R₇)(C₁₋₄ alkyl);
A is a substituted or unsubstituted C₁-C₃ alkylene;
B is a substituted or unsubstituted C₁-C₃ alkylene;
E is a bond, or a substituted or unsubstituted C₁-C₃ alkylene;
G is H, —Ar, —C(═O)—Ar, —C(═O)O—Ar, substituted or unsubstituted —C(═O)O—C₁₋₆ alkyl, —C(═O)N(R₇)(Ar), substituted or unsubstituted —C(═O)N(R₇)(C₁₋₆ alkyl), —S(═O)₂—Ar, substituted or unsubstituted —S(═O)₂—C₁₋₆ alkyl, substituted or unsubstituted C₁₋₆ alkyl, substituted or unsubstituted C₁₋₆ alkyl-Ar, substituted or unsubstituted —C(═O)C₁₋₆ alkyl-Ar, or substituted or unsubstituted —C(═O)C₁₋₆ alkyl;
W is N or —CR₃—;
X is N or —CR₄—;
Y is N or —CR₅—;
Z is N or —CR₆—;
R₂, R₂′, R₃, R₄, R₅, R₆ and R₇ are at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, —C(═O)—O—C₁₋₆ alkyl, —O—C₁₋₆ alkyl, —C₁₋₆ alkyl-O—C₁₋₆ alkyl, —C₁₋₆ alkyl-NH₂, —C₀₋₆ alkyl-C(═O)—NH(C₁₋₆ alkyl), —C₀₋₆ alkyl-C(═O)—N(C₁₋₆ alkyl)(C₁₋₆ alkyl), —C₁₋₆ alkyl-NH—C(═O)—C₁₋₆ alkyl, —C₁₋₆ alkyl-S(═O)—C₁₋₆ alkyl, —C₀₋₆ alkyl-O—S(═O)₂—C₁₋₆ alkyl, —C₁₋₆ alkyl-S(═O)₂—C₁₋₆ alkyl, —C₁₋₆ alkyl-NR′—S(═O)₂—R′, —C₁₋₆ alkyl-SH, —C₁₋₆ alkyl-S—C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —(C(R′)₂)₀₋₆—O—(C(R′)₂)₁₋₅C(R′)₃, —(C(R′)₂)₁₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂—(C(R′)₂)₁₋₅C(R′)₃;
o is 0 or 1;
R′ is at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted aryl, or substituted or unsubstituted C₃₋₈ cycloalkyl; and
Ar is substituted or unsubstituted aryl, substituted or unsubstituted C₃₋₇ cycloalkyl, substituted or unsubstituted C₈₋₁₄ bicycloalkyl, substituted or unsubstituted C₈₋₁₄ tricycloalkyl, substituted or unsubstituted -(3 to 7) membered heterocycle, substituted or unsubstituted -(7 to 10) membered bicycloheterocycle, or substituted or unsubstituted -(5 to 10 membered)heteroaryl.

48. The method of claim 47, wherein E is —(CH₂)ₚ— and p is 0, 1 or 2.

49. The method of claim 48, wherein the disease or disorder is a vascular or cardiovascular disease or disorder.

50. The method of claim 49, wherein the vascular or cardiovascular disease or disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine.

51. The method of claim 47 or 48, wherein the disease or disorder is a neurological disease or disorder.

52. The method of claim 51, wherein the neurological disease or disorder is diabetic peripheral neuropathy, pain, stroke, cerebral ischemia or Parkinson's disease.

53. A method for inhibiting Mas receptor function in a cell, comprising contacting a cell capable of expressing the Mas receptor with an effective amount of a compound of Formula (I): 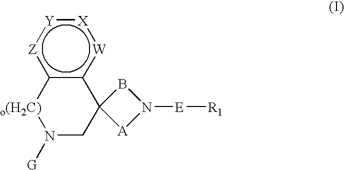
or a pharmaceutically acceptable salt, free base, solvate, hydrate or stereoisomer, thereof, wherein:
R₁ is H, halogen, hydroxy, nitro, cyano, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, substituted or unsubstituted -(3 to 7) membered heterocycle, substituted or unsubstituted -(7 to 10) membered bicycloheterocycle, substituted or unsubstituted -(5 to 10) membered heteroaryl, —NR₂R₂′, —C(═O)—R₇, —S(═O)₂—R₇, —C(═O)O—R₇, or —C(═O)N(R₇)(C₁₋₆ alkyl);
A is a substituted or unsubstituted C₁-C₃ alkylene;
B is a substituted or unsubstituted C₁-C₃ alkylene;
E is a bond, or a substituted or unsubstituted C₁-C₃ alkylene;
G is H, —Ar, —C(═O)—Ar, —C(═O)O—Ar, substituted or unsubstituted —C(═O)O—C₁₋₆ alkyl, —C(═O)N(R₇)(Ar), substituted or unsubstituted —C(═O)N(R₇)(C₁₋₆ alkyl), —S(═O)₂—Ar, substituted or unsubstituted —S(═O)₂—C₁₋₆ alkyl, substituted or unsubstituted C₁₋₆ alkyl, substituted or unsubstituted C₁₋₆ alkyl-Ar, substituted or unsubstituted —C(═O)C₁₋₆ alkyl-Ar, or substituted or unsubstituted —C(═O)C₁₋₆ alkyl;
W is N or —CR₃—;
X is N or —CR₄—;
Y is N or —CR₅—;
Z is N or —CR₆—;
R₂, R₂′, R₃, R₄, R₅, R₆ and R₇ are at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted C₈₋₁₄ bicycloalkyl, substituted or unsubstituted C₈₋₁₄ tricycloalkyl, substituted or unsubstituted aryl, —C(═O)—O—C₁₋₆ alkyl, —O—C₁₋₆ alkyl, —C₁₋₆ alkyl-O—C₁₋₆ alkyl, —C₁₋₆ alkyl-NH₂, —C₀₋₆ alkyl-C(═O)-NH(C₁₋₆ alkyl), —C₀₋₆ alkyl-C(═O)—N(C₁₋₆ alkyl)(C₁₋₆ alkyl), —C₁₋₆ alkyl-NH—C(═O)—C₁₋₆ alkyl, —C₁₋₆ alkyl-S(═O)—C₁₋₆ alkyl, —C₀₋₆ alkyl-O—S(═O)₂—C₁₋₆ alkyl, —C₁₋₆ alkyl-S(═O)₂—C₁₋₆ alkyl, —C₁₋₆ alkyl-NR′—S(═O)₂—R′, —C₁₋₆ alkyl-SH, —C₁₋₆ alkyl-S-C₁₋₆ alkyl, —C₁₋₆ alkyl-NH—C(═S)—NH—C₁₋₆ alkyl, —C₁₋₆ alkyl-NH—C(═O)—NH—C₁₋₆ alkyl, —C₀₋₆ alkyl-N(R′)₂, —C₀₋₆ alkyl-NHOH, —C₀₋₆ alkyl-C(═O)O—C₁₋₆ alkyl, —(C(R′)₂)₀₋₆—O—(C(R′)₂)₁₋₅C(R′)₃, —(C(R′)₂)₁₋₅C(R′)₃, —(C(R′)₂)₀₋₆—S—(C(R′)₂)₁₋₅C(R′)₃, —(C(R′)₂)₀₋₆—S(═O)—(C(R′)₂)₁₋₅C(R′)₃ or —(C(R′)₂)₀₋₆—S(═O)₂—(C(R′)₂)₁₋₅C(R′)₃;
o is 0 or 1;
R′ is at each occurrence independently H, halogen, hydroxy, amino, cyano, nitro, substituted or unsubstituted C₁₋₈ alkyl, substituted or unsubstituted C₂₋₆ alkenyl, substituted or unsubstituted C₂₋₆ alkynyl, substituted or unsubstituted aryl, or substituted or unsubstituted C₃₋₈ cycloalkyl; and
Ar is substituted or unsubstituted aryl, substituted or unsubstituted C₃₋₇ cycloalkyl, substituted or unsubstituted C₈₋₁₄ bicycloalkyl, substituted or unsubstituted C₈₋₁₄ tricycloalkyl, substituted or unsubstituted -(3 to 7) membered heterocycle, substituted or unsubstituted -(7 to 10) membered bicycloheterocycle, or substituted or unsubstituted -(5 to 10 membered)heteroaryl.

54. A pharmaceutical composition comprising a compound of claim 1 or a pharmaceutically acceptable salt of a compound of claim 1 and a pharmaceutically acceptable excipient.

55. (canceled)

56. (canceled)

57. (canceled)

58. (canceled)

59. A method for identifying a cardio-protective compound, comprising:
a) contacting a candidate compound with a Mas receptor,
b) determining whether the receptor functionality is decreased,
wherein a decrease in receptor functionality is indicative of the candidate compound being a cardio-protective compound.

60. The method of claim 59, wherein said Mas receptor is human.

61. The method of claim 59, wherein said cardio-protective compound is an inverse agonist or antagonist of the Mas receptor.

62. The method of claim 59, wherein said cardio-protective compound is an inverse agonist of the Mas receptor.

63. The method of claim 59, wherein said determining comprises using an IP₃ assay.

64. The method of claim 59, further comprising determining the effect of said candidate compound on blood pressure, wherein no significant increase in blood pressure is indicative of the candidate compound being a cardio-protective compound.

65. A cardio-protective compound identified according to the method of claim 59.

66. The cardio-protective compound of claim 65, wherein said compound is an inverse agonist.

67. The cardio-protective compound of claim 66, wherein said inverse agonist does not significantly increase blood pressure.

68. A method for inhibiting Mas receptor function in a cell, comprising contacting a cell capable of expressing Mas with an effective amount of the cardio-protective compound of claim 65.

69. A method for preparing a composition which comprises identifying a cardio-protective compound and then admixing said modulator and carrier, wherein the modulator is identifiable by the method of claim 59.

70. A pharmaceutical composition comprising, consisting essentially of, or consisting of the inverse agonist of claim 66.

71. A method for effecting cardio protection in an individual in need of said cardioprotection, comprising administering to said individual an effective amount of the compound of claim 70.

72. A method for treating or preventing a vascular or cardiovascular disease or disorder in an individual in need of said treating or preventing, comprising administering an effective amount of the compound of claim 70 to said individual.

73. The method of claim 72, wherein said vascular or cardiovascular disease or disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine.

74. The method of claim 72, wherein said vascular or cardiovascular disease or disorder is reperfusion injury, acute myocardial infarction, acute or chronic congestive heart failure, left ventricular hypertrophy or vascular hypertrophy.

75. A method of effecting a needed change in cardiovascular function in an individual in need of said change, comprising administering an effective amount of a compound of claim 70, wherein said needed change in cardiovascular function is an increase in ventricular contractile function.

76. (canceled)

77. (canceled)

78. A method for selectively inhibiting Mas receptor activity in a human host, comprising administering a compound that selectively inhibits activity of the Mas receptor gene product to a human host in need of such treatment.

79. A method for selectively inhibiting Mas receptor activity in a human host, comprising administering a compound of claim 1 that selectively inhibits activity of the Mas receptor gene product to a human host in need of such treatment.

80. A method for effecting cardio protection in an individual in need of said cardio protection, comprising administering to said individual an effective amount of the pharmaceutical composition comprising, an inverse agonist identified according to the method of a) contacting a candidate compound with a Mas receptor, and b) determining whether the receptor functionality is decreased, wherein a decrease in receptor functionality is indicative of the candidate compound being a cardio-protective compound.

81. A method for treating or preventing a vascular or cardiovascular disease or disorder in an individual in need of said treating or preventing, comprising administering to said individual an effective amount of the pharmaceutical composition comprising, an inverse agonist identified according to the method of a) contacting a candidate compound with a Mas receptor, and b) determining whether the receptor functionality is decreased, wherein a decrease in receptor functionality is indicative of the candidate compound being a vascular- or cardio-protective compound.


SEQUENCE LISTING (2 sequences)

SEQ ID NO: 1 - DNA, 978 bp, Homo sapien
atggatgggtcaaacgtgacatcatttgttgttgaggaacccacgaacatctcaactggcaggaacgcctcagtcgggaatgcacatcggcaaatccccatcgtgcactgggtcattatgagcatctccccagtggggtttgttgagaatgggattctcctctggttcctgtgcttccggatgagaagaaatcccttcactgtctacatcacccacctgtctatcgcagacatctcactgctcttctgtattttcatcttgtctatcgactatgctttagattatgagctttcttctggccattactacacaattgtcacattatcagtgacttttctgtttggctacaacacgggcctctatctgctgacggccattagtgtggagaggtgcctgtcagtcctttaccccatctggtaccgatgccatcgccccaagtaccagtcggcattggtctgtgcccttctgtgggctctttcttgcttggtgaccaccatggagtatgtcatgtgcatcgacagagaagaagagagtcactctcggaatgactgccgagcagtcatcatctttatagccatcctgagcttcctggtcttcacgcccctcatgctggtgtccagcaccatcttggtcgtgaagatccggaagaacacgtgggcttcccattcctccaagctttacatagtcatcatggtcaccatcattatattcctcatcttcgctatgcccatgagactcctttacctgctgtactatgagtattggtcgacctttgggaacctacaccacatttccctgctcttctccacaatcaacagtagcgccaaccctttcatttacttctttgtgggaagcagtaagaagaagagattcaaggagtccttaaaagttgttctgaccagggctttcaaagatgaaatgcaacctcggcgccagaaagacaattgtaatacggtcacagttgagactgtcgtctaa

SEQ ID NO: 2 - protein, 325 aa, Homo sapien
MDGSNVTSFVVEEPTNISTGRNASVGNAHRQIPIVHWVIMSISPVGFVENGILLWFLCFRMRRNPFTVYITHLSIADISLLFCIFILSIDYALDYELSSGHYYTIVTLSVTFLFGYNTGLYLLTAISVERCLSVLYPIWYRCHRPKYQSALVCALLWALSCLVTTMEYVMCIDREEESHSRNDCRAVIIFIAILSFLVFTPLMLVSSTILVVKIRKNTWASHSSKLYIVIMVTIIIFLIFAMPMRLLYLLYYEYWSTFGNLHHISLLFSTINSSANPFIYFFVGSSKKKRFKESLKVVLTRAFKDEMQPRRQKDNCNTVTVETVV